(19) United States
(12) Patent Application Publication
(10) Pub. No.: US 2011/0009628 A1
(43) Pub. Date: Jan. 13, 2011

(54) Compounds and Compositions for Modulating Lipid Levels and Methods of Preparing Same

(76) Inventors: Haiyan Liu, Mountain View, CA (US); Gaoping Li, Mountain View, CA (US); Junbo Wang, Shanghai (CN); Jingwen Liu, Mountain View, CA (US)

Correspondence Address: FOLEY & LARDNER LLP, 150 EAST GILMAN STREET, P.O. BOX 1497, MADISON, WI, 53701-1497, US

(21) Appl. No.: 12/832,852

(22) Filed: Jul. 8, 2010

Related U.S. Application Data
(60) Provisional application No. 61/223,782, filed on Jul. 8, 2009.

(51) Int. Cl. C07D 413/14; C07D 221/18; C07D 491/147

(52) U.S. Cl. 544/125; 546/73; 546/48

(57) ABSTRACT

The present technology relates to compounds of Formulas I-VI and methods of making and using such compounds. Methods of use include prevention and treatment of hyperlipidemia, hypercholesterolemia, hypertriglyceridemia, hepatic steatosis, and metabolic syndrome. Compounds disclosed herein also increase HDL-C, lower total cholesterol, LDL-cholesterol, and triglycerides and increase hepatic LDL receptor expression, inhibit PCSK9 expression, and activate AMP-activated protein kinase.

### CROSS REFERENCES TO RELATED APPLICATIONS

The present application claims priority to U.S. Provisional Application No. 61/223,782, filed Jul. 8, 2009, and incorporated by reference in its entirety herein and for all purposes.

### FIELD OF THE TECHNOLOGY

The present technology is related to compounds and compositions for modulating lipid levels, including treating hyperlipidemia, such as hypertriglyceridemia and hypercholesterolemia, as well as hepatic steatosis and metabolic syndrome. In addition the present technology provides methods of preparing such compounds and compositions.

### SUMMARY

In accordance with one aspect, the present technology provides compounds that may be used as lipid lowering agents such as compounds of Formula I:

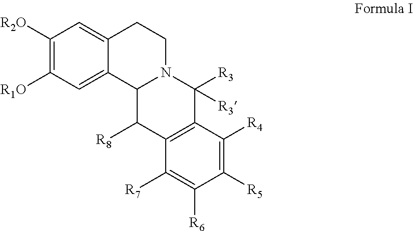

or stereoisomers thereof, tautomers thereof, solvates thereof, and pharmaceutically acceptable salts thereof; wherein

R₁ and R₂ are independently —H, —(CH₂)₀₋₆COOR′, —C(O)R″, or a substituted or unsubstituted alkyl, cycloalkyl, cycloalkylalkyl, alkenyl, aryl, aralkyl, heteroaryl, heteroarylalkyl, heterocyclyl, or heterocyclylalkyl group; or R₁ and R₂ together are a methylene group;

R₃ and R₈ are independently —H, —OH, —Cl, —Br, —F, —I, —CN, —NH₂, —C(O)NH₂, —COOH, or a substituted or unsubstituted alkyl, alkoxy, alkenyl, or aralkyl group;

R₃′ is H, or R₃ and R₃′ together are an oxo group;

R₄ is —H, halogen, —OR′, —OSO₂R″, —OC(O)R″, —OC(O)OR″, —OC(O)NR′R″, —O-alkylene-NR′R′, —O-alkylene-OSO₂R″, —O-alkylene-S(O)₀₋₂R″, —O-alkylene-NR′SO₂R″, —O-alkylene-N(R′)C(O)R′, or a substituted or unsubstituted alkyl group;

R₅ and R₆ are independently —H, halogen, —OH, or a substituted or unsubstituted alkoxy group; or R₄ and R₅ together are a methylenedioxy group, or R₅ and R₆ together are a methylenedioxy group;

R₇ is H, halogen, OH, or a substituted or unsubstituted alkyl or alkoxy group;

R₉ is H or a substituted or unsubstituted alkyl group;

each R′ is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;

each R″ is independently a substituted or unsubstituted alkyl, alkenyl, cycloalkyl, cycloalkylalkyl, aryl, aralkyl, heteroaryl, heteroarylalkyl, heterocyclyl, or heterocyclylalkyl group.

In other embodiments, there are provided a second group of compounds of Formula I (see structure above), stereoisomers thereof, tautomers thereof, solvates thereof, and pharmaceutically acceptable salts thereof; wherein

R₁ and R₂ are independently —H, —(CH₂)₀₋₆COOR′, —C(O)R″, or a substituted or unsubstituted alkyl, cycloalkyl, cycloalkylalkyl, alkenyl, aryl, aralkyl, heteroaryl, heteroarylalkyl, heterocyclyl, or heterocyclylalkyl group; or R₁ and R₂ together are a methylene group;

R₃ and R₈ are independently —H, —OH, —Cl, —Br, —F, —I, —CN, —NH₂, —C(O)NH₂, —COOH, or a substituted or unsubstituted alkyl, alkenyl, alkoxy or aralkyl group;

R₃′ is —H, or R₃ and R₃′ together are an oxo group;

R₄ is —H, halogen, —OR′, —OSO₂R″, —OC(O)R″, —OC(O)OR″, —OC(O)NR′R″, —O-alkylene-NR′R′, —O-alkylene-OSO₂R″, —O-alkylene-S(O)₀₋₂R″, —O-alkylene-NR′SO₂R″, —O-alkylene-N(R′)C(O)R′, or a substituted or unsubstituted alkyl group;

R₅ and R₆ are independently —H, halogen, —OH, or a substituted or unsubstituted alkoxy group; or R₄ and R₅ together are a methylenedioxy group, or R₅ and R₆ together are a methylenedioxy group;

R₇ is —H, halogen, —OH, or a substituted or unsubstituted alkyl or alkoxy group;

each R′ is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;

each R″ is independently a substituted or unsubstituted alkyl, alkenyl, cycloalkyl, cycloalkylalkyl, aryl, aralkyl, heteroaryl, heteroarylalkyl, heterocyclyl, or heterocyclylalkyl group;

with the proviso that when R₄ is —H, —OH or a C₁₋₄ alkoxy group, then R₅ is not —H, —OH or a C₁₋₄ alkoxy group; and when R₁ and R₂ are both —CH₃ or when R₁ and R₂ together are a methylene group, then R₅ is not OH or a C₁₋₂ alkoxy group, and R₄ and R₅ together are not a methylenedioxy group; and when R₄ is OC(O)R″, then R₅ is not OC(O)R″ or methoxy.

In another aspect, a lipid lowering agent of the present technology is part of a pharmaceutical composition containing one or more excipients, carriers, or fillers. In one embodiment, the pharmaceutical composition is packaged in unit dosage form. The unit dosage form is effective in lowering lipid levels (e.g., at least one of total cholesterol, LDL-cholesterol, triglyceride, and unesterified long chain fatty acids) in the bloodstream and/or in the liver when administered to a subject in need thereof.

Still another aspect of the present technology is a pharmaceutical pack or kit containing a lipid lowering agent according to the present technology and a second agent. The second agent can be a cholesterol uptake inhibitor, a cholesterol synthesis inhibitor, a cholesterol absorption inhibitor, a bile acid sequestrant, a vitamin, an antihypertensive agent, or a platelet aggregation inhibitor. The second agent alternatively can be an HMG-CoA reductase inhibitor, an HMG-CoA synthase inhibitor, a squalene epoxidase inhibitor, an acyl-CoA cholesterol acyltransferase (ACAT) inhibitor, a microsomal triglyceride transfer protein (MTP) inhibitor, a peroxisome proliferator-activated receptor (PPAR) agonist, or an AMP-activated protein kinase (AMPK) activator. The second agent can also be an agent that increases low density lipoprotein receptor (LDLR) expression. The second agent can be a berberine compound, such as tetrahydroberberine.

In another aspect, the present technology provides methods of synthesizing compounds of formula EE,

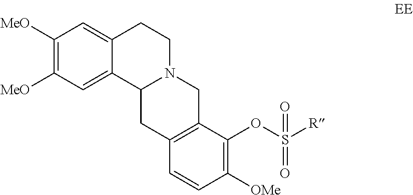

salts thereof and enantiomers thereof starting from Berberine; wherein

R″ is a substituted or unsubstituted alkyl, cycloalkyl, cycloalkylalkyl, alkenyl, aryl, aralkyl, heterocyclyl, heterocyclylalkyl, heteroaryl, or heteroarylalkyl group.

### DESCRIPTION OF THE DRAWINGS

FIG. 1 shows the potent and dose-dependent effects of (+)-CLMD, 14R-(+)-CRPM, 14R,13S-(+)-CRDL, and 14R-(+)-THP on LDLR mRNA expression in HepG2 cells by a semi-quantitative RT-PCR analysis.

FIG. 2A shows the determination of the specific stereochemical requirements of +/−THP in the upregulation of LDLR mRNA expression. FIG. 2B shows that enantiomers of compounds disclosed herein with dextrorotary optical rotation elevate LDLR mRNA levels.

FIG. 3 shows that compounds disclosed herein strongly inhibit the mRNA expression of PCSK9.

FIG. 4A shows a LDLR mRNA level vs. concentration curve for compound 91 and simvastatin and FIG. 4B shows a PCSK9 mRNA levels vs. concentration curve for curve compound 91.

FIG. 5 is a Western blot demonstrating enhanced LDLR expression and reduced PCSK9 expression in the presence of compounds of the present technology. Actin is a positive control showing equal protein loading levels.

FIGS. 6A and 6B show that Compounds 162 and 163 upregulate LDLR mRNA while inhibiting PCSK9 mRNA expression. In FIG. 6A HepG2 cells were exposed to Compounds 162 and 163 at the indicated concentrations (x-axis) for 24 h and plotted against normalized LDLR mRNA levels (fold of control in absence of compound). In FIG. 6B, HepG2 cells were treated with 20 μM of each compound for the indicted times (x-axis) and plotted against normalized LDLR mRNA levels (fold of control at 24 h).

FIG. 7 is a Western blot analysis of LDLR and PCSK9 expression in HepG2 cells. HepG2 cells seeded in 12-well culture plates were cultured in 0.5% FBS MEM overnight. Compounds 162 and 163 at 20 μM were added to cells for the indicated times. Untreated and treated cells were harvested at the same indicated time points. Total cell lysates were isolated and 50 μg per sample was analyzed for LDLR and PCSK9 protein abundance by western blotting. The membrane was reprobed with anti-actin antibody to examine the equal loading of cell lysates.

FIG. 8 shows that Compound 162 administration reduces plasma TC and TG while increasing HDL-C in hamsters. Male hamsters were fed a cholesterol-enriched diet for 14 days before the daily oral feeding of Compound 162 at 30, 60, and 100 mg/kg, or an equal volume of vehicle for 2-weeks. Serum samples were collected after 2 weeks of treatment and analyzed for TC, TG, and HDL-C. The data shown are mean±SEM of 10 hamsters per group (* p<0.05, ** p<0.01 compared to untreated control group).

FIGS. 9A and 9B show the results of HPLC analysis of pooled serum samples of plasma lipoprotein associated cholesterol (9A) and triglyceride (9B) profiles of vehicle control and Compound 162 treated animals. (CM, chylomicron).

### DETAILED DESCRIPTION

In various aspects, the present technology provides compounds, methods of making the compounds and methods for reducing plasma and/or hepatic lipid levels, and methods for treating hyperlipidemia, hypercholesterolemia, hypertriglyceridemia, hepatic steatosis and metabolic syndrome using the compounds. The compounds provided herein can be formulated into pharmaceutical compositions and medicaments that are useful in the disclosed methods. Also provided are the use of the compounds in preparing pharmaceutical formulations and medicaments, the use of the compounds in reducing plasma and/or hepatic lipid levels, and the use of the compounds in treating hyperlipidemia, hypercholesterolemia, hypertriglyceridemia, hepatic steatosis and metabolic syndrome.

The following terms are used throughout as defined below.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, butadienyl, pentadienyl, and hexadienyl.

Cycloalkenylalkyl groups are alkyl groups as defined above in which a hydrogen or carbon bond of the alkyl group is replaced with a bond to a cycloalkenyl group as defined above. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR³⁰ groups. R³⁰ is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., —C(O)NR³¹R³², and —NR³¹C(O)R³² groups, respectively. R³¹ and R³² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR³¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR³³C(O)OR³⁴ and —OC(O)NR³³R³⁴ groups, respectively. R³³ and R³⁴ are independently a substituted or unsubstituted alkyl, alkenyl, alkynyl, cycloalkyl, aryl, aralkyl, heterocyclylalkyl, or heterocyclyl group as defined herein. R³³ may also be H.

The term “amine” (or “amino”) as used herein refers to —NR³⁵R³⁶ groups, wherein R³⁵ and R³⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR³⁸R³⁹ and —NR³⁸SO₂R³⁹ groups, respectively. R³⁸ and R³⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while sulfides include —SR⁴⁰ groups, sulfoxides include —S(O)R⁴¹ groups, sulfones include —SO₂R⁴² groups, and sulfonyls include —SO₂OR⁴³. R⁴⁰, R⁴¹, R⁴², and R⁴³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁴⁴—C(O)—NR⁴⁵R⁴⁶ groups. R⁴⁴, R⁴⁵, and R⁴⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁴⁷)NR⁴⁸R⁴⁹ and —NR⁴⁷C(NR⁴⁸)R⁴⁹, wherein R⁴⁷, R⁴⁸, and R⁴⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁵⁰C(NR⁵¹)NR⁵²R⁵³, wherein R⁵⁰, R⁵¹, R⁵² and R⁵³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁵⁴)═C(R⁵⁵)NR⁵⁶R⁵⁷ and —NR⁵⁴C(R⁵⁵)═C(R⁵⁶)R⁵⁷, wherein R⁵⁴, R⁵⁵, R⁵⁶ and R⁵⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxy’ as used herein can refer to —OH or its ionized form,

The term “imide” refers to —C(O)NR⁵⁸C(O)R⁵⁹, wherein R⁵⁸ and R⁵⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR⁶⁰(NR⁶¹) and —N(CR⁶⁰R⁶¹) groups, wherein R⁶⁰ and R⁶¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R⁶⁰ and R⁶¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g. Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, imidazoles may exhibit the following isomeric forms, which are referred to as tautomers of each other:

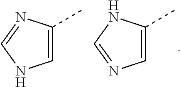

As readily understood by one skilled in the art, a wide variety of functional groups and other structures may exhibit tautomerism, and all tautomers of compounds as described herein are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Lipids include synthetic or naturally-occurring fat-soluble compounds, and include both neutral and amphipathic molecules. Amphipathic lipids typically comprise a hydrophilic component and a hydrophobic component. Exemplary lipids include fatty acids, triglycerides, neutral fats, phosphatides, glycolipids, aliphatic alcohols, waxes, terpenes, steroids such as cholesterol, and surfactants.

A “lipid lowering agent” as used herein refers to compounds that have one or more of the following effects when administered to a subject: increasing the hepatic expression of LDLR; increasing the half-life of LDLR mRNA in hepatocytes; increasing hepatic uptake of plasma LDL, cholesterol, or triglycerides; enhancing hepatic fatty acid oxidation, reducing hepatic triglyceride synthesis and secretion, and reducing the plasma and/or hepatic levels of total cholesterol, LDL-cholesterol, VLDL-cholesterol, or triglycerides. Lipid lowering agents as disclosed herein include compounds of Formulas I and EE.

A “compound” or “derivative” as used herein refers to a chemical compound, either in partially purified or substantially pure form, which either has been obtained from a plant extract, such as a Corydalis extract, by one or more purification steps or which has been produced by chemical synthesis from any desired starting materials. A compound or derivative according to the present technology can be used either as a racemic mixture or as a pure stereoisomer. In some embodiments, the compound or derivative is a pure stereoisomer which has activity as a lipid lowering agent.

A “partially purified” compound or derivative as used herein refers to a compound or derivative thereof which is present in a chemical mixture that has been subjected to at least one separation or purification step resulting in the removal of at least one other chemical substance originally present in the initial mixture containing the compound or derivative. A “substantially pure” compound or derivative is one which has been separated or purified to render the compound or derivative as the major chemical component of the substantially pure compound or derivative, i.e., comprising at least 50%, or in some embodiments at least 70%, at least 90%, or at least 95% or 99% on a molar basis.

In one aspect, the present technology provides methods of reducing plasma and/or hepatic lipid levels in a subject in need thereof, which comprises administering to said subject a lipid-lowering effective amount of a compound or composition as described herein. The lipid level to be reduced can be one or more of total cholesterol, LDL-cholesterol (LDL-c), triglycerides (TG), and unesterified long chain fatty acids.

In another aspect, the present technology provides methods for raising HDL-cholesterol levels in a subject in need thereof including administering to the subject an HDL-cholesterol raising effective amount of a compound or composition described herein. The raised HDL-cholesterol levels may be plasma, hepatic and/or systemic HDL-cholesterol levels.

The compounds and compositions described herein may be used in the treatment or prophylaxis of diseases that include, for example, hyperlipidemia, hypercholesterolemia, hypertriglyceridemia, fatty liver (hepatic steatosis), and metabolic syndrome. Methods of treatment include administering to a subject in need thereof a therapeutically effective amount of a compound or composition described herein. The compounds of the present technology can also be used in the treatment or prophylaxis of a disease state or malady characterized by or associated with elevated plasma or hepatic cholesterol or triglycerides. Generally, prophylactic or prophylaxis relates to a reduction in the likelihood of the patient developing a disorder such as hyperlipidemia, hypercholesterolemia, hypertriglyceridemia, fatty liver, or metabolic syndrome or proceeding to a diagnosis state for the disorder. For example, the compounds of the present technology can be used prophylactically as a measure designed to preserve health and prevent the spread or maturation of disease in a patient. It is also appreciated that the various modes of treatment or prevention of a disease such as hyperlipidemia, hypercholesterolemia, hypertriglyceridemia, fatty liver, or metabolic syndrome can mean “substantial” treatment or prevention, which includes total but also less than total treatment or prevention, and in which some biologically or medically relevant result is achieved. Furthermore, treatment or treating as well as alleviating can refer to therapeutic treatment and prophylactic or preventative measures in which the object is to prevent, slow down (lessen) a disease state, condition or malady. For example, a subject can be successfully treated for hypercholesterolemia if, after receiving through administration an effective or therapeutic amount of one or more compounds described herein, the subject shows observable and/or measurable reduction in or absence of one or more signs and symptoms of the particular disease such as, but not limited to, reduced plasma total cholesterol, reduced plasma LDL-cholesterol, increased hepatic expression of LDL receptor (LDLR), reduced plasma triglycerides, reduced morbidity and mortality, or improvement in quality of life issues. The present technology also provides for methods of administering one or more compounds of the described herein to a patient in an effective amount for the treatment or prophylaxis of a disease such as, for example, hyperlipidemia, hypercholesterolemia, hypertriglyceridemia, fatty liver, or metabolic syndrome.

While not wishing to be bound by theory, it is believed that the compounds, disclosed herein reduce lipid levels by increasing the hepatic expression of LDLR by increasing the stability of LDLR mRNA, by increasing LDLR gene transcription, by inhibiting the degradation of LDLR protein mediated through the proprotein convertase subtilisin/kexin type 9 (PCSK9), or all of the above potential cellular mechanisms. Increasing LDLR levels in the liver increases the uptake and processing of plasma LDL-c, resulting in reduced plasma levels of cholesterol, LDL-c, and triglycerides. In addition, the compounds may increase phosphorylation of acetyl CoA carboxylase (ACC) via the activation of AMP-activated protein kinase (AMPK). Increased phosphorylation of ACC enhances fatty acid oxidation in the liver, leading to reduced hepatic TG accumulation and secretion of TG in the form of VLDL, which also contributes to the decreased plasma levels of TG, LDL-c, total cholesterol, and unesterified long chain fatty acids, resulting in the prevention or treatment of diseases related to hyperlipidemia.

Hence, in another aspect, the present technology provides methods of increasing LDLR expression, comprising administering to a subject in need thereof a therapeutically effective amount of a compound or composition as described herein, whereby LDLR expression in said subject is increased. In another aspect of the present technology, there are provided methods of decreasing plasma LDL-cholesterol and/or plasma triglycerides, comprising administering to a subject in need thereof a therapeutically effective amount of a compound or composition as described herein, whereby plasma LDL-cholesterol in said subject is decreased.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, the treatment or prophylaxis of hyperlipidemia, hypercholesterolemia, hypertriglyceridemia, fatty liver, or metabolic syndrome. Another example of an effective amount includes amounts or dosages that are capable of preventing elevated plasma or hepatic cholesterol or triglycerides.

As used herein, a “subject” or “patient” is a mammal, such as a cat, dog, rodent or primate. Typically the subject is a human, and, preferably, a human suspected of having a disease associated with elevated plasma or hepatic cholesterol or triglycerides such as hyperlipidemia, hypercholesterolemia, hypertriglyceridemia, fatty liver, or metabolic syndrome. Subjects may further include mammals with elevated LDL levels, elevated VLDL levels, or diseases aggravated or triggered by hyperlipidemia such as cardiovascular diseases, including, atherosclerosis, coronary artery disease, angina pectoris, carotid artery disease, strokes, cerebral arteriosclerosis, myocardial infarction, cerebral infarction, restenosis following balloon angioplasty, intermittent claudication, high blood pressure, dyslipidemia post-prandial lipidemia and xanthoma. The term “subject” and “patient” can be used interchangeably.

In another embodiment, there are provided a first group of compounds of Formula I as well as stereoisomers thereof, tautomers thereof, solvates thereof, and pharmaceutically acceptable salt thereof.

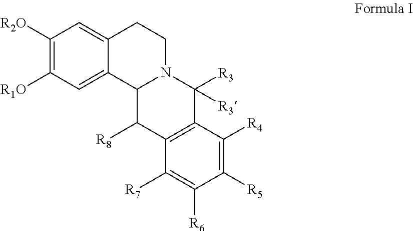

In compounds of Formulas I,

R₁ and R₂ are independently —H, —(CH₂)₀₋₆COOR′, —C(O)R″, or a substituted or unsubstituted alkyl, cycloalkyl, cycloalkylalkyl, alkenyl, aryl, aralkyl, heteroaryl, heteroarylalkyl, heterocyclyl, or heterocyclylalkyl group; or R₁ and R₂ together are a methylene group;

R₃ and R₈ are independently —H, —OH, —Cl, —Br, —F, —I, —CN, —NH₂, —C(O)NH₂, —COOH, or a substituted or unsubstituted alkyl, alkoxy, alkenyl, or aralkyl group;

R₃′ is —H, or R₃ and R₃′ together are an oxo group;

R₄ is —H, halogen, —OR′, —OSO₂R″, —OC(O)R″, —OC(O)OR″, —OC(O)NR′R″, —O-alkylene-NR′R′, —O-alkylene-OSO₂R″, —O-alkylene-S(O)₀₋₂R″, —O-alkylene-NR′SO₂R″, —O-alkylene-N(R′)C(O)R′, or a substituted or unsubstituted alkyl group;

R₅ and R₆ are independently —H, halogen, —OH, or a substituted or unsubstituted alkoxy group; or R₄ and R₅ together are a methylenedioxy group, or R₅ and R₆ together are a methylenedioxy group;

R₇ is —H, halogen, —OH, or a substituted or unsubstituted alkyl or alkoxy group;

R₉ is —H or a substituted or unsubstituted alkyl group;

each R′ is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;

each R″ is independently a substituted or unsubstituted alkyl, alkenyl, cycloalkyl, cycloalkylalkyl, aryl, aralkyl, heteroaryl, heteroarylalkyl, heterocyclyl, or heterocyclylalkyl group.

In some embodiments of the first group of compounds of Formula I,

R₁ and R₂ are independently —H, —(CH₂)₀₋₆COOR′, —C(O)R″, or a substituted or unsubstituted alkyl, cycloalkyl, cycloalkylalkyl, alkenyl, aryl, aralkyl, heteroaryl, heteroarylalkyl, heterocyclyl, or heterocyclylalkyl group; or R₁ and R₂ together are a methylene group;

R₃ and R₈ are independently —H, —OH, —Cl, —Br, —F, —I, —CN, —NH₂, —C(O)NH₂, —COOH, or a substituted or unsubstituted alkyl, alkenyl, alkoxy, or aralkyl group;

R₃′ is —H, or R₃ and R₃′ together are an oxo group;

R₄ is —H, —OR′, —OSO₂R″, —OC(O)R″, —OC(O)OR″, —OC(O)NR′R″, —O-alkylene-NR′R′, —O-alkylene-OSO₂R″, —O-alkylene-S(O)₀₋₂R″, —O-alkylene-NR′SO₂R″, —O-alkylene-N(R)C(O)R′, or a substituted or unsubstituted alkyl group;

R₅ and R₆ are independently —H, halogen, —OH, or a substituted or unsubstituted alkoxy group; or R₄ and R₅ together are a methylenedioxy group, or R₅ and R₆ together are a methylenedioxy group;

R⁷ is —H, —Br, —Cl, or —F;

each R′ is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;

each R″ is independently a substituted or unsubstituted alkyl, alkenyl, cycloalkyl, cycloalkylalkyl, aryl, aralkyl, heteroaryl, heteroarylalkyl, heterocyclyl, or heterocyclylalkyl group.

In other embodiments of the first group of compounds of Formula I,

R₁ and R₂ are independently —H, —(CH₂)₀₋₂COOR′, —C(O)(CH₂)₀₋₂R″, or a unsubstituted C₁₋₆ alkyl group; or R₁ and R₂ together are a methylene group;

R₃ and R₃′ are each —H, or R₃ and R₃′ together are an oxo group;

R₄ is —H, —OH, or a substituted or unsubstituted C₁₋₆ alkoxy, C₇₋₁₄ aralkoxy, —OC(O)—(C₁₋₆ alkyl), —OC(O)-(aryl), —OC(O)O-(aryl), —OC(O)—NH-(aryl), —I—(C₂₋₆ alkylene)-NH—(C₂₋₆ alkyl), —O—(C₂₋₆ alkylene)-NH-(tetrahydropyran), —O—(C₂₋₆ alkylene)-NH-(thiomorpholine dioxide), —O—(C₂₋₆ alkylene)-NH-(piperidinyl), —O—(C₂₋₆ alkylene)-NH-(piperazinyl), —O—(C₂₋₆ alkylene)-NH-(morpholinyl), —O—(C₂₋₆ alkylene)-NH-(aralkyl), —O—(C₂₋₆ alkylene)-NH-(cyclopropyl), —OSO₂—(C₃₋₆ cycloalkyl), —OSO₂-(aryl), —O—(C₂₋₆ alkylene)-OSO₂-(aryl), —OSO₂-(aralkyl), —O—(C₂₋₆ alkylene)-OSO₂-(heteroaryl), —OSO₂—(C₁₋₆ alkyl), —OSO₂-(pyridyl), —OSO₂-(thiazolyl), —O—(C₂₋₆ alkylene)-NHSO₂-(aryl), —O—(C₂₋₆ alkylene)-NHSO₂-(heteroaryl), —O—(C₂₋₆ alkylene)-NHC(O)-(aryl), —O—(C₂₋₆ alkylene)-NHC(O)-(heteroaryl), —O—(C₀₋₄ alkyl)pyridyl, —O—(C₀₋₄ alkyl)pyrimidinyl, —O—(C₀₋₄ alkyl)morpholinyl, —O—(C₀₋₄ alkyl)thiomorpholinyl, —O—(C₀₋₄ alkyl)imidazolyl, —O—(C₀₋₄ alkyl)thienyl, —O—(C₀₋₄ alkyl)tetrahydropyranyl, —O—(C₀₋₄ alkyl)tetrahydrofuranyl, —O—(C₀₋₄ alkyl)pyrrolidinyl, —O—(C₀₋₄ alkyl)piperidinyl, or —O—(C₀₋₄ alkyl)piperazinyl group;

R₅ and R₆ are independently —H, —OH, or an unsubstituted C₁₋₆ alkoxy group; or R₄ and R₅ together are a methylenedioxy group, or R₅ and R₆ together are a methylenedioxy group; and

R₈ is —H, —OH, —COOH, or an unsubstituted alkyl or —(CH₂)₁₋₆-phenyl group.

In another embodiment, the present technology provides a second group of compounds of Formula I,

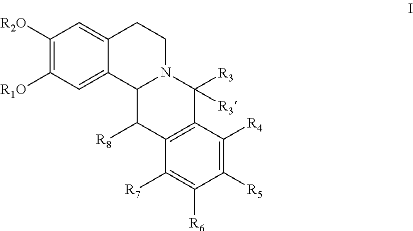

stereoisomers thereof, tautomers thereof, solvates thereof, and pharmaceutically acceptable salts thereof; wherein

R₁ and R₂ are independently —H, —(CH₂)₀₋₆COOR′, —C(O)R″, or a substituted or unsubstituted alkyl, cycloalkyl, cycloalkylalkyl, alkenyl, aryl, aralkyl, heteroaryl, heteroarylalkyl, heterocyclyl, or heterocyclylalkyl group; or R₁ and R₂ together are a methylene group;

R₃ and R₈ are independently —H, —OH, —Cl, —Br, —F, —I, —CN, —NH₂, —C(O)NH₂, —COOH, or a substituted or unsubstituted alkyl, alkenyl, alkoxy or aralkyl group;

R₃′ is —H, or R₃ and R₃′ together are an oxo group;

R₄ is —H, halogen, —OR′, —OSO₂R″, —OC(O)R″, —OC(O)OR″, —OC(O)NR′R″, —O-alkylene-NR′R′, —O-alkylene-OSO₂R″, —O-alkylene-S(O)₀₋₂R″, —O-alkylene-NR′SO₂R″, —O-alkylene-N(R′)C(O)R′, or a substituted or unsubstituted alkyl group;

R₅ and R₆ are independently —H, halogen, —OH, or a substituted or unsubstituted alkoxy group; or R₄ and R₅ together are a methylenedioxy group, or R₅ and R₆ together are a methylenedioxy group;

R₇ is —H, halogen, —OH, or a substituted or unsubstituted alkyl or alkoxy group;

each R′ is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;

each R″ is independently a substituted or unsubstituted alkyl, alkenyl, cycloalkyl, cycloalkylalkyl, aryl, aralkyl, heteroaryl, heteroarylalkyl, heterocyclyl, or heterocyclylalkyl group;

with the proviso that when R₄ is —H, —OH or a C₁₋₄ alkoxy group, then R₅ is not —H, —OH or a C₁₋₄ alkoxy group; and when R₁ and R₂ are both —CH₃ or when R₁ and R₂ together are a methylene group, then R₅ is not OH or a C₁₋₂ alkoxy group, and R₄ and R₅ together are not a methylenedioxy group; and when R₄ is OC(O)R″, then R₅ is not OC(O)R″ or methoxy.

In some embodiments of the first and second groups of compounds of Formula I (collectively, “compounds of Formula I”), R₁ and R₂ are independently —H, —(CH₂)₀₋₂COOR′, —C(O)(CH₂)₀₋₂R″, or a unsubstituted C₁₋₆ alkyl group; or R₁ and R₂ together are a methylene group. In other embodiments, R₁ and R₂ together are a methylene group.

In some embodiments of the compounds of Formula I, R₃ and R₃′ are each —H, or R₃ and R₃′ together are an oxo group.

In some embodiments of compounds of Formula I, R₄ is —H, —OR′, —OSO₂R″, —OC(O)OR″, —OC(O)NR′R″, —O-alkylene-OSO₂R″, or —O-alkylene-NR′R′. In other embodiments, R₄ is —H, —OH, or a substituted or unsubstituted C₁₋₆ alkoxy, C₇₋₁₄ aralkoxy, —OC(O)—(C₁₋₆ alkyl), —OC(O)-(aryl), —OC(O)O-(aryl), —OC(O)—NH-(aryl), —O—(C₂₋₆ alkylene)-NH—(C₂₋₆ alkyl), —O—(C₂₋₆ alkylene)-NH-(tetrahydropyran), —O—(C₂₋₆ alkylene)-NH-(thiomorpholine dioxide), —O—(C₂₋₆ alkylene)-NH-(piperidinyl), —O—(C₂₋₆ alkylene)-NH-(piperazinyl), —O—(C₂₋₆ alkylene)-NH-(morpholinyl), —O—(C₂₋₆ alkylene)-NH-(aralkyl), —O—(C₂₋₆ alkylene)-NH-(cyclopropyl), —OSO₂—(C₃₋₆ cycloalkyl), —OSO₂-(aryl), —O—(C₂₋₆ alkylene)-OSO₂-(aryl), —OSO₂-(aralkyl), —O—(C₂₋₆ alkylene)-OSO₂-(heteroaryl), —OSO₂—(C₁₋₆ alkyl), —OSO₂-(pyridyl), —OSO₂-(thiazolyl), —O—(C₂₋₆ alkylene)-NHSO₂-(aryl), —O—(C₂₋₆ alkylene)-NHSO₂-(heteroaryl), —O—(C₂₋₆ alkylene)-NHC(O)-(aryl), —O—(C₂₋₆ alkylene)-NHC(O)-(heteroaryl), —O—(C₀₋₄ alkyl)pyridyl, —O—(C₀₋₄ alkyl)pyrimidinyl, —O—(C₀₋₄ alkyl)morpholinyl, —O—(C₀₋₄ alkyl)thiomorpholinyl, —O—(C₀₋₄ alkyl)imidazolyl, —O—(C₀₋₄ alkyl)thienyl, —O—(C₀₋₄ alkyl)tetrahydropyranyl, —O—(C₀₋₄ alkyl)tetrahydrofuranyl, —O—(C₀₋₄ alkyl)pyrrolidinyl, —O—(C₀₋₄ alkyl)piperidinyl, or —O—(C₀₋₄ alkyl)piperazinyl group.

In some embodiments, R⁴ is —OSO₂R″.

In other embodiments of compounds of Formula I, R₅ is OH or unsubstituted alkoxy and R₆ is H.

In some embodiments of compounds of Formula I, R₈ is —H, —OH, —COOH, or an unsubstituted alkyl or —(CH₂)₁₋₆-phenyl group.

In certain embodiments of compounds of Formula I,

R₁ and R₂ are independently —H, —(CH₂)₀₋₂COOR′, —C(O)(CH₂)₀₋₂R″, or a unsubstituted C₁₋₆ alkyl group; or R₁ and R₂ together are a methylene group;

R₃ and R₃′ are each —H, or R₃ and R₃′ together are an oxo group;

R₄ is —H, —OH, or a substituted or unsubstituted C₁₋₆ alkoxy, C₇₋₁₄ aralkoxy, —OC(O)—(C₁₋₆ alkyl), —OC(O)-(aryl), —OC(O)O-(aryl), —OC(O)—NH-(aryl), —O—(C₂₋₆ alkylene)-NH—(C₂₋₆ alkyl), —O—(C₂₋₆ alkylene)-NH-(tetrahydropyran), —O—(C₂₋₆ alkylene)-NH-(thiomorpholine dioxide), —O—(C₂₋₆ alkylene)-NH-(piperidinyl), —O—(C₂₋₆ alkylene)-NH-(piperazinyl), —O—(C₂₋₆ alkylene)-NH-(morpholinyl), —O—(C₂₋₆ alkylene)-NH-(aralkyl), —O—(C₂₋₆ alkylene)-NH-(cyclopropyl), —OSO₂—(C₃₋₆ cycloalkyl), —OSO₂-(aryl), —O—(C₂₋₆ alkylene)-OSO₂-(aryl), —OSO₂-(aralkyl), —O—(C₂₋₆ alkylene)-OSO₂-(heteroaryl), —OSO₂—(C₁₋₆ alkyl), —OSO₂-(pyridyl), —OSO₂-(thiazolyl), —O—(C₂₋₆ alkylene)-NHSO₂-(aryl), —O—(C₂₋₆ alkylene)-NHSO₂-(heteroaryl), —O—(C₂₋₆ alkylene)-NHC(O)-(aryl), —O—(C₂₋₆ alkylene)-NHC(O)-(heteroaryl), —O—(C₀₋₄ alkyl)pyridyl, —O—(C₀₋₄ alkyl)pyrimidinyl, —O—(C₀₋₄ alkyl)morpholinyl, —O—(C₀₋₄ alkyl)thiomorpholinyl, —O—(C₀₋₄ alkyl)imidazolyl, —O—(C₀₋₄ alkyl)thienyl, —O—(C₀₋₄ alkyl)tetrahydropyranyl, —O—(C₀₋₄ alkyl)tetrahydrofuranyl, —O—(C₀₋₄ alkyl)pyrrolidinyl, —O—(C₀₋₄ alkyl)piperidinyl, or —O—(C₀₋₄ alkyl)piperazinyl group;

R₅ and R₆ are independently —H, —OH, or an unsubstituted C₁₋₆ alkoxy group; or R₄ and R₅ together are a methylenedioxy group, or R₅ and R₆ together are a methylenedioxy group; and

R₈ is —H, —OH, —COOH, or an unsubstituted alkyl or —(CH₂)₁₋₆-phenyl group.

In other embodiments of the compounds of Formula I,

R₁ and R₂ are independently —H, —CH₃, —CH₂COOH, —CH₂C(O)OCH₂CH₃, allyl, or R₁ and R₂ together are a methylene group;

R₃ and R₃′ are each —H, or R₃ and R₃′ together are an oxo group;

R₄ is —H, —OH, OCH₃, —OCH₂CH₃, —O(CH₂)₂OH, —OCH₂COOH, —OCH₂COOCH₂CH₃, —O(CH₂)₂COOH, —O(CH₂)₂CH₂Br, —O-acetyl, —O-benzoyl, —O—(CH₂)₂—NH—(CH₂)₂—N(CH₃)₂, —O—(CH₂)₂—NH—(CH₂)₂—OCH₃, —O—(CH₂)₂—NH—(CH₂)₂—SCH₃, —O—(CH₂)₂—NH-morpholinyl, —O—(CH₂)₂—NH—(CH₂)₃—N(CH₃)₂, —O—(CH₂)₂—NH-benzyl, —O—(CH₂)₂—NH—(CH₂)₃-(thiomorpholine dioxide), —O—(CH₂)₂—NH—(CH₂)₃-morpholinyl, —O—(CH₂)₂—NH—(CH₂)₃-tetrahydropyranyl, —O-pyridyl optionally substituted with one or two substituents selected from the group consisting of C₁₋₄ alkyl, —NO₂, and NH₂, —O—(CH₂)₂—S-phenyl, —OSO₂-naphthyl optionally substituted with di(C₁₋₄ alkyl), —OSO₂—CF₃, —OSO₂-thiaolyl optionally substituted with acetamido, —O—(CH₂)₀₋₂SO₂-phenyl wherein the phenyl group is optionally substituted with one or two substituents selected from the group consisting of methyl, methoxy, fluoro, chloro, trifluoromethyl, and nitro, —OSO₂-cyclopentyl, —OSO₂-thienyl, —OSO₂-benzyl, —(CH₂)₂-cyclopropyl, —(CH₂)₂-morpholinyl, —(CH₂)₂-imidazolyl, —(CH₂)₂-pyrrolidinyl, or —(CH₂)₂-piperazinyl group, wherein the piperazinyl group is optionally substituted with methyl, isopropyl, or methoxyethyl;

R₅ and R₆ are independently —H, —OH, or —OCH₃; and

R₈ is —H, methyl, ethyl, —COOH, or benzyl.

Compounds of Formula I may have either stereochemical configuration at position 14 and both generally exhibit lipid-lowering activity. In some embodiments the compounds have the R-(+) stereochemical configuration and in others the compounds have the S-(−) stereochemical configuration at position 14. For Example, compounds of Formula I include compounds of Table 4 in the Examples, e.g., compounds 162 and 163. Compounds of Formula I can be racemic at position 14 or can be a mixture of enantiomers having from 1% to 99% enantiomeric excess (e.e.) with respect to the to R-(+) stereochemical configuration. For example, the compound of Formula I may have at least 1%, at least 5%, at least 10%, at least 20%, at least 30%, at least 40%, at least 50%, at least 60%, at least 70%, at least 80%, at least 90%, at least 95%, at least 96%, at least 97%, at least 98%, or at least 99% e.e. Production and/or separation of either optical isomer of compounds of Formula I is within the skill in the art in view of the guidance provided herein.

In another aspect, the present technology provides compounds of Formula II, as well as stereoisomers thereof, tautomers thereof, solvates thereof, and pharmaceutically acceptable salt thereof.

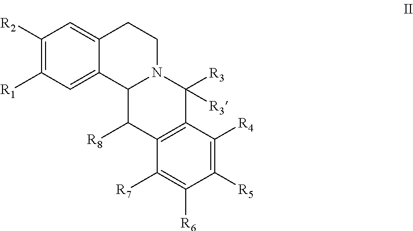

In compounds of Formula II,

R₁ and R₂ are independently —H, —(CH₂)₀₋₆COOR′, —C(O)R″, —OR′, —NR₁₀R₁₁, —C(O)NR₁₀R₁₁, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group; or R₁ and R₂ together are a 1,2-dioxyethylene group; provided that R₁ and R₂ are not both —OR;

R₃ and R₈ are independently —H, —OH, —Cl, —Br, —F, —I, —CN, —NH₂, —C(O)NH₂, —COOH, or a substituted or unsubstituted alkyl, alkenyl, alkoxy or aralkyl group;

R₃′ is —H, or R₃ and R₃′ together are an oxo group;

R₄ is —H, halogen, —OR′, —OSO₂R″, —OC(O)R″, —OC(O)OR″, —OC(O)NR′R″, —O-alkylene-NR′R′, —O-alkylene-OSO₂R″, —O-alkylene-S(O)₀₋₂R″, —O-alkylene-NR′SO₂R″, —O-alkylene-N(R′)C(O)R′, or a substituted or unsubstituted alkyl group;

R₅ and R₆ are independently —H, halogen, —OH, or a substituted or unsubstituted alkoxy group; or R₄ and R₅ together are a methylenedioxy group, or R₅ and R₆ together are a methylenedioxy group;

R₇ is —H, halogen, —OH, or a substituted or unsubstituted alkyl or alkoxy group;

R₁₀ and R₁₁ are independently H, —C(O)OR″, or a substituted or unsubstituted alkyl group;

each R′ is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;

each R″ is independently a substituted or unsubstituted alkyl, alkenyl, cycloalkyl, cycloalkylalkyl, aryl, aralkyl, heteroaryl, heteroarylalkyl, heterocyclyl, or heterocyclylalkyl group;

with the proviso that when R₁ and R₂ are both H, then R₄ is halogen, —OSO₂R″, —OC(O)R″, —OC(O)OR″, —OC(O)NR′R″, —O-alkylene-NR′R′, —O-alkylene-OSO₂R″, —O-alkylene-S(O)₀₋₂R″, —O-alkylene-NR′SO₂R″, —O-alkylene-N(R)C(O)R′, or a substituted or unsubstituted alkyl group.

In some embodiments of the compounds of Formula II, R₁ and R₂ are independently —H, —(CH₂)₀₋₆COOR′, —NR₁₀R₁₁, —C(O)NR₁₀R₁₁, or a substituted or unsubstituted alkyl group; or R₁ and R₂ together are an ethylene group. In other embodiments, one of R₁ and R₂ is OR′ and the other is —H, 4-CH₂)₀₋₆COOR′, —NR₁₀R₁₁, —C(O)NR₁₀R₁₁, or a substituted or unsubstituted alkyl group; or R₁ and R₂ together are an ethylene group. In some embodiments, R₁ and R₂ together are an ethylene group.

In some embodiments of the compounds of Formula II, R₁₀ and R₁₁ are independently H, C₁₋₆ alkyl optionally substituted with a hydroxy group.

In some embodiments of the compounds of Formula II, R₃ and R₃′ are each —H, or R₃ and R₃′ together are an oxo group.

In some embodiments of the compounds of Formula II, R₄ is —H, —OR′, —OSO₂R″, —OC(O)OR″, —OC(O)NR′R″, —O-alkylene-OSO₂R″, or —O-alkylene-NR′R′. In other embodiments, R₄ is —H, —OR′, —OSO₂R″, or —OC(O)R″. In some embodiments, R₄ is —H, —OH, or a substituted or unsubstituted C₁₋₆ alkoxy, C₇₋₁₄ aralkoxy, —OC(O)—(C₁₋₆ alkyl), —OC(O)-(aryl), —OC(O)O-(aryl), —OC(O)—NH-(aryl), —O—(C₂₋₆ alkylene)-NH—(C₂₋₆ alkyl), —O—(C₂₋₆ alkylene)-NH-(tetrahydropyran), —O—(C₂₋₆ alkylene)-NH-(thiomorpholine dioxide), —O—(C₂₋₆ alkylene)-NH-(piperidinyl), —O—(C₂₋₆ alkylene)-NH-(piperazinyl), —O—(C₂₋₆ alkylene)-NH-(morpholinyl), —O—(C₂₋₆ alkylene)-NH-(aralkyl), —O—(C₂₋₆ alkylene)-NH-(cyclopropyl), —OSO₂—(C₃₋₆ cycloalkyl), —OSO₂-(aryl), O—(C₂₋₆ alkylene)-OSO₂-(aryl), —OSO₂-(aralkyl), —O—(C₂₋₆ alkylene)-OSO₂-(heteroaryl), —OSO₂—(C₁₋₆ alkyl), —OSO₂-(pyridyl), —OSO₂-(thiazolyl), —O—(C₂₋₆ alkylene)-NHSO₂-(aryl), —O—(C₂₋₆ alkylene)-NHSO₂-(heteroaryl), —O—(C₂₋₆ alkylene)-NHC(O)-(aryl), —O—(C₂₋₆ alkylene)-NHC(O)-(heteroaryl), —O—(C₀₋₄ alkyl)pyridyl, —O—(C₀₋₄ alkyl)pyrimidinyl, —O—(C₀₋₄ alkyl)morpholinyl, —O—(C₀₋₄ alkyl)thiomorpholinyl, —O—(C₀₋₄ alkyl)imidazolyl, —O—(C₀₋₄ alkyl)thienyl, —O—(C₀₋₄ alkyl)tetrahydropyranyl, —O—(C₀₋₄ alkyl)tetrahydrofuranyl, —O—(C₀₋₄ alkyl)pyrrolidinyl, —O—(C₀₋₄ alkyl)piperidinyl, or —O—(C₀₋₄ alkyl)piperazinyl group. In some embodiments, R₄ is —H, —OH, or a substituted or unsubstituted C₁₋₆ alkoxy, —OC(O)—(C₁₋₆ alkyl)-biotin, —OSO₂-(aryl), —O—(C₂₋₆ alkylene)-OSO₂-(aryl), or —OSO₂-(aralkyl).

In some embodiments of the compounds of Formula II, the 14-position in Formula II is the R-(+) stereochemical configuration.

In some embodiments of the compounds of Formula II, R₅ is OH or unsubstituted alkoxy and R₆ is H. In other embodiments, R₆ is H, and R₇ is H.

In some embodiments of the compounds of Formula II, R₈ is —H, —OH, —COOH, or an unsubstituted alkyl or —(CH₂)₁₋₆-phenyl group.

In some embodiments of the compounds of Formula II,

R₁ and R₂ are independently —H, —(CH₂)₀₋₆COOR′, —C(O)NR₁₀R₁₁, or a substituted or unsubstituted alkyl group; or R₁ and R₂ together are an ethylene group;

R₃ and R₃′ are each —H, or R₃ and R₃′ together are an oxo group;

R₄ is —H, —OH, or a substituted or unsubstituted C₁₋₆ alkoxy, C₇₋₁₄ aralkoxy, —OC(O)—(C₁₋₆ alkyl), —OC(O)-(aryl), —OC(O)O-(aryl), —OC(O)—NH-(aryl), —O—(C₂₋₆ alkylene)-NH—(C₂₋₆ alkyl), —O—(C₂₋₆ alkylene)-NH-(tetrahydropyran), —O—(C₂₋₆ alkylene)-NH-(thiomorpholine dioxide), —O—(C₂₋₆ alkylene)-NH-(piperidinyl), —O—(C₂₋₆ alkylene)-NH-(piperazinyl), —O—(C₂₋₆ alkylene)-NH-(morpholinyl), —O—(C₂₋₆ alkylene)-NH-(aralkyl), —O—(C₂₋₆ alkylene)-NH-(cyclopropyl), —OSO₂—(C₃₋₆ cycloalkyl), —OSO₂-(aryl), —O—(C₂₋₆ alkylene)-OSO₂-(aryl), —OSO₂-(aralkyl), —O—(C₂₋₆ alkylene)-OSO₂-(heteroaryl), —OSO₂—(C₁₋₆ alkyl), —OSO₂-(pyridyl), —OSO₂-(thiazolyl), —O—(C₂₋₆ alkylene)-NHSO₂-(aryl), —O—(C₂₋₆ alkylene)-NHSO₂-(heteroaryl), —O—(C₂₋₆ alkylene)-NHC(O)-(aryl), —O—(C₂₋₆ alkylene)-NHC(O)-(heteroaryl), —O—(C₀₋₄ alkyl)pyridyl, —O—(C₀₋₄ alkyl)pyrimidinyl, —O—(C₀₋₄ alkyl)morpholinyl, —O—(C₀₋₄ alkyl)thiomorpholinyl, —O—(C₀₋₄ alkyl)imidazolyl, —O—(C₀₋₄ alkyl)thienyl, —O—(C₀₋₄ alkyl)tetrahydropyranyl, —O—(C₀₋₄ alkyl)tetrahydrofuranyl, —O—(C₀₋₄ alkyl)pyrrolidinyl, —O—(C₀₋₄ alkyl)piperidinyl, or —O—(C₀₋₄ alkyl)piperazinyl group;

R₅ and R₆ are independently —H, —OH, or an unsubstituted C₁₋₆ alkoxy group; or R₄ and R₅ together are a methylenedioxy group, or R₅ and R₆ together are a methylenedioxy group; and

R₈ is —H, —OH, —COOH, or an unsubstituted alkyl or —(CH₂)₁₋₆-phenyl group.

In some embodiments of the compounds of Formula II,

R₁ and R₂ are independently —H, —CH₃, —CH₂OH, —OH, —OCH₃, —OCH₂CH₃, —OCH₂CH₂OH, —COOH, —C(O)N(CH₃)₂, —C(O)NH(CH₂CH₂OH), —C(O)OCH₃, —NHCH₃, —N(CH₃)₂, —NC(O)OCH₂CH₃, benzyloxy, or R₁ and R₂ together are an ethylene group;

R₃ and R₃′ are each —H, or R₃ and R₃′ together are an oxo group;

R₄ is —H, —OH, OCH₃, —OCH₂CH₃, —O(CH₂)₂OH, —OCH₂COOH, —OCH₂COOCH₂CH₃, —O(CH₂)₂COOH, —O(CH₂)₂CH₂Br, —O-acetyl, —O-benzoyl, —O—(CH₂)₂—NH—(CH₂)₂—N(CH₃)₂, —O—(CH₂)₂—NH—(CH₂)₂—OCH₃, —O—(CH₂)₂—NH—(CH₂)₂—SCH₃, —O—(CH₂)₂—NH-morpholinyl, —O—(CH₂)₂—NH—(CH₂)₃—N(CH₃)₂, —O—(CH₂)₂—NH-benzyl, —O—(CH₂)₂—NH—(CH₂)₃-(thiomorpholine dioxide), —O—(CH₂)₂—NH—(CH₂)₃-morpholinyl, —O—(CH₂)₂—NH—(CH₂)₃-tetrahydropyranyl, —O-pyridyl optionally substituted with one or two substituents selected from the group consisting of C₁₋₄ alkyl, —NO₂, and NH₂, —O—(CH₂)₂—S-phenyl, —OSO₂-naphthyl optionally substituted with di(C₁₋₄ alkyl), —OSO₂—CF₃, —OSO₂-thiaolyl optionally substituted with acetamido, —O—(CH₂)₀₋₂SO₂-phenyl wherein the phenyl group is optionally substituted with one or two substituents selected from the group consisting of methyl, methoxy, fluoro, chloro, trifluoromethyl, and nitro, —OSO₂-cyclopentyl, —OSO₂-thienyl, —OSO₂-benzyl, —(CH₂)₂-cyclopropyl, —(CH₂)₂-morpholinyl, —(CH₂)₂-imidazolyl, —(CH₂)₂-pyrrolidinyl, or —(CH₂)₂-piperazinyl group, wherein the piperazinyl group is optionally substituted with methyl, isopropyl, or methoxyethyl;

R₅ and R₆ are independently —H, —OH, or —OCH₃; and

R₈ is —H, methyl, ethyl, —COOH, or benzyl.

In some embodiments of compounds of Formula II, R₄ is —O—(CH₂)₂—S-phenyl, wherein the phenyl group is optionally substituted with one or two substituents selected from the group consisting of methyl, methoxy, fluoro, chloro, trifluoromethyl, and nitro.

In accordance with another aspect, the present technology provides methods of synthesizing 14R-tetrahydropalmatine. The method includes treating berberine with boron trichloride in methylene chloride, methylating the product with methyl iodide and potassium carbonate in dry acetone, and hydrogenating the product using an asymmetric hydrogenation catalyst to yield 14R-tetrahydropalmatine.

Compounds of Formulas I, II and EE may be prepared using procedures such as those described in published PCT applications WO 2009/002873 and WO2010/075469, each of which is incorporated by reference in its entirety herein and for all purposes. Compounds of Formula EE (and EE′) may be prepared in accordance with the synthetic schemes described herein.

Thus, methods for synthesizing compounds of Formula EE are provided. Compound of Formula EE have the structure:

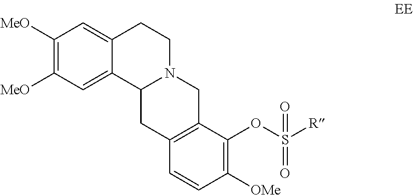

and salts and stereoisomers thereof, wherein

R″ is a substituted or unsubstituted alkyl, cycloalkyl, cycloalkylalkyl, alkenyl, aryl, aralkyl, heterocyclyl, heterocyclylalkyl, heteroaryl, or heteroarylalkyl group.

In one aspect, compounds of Formula EE may be prepared as depicted in Scheme 1. This route has the advantages of being relatively short and therefore efficient. The starting material is an inexpensive natural product, while the relatively more costly sulfonyl-containing group is introduced only at the end of the synthesis.

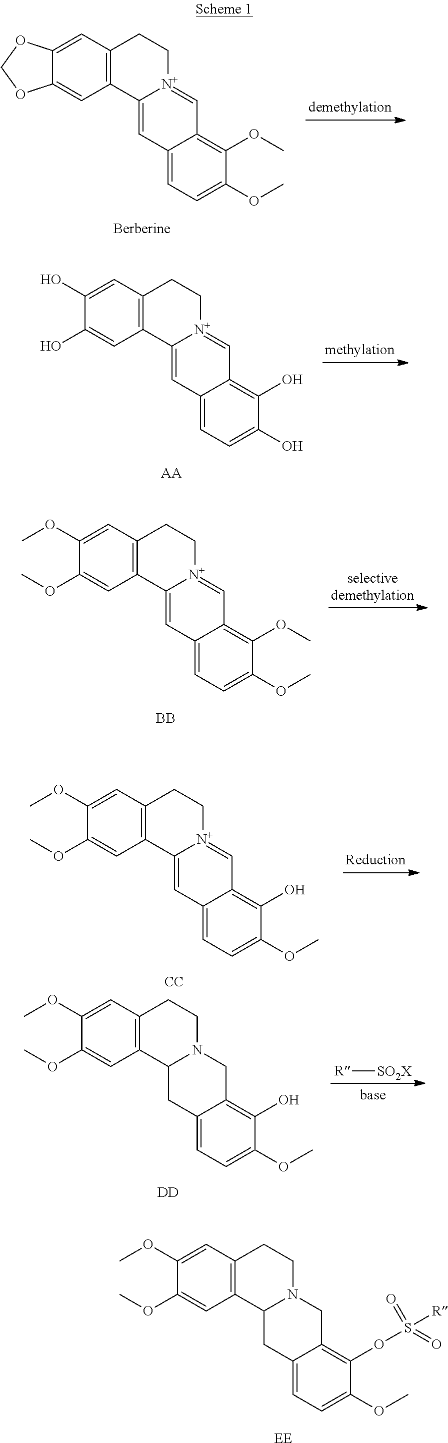

In accordance with Scheme 1, in some embodiments, the method comprises exposing a compound of structure DD,

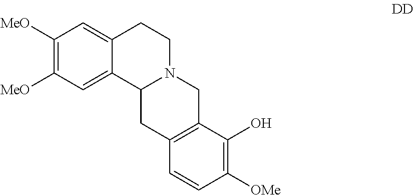

to R″—SO₂X under sulfonylation conditions to give a compound of structure EE;

wherein

R″ is a substituted or unsubstituted alkyl, cycloalkyl, cycloalkylalkyl, alkenyl, aryl, aralkyl, heterocyclyl, heterocyclylalkyl, heteroaryl, or heteroarylalkyl group; and

X is a leaving group.

Sulfonylation conditions are known in the art and are those conditions under which a hydroxyl group may be converted to a sulfonic ester using a sulfonylating reagent. In the present reaction, the sulfonylating reagent used is R″SO₂X, where R″ is as defined in Structure EE and X is a leaving group. In some embodiments of the present method, R″ is a substituted or unsubstituted alkyl, cycloalkyl, aryl or aralkyl group. In others, R″ is a substituted or unsubstituted C₁₋₆ alkyl, C₃₋₆ cycloalkyl, C₆₋₁₀ aryl or C₇₋₁₄ aralkyl group. In still others, R″ is a substituted or unsubstituted heteroaryl group selected from substituted or unsubstituted thienyl, pyridinyl, quinolinyl, or thiazolyl group. In still others, R″ is phenyl or naphthyl, each optionally substituted with 1, 2 or 3 substituents selected from the group consisting of F, Cl, Br, CN, NO₂, C₁₋₆ alkyl optionally substituted with 1 or more halo (e.g., F, Cl, Br), C₁₋₆ alkoxy optionally substituted with 1 or more halo (e.g., F, Cl, Br), hydroxy, sulfonamido, sulfone, amino, C₁₋₆ alkylamino, and di(C₁₋₆ alkyl)amino. In illustrative embodiments, R″ is phenyl or 3-fluorophenyl. X is a leaving group as that term is understood by those skilled in the art (i.e., an electronegative group that may be replaced by a nucleophilic group), including, e.g., a halide, cyanide or anhydride. In some embodiments X is a halide such as chloride or bromide.

Sulfonylation conditions may include a suitable base, i.e., a base which allows for the production of the sulfonic ester. A suitable base may be organic or inorganic but preferably does not result in side products with the sulfonylating reagent (e.g., does not form sulfonamides) or compound EE. In some embodiments, the suitable base is a tertiary organoamine (e.g., triethylamine, diisopropylethylamine), a pyridine (e.g., pyridine, dimethylaminopyridine), a carbonate (e.g., sodium or potassium carbonate) or a bicarbonate (e.g., sodium or potassium bicarbonate).

Sulfonylation conditions may include a suitable solvent (one which dissolves the reactants sufficiently to allow formation of product) such as dichloromethane, chloroform, ether, THF, DMF, and the like. While sulfonylation reactions are typically carried out at ambient temperature, it is within the skill in the art to cool (e.g., ice bath or 10-15° C.) or heat such reactions as necessary in order to optimize yields of sulfonylated product.

In some embodiments, the methods further comprises exposing a compound of structure CC,

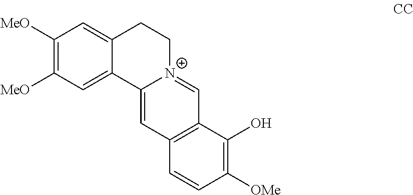

to reducing conditions to give the compound of structure DD.

Reducing conditions known in the art can be employed to produce compound DD. In some embodiments of the method, the reducing conditions comprise using a borohydride in an alcohol (e.g., methanol or ethanol) or a transition metal catalyst in the presence of hydrogen gas. Typical borohydrides which may be used as the reducing agent include, but are not limited to sodium borohydride, sodium cyanoborohydride, or sodium triacetoxyborohydride. In some embodiments, the borohydride is sodium borohydride and the alcohol is methanol. In some embodiments, the transition metal catalyst is palladium, palladium hydroxide, platinum or platinum oxide. The transition metal is optionally supported on carbon or another inert support. Transition metals with chiral ligands may be used in order to effect a stereselective reduction. Depending on the reducing conditions selected, the present reductions may be carried out at room temperature, below room temperature or at elevated temperatures. The reductions may be monitored by know techniques such as, but not limited to TLC and HPLC, and stopped when such techniques indicate, e.g., complete consumption of starting material. It is within the skill in the art to select a suitable temperature and reaction time for the reduction.

In some embodiments, the method further comprises heating a compound of structure BB

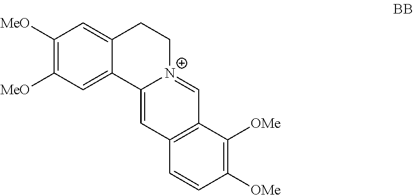

to give a compound of structure CC.

Selective demethylation of compound BB can be conducted by methods known in the art. In some embodiments of the method, the compound of structure BB is heated in the presence of N,N-dimethylformamide, urea or in vacuo to effect selective demethylation of the desired methoxy group. For example, the reaction may be heated to temperatures of about 190° C. to about 250° C. The reaction may also be carried out under reduced pressures from about 20 to about 30 Torr.

In some embodiments, the present methods further comprise exposing a compound of structure AA

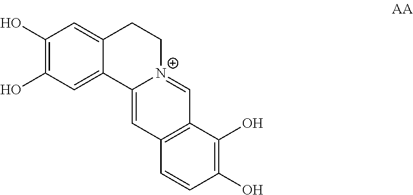

to a methylating agent to give a compound of structure BB.

Any suitable methylating agents known in the art can be employed to methylate the compound of formula AA. In some embodiments of the method, the methylating agent is dimethylsulfate or methyl iodide. The methylation may be carried out in the presence of a base and solvent. For example, inorganic bases such as potassium or sodium carbonate may be used. Suitable solvents for this step include acetone, methanol, ethanol, dimethylformamide, acetonitrile, DMSO, NMP, dichloromethane, or ethyl acetate. Optionally, the reaction mixture may be heated; for example, acetone may be heated to reflux until the reaction is complete.

In some embodiments, the method further comprises exposing beberine to an acid of sufficient strength to provide a compound of structure AA. In illustrative embodiments, the acid is HBr, BBr₃, or AlCl₃.

A variety of solvents can be used for the above reactions including but not limited to chlorinated solvents such as chloroform and dichloromethane, ketones such as acetone, and alcohols such as methanol, ethanol and the like.

In an alternate embodiment, compounds of Formula EE may be prepared as depicted in Scheme 2, in which the steps of Scheme 1 are performed in a different order. Reaction conditions used in various steps of Scheme 1 may be utilized for analogous steps described in Scheme 2. Scheme 2 retains the advantages of Scheme 1 in that it again begins with an inexpensive natural product and is short. Unexpectedly, the sulfonyl group is stable to the other conditions in the synthesis. Thus, where early introduction of the sulfonyl could be advantageous in terms of isolation or purification of the intermediate compounds, the route of Scheme 2 may be used.

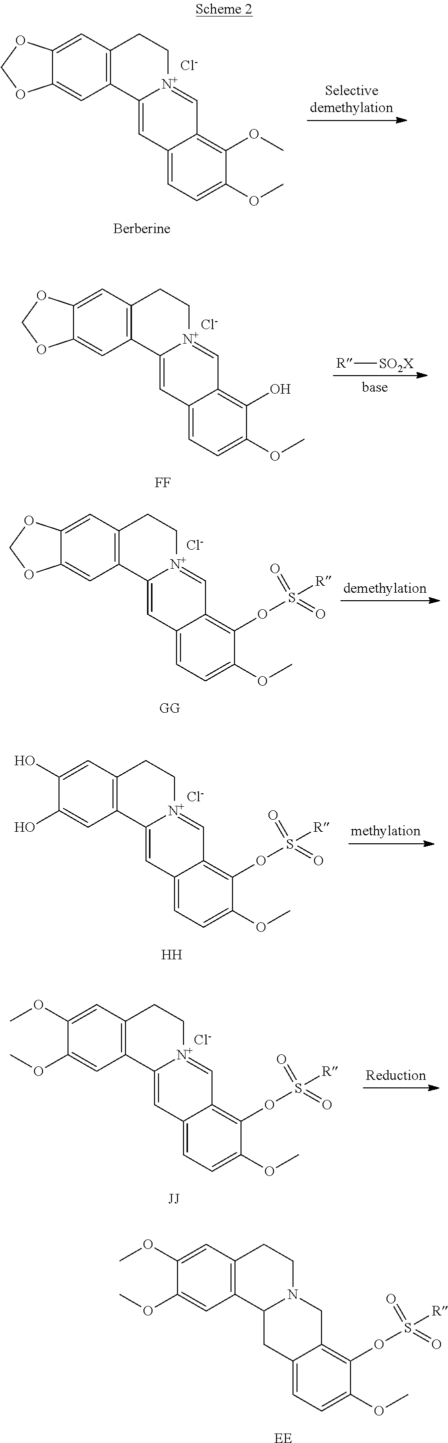

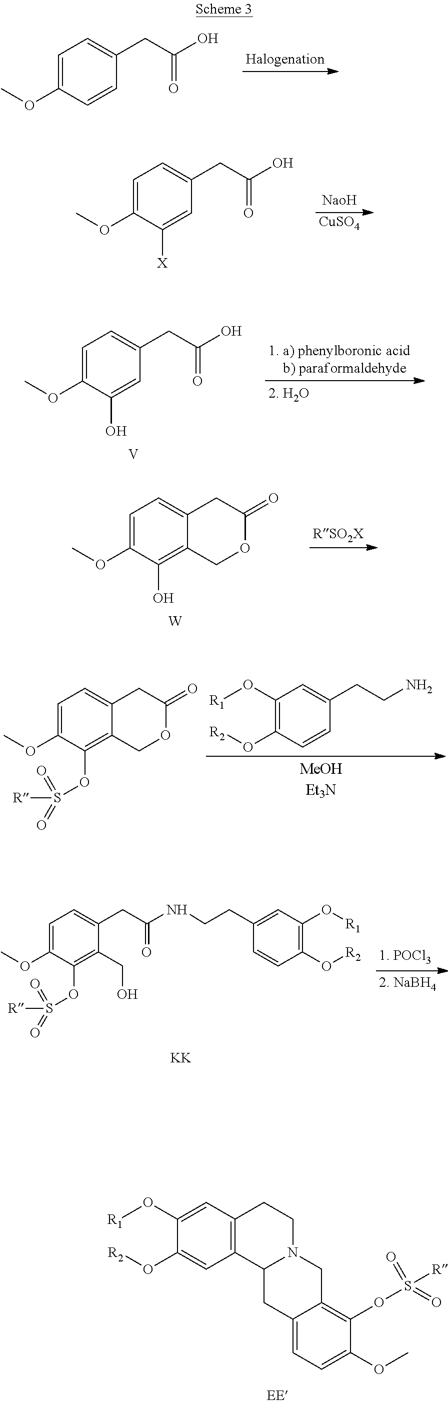

Alternatively, Scheme 3 shows another general synthetic route to compounds of Formula EE′ in which R₁ and R₂ may be defined as herein for compounds of Formula I. Phenyl acetic acid derivative V may be exposed consecutively to phenylboronic acid, followed by paraformaldehyde. Both stages of the reaction are typically heated, and the reaction with paraformaldehyde may be carried out under pressure in, e.g., a stainless steel bomb. Suitable solvents for this reaction include aromatic solvents such as toluene. The resulting boronate is hydrolyzed with water to give compound W. The latter compound may be sulfonylated with R″—SO₂X as described above (in Scheme 1). The sulfonylated isochromanone derivative may be reacted with a phenethylamine compound as shown to give compound KK. Ring closure using POCl₃ in a suitable solvent, such as toluene, followed by reduction with a suitable reducing agent such as e.g. sodium borohydride, gives compound of structure EE′.

In another embodiment, compounds of Formula EE′ (in which R₁ and R₂ may be defined as herein for compounds of Formula I) may be prepared as depicted in Scheme 4

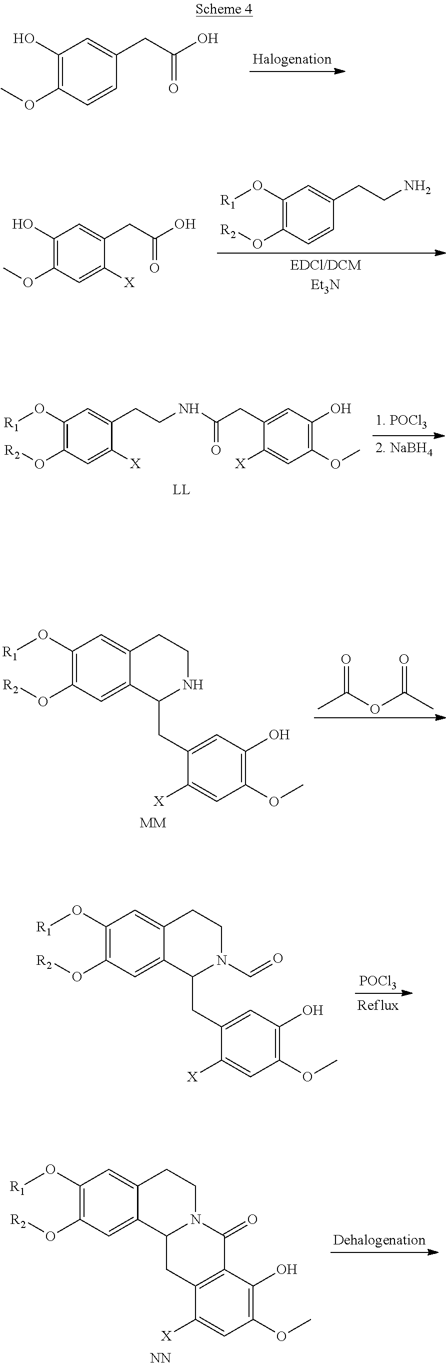

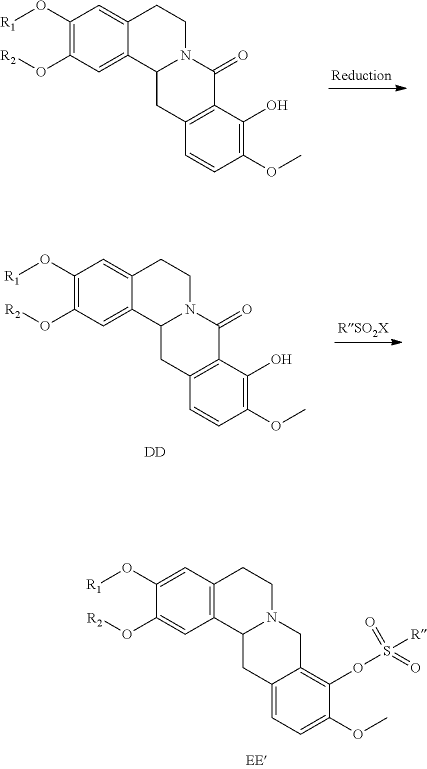

As shown in Scheme 4, halogenated phenyl acetic acid derivatives may be coupled to a phenethylamine compound using coupling agents (e.g., carbodiimides such as EDCI or DCC) or other standard amide bond forming conditions (e.g., mixed anhydride, acid halides) to give compound LL. Ring closure using POCl₃ in a suitable solvent, such as toluene, followed by reduction with a suitable reducing agent, as described above for the reduction of the compound CC to DD, gives the compound of structure MM. This compound may be treated with acetic anhydride followed by ring closure with POCl₃ in a suitable solvent, such as toluene to give the halogenated isoquinolinone derivative compound NN. The latter compound may be dehalogenated and reduced using the reducing conditions described above to give compound DD. In some embodiments of the method, the reducing conditions comprise using lithium aluminum hydride in ether or tetrahydrofuran. Finally, compound DD may be sulfonylated with R″—SO₂X as described herein above (in Scheme 1) to give the compound of formula EE′ (in which R₁ and R₂ may be defined as herein for compounds of Formula I).

Alternatively, in some embodiments, compounds of Formula EE′ (in which R₁ and R₂ may be defined as herein for compounds of Formula I) may be prepared as depicted in Scheme 5.

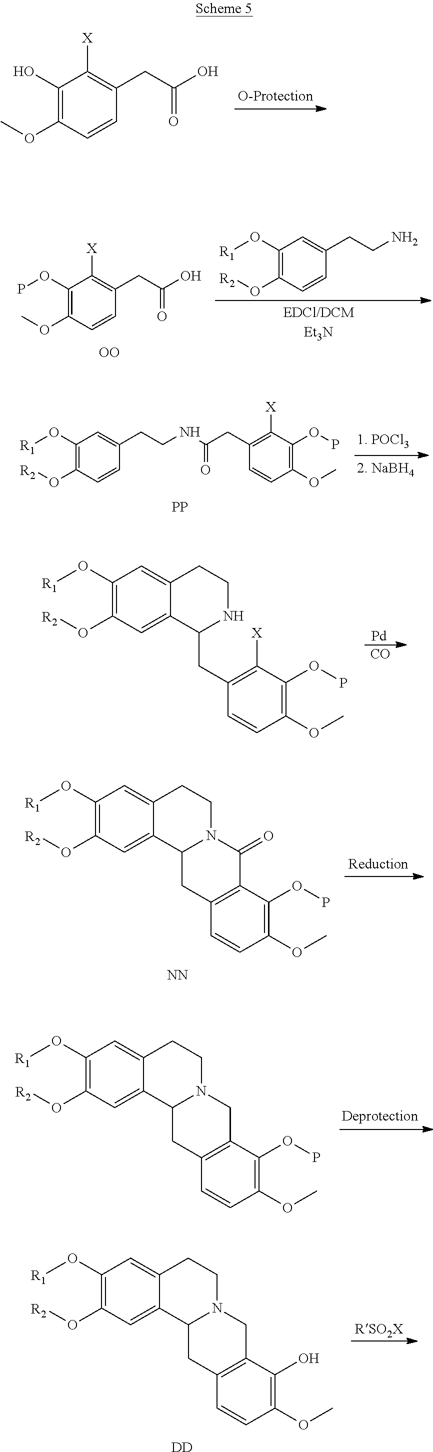

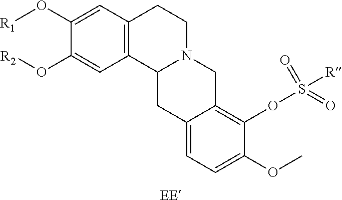

In accordance with Scheme 5, O-protected halogenated phenyl acetic acid derivative OO may be coupled with a phenethylamine compound using standard techniques (see, above) to give compound PP. In some embodiments, the protecting group P may be a benzyl group or an allyl group. Ring closure of compound PP using POCl₃ in a suitable solvent, such as toluene, and reduction with a suitable reducing agent such as e.g. sodium borohydride, followed by palladium catalyzed reaction with carbon monoxide gives compound of structure NN. The latter compound may be reduced using reducing conditions known in the art and deprotected to produce compound DD. In some embodiments of the method, the reducing conditions may comprise using lithium aluminum hydride in ether or tetrahydrofuran and the deprotection may be conducted using Pd/C-catalyzed hydrogenation. Finally, compound DD may be sulfonylated with R″—SO₂X as described above (in Scheme 1) to give the compound of formula EE′ (in which R₁ and R₂ may be defined as herein for compounds of Formula I).

In other embodiments, compounds of Formula EE′ (in which R₁ and R₂ may be defined as herein for compounds of Formula I) may be prepared as depicted in Scheme 6, using procedures adapted from Bioorganic & Medicinal Chemistry Letters, 2006, 16(5), 1380-1383.

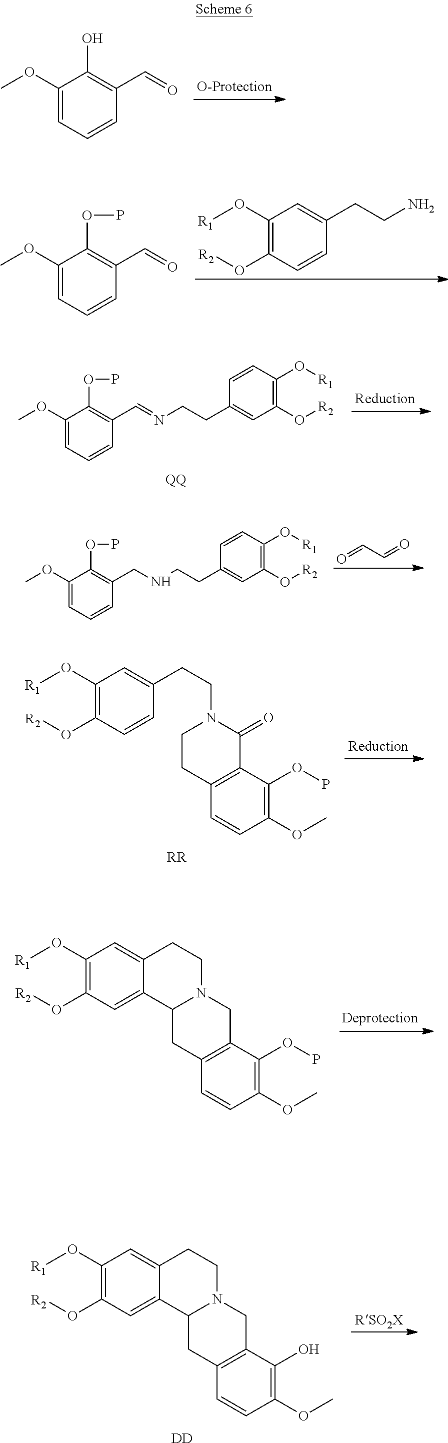

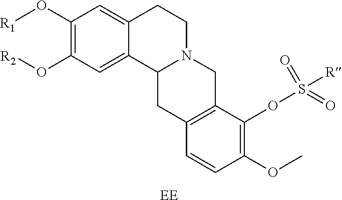

As shown in Scheme 6, O-protected, 2-hydroxy-3-methoxybenzaldehyde may be reacted with a phenethylamine compound as shown to give compound QQ. In some embodiments, the O-protecting group may be a benzyl or allyl group. The imine bond in compound QQ is then reduced using standard reducing conditions (e.g. NaBH₄ in MeOH) and subsequently reacted with oxalaldehyde to give the compound RR. The latter compound may be reduced using standard reducing conditions such as described above and deprotected to produce compound DD. In some embodiments of the method, the reducing conditions may comprise using lithium aluminum hydride in ether or tetrahydrofuran and the deprotection may be conducted using Pd/C-catalyzed hydrogenation or, in the case of allyl, Pd(0) catalysis under basic conditions. Finally, compound DD may be sulfonylated with R″—SO₂X as described above (in Scheme 1) to give the compound of formula EE.

In another embodiment, compounds of Formula EE′ may be prepared as depicted in Scheme 7

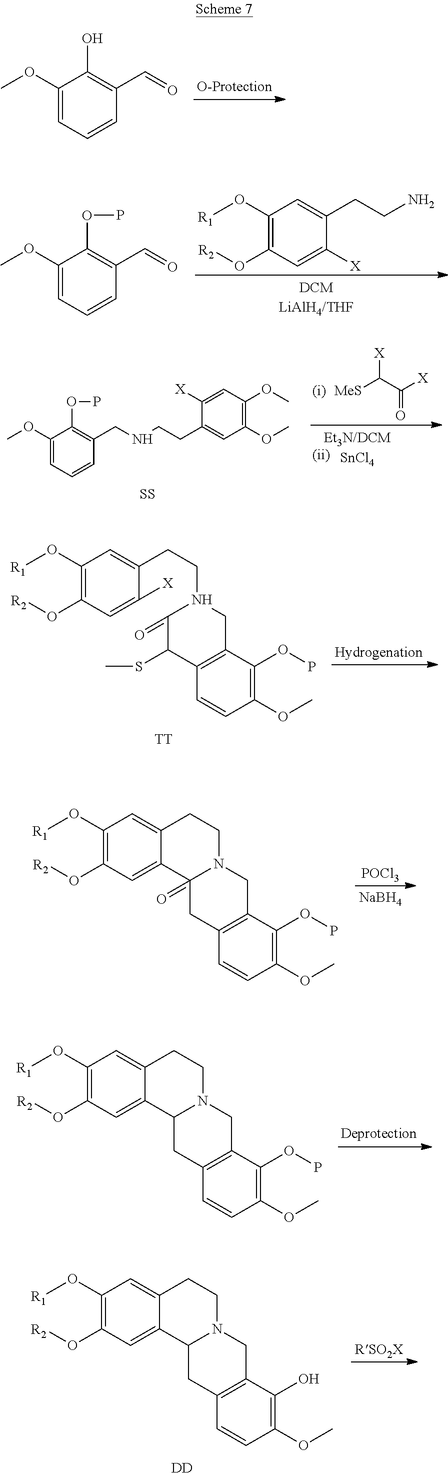

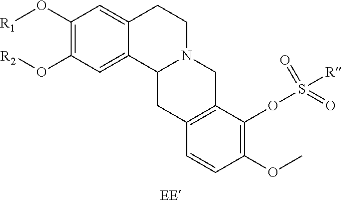

In Scheme 7, O-protected, 2-hydroxy-3-methoxybenzaldehyde may be reductively aminated with a halogenated phenethylamine compound as shown to give compound SS. In some embodiments, the O-protecting group may be a benzyl group. Compound SS is reacted with 2-halo-2-(methylthio)acetyl halide in presence of a base such as triethylamine in a suitable solvent such as dichloromethane to give the compound of formula TT. The latter compound may be hydrogenated using metal catalyst such as Raney Ni to remove the halogen and the MeS side chain. The resulting compound may be subjected to ring closure using POCl₃ in a suitable solvent, such as toluene, followed by reduction with a suitable reducing agent such as e.g. sodium borohydride, and subsequent O-group deprotection using Pd/C-catalyzed hydrogenation gives compound DD. Finally, compound DD may be sulfonylated with R″—SO₂X as described herein above (in Scheme 1) to give the compound of formula EE′ (in which R₁ and R₂ may be defined as herein for compounds of Formula I). This procedure as outlined in Scheme 7 is adapted from Chem. Pharm. Bull., 2000, 48(3), 399-404.

In an alternate embodiment, compounds of Formula EE′ (in which R₁ and R₂ may be defined as herein for compounds of Formula I) may be prepared as depicted in Scheme 8 and adapted from J. Org. Chem., 1990, 55 (6), pp 1932-1936.

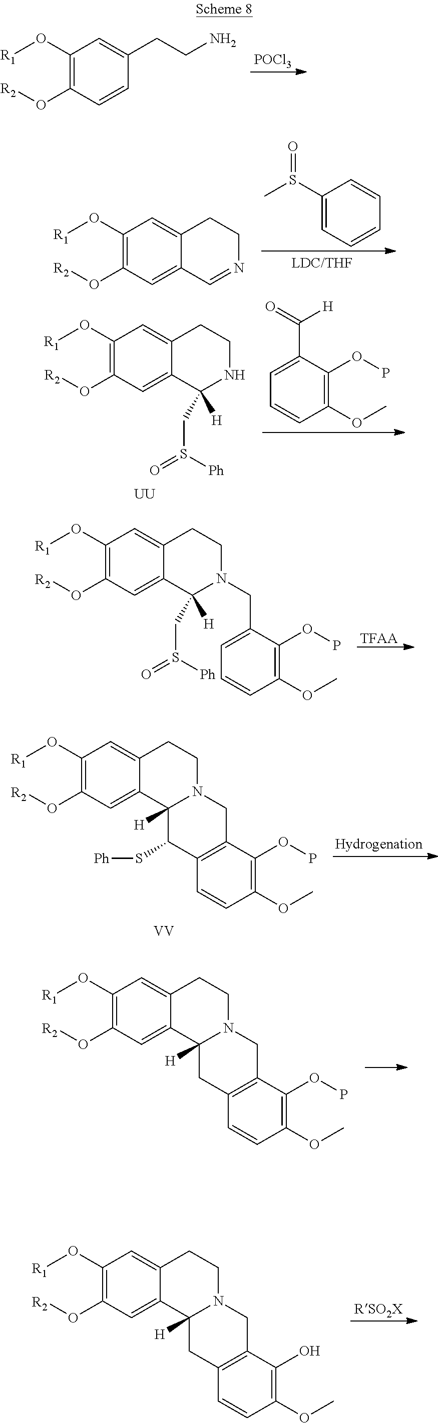

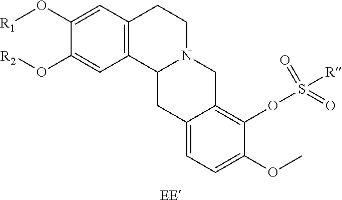

In Scheme 8, a phenethylamine derivative may be subjected to ring closure using POCl₃ in a suitable solvent, such as toluene, followed by reaction with methylsulfinylbenzene as shown to give compound UU. Compound UU may then be treated with O-protected 2-hydroxy-3-methoxybenzaldehyde followed by ring closure with trifluoroacetic anhydride to produce compound of formula VV. The latter compound may be hydrogenated using a metal catalyst such as Raney Ni to remove the sulfinylbenzene side chain and the resulting compound may be deprotected using Pd/C-catalyzed hydrogenation to give compound DD. Finally, compound DD may be sulfonylated with R″—SO₂X as described herein above (in Scheme 1) to give the compound of formula EE′.

In yet another embodiment, compounds of Formula EE′ (in which R₁ and R₂ may be defined as herein for compounds of Formula I) may be prepared as depicted in Scheme 9 and adapted from J. Org. Chem., 1996, 61 (2), pp 573-580.

In accordance with Scheme 9, phenyl acetic acid derivative V may be reacted with dimethylamine in presence of oxalyl chloride to give the corresponding amide. This amide on reduction with standard reducing agents such as LiAlH₄, followed by reaction with ethylchloroacetate in presence of BuLi gives the corresponding ethylbenzoate derivative WW. Compound WW may be reacted with compound XX in presence of n-BuLi to give a compound of formula YY. Compound XX may be prepared starting from phenethylamine compound which is subjected to ring closure in presence of formaldehyde-formic acid reagent to give the isoquinoline derivative which may be further reacted with di-tert-butyl dicarbonate to give the compound of formula XX. Reaction of compound XX and compound WW yields arylmethyl-isoquinoline compound YY which may be exposed to any suitable acid such as a mineral acid e.g. HCl, to give the compound of formula NN. The latter compound may be reduced using reducing conditions known in the art and deprotected to produce compound DD. In some embodiments of the method, the reducing conditions may comprise lithium aluminum hydride in tetrahydrofuran or ether and the deprotection may be conducted using Pd/C-catalyzed hydrogenation. Finally, compound DD may be sulfonylated with R″—SO₂X as described herein above (in Scheme 1) to give the compound of formula EE′.

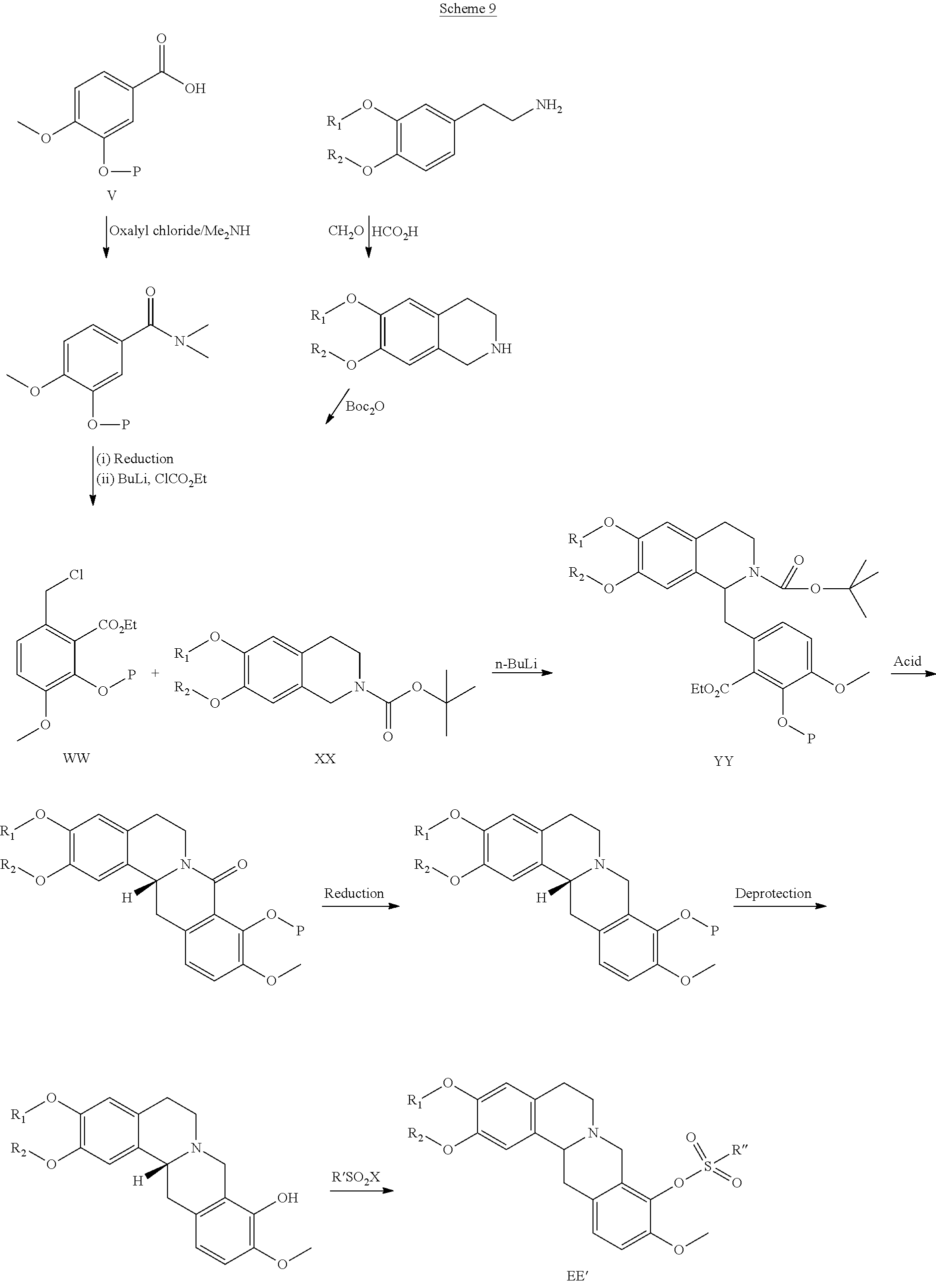

In another aspect, the instant present technology provides pharmaceutical compositions and medicaments comprising any of the compounds disclosed herein (e.g., compounds of Formulas I, II, EE or EE′) and a pharmaceutically acceptable carrier or one or more excipients or fillers. In some embodiments, there are provided pharmaceutical compositions for treating a condition selected from the group consisting of hyperlipidemia, hypercholesterolemia, hypertriglyceridemia, hepatic steatosis, and metabolic syndrome. Such compositions include a lipid-lowering effective amount of any compound as described herein, including but not limited to a compound of Formula I, Formula II or Formulas EE or EE′. In one embodiment, the pharmaceutical composition is packaged in unit dosage form. The unit dosage form is effective in lowering lipid levels (e.g., at least one of total cholesterol, LDL-cholesterol, triglyceride, and unesterified long chain fatty acids) in the bloodstream and/or in the liver when administered to a subject in need thereof.

The pharmaceutical compositions may be prepared by mixing one or more compounds described herein, pharmaceutically acceptable salts thereof, stereoisomers thereof, tautomers thereof, or solvates thereof, with pharmaceutically acceptable carriers, excipients, binders, diluents or the like to prevent and treat disorders associated with the effects of increased plasma and/or hepatic lipid levels. The compounds and compositions described herein may be used to prepare formulations and medicaments that prevent or treat a variety of disorders associated with increased plasma and/or hepatic lipid levels, e.g., hyperlipidemia, hypercholesterolemia, hepatic steatosis, and metabolic syndrome. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant technology.

For oral, buccal, and sublingual administration, powders, suspensions, granules, tablets, pills, capsules, gelcaps, and caplets are acceptable as solid dosage forms. These can be prepared, for example, by mixing one or more compounds of the present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of inventive compounds by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the inventive compound across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the present technology.

Those skilled in the art are readily able to determine an effective amount by simply administering a compound of the present technology to a patient in increasing amounts until the elevated plasma or hepatic cholesterol or triglycerides or progression of the disease state is decreased or stopped. The progression of the disease state can be assessed using in vivo imaging, as described, or by taking a tissue sample from a patient and observing the target of interest therein. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art.

Various assays and model systems can be readily employed to determine the therapeutic effectiveness of antihyperlipidemia treatment according to the present technology. For example, blood tests to measure total cholesterol as well as triglycerides, LDL and HDL levels are routinely given. Individuals with a total cholesterol level of greater than 200 mg/dL are considered borderline high risk for cardiovascular disease. Those with a total cholesterol level greater than 239 mg/dL are considered to be at high risk. An LDL level of less than 100 mg/dL is considered optimal. LDL levels between 130 to 159 mg/dL are borderline high risk. LDL levels between 160 to 189 mg/dL are at high risk for cardiovascular disease and those individuals with an LDL greater than 190 mg/dL are considered to be at very high risk for cardiovascular disease. Triglyceride levels of less than 150 mg/dL is considered normal. Levels between 150-199 mg/dL are borderline high and levels above 200 mg/dL are considered to put the individual at high risk for cardiovascular disease. Lipid levels can be determined by standard blood lipid profile tests. Effective amounts of the compositions of the present technology will lower elevated lipid levels by at least 10%, 20%, 30%, 50% or greater reduction, up to a 75-90%, or 95% or greater. Effective amounts will also move the lipid profile of an individual towards the optimal category for each lipid, i.e., decrease LDL levels from 190 mg/dL to within 130 to 159 mg/dL or even further to below 100 mg/dL. Effective amounts may further decrease LDL or triglyceride levels by about 10 to about 70 mg/dL, by about 20 to about 50 mg/dL, by about 20 to about 30 mg/dL, or by about 10 to about 20 mg/dL.

A variety of hyperlipidemia classification systems are known to persons of skill in the art. One such classification system is the Frederickson classification, which is summarized in Table 1 below.

TABLE 1

| | | | | | Relative |
|---|---|---|---|---|---|
| | Elevated | Elevated | Plasma | Plasma | Frequency |
| Phenotype | Lipoproteins | Lipid Levels | TC | TG | (%)* |
| I | Chylomicrons | TG | N to ↑ | ↑↑↑↑ | <1 |
| IIa | LDL | TC | ↑↑ | N | 10 |
| IIb | LDL & | TC, TG | ↑↑ | ↑↑↑ | 40 |
| | VLDL | | | | |
| III | IDL | TC, TG | ↑↑ | ↑↑ | <1 |
| IV | VLDL | TG, TC | N to ↑ | ↑↑ | 45 |
| V | VLDL & | TG, TC | ↑ to ↑↑ | ↑↑↑↑ | 5 |
| | chylomicron | | | | |

IDL, intermediate-density lipoprotein;
LDL, low-density lipoprotein;
N, normal;
TC, total cholesterol;
TG, triglyceride;
VLDL, very-low-density lipoprotein
*Approximate % of patients in the United States with hyperlipidemia.

Individuals may also be evaluated using a hs-CRP (high-sensitivity C-reactive protein) blood test. Those with a hs-CRP result of less than 1.0 mg/L are at low risk for cardiovascular disease. Individuals with a hs-CRP result between about 1.0 to 3.0 mg/L are at average risk for cardiovascular disease. Those with a hs-CRP result greater than 3.0 mg/L are at high risk of cardiovascular disease. Effective amounts of the compositions of the present present technology will lower hs-CRP results below 3.0 mg/L. Effective amounts of the compositions of the present technology can lower hs-CRP results by about 0.5 to about 3.0 mg/L, and further by about 0.5 to about 2.0 mg/L.

Effectiveness of the compositions and methods of the present technology may also be demonstrated by a decrease in the symptoms of cardiovascular disease, edema, diabetes insipidus, hypertension, myocardial ischemia, congestive heart failure, arrhythmia, and hyperlipoproteinemia, the symptoms including shortness of breath, chest pain, leg pain, tiredness, confusion, vision changes, blood in urine, nosebleeds, irregular heartbeat, loss of balance or coordination, weakness, or vertigo.

For each of the indicated conditions described herein, test subjects will exhibit a 10%, 20%, 30%, 50% or greater reduction, up to a 75-90%, or 95% or greater, reduction, in one or more symptom(s) caused by, or associated with, hyperlipidemia, elevated cholesterol, elevated triglyceride, and/or a targeted cardiovascular disease or condition in the subject, compared to placebo-treated or other suitable control subjects.

The compounds of the present technology can also be administered to a patient along with other conventional therapeutic agents that may be useful in the treatment or prophylaxis of hyperlipidemic diseases. In one aspect, a method is provided for administering an effective amount of one or more compounds of the present technology to a patient suffering from or believed to be at risk of suffering from a disease characterized by elevated plasma or hepatic cholesterol or triglycerides. Moreover, the present technology relates to treating a hyperlipidemic disease by administering an effective amount of one or more compounds to a patient in need thereof. The methods of the present technology can also comprise administering, either sequentially or in combination with one or more compounds of the present technology, a conventional therapeutic agent in an amount that can potentially or synergistically be effective for the treatment or prophylaxis of a hyperlipidemic disease. Exemplary therapeutic agents for use in combination therapies with one or more compounds of the present technology include, but are not limited to, anti-inflammatory drugs, therapeutic antibodies and cholesterol lowering drugs such as, for example, statins.

In one aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

Useful adjunctive therapeutic agents in combinatorial formulations and coordinate treatment methods include, for example, antihyperlipidemic agents; antidyslipidemic agents; antidiabetic agents, including, but not limited to metformin, rosiglitazone, plasma HDL-raising agents, including, but not limited to, nicotinic acid, fibrates; antihypercholesterolemic agents, including, but not limited to, cholesterol-uptake inhibitors; cholesterol biosynthesis inhibitors, e.g., HMG-CoA reductase inhibitors (also referred to as statins, such as lovastatin, simvastatin, pravastatin, fluvastatin, rosuvastatin, pitavastatin, and atorvastatin); HMG-CoA synthase inhibitors; squalene epoxidase inhibitors or squalene synthetase inhibitors (also known as squalene synthase inhibitors); microsomal triglyceride transfer protein (MTP) inhibitor; acyl-coenzyme A cholesterol acyltransferase (ACAT) inhibitors, including, but not limited to, melinamide; probucol; nicotinic acid and the salts thereof; niacinamide; cholesterol absorption inhibitors, including, but not limited to, beta-sitosterol or ezetimibe; bile acid sequestrant anion exchange resins, including, but not limited to cholestyramine, colestipol, colesevelam or dialkylaminoalkyl derivatives of a cross-linked dextran; LDL receptor inducers; fibrates, including, but not limited to, clofibrate, bezafibrate, fenofibrate and gemfibrozil; vitamin B6 (pyridoxine) and the pharmaceutically acceptable salts thereof, such as the HCl salt; vitamin B12 (cyanocobalamin); vitamin B3 (nicotinic acid and niacinamide); anti-oxidant vitamins, including, but not limited to, vitamin C and E and beta carotene; beta blockers; angiotensin II receptor (AT₁) antagonist; angiotensin-converting enzyme inhibitors, renin inhibitors; platelet aggregation inhibitors, including, but not limited to, fibrinogen receptor antagonists, i.e., glycoprotein IIb/IIIa fibrinogen receptor antagonists; hormones, including but not limited to, estrogen; insulin; ion exchange resins; omega-3 oils; benfluorex; ethyl icosapentate; and amlodipine. Adjunctive therapies may also include increase in exercise, surgery, and changes in diet (e.g., to a low cholesterol diet). Some herbal remedies may also be employed effectively in combinatorial formulations and coordinate therapies for treating hyperlipidemia, for example curcumin, gugulipid, garlic, soy, soluble fiber, fish oil, green tea, carnitine, chromium, coenzyme Q10, grape seed extract, pantothine, red yeast rice, and royal jelly.

Berberine and related compounds also can be employed as second therapeutic agents together with a compound of the present technology. For example, berberine sulfate, berberine hydrochloride, berberine chloride, oxyberberine, dihydroberberine, 8-cyanodihydroberberine, tetrahydroberberine N-oxide, tetrahydroberberine, 6-protoberberine, 9-ethoxycarbonyl berberine, 9-N,N-dimethylcarbamoyl berberine and 12-bromo berberine, berberine azide, and berberine betaine can be used. Berberine compounds that are effective in raising the expression level of LDLR are described in US 2006/0223838, which is hereby incorporated by reference in its entirety.

Another class of compounds that can be used as second therapeutic agents together with a compound of the present technology is the SCAP antagonists. These compounds bind to SREBP-cleavage activating protein and prevent its physical interaction with SREBP, resulting in activation of the LDLR promoter and increased expression of LDLR. Suitable compounds are described in U.S. Pat. No. 6,673,555 (which is hereby incorporated by reference in its entirety).

In some embodiments a compound of the present technology is combined with one or more sterol 14-reductase inhibitors as second agents. Such inhibitors will reduce the synthesis of cholesterol in the liver, and consequently contribute to the reduction of total cholesterol and LDL-cholesterol. A series of suitable 14-reductase inhibitors based on Corydalis alkaloids is described in U.S. Pat. No. 6,255,317 and U.S. Pat. No. 6,239,139, both of which are incorporated by reference in their entirety. It is noteworthy that the Corydalis alkaloids which function as 14-reductase inhibitors differ from the Corydalis lipid lowering agents of the present technology in having a double bond at the 13-14 position. In some embodiments of the present technology, however, the additional effect of inhibiting cholesterol synthesis may be undesired. In such cases, 14-reductase inhibitors, particularly those Corydalis alkaloids having a double bond at the 13-14 position, are specifically excluded from use with a Corydalis lipid lowering agent of the present technology.

A compound of the present technology can bind to one or more targets of interest with a dissociation constant (for example, an equilibrium dissociation constant, K_{d}) from, for example, about 0.0001 to 10 μM (or from 0.0001 to 7 μM, 0.0001 to 5 μM, 0.0001 to 1 μM, 0.001 to 5 μM, 0.01 to 5 μM and/or 0.1 to 5 μM) as measured by any suitable techniques routine to those of ordinary skill in the art. The present technology contemplates measurement of a dissociation constant (for example, K_{d} and Kᵢ) or performing competition, saturation and kinetics experiments by conventional techniques routine to one of ordinary skill in the art. Moreover, a compound of the present technology can compete with a reference compound for binding to and/or with targets of interest with a dissociation constant of inhibition (for example, Kᵢ) from, for example, about 0.01 nM to >10,000 nM (or from 0.001 to 7,000 nM, 0.001 to 5,000 nM, 0.001 to 1,000 nM, 0.01 to 5,000 nM, 0.01 to 2,000 nM and/or 0.1 to 5,000 nM).

In one aspect, binding, interaction or association with can mean the contact between a compound (or analogs, salts, pharmaceutical compositions, derivatives, metabolites, prodrugs or racemic, tautomers mixtures thereof) and a target of interest with a binding affinity of at least 10⁻⁶ M, preferably, at least about 10⁻⁷ M, and more preferably 10⁻⁸ M to 10⁻⁹ M, 10⁻¹⁰ M, 10⁻¹¹ M, or 10⁻¹² M. In one aspect, binding affinities include those with a dissociation constant or K_{d} less than, but not limited to, 5×10⁻⁶ M, 10⁻⁶ M, 5×10⁻⁷ M, 10⁻⁷ M, 5×10⁻⁸ M, 10⁻⁸ M, 5×10⁹ M, 10⁻⁹ M, 5×10⁻¹⁰ M, 10⁻¹⁰ M, 5×10⁻¹¹ M 10⁻¹¹ M, 5×10⁻¹² M 10⁻¹² M, 5×10⁻¹³ M, 10⁻¹³ M, 5×10⁻¹⁴ M, 10⁻¹⁴ M, 5×10⁻¹⁵ M, and 10⁻¹⁵ M.

A compound of the present technology can also be modified, for example, by the covalent attachment of an organic moiety or conjugate to improve pharmacokinetic properties, toxicity or bioavailability (e.g., increased in vivo half-life). The conjugate can be a linear or branched hydrophilic polymeric group, fatty acid group or fatty acid ester group. A polymeric group can comprise a molecular weight that can be adjusted by one of ordinary skill in the art to improve, for example, pharmacokinetic properties, toxicity or bioavailability. Exemplary conjugates can include a polyalkane glycol (e.g., polyethylene glycol (PEG), polypropylene glycol (PPG)), carbohydrate polymer, amino acid polymer or polyvinyl pyrrolidine and a fatty acid or fatty acid ester group, each of which can independently comprise from about eight to about seventy carbon atoms. Conjugates for use with a compound of the present technology can also serve as linkers to, for example, any suitable substituents or groups, radiolabels (marker or tags), halogens, proteins, enzymes, polypeptides, other therapeutic agents (for example, a pharmaceutical or drug), nucleosides, dyes, oligonucleotides, lipids, phospholipids and/or liposomes. In one aspect, conjugates can include polyethylene amine (PEI), polyglycine, hybrids of PEI and polyglycine, polyethylene glycol (PEG) or methoxypolyethylene glycol (mPEG). A conjugate can also link a compound of the present technology to, for example, a label (fluorescent or luminescent) or marker (radionuclide, radioisotope and/or isotope) to comprise a probe of the present technology. Conjugates for use with a compound of the present technology can, in one aspect, improve in vivo half-life. Other exemplary conjugates for use with a compound of the present technology as well as applications thereof and related techniques include those generally described by U.S. Pat. No. 5,672,662, which is hereby incorporated by reference herein.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

The examples herein are provided to illustrate advantages of the present technology and to further assist a person of ordinary skill in the art with preparing or using the compounds of the present technology or salts, pharmaceutical compositions, derivatives, metabolites, prodrugs, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

Chemicals were purchased from the Sigma-Aldrich Chemical Company (Milwaukee, Wis.) or similar commercial suppliers. Solvents were purchased from VWR International (Brisbane, Calif.) or similar commercial suppliers. Proton and ¹³C NMR spectra were performed on a 300 MHz Bruker AC-300 plus NMR spectrometer with a TCPLink PC upgrade (INAC Computer, GmbH, Malsch, Germany) or comparable instrument. The NMR solvent was CDCl₃ unless otherwise specified. HPLC was performed using Waters 600 pumps and controller with a Waters 996 photodiode array detector or a comparable instrument. Solvent A was 0.05% trifluoroacetic acid in water. Solvent B was 0.04% trifluoroacetic acid in acetonitrile. The gradient was 0 to 100% B over 30 minutes, 2 mL/min. flow rate. The column was a C-18 reverse phase Vydac 254TP18 column of 25×0.46 cm or comparable reverse-phase columns. Flash chromatography was performed on a Teledyne Isco (Lincoln, Nebr.) CombiFlash Companion automated workstation or manually. FT-IR spectra were obtained on a Perkin-Elmer FT-1600 spectrophotometer or comparable instrument, and melting points were determined on a Cole Palmer Kofler block melting point apparatus or comparable instrument. Optical rotations were determined on a Perkin-Elmer 241 Polarimeter or comparable instrument; optical rotation samples were dissolved in a suitable solvent such as, e.g., ethanol prior to measurement.

### Example 1

### Synthesis of 14R-Tetrahydropalmatine (THP) from Berberine (BBR)

14R-THP was prepared from BBR in four steps (see scheme below) starting by treating BBR with boron trichloride in methylene chloride. This deprotected only the methylene bridged catechol leaving the methoxy groups untouched. Methylation with methyl iodide and potassium carbonate in dry acetone then afforded the tetra-O-Me compound that was subsequently subjected to asymmetric hydrogenation with a suitable asymmetric hydrogenation catalyst to afford 14R-THP. The S-enantiomer may be similarly obtained. In addition, acid addition salts of 14R-THP may be prepared by exposure to acid during the hydrogenation or afterwards as a separate step.

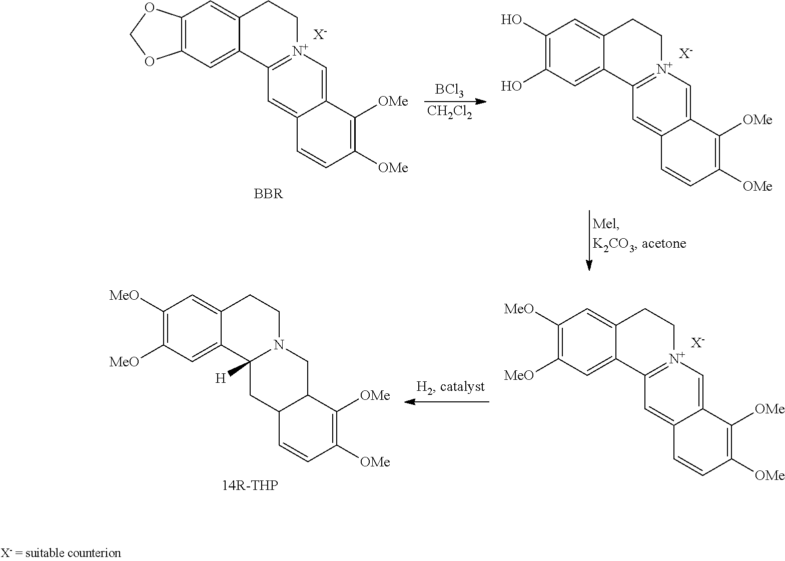

Exemplary catalysts that can be used for the synthesis are generally described by: Bunlaksananusorn, T., Polborn, K., Knochel, P., “New P,N ligands for asymmetric Ir-catalyzed reactions,” Angew. Chemie, Intl. Ed. (2003), 42(33), 941-3943; Lu, S.-M., Han, X.-W., Zhou, Y.-G., “Asymmetric hydrogenation of quinolines catalyzed by iridium with chiral ferrocenyloxazoline derived N,P ligands,” Advanced Synthesis & Catalysis (2004), 346(8), 909-912; Lu, S.-M., Wang, Y.-Q., Han, X.-W., Zhou, Y.-G., “Asymmetric hydrogenation of quinolines and isoquinolines activated by chloroformates,” Angew. Chemie, Intl. Ed. (2006), 45(14), 2260-2263; Wang, D.-W., Zeng, W.; Zhou, Y.-G., “Iridium-catalyzed asymmetric transfer hydrogenation of quinolines with Hantzsch esters,” Tetrahedron: Asymmetry (2007), 18(9), 1103-1107; Xu Lijin; Lam Kim Hung; Ji Jianxin; Wu Jing; Fan Qing-Hua; Lo Wai-Hung; Chan Albert S C “Air-stable Ir-(P-Phos) complex for highly enantioselective hydrogenation of quinolines and their immobilization in poly(ethylene glycol) dimethyl ether (DMPEG),” Chem. Comm. (Cambridge, England) (2005), (11), 1390-2; and Wang, Y., Weissensteiner, W., Spindler, F., Anion, V. B., and Mereiter, K., “Synthesis and Use in Asymmetric Hydrogenations of Solely Planar Chiral 1,2-Disubstituted and 1,2,3-Trisubstituted Ferrocenyl Diphosphines: A Comparative Study,” Organometallics, 2007, each of which is incorporated herein by reference in their entirety.

### Example 2

### Design and Synthesis of Compounds

1. Preparation of Berberrubine from Berberine.

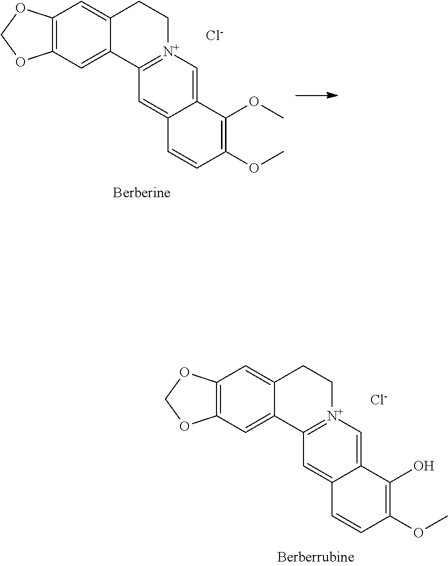

Berberine (1.0 g, 2.68 mmol) was heated at 190° in a dry oven under vacuum for 30 minutes. The crude product was recrystallized from EtOH to give berberrubine (0.6 g, yield 60%, confirmed by ¹HNMR).

2. Preparation of Compound 67 from Corypalmine.

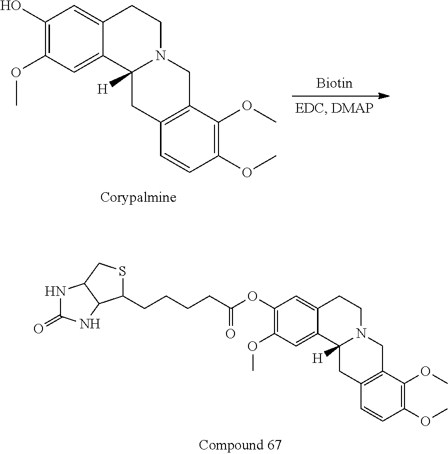

D-Biotin (105 mg, 0.43 mmol), EDC•HCl (125 mg, 0.65 mmol) and DMAP (19 mg, 0.16 mmol) were dissolved together in a flask with a minimum volume of DMF (4.5 mL). Then corypalmine (25 mg, 0.073 mmol) was dissolved in this solution. After stirring for 3 hours, 0.5 mL sample of the reaction solution was taken out for testing. The sample was added to 10 mL H₂O and extracted with 10 mL EtOAc. The organic layer was dried with MgSO₄, filtered, and concentrated under vacuum. The product was subjected to LC-MS. The LC-MS information suggested formation of compound 67. The remaining reaction mixture was stirred overnight. The reaction mixture was subsequently extracted with EtOAc. The organic layer was dried with MgSO₄ and evaporated in vacuo. The yellow residue was isolated by preparative TLC to afford compound 67. Preparative HPLC was used to purify compound, 67. The purified product was confirmed by MS and HPLC to confirm the structure and the purity (92.1% and 93.1%).

3. Preparation of Compound I2 from Corypalmine.

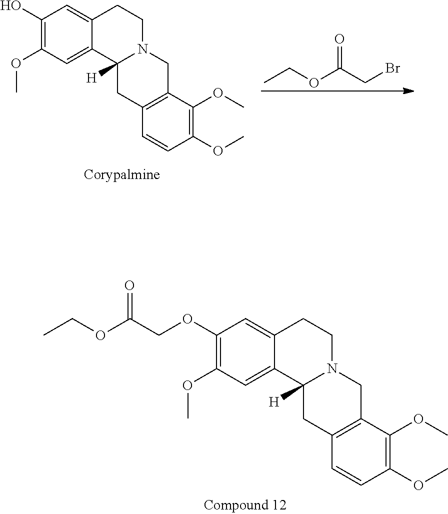

Corypalmine (0.068 g, 0.2 mmol) was added to 20 mL acetone and 5 mL ethanol. The suspension was refluxed for 1 hour to dissolve the starting material. Then 0.068 mg K₂CO₃ was added and the suspension was refluxed for 1 hour. Ethyl 2-bromoacetate (0.0244 mL, 0.22 mmol) was dissolved in 1 mL acetone and added into the reaction suspension in portions over 30 minutes. The resulting suspension was refluxed for 2 hours. The reaction was monitored by LC-MS. Part of the product was purified by preparative TLC.

4. Preparation of Compound 68 from Compound I2.

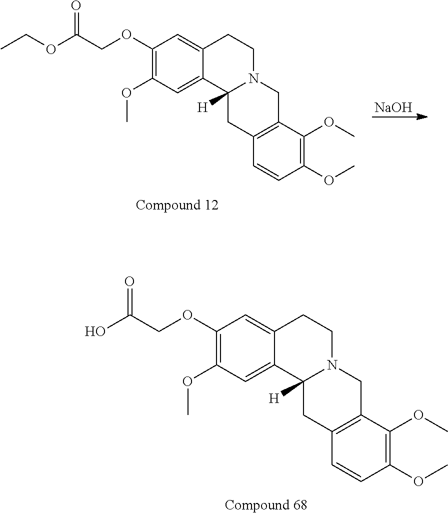

Compound I2, prepared according to procedure 4 was used without purification, and saponified with NaOH to prepare compound 68. This reaction was monitored by LC-MS. After standard workup.

5. Preparation for Compound I3 from Berberrubine.

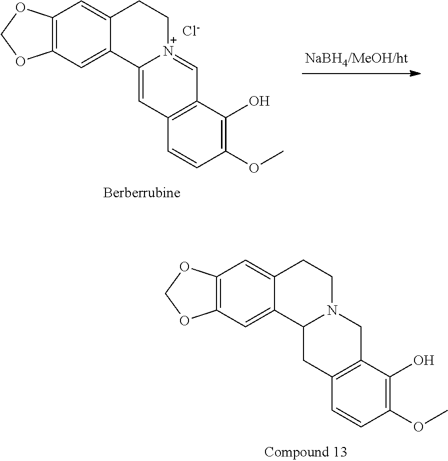

Berberrubine (60 mg, 0.1 mmol) was added to 7 mL hot MeOH and stirred for 15 minutes at 60° C. Then NaBH₄ (8 mg, 0.21 mmol) was added to the mixture and the mixture was stirred at 60° for 15 minutes. Five mL H₂O was added to the solution to quench the reaction. The product I3 was extracted from the solution with CHCl₃ (10 mL×3). 10 mg grey solid was obtained, yield 20%. The isolated material gave the expected peak in MS and confirmed the structure. HPLC analysis suggested the purity was satisfactory for use without further purification.

6. Preparation for Compound 77 from 14R-(+)-THP.

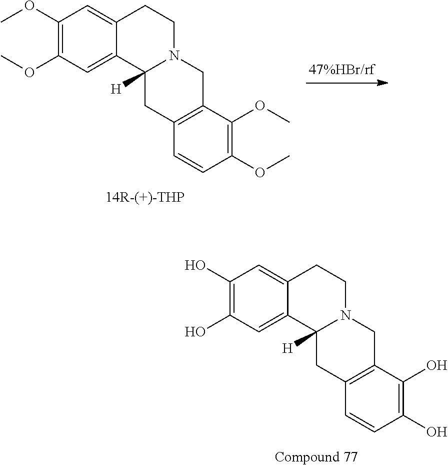

14R-(+)-THP (250 mg, 0.70 mmol) was added to 15 mL 47% HBr and stirred overnight at 100° C. Then the solution was cooled to room temperature, and the product was filtered off to give compound 77 as the hydrobromide salt.

7. Preparation for Compound 78 from 14R,13S-(+)-CDRL.

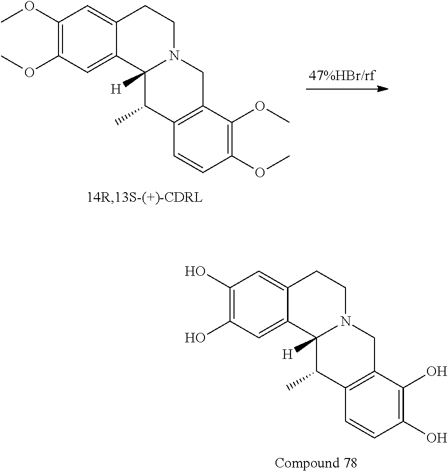

14R,13S-CDRL (200 mg, 0.54 mmol) was added to 10 mL 47% HBr and stirred overnight at 100° C. Then the solution was cooled to rt, and the product was filtered off to give compound 78 as the hydrobromide salt.

8. Preparation for Compound 79 from Berberrubine.

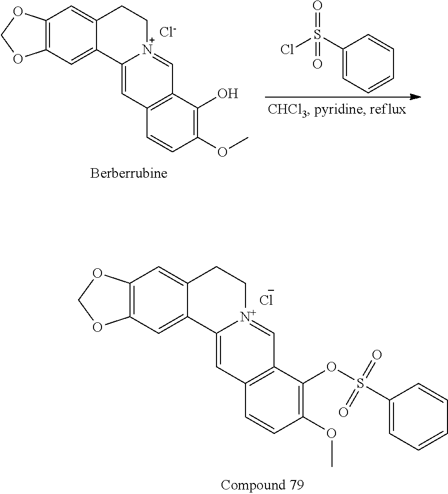

Berberrubine (248 mg, 0.693 mmol) was added to 5 mL CHCl₃ in a flask. The mixture was stirred and refluxed. Then benzenesulfonyl chloride (760 mg, 4.30 mmol) and pyridine (0.1 mL) were slowly added in. The reaction mixture was stirred at the same temperature for 2 hours, and subsequently cooled down to room temperature. The mixture was filtered to afford a yellow solid. The solid was washed with CHCl₃ 3 times and dried under vacuum to provide the final product, compound 79, (yellow solid, 204 mg). Compound 79 was used directly for the next step without purification.

9. Preparation for Compound 69 from Compound 79.

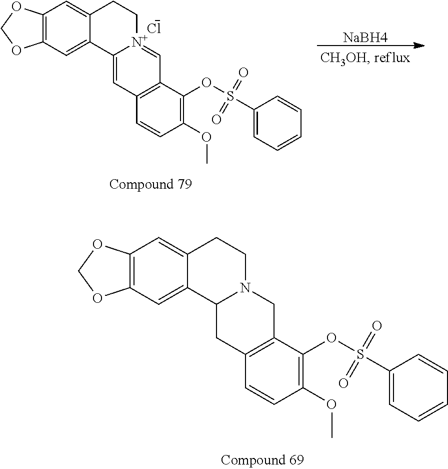

Compound 79 (132 mg, 0.265 mmol) was added in a flask with methanol (5 mL). Then the reaction mixture was heated to reflux to dissolve the starting material. Then NaBH₄ (42 mg, 1.11 mmol) was added slowly to the flask. The reaction mixture was stirred at the same temperature for 1 hour. Then it was cooled down to room temperature and then cooled in refrigerator for 4 hours. The mixture was filtered to afford light yellow crystals. The crystals were washed with H₂O 3 times and then dried under vacuum to provide the final product compound 69 (light yellow solid, 28.7 mg). NMR & MS analyses were consistent with the structure of compound 69.

Analogs of compound 69 may be readily made using commercially available substituted phenyl sulfonyl chlorides.

10. Preparation for Compound 80 from Berberrubine.

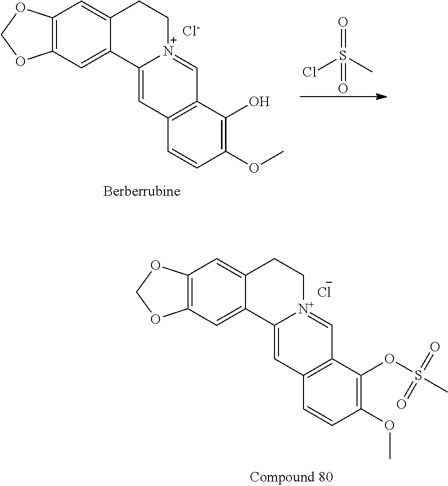

Berberrubine (0.5 g, 1.4 mmol) was dissolved in 40 mL CHCl₃ by refluxing. After stirring for about 30 minutes, the methanesulfonyl chloride (0.33 mL, 4.2 mmol) was added to the solution dropwise over 30 seconds. 10 minutes later, yellow solid appeared. The suspension was refluxed for 3 hours. After cooling, the suspension was filtered to provide a yellow solid. This intermediate (compound 80) was used directly for the next step without purification.

11. Preparation for Compound 71 from Compound 80.

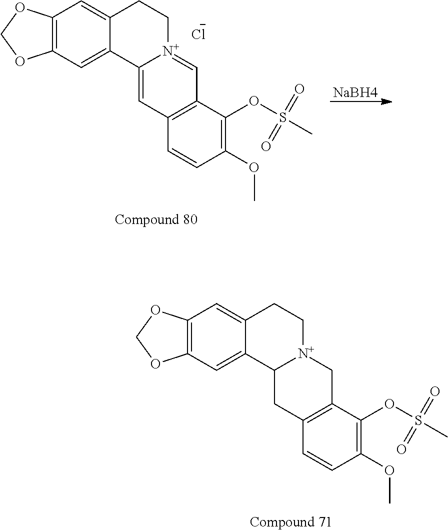

Compound 80 was dissolved in 30 mL MeOH at reflux. NaBH₄ (0.052 g, 1.3 mmol) was added to the reaction. The reaction occurred immediately. The solution was refluxed for 2 hours and cooled down. Analysis by TLC suggested the transformation was complete. The reaction was left overnight, and a gray solid appeared the next morning. The suspension was filtered to afford the gray solid. NMR & MS were consistent with the structure of the compound 71.

12. Preparation of Compound I9 from Berberrubine.

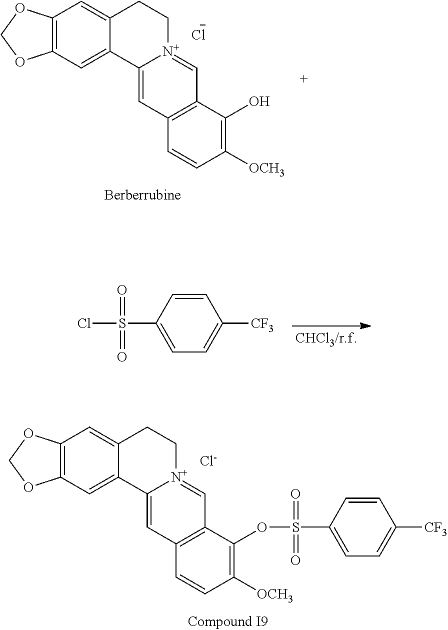

Berberrubine (300 mg, 0.84 mmol) was dissolved in 30 mL CHCl₃ at reflux. Then 4-(trifluoromethyl)benzene-1-sulfonyl chloride (300 mg, 1.23 mmol) was added to the solution and stirred for 7 hours. Then product was filtered, washed with CHCl₃ and dried. 400 mg product was obtained as yellow solid (yield: 84.2%). The intermediate was used directly without further purification.

13. Preparation of Compound 85 from Compound I9.

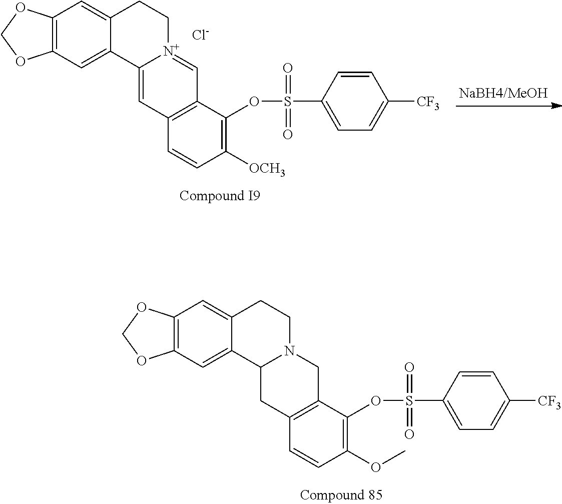

Compound I9 (140 mg, 0.247 mmol) was dissolved in 80 mL MeOH at reflux. NaBH₄ (50 mg, 1.322 mmol) was added to the solution and stirred for 1 hour. Then product was filtered, washed with MeOH, H₂O and hexane. 45 mg product was obtained as gray solid (yield: 34%). The MS & ¹H NMR analysis of the product was consistent with the structure of compound 85.

14. Preparation of Compound I10 from Berberrubine.

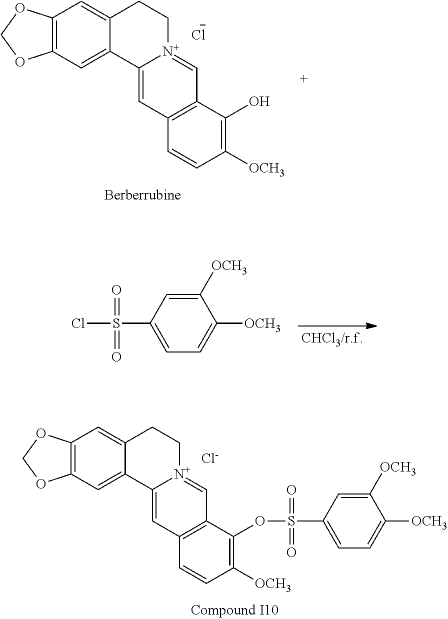

Berberrubine (300 mg, 0.84 mmol) was dissolved in 30 mL CHCl₃ at 70° C. Then 3,4-dimethoxybenzene-1-sulfonyl chloride (300 mg, 1.27 mmol) was added to the solution and stirred for 7 hours. Then product was filtered, washed by CHCl₃ and dried. 40 mg product was obtained as yellow solid. (yield: 17%). The intermediate was used directly without further purification.

15. Preparation of Compound 86 from Compound I10.

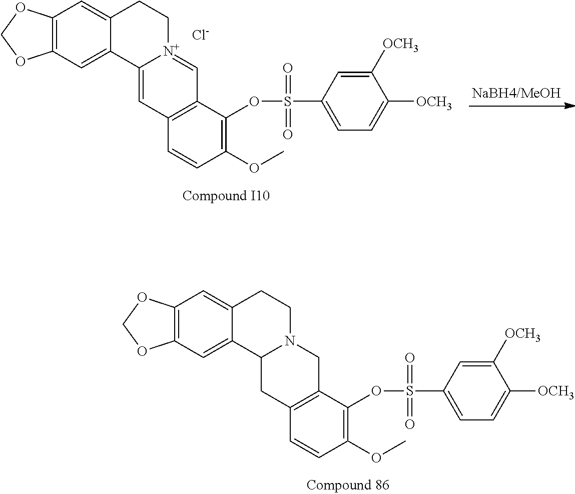

Compound I10 (80 mg, 0.14 mmol) was dissolved in 80 mL MeOH at 70° C. NaBH₄ (50 mg, 1.32 mmol) was added to the solution and stirred for 1 hours. Then product was filtered, washed with MeOH, H₂O and hexane. 25 mg product was obtained as gray solid. (MC0236-38-1; yield: 34%). The MS & ¹H NMR information suggested the product was the desired structure, compound 86.

16. Preparation of Compound I11 from Berberrubine.

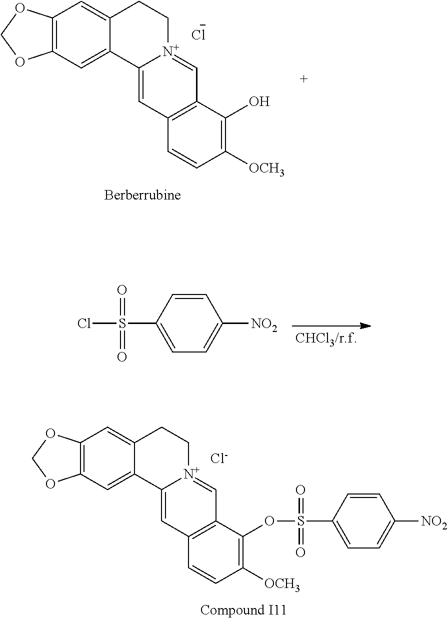

Berberrubine (300 mg, 0.84 mmol) was dissolved in 30 mL CHCl₃ at 70° C. Then 4-nitrobenzene-1-sulfonyl chloride (300 mg, 1.35 mmol) was added to the solution and stirred for 7 hours. Then product was filtered, washed by CHCl₃ and dried. 400 mg product was obtained as yellow solid. (yield: 87.7%) The intermediate was used directly without further purification.

17. Preparation of Compound 106 from Compound I11.

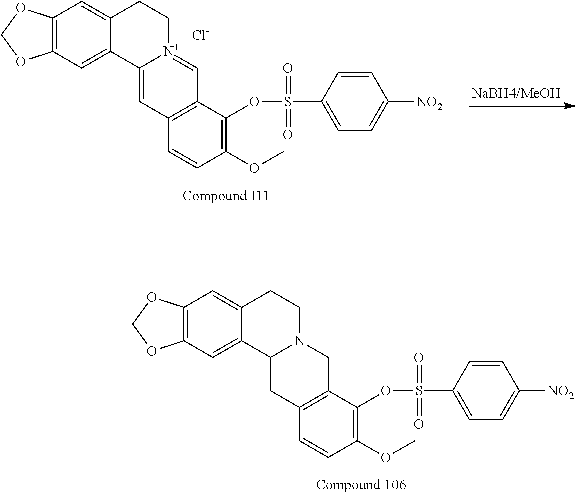

Compound I11 (110 mg, 0.202 mmol) was dissolved in CH₃OH (150 mL) at reflux in a 250 mL three-neck flask. Then sodium borohydride (100 mg, 2.64 mmol) was added carefully to the reaction. The mixture was refluxed for 3 hours. The solution was concentrated to 10 mL in vacuum, cooled in refrigerator, filtered, washed by water and n-hexane, dried in vacuum. 35 mg product was obtained as cyan solid. (yield: 33.9%)

18. Preparation of Compound I13 from Berberrubine.

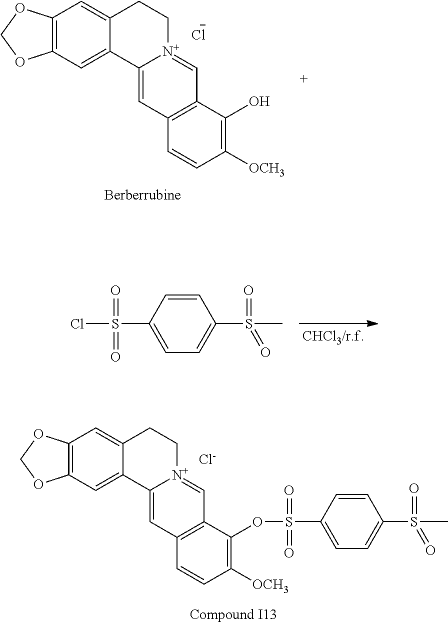

Berberrubine (200 mg, 0.56 mmol) was dissolved in 30 mL CHCl₃ at 70° C. Then 4-(methylsulfonyl)benzene-1-sulfonyl chloride (350 mg, 1.37 mmol) was added to the solution and stirred for 7 hours at 70° C. Then product was filtered, washed by CHCl₃ and dried. 200 mg product was obtained as yellow solid. (yield: 62%) The intermediate compound 113 was used directly without further purification.

19. Preparation of Compound 87 from Compound I13.

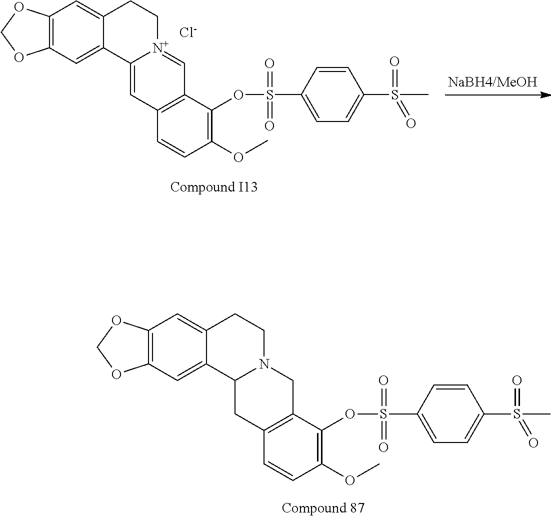

Compound I13 (110 mg, 0.19 mmol) was dissolved in 80 mL MeOH at reflux. Then NaBH₄ (70 mg, 1.85 mmol) was added to the solution and stirred for 1 hour at 70° C. Then most of solvent was removed, and product was filtered, washed by MeOH, H₂O and hexane. 40 mg product was obtained as gray solid (yield: 38.8%).

20. Preparation of Compound I14 from Berberrubine.

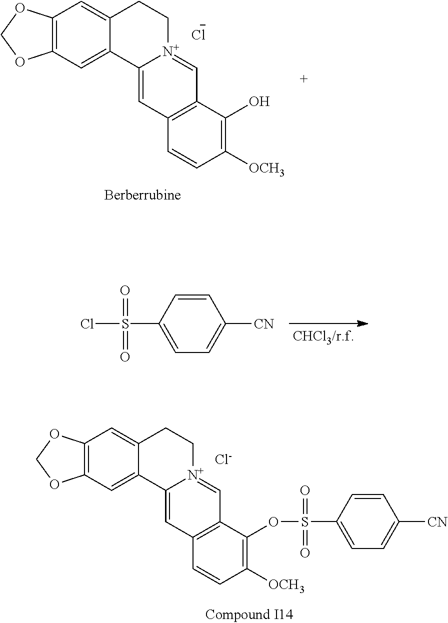

Berberrubine (220 mg, 0.61 mmol) was dissolved in 30 mL CHCl₃ at 70° C. Then 4-cyanobenzene-1-sulfonyl chloride (340 mg, 1.68 mmol) was added to the solution and stirred for 7 hours at 70° C. Then product was filtered, washed by CHCl₃ and dried. 110 mg product was obtained as yellow solid. (yield: 35%) The intermediate compound I14 was used directly without further purification.

21. Preparation of Compound 108 from Compound I14.

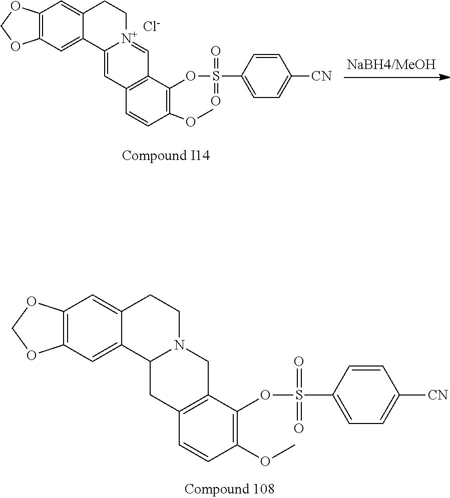

### 22. Preparation of Compound 1.

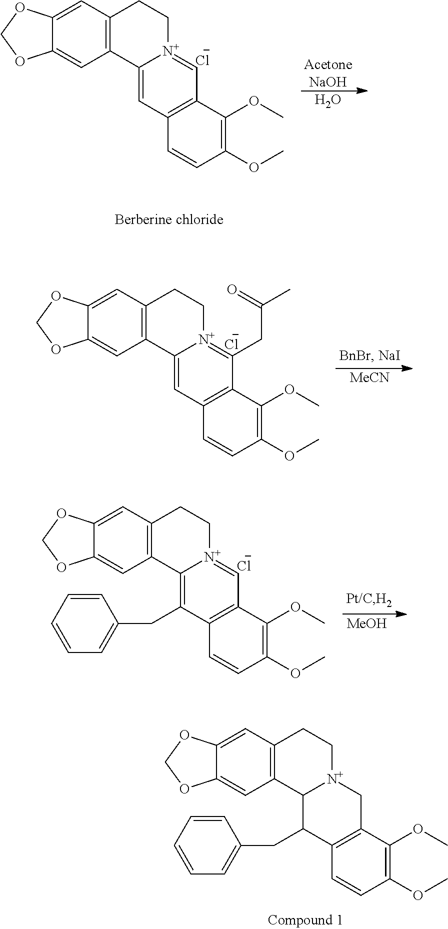

### Berberine Chloride→B

To a stirred solution of NaOH (4 g, 100 mmol) in water (20 mL) was added berberine chloride (2.0 g, 5.4 mmol) at room temperature. Then acetone (1.6 mL) was added slowly at same temperature and stirred for 1 hour. TLC analysis indicated the completion of the reaction. The reaction solution was filtered, sufficiently washed with 80% methanol and dried to give 1.7 g of B.

### B→C

To a stirred solution of B (1.7 g, 4.3 mmol) in MeCN (20 mL) was added KI (450 mg, 2.7 mmol) at room temperature. The reaction mixture was heated to reflux and BnBr (1.5 mL, 12.7 mmol) was added. Then the reaction was refluxed for 4 hours with stirring. TLC analysis indicated the completion of the reaction. The solvent was removed and residue was purified by column chromatography to give 1.6 g of C.

### C→Compound 1

To a stirred solution of C (1.6 g, 3.7 mmol) in MeOH was added Pt/C (200 mg) and stirred overnight under H₂ at ambient temperature. After filtering, the filtrate was concentrated to give crude product. The solid was washed with MeOH to give 500 mg of pure Compound 1. ¹H NMR (CDCl₃): δ 7.20-7.14 (m, 3H); 6.88-6.85 (m, 2H); 6.76 (s, 1H); 6.61 (s, 1H); 6.52 (d, 1H, J=8.4 Hz); 6.00 (d, 1H, J=8.4 Hz); 5.94 (s, 2H); 4.28 (d, 1H, J=16.2 Hz); 3.86 (s, 3H); 3.80 (s, 3H); 3.77 (br, 1H); 3.56 (d, 1H, J=16.2 Hz); 3.25-3.07 (m, 3H); 2.73-2.57 (m, 4H). MS: m/z (APCI-ESI) 430.2 (M⁺+1).

### 23. Preparation of Compounds, Salt Formation.

General Procedure: 14R,13S-(+)-CRDL or 14R-(+)-THP was dissolved in solvent and mixed with a suitable amount of acid to form the corresponding acid addition salt.

### Hydrochloride Salts (Compounds 2 and 6)

To a stirred solution of MeOH (2 mL) and DCM (2 mL) contained HCl (2 mmol) was added 14R-(+)-THP (100 mg, 0.28 mmol) or 14R,13S-(+)-CDRL (100 mg, 0.27 mmol) at room temperature and stirred for 2 hours. The solvent was removed to give 110 mg of the salt.

### Sulfate Salts (Compounds 3 and 7)

To a stirred solution of MeOH (2 mL) and DCM (2 mL) contained H₂SO₄ (15 μL, 0.28 mmol) was added 14R-(+)-THP (100 mg, 0.28 mmol) or 14R,13S-(+)-CDRL (100 mg, 0.27 mmol) at room temperature and stirred for 2 hours. The solvent was removed to give 110 mg of the salt.

### Citrate Salts (Compounds 4 and 8)

To a stirred solution of MeOH (2 mL) and DCM (2 mL) contained citric acid (19.6 mg, 0.093 mmol) was added 14R-(+)-THP (100 mg, 0.28 mmol) or 14R,13S-(+)-CDRL (100 mg, 0.27 mmol) at room temperature and stirred for 2 hours. The solvent was removed to give 110 mg of the salt.

### Maleate Salts (Compounds 5 and 9)

To a stirred solution of MeOH (2 mL) and DCM (2 mL) contained maleic acid (16.2 mg, 0.14 mmol) was added 14R-(+)-THP (100 mg, 0.28 mmol) or 14R,13S-(+)-CDRL (100 mg, 0.27 mmol) at room temperature and stirred for 2 hours. The solvent was removed to give 110 mg of the salt.

### 24. Preparation of Compounds 27 and 28.

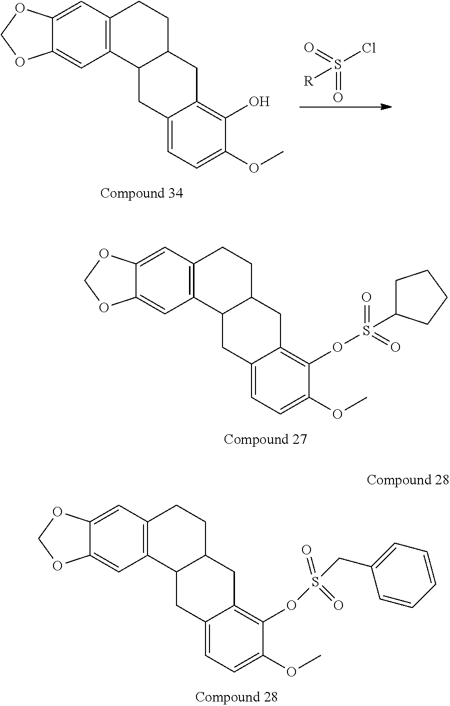

To a solution of compound 34 (50 mg, 0.154 mmol) in CH₂Cl₂ was added sulfonyl chloride (0.185 mmol), and Na₂CO₃ (20 mg), then the mixture was stirred overnight at room temperature in small ampoule. Then it was purified by preparative TLC to afford the product.

Compound 27: ¹H NMR (CD₃OH, 300 MHz): δ 7.03 (d, J=8.4 Hz, 1H), 6.83 (d, J=8.7 Hz, 1H), 6.71 (s, 1H), 6.59 (s, 1H), 5.91 (dd, J=1.5, 2.1 Hz, 2H), 4.27 (d, J=16.2 Hz, 1H), 4.00-3.89 (m, 1H), 3.87 (s, 3H), 3.83-3.60 (m, 2H), 3.27-3.16 (m, 3H), 2.85-2.64 (m, 3H), 2.32-2.10 (m, 4H), 2.95-2.65 (m, 4H); MS: m/z=326.1 (M⁺+1).

Compound 28: ¹H NMR (CDCl₃, 300 MHz): δ 7.55-7.7.51 (m, 5H), 7.06 (d, J=8.4 Hz, 1H), 6.87 (d, J=8.4 Hz, 1H), 6.70 (s, 1H), 6.57 (s, 1H), 5.08 (dd, J=1.2, 2.1 Hz, 2H), 4.75 (q, J=16.5 Hz, 2H), 4.19 (d, J=15.9 Hz, 3H), 3.64-3.54 (m, 2H), 3.26-3.3.02 (m, 3H), 2.86-2.77 (m, 1H), 2.65-2.55 (m, 2H); MS: m/z=480.0 (M⁺+1).

### 25. Preparation of Compounds 37, 38 and 42.

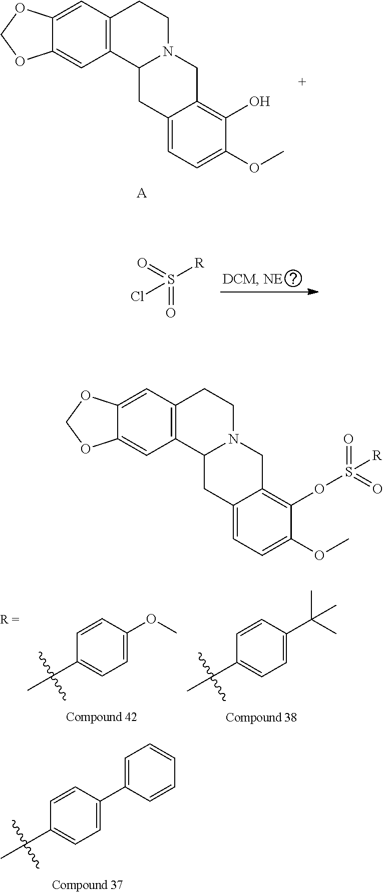

To a solution of A (50 mg, 0.15 mmol) in CH₂Cl₂ was added chlorosulfone (0.20 mmol), and two drops of Et₃N, then the mixture was stirred for 3 hours at room temperature in a small ampoule. Then it was purified by preparative TLC to afford the title compounds.

Compound 37: ¹H NMR (CDCl₃, 300 MHz): δ 8.09-7.44 (m, 9H), 7.02 (d, J=8.4 Hz, 1H), 6.72 (d, J=8.4 Hz, 1H), 6.71 (s, 1H), 6.59 (s, 1H), 5.92 (s, 2H), 4.25 (d, J=15.9 Hz, 1H), 3.68-3.56 (m, 2H), 3.45 (s, 3H), 3.28-3.08 (m, 3H), 2.87-2.60 (m, 3H); MS: m/z=542.1 (M⁺+1).

Compound 38: ¹H NMR (CDCl₃, 300 MHz): δ 7.93-7.55 (m, 4H), 7.01 (d, J=8.4 Hz, 1H), 6.72-6.69 (m, 3H), 5.91 (s, 2H), 4.19 (d, J=19.5 Hz, 1H), 3.61-3.54 (m, 2H), 3.43 (s, 3H), 3.24 (dd, J=3.9, 15.9 Hz, 1H), 3.12-3.02 (m, 2H), 2.86-2.51 (m, 3H); MS: m/z=522.1 (M⁺+1).

Compound 42: ¹H NMR (CDCl₃, 300 MHz): δ 7.93 (d, J=2.1 Hz, 1H), 7.91-7.90 (d, J=2.4 Hz, 1H), 7.02-6.99 (m, 3H), 6.72 (d, J=9.6 Hz, 2H), 6.58 (s, 1H), 5.91 (s, 2H), 4.23 (d, J=17.1 Hz, 1H), 3.90 (s, 3H), 3.64-3.59 (m, 2H), 3.50 (s, 3H), 3.27-3.21 (m, 1H), 3.15-3.10 (m, 2H), 2.85-2.59 (m, 3H); MS: m/z=490.0 (M⁺+1).

### 26. Preparation of Compounds 49-53 and 56-60.

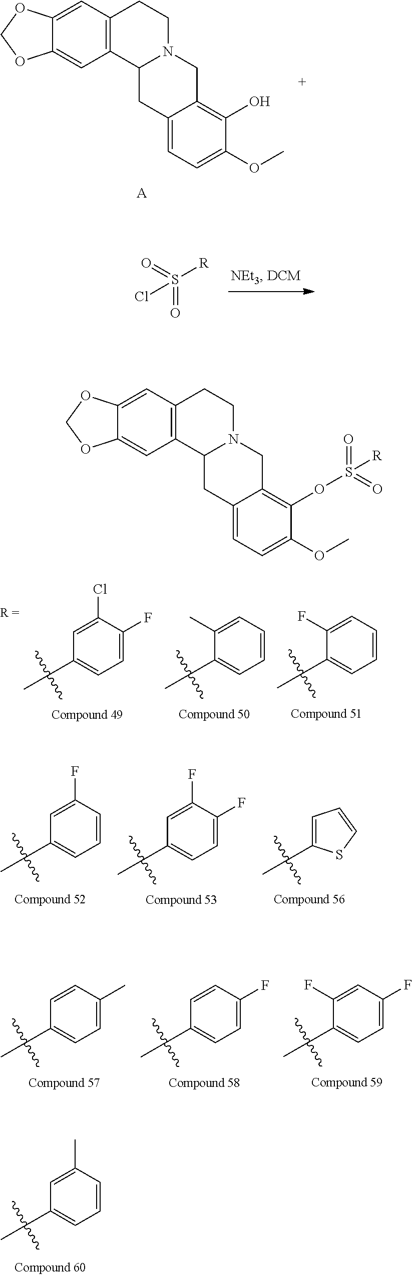

To a solution of A (50 mg, 0.15 mmol) in CH₂Cl₂ was added chlorosulfone (0.20 mmol), and two drops of Et₃N; then the mixture was stirred for 3 hours at room temperature in a small ampoule. The reaction mixture was purified by preparative TLC to afford the title compounds.

Compound 49: ¹H NMR (CDCl₃, 300 MHz): δ 8.00 (dd, J=5.7, 9.0 Hz, 1H), 7.38 (dd, J=2.4, 8.4 Hz, 1H), 7.13-7.07 (m, 1H), 7.05 (d, J=8.4 Hz, 1H), 6.71-6.68 (m, 2H), 6.59 (s, 1H), 5.91 (s, 2H), 4.26 (d, J=15.6 Hz, 1H), 3.65-3.56 (m, 2H), 3.39 (s, 3H), 3.26-3.09 (m, 3H), 2.69-2.61 (m, 3H); MS: m/z=517.9 (M⁺+1).

Compound 50: ¹H NMR (CDCl₃, 300 MHz): δ 7.89-7.86 (dd, J=1.2, 7.6 Hz, 1H), 7.55-7.50 (m, 1H), 7.41 (d, J=7.5 Hz, 1H), 7.32-7.27 (m, 1H), 6.99 (d, J=8.4 Hz, 1H), 6.70-6.66 (m, 2H), 6.58 (s, 1H), 5.91 (s, 2H), 4.18 (d, J=15.9 Hz, 1H), 3.59-3.54 (m, 2H), 3.32 (s, 3H), 3.26-3.07 (m, 3H), 2.82 (s, 3H), 2.66-2.56 (m, 3H); MS: m/z=480.0 (M⁺+1).

Compound 51: ¹H NMR (CDCl₃, 300 MHz): δ 7.88-7.82 (m, 1H), 7.71-7.63 (m, 1H), 7.34-7.28 (m, 2H), 7.01 (d, J=8.7 Hz, 1H), 6.68 (s, 1H), 6.68 (d, J=8.1 Hz, 1H), 6.59 (s, 1H), 5.91 (s, 2H), 4.27 (d, J=15.9 Hz, 1H), 3.68-3.56 (m, 2H), 3.35 (s, 3H), 3.26-3.08 (m, 3H), 2.86-2.58 (m, 3H); MS: m/z=484.0 (M⁺+1).

Compound 52: ¹H NMR (CDCl₃, 300 MHz): δ 7.82-7.78 (m, 1H), 7.74-7.70 (m, 1H), 7.59-7.52 (m, 1H), 7.42-7.36 (m, 1H), 7.03 (d, J=8.1 Hz, 1H), 6.74-6.70 (m, 2H), 6.59 (s, 1H), 5.91 (s, 2H), 4.24 (d, J=15.9 Hz, 1H), 3.67-3.57 (m, 2H), 3.45 (s, 3H), 3.28-3.09 (m, 3H), 2.86-2.59 (m, 3H); MS: m/z=484.0 (M⁺+1).

Compound 53: ¹H NMR (CDCl₃, 300 MHz): δ 7.91-7.77 (m, 2H), 7.40-7.32 (m, 1H), 7.05 (d, J=8.4 Hz, 1H), 6.75-6.71 (m, 2H), 6.59 (s, 1H), 5.91 (s, 2H), 4.25 (d, J=15.6 Hz, 1H), 3.70-3.58 (m, 2H), 3.50 (s, 3H), 3.27-3.05 (m, 3H), 2.88-2.61 (m, 3H); MS: m/z=502.0 (M⁺+1).

Compound 56: ¹H NMR (CDCl₃, 300 MHz): δ 7.78-7.73 (m, 2H), 7.16-7.13 (m, 1H), 7.04 (d, J=8.4 Hz, 1H), 6.77-6.70 (m, 2H), 6.59 (s, 1H), 5.91 (s, 2H), 4.22 (d, J=15.9 Hz, 1H), 3.63-3.55 (m, 2H), 3.59 (s, 3H), 3.27-3.04 (m, 3H), 2.86-2.60 (m, 3H); MS: m/z=472.0 (M⁺+1)

Compound 57: ¹H NMR (CDCl₃, 300 MHz): δ 7.89-7.85 (m, 2H), 7.36-7.33 (m, 2H), 7.01 (d, J=8.7 Hz, 1H), 6.71 (d, J=7.2 Hz, 1H), 6.70 (s, 1H), 6.58 (s, 1H), 5.91 (s, 2H), 4.20 (d, J=15.9 Hz, 1H), 3.62-3.54 (m, 2H), 3.45 (s, 3H), 3.28-3.02 (m, 3H), 2.85-2.53 (m, 3H), 2.47 (s, 3H); MS: m/z=480.0 (M⁺+1)

Compound 58: ¹H NMR (CDCl₃, 300 MHz): δ 8.04-7.99 (m, 2H), 7.27-7.21 (m, 2H), 7.02 (d, J=8.4 Hz, 1H), 6.71 (d, J=7.5 Hz, 1H), 6.70 (s, 1H), 6.59 (s, 1H), 5.91 (s, 2H), 4.24 (d, J=15.9 Hz, 1H), 3.67-3.56 (m, 2H), 3.46 (s, 3H), 3.28-3.03 (m, 3H), 2.86-2.59 (m, 3H); MS: m/z=484.0 (M⁺+1)

Compound 59: ¹H NMR (CDCl₃, 300 MHz): δ 7.90-7.82 (m, 1H), 7.08-6.97 (m, 3H), 6.71-6.68 (m, 2H), 6.59 (s, 1H), 5.91 (s, 2H), 4.27 (d, J=15.9 Hz, 1H), 3.69-3.55 (m, 2H), 3.40 (s, 3H), 3.26-3.05 (m, 3H), 2.85-2.59 (m, 3H); MS: m/z=502.0 (M⁺+1)

Compound 60: ¹H NMR (CDCl₃, 300 MHz): δ 7.82-7.76 (m, 2H), 7.49-7.41 (m, 2H), 7.01 (d, J=8.7 Hz, 1H), 6.72 (d, J=9.0 Hz, 1H), 6.70 (s, 1H), 6.58 (s, 1H), 5.91 (s, 2H), 4.20 (d, J=15.9 Hz, 1H), 3.61-3.54 (m, 2H), 3.46 (s, 3H), 3.27-3.04 (m, 3H), 2.85-2.55 (m, 3H), 2.45 (s, 3H); MS: m/z=480.0 (M⁺+1)

### 27. Preparation of Compounds 63, 65 and 66.

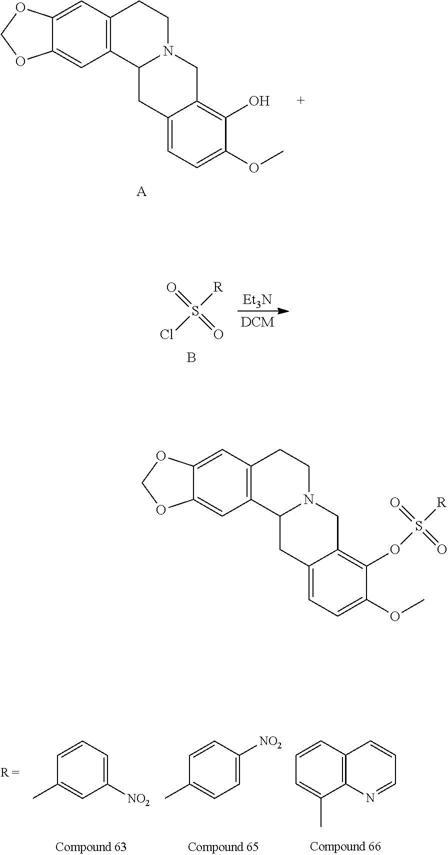

To a stirred solution of A (50 mg, 0.15 mmol) in DCM (2 mL) was added B (0.17 mmol) at room temperature, Et₃N (0.1 mL) was added and stirred for 4 hours. When TLC analysis indicated the completion of the reaction, the solvent was removed in reduced pressure. The residue was further purified by preparative TLC to provide title compounds.

Compound 63: ¹H NMR (CDCl₃, 300 MHz) δ 8.92 (t, J=2.1 Hz, 1H), 8.56-8.54 (m, 1H), 8.39-8.34 (m, 1H), 7.80 (t, J=8.4 Hz, 1H), 7.38 (d, J=8.7 Hz, 1H), 6.76-6.72 (m, 2H), 6.60 (s, 1H), 5.92 (s, 2H), 4.30 (d, J=15.6 Hz, 1H), 3.72 (d, J=15.3 Hz, 1H), 3.62-3.59 (m, 1H), 3.50 (s, 3H), 3.28-3.05 (m, 3H), 2.89-2.79 (m, 1H), 2.69-2.60 (m, 2H). MS: m/z=511.0 (M⁺+1).

Compound 65: ¹H NMR (CDCl₃, 300 MHz) δ 8.52-8.47 (m, 2H), 8.24-8.20 (m, 2H), 7.11 (d, J=8.4 Hz, 1H), 6.94 (d, J=8.4 Hz, 1H), 6.92 (s, 1H), 6.68 (s, 1H), 5.96 (s, 2H), 4.05 (d, J=15.9 Hz, 1H), 3.50-3.42 (m, 2H), 3.36 (s, 3H), 3.06-2.84 (m, 2H), 2.67-2.42 (m, 4H); MS: m/z=511.0 (M⁺+1).

Compound 66: ¹H NMR (CDCl₃, 300 MHz) δ 9.26-9.24 (m, 1H), 8.41-8.12 (m, 3H), 7.65-7.57 (m, 2H), 6.96 (d, J=8.4 Hz, 1H), 6.68 (s, 1H), 6.58 (d, J=9.9 Hz, 1H), 6.57 (s, 1H), 5.90 (s, 2H), 4.30 (d, J=15.9 Hz, 1H), 3.64-3.52 (m, 2H), 3.23-3.00 (m, 3H), 3.02 (s, 3H), 2.83-2.49 (m, 3H); MS: m/z=517.0 (M⁺+1).

### 28. Preparation of Compound 64.

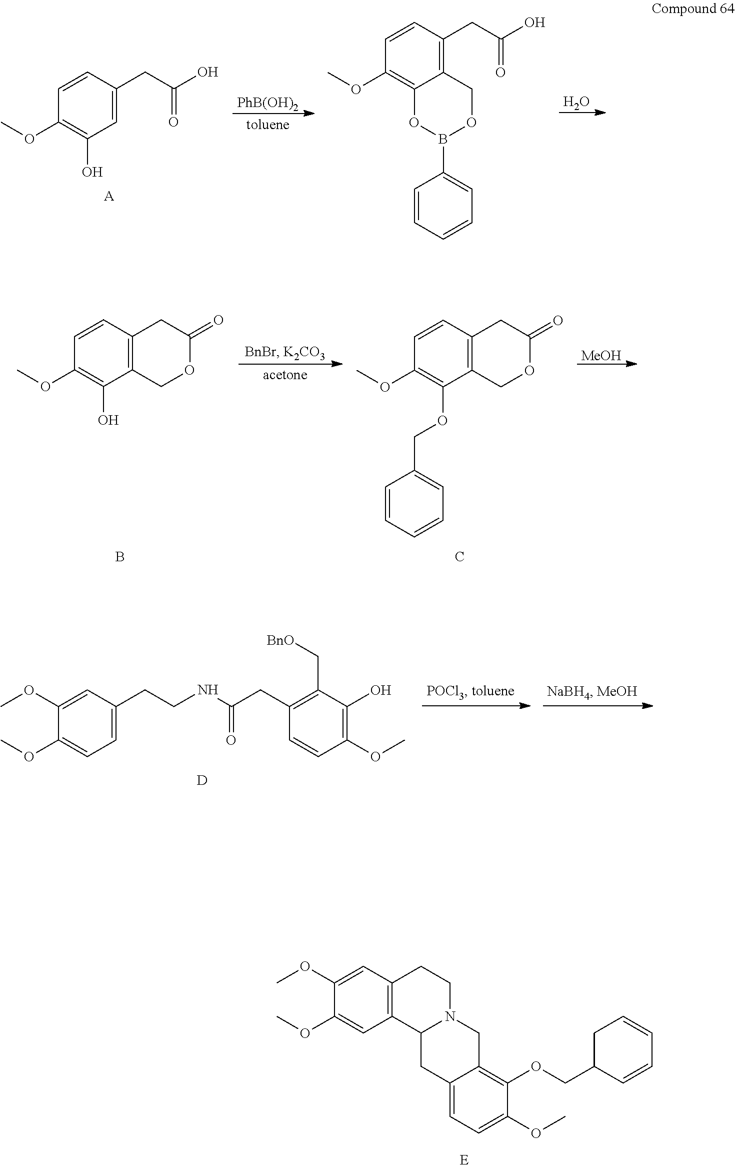

To 5.2 g of A was added phenylboric acid and toluene. The mixture was heated at reflux for 1 hour, and water was collected in a Dean-stark trap. The hot solution was poured over molecular sieves (3.7 g) in a stainless steel bomb. Paraformaldehyde (6.4 g) was added. The bomb was sealed and heated on an oil-bath at 110° C. for 48 hours. The bomb was opened and the hot solution filtered. The toluene was evaporated, and water was added to the residue. After heating at reflux for 2 hours, the mixture was cooled to room temperature and extracted with DCM. The solution was dried and the solvent was removed. The residue was washed with ether to obtained 2.5 g of B.

To a solution of B (1.0 g, 5.2 mmol) in 20 mL of acetone was added K₂CO₃ (810 mg, 5.8 mmol), followed by BnBr (0.7 mL, 5.8 mmol). The mixture was stirred at room temperature overnight. Then the mixture was diluted with EtOAc, washed with water, brine, and dried over anhydrous Na₂SO₄. The solvent was removed under vacuum. The residue was purified by flash chromatography to provide 1.2 g of C.

To a solution of C (200 mg, 0.7 mmol) in 6 mL of MeOH was added 3, 4-dimethoxy phenethylamine (0.3 mL, 1.8 mmol) dropwise. The mixture was stirred at room temperature for 3 hours. The reaction mixture was diluted with EtOAc, washed with H₂O, brine, dried over anhydrous Na₂SO₄. Then the solvent was removed under vacuum. The residue was purified with flash chromatography to give 310 mg of D.

Compound D (101 mg, 0.25 mmol) was suspended in 2 mL of toluene. The mixture was stirred under nitrogen. Then 0.15 mL of phosphoryl chloride was added in one portion, and the mixture was heated under reflux for 2 hours. The reaction mixture was cooled under nitrogen. Excess phosphoryl chloride and toluene was evaporated under vacuum. The residue was dissolved in methanol, then 100 mg of NaBH₄ was added in portions. The mixture was stirred at room temperature for 30 minutes. Then the solvent was removed, the residue was diluted with EtOAc, washed with H₂O, brine, dried over anhydrous Na₂SO₄. Then the solvent was removed under vacuum. The residue was purified with flash chromatography to give 42 mg of E (compound 64).

Compound 64: ¹H NMR (CDCl₃, 300 MHz), δ 7.50-7.30 (m, 5H), 6.86 (dd, J=8.4, 23.7 Hz, 2H), 6.73 (s, 1H), 6.61 (s, 1H), 5.04 (dd, J=11.4, 33.9 Hz, 2H), 4.22 (d, J=15.9 Hz, 1H), 3.89 (s, 3H), 3.87 (s, 3H), 3.86 (s, 3H), 3.55-3.42 (m, 2H), 3.30-3.11 (m, 3H), 2.89-2.58 (m, 3H); MS: m/z=432.1 (M⁺+1).

### 29. Preparation of Biotin-Labeled Compounds Including 156, and 120.

(1) Synthesis of 156

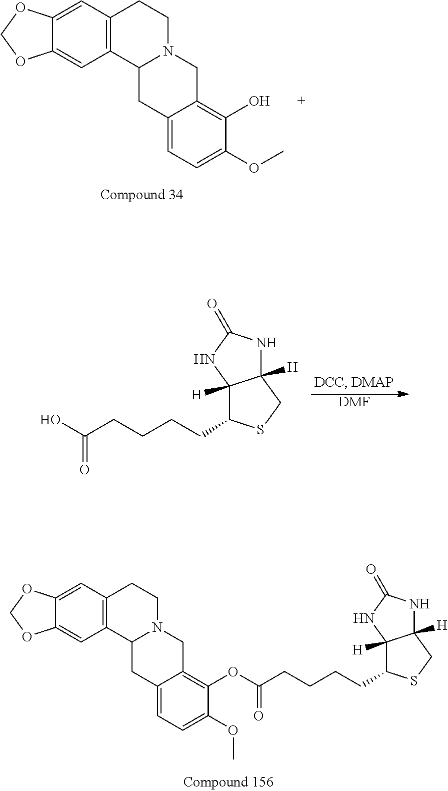

A solution of 34 (325 mg, 1.0 mmol), Biotin (245 mg, 1.0 mmol), DCC (210 mg, 1.0 mmol), and DMAP (125 mg, 1.0 mmol) in DMF was stirred at room temperature overnight. Then the reaction mixture was diluted with DCM, washed with water (3 times), brine, and dried over anhydrous Na₂SO₄. The solvent was removed under vacuum. The residue was purified by flash column silica gel chromatography, then preparative TLC to afford 15 mg of the title compound.

Compound 156: ¹H NMR (CDCl₃, 300 MHz) δ 6.99 (d, J=8.4 Hz, 1H), 6.81 (d, J=8.4 Hz, 1H), 6.70 (s, 1H), 6.57 (s, 1H), 6.00 (d, J=8.1 Hz, 1H), 5.90 (s, 2H), 5.35 (s, 1H), 4.47-4.45 (m, 1H), 4.33-4.30 (m, 1H), 3.98 (d, J=15.6 Hz, 1H), 3.79 (s, 3H), 3.57-3.54 (m, 1H), 3.43 (d, J=15.6 Hz, 1H), 3.24-2.59 (m, 9H), 2.15-1.95 (m, 2H), 1.86-1.53 (m, 6H); MS: m/z=552.1 (M⁺+1).

(4) Preparation of Compound 120

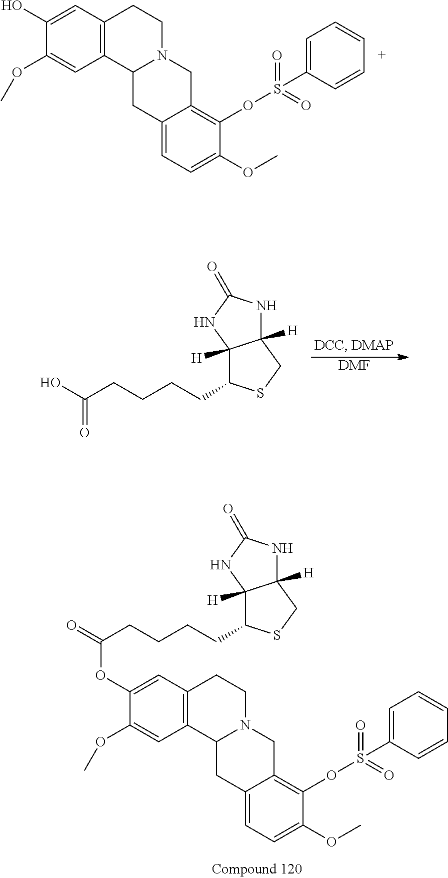

A solution of 102 (30 mg, 0.06 mmol), Biotin (15 mg, 0.06 mmol), DCC (15 mg, 0.06 mmol), and DMAP (10 mg, 0.08 mmol) in DMF was stirred at room temperature overnight. Then the reaction mixture was diluted with DCM, washed with water (3 times), brine, and dried over anhydrous Na₂SO₄. The solvent was removed under vacuum. The residue was purified by flash column silica gel chromatography, then preparative TLC to afford 10 mg of the title compound.

Compound 120: ¹H NMR (CDCl₃, 300 MHz) δ 8.01-7.53 (m, 5H), 7.03 (d, J=8.7 Hz, 1H), 6.79 (s, 2H), 6.72 (d, J=8.7 Hz, 1H), 5.78 (s, 1H), 5.20 (s, 1H), 4.48-4.46 (m, 1H), 4.34-4.30 (m, 1H), 4.20 (d, J=16.2 Hz, 1H), 3.83 (s, 3H), 3.65-3.57 (m, 2H), 3.44 (s, 1H), 3.33-2.58 (m, 11H), 1.89-1.53 (m, 6H); MS: m/z=694.1 (M⁺+1).

### 30. Preparation of R₁/R₂ Analogs.

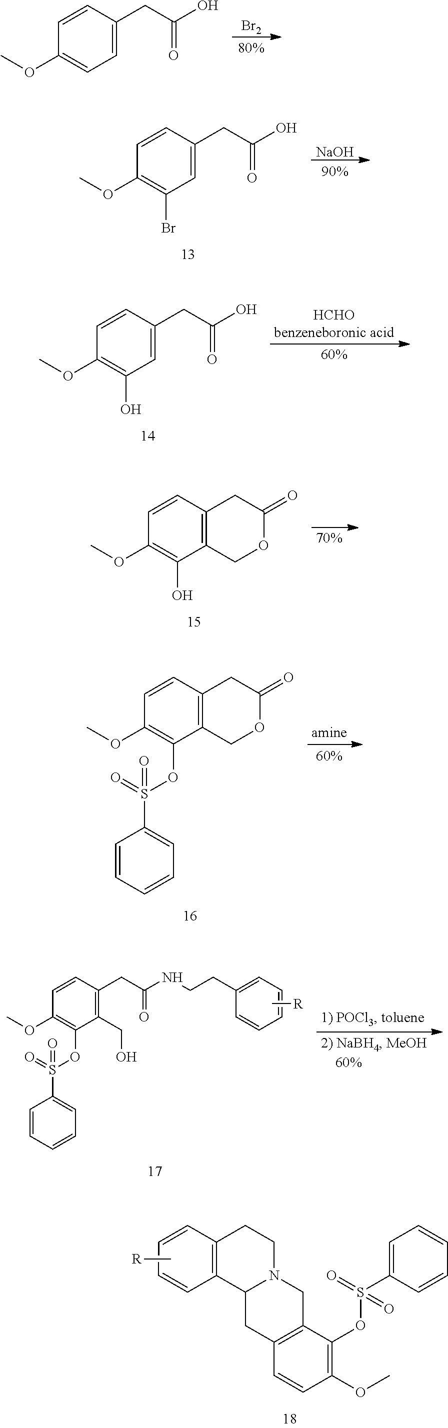

The most important of this part of work is the preparation of various intermediates. The preparation of several intermediates amines and the synthetic routes of desired compounds are shown in the Scheme below.

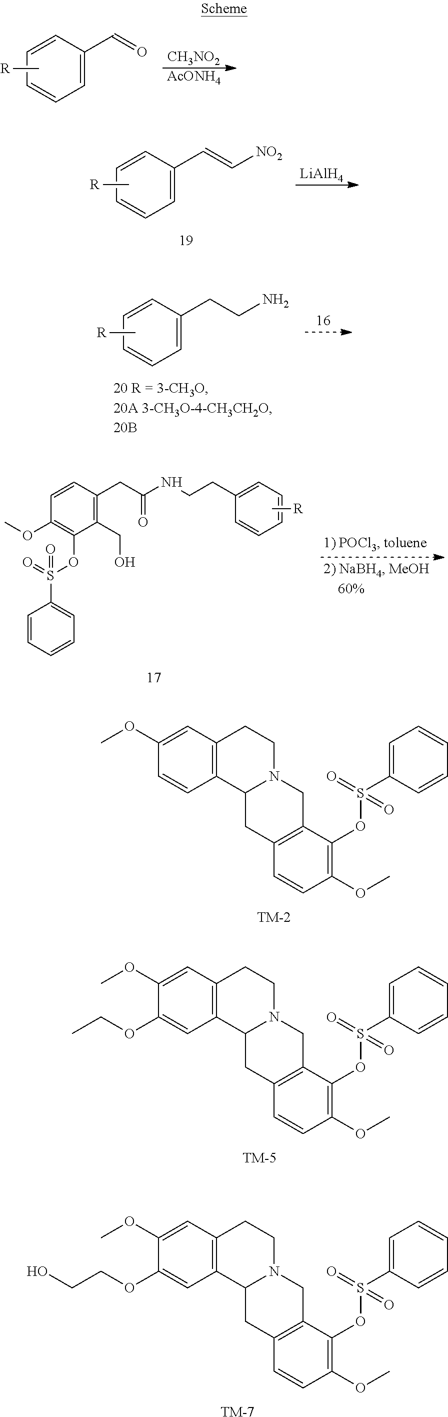

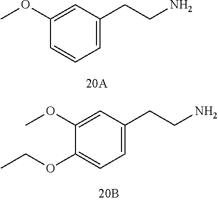

For analog TM-6, different synthetic route was developed as shown in the scheme below.

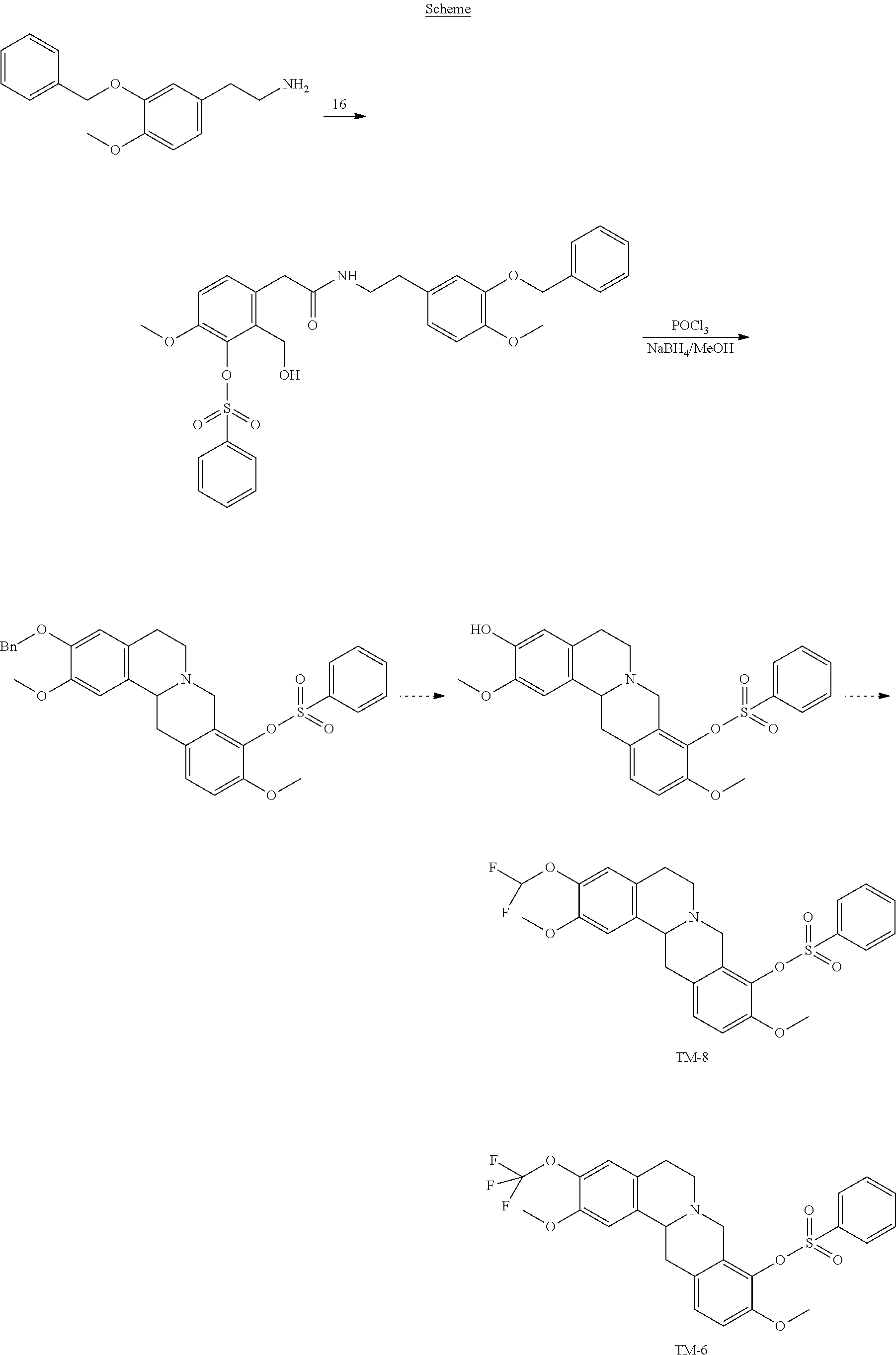

In addition, the COOH group was introduced into the desired compounds to improve the solubility of this series compounds. Some intermediates with the amines such as 21 and 22 were prepared with carboxyl acid group, as shown in the scheme below.

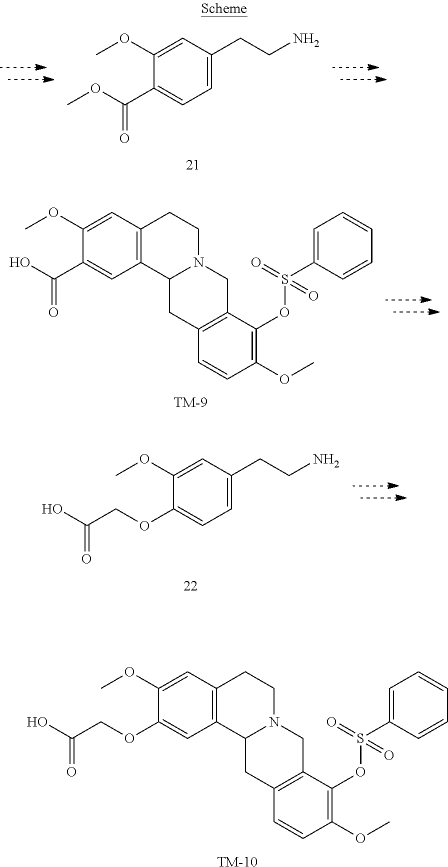

The synthetic route for making the intermediate compound 20C was designed as follows:

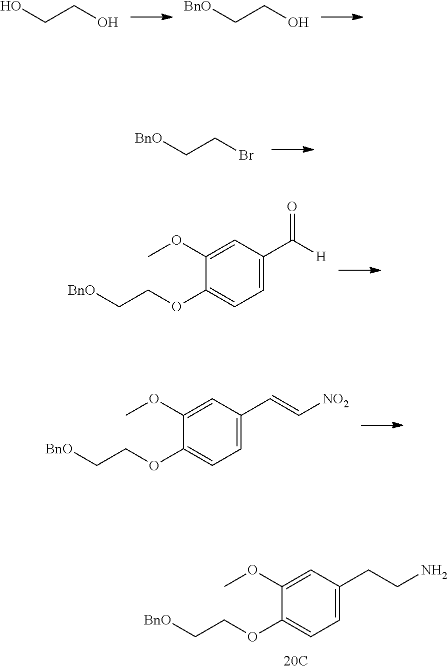

### 31. Preparation of Compound 113

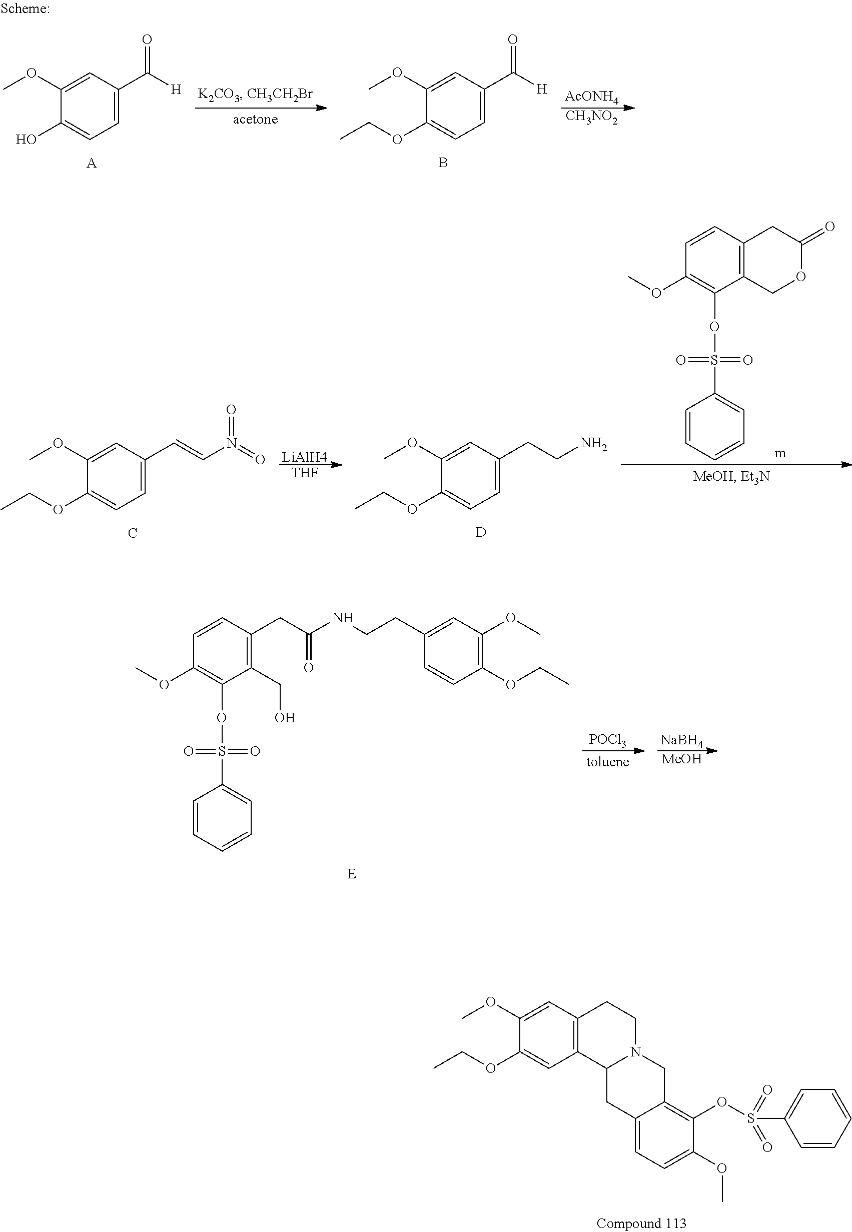

### A to B

To a mixture of A (2.0 g, 13.1 mmol) and K₂CO₃ (2.17 g, 15.7 mmol) in 100 mL of acetone, CH₃CH₂Br (1.5 mL, 20.0 mmol) was added. After heating at reflux temperature for one day, the reaction mixture was cooled down. Acetone was removed. Then the reaction mixture was diluted with DCM, washed with water, dried over anhydrous Na₂SO₄, the solvent evaporated and the residue purified on silica chromatography with eluent (EtOAc: PE=1:8) to afford 2.49 g of B.

### B to C

B (2.49 g, 13.8 mmol) and AcONH₄ (0.16 g, 1.4 mmol) was dissolved in 20 mL CH₃NO₂. The mixture was stirred at reflux temperature for 2 h. Upon completion, the reaction mixture was cooled to rt. The solvent was removed under vacuum, and the residue was diluted with DCM, washed with water, dried over anhydrous Na₂SO₄. Solvent was removed to afford 1.60 g of C.

### C to D

C (1.60 g, 7.2 mmol) in 50 mL THF was added to a solution of LiAlH₄ (1.36 g, 35.9 mmol) in 80 mL THF. The mixture was stirred at reflux temperature for 8 h. Upon completion, the reaction mixture was cooled to rt. 1.4 mL water, 1.4 mL 15% NaOH (aq) and 4.0 mL water was added. The solid was filtered. Solvent was removed and purified on silica chromatography with eluent (MeOH: DCM=1:12) to afford 0.8 g of D.

### D to E

MeOH 20 mL was added to a mixture of m (300 mg, 0.9 mmol), D (193 mg, 1.0 mmol) and 1 mL Et₃N. The resulting solution was stirred at reflux temperature for 8 h. Then the reaction mixture was diluted with DCM, washed with water, aq. NaCl, dried over anhydrous Na₂SO₄. The solvent was removed under vacuum; the residue was purified by silica gel chromatography to afford 300 mg of E.

### E to Compound 113

A mixture of E (150 mg, 0.3 mmol) and POCl₃ (1 mL) in toluene (5 mL) was stirred at 120° C. for 2 h. The reaction was monitored by TLC (DCM: MeOH=10:1, v/v). Upon completion, the reaction mixture was cooled to rt. The solvent was removed under vacuum. 53 mg of NaBH₄ was added in portions under 5° C. The reaction mixture was stirred at room temperature for another 30 minutes. The solvent was removed, and the residue was diluted with DCM. The solution was washed with aq. NaCl, water, dried over anhydrous Na₂SO₄. The solvent was removed under vacuum; the residue was purified by silica gel chromatography to afford 20 mg of compound 113.

Compound 113: ¹H NMR (CDCl₃, 300 MHz) δ 7.99-8.02 (m, 2H), 7.65-7.71 (m, 1H), 7.54-7.60 (m, 2H), 7.03 (d, J=8.4 Hz, 1H), 6.70-6.73 (m, 2H), 6.62 (s, 1H), 4.21 (d, J=15.9 Hz, 1H), 4.05-4.15 (m, 2H), 3.86 (s, 3H), 3.56-3.63 (m, 2H), 3.44 (s, 3H), 3.23-3.30 (m, 1H), 3.07-3.16 (m, 2H), 2.78-2.89 (m, 1H), 2.56-2.68 (m, 2H), 1.47-1.49 (t, 3H). MS: m/z=496.1 (M⁺+1).

### 32. Preparation of Compound 114

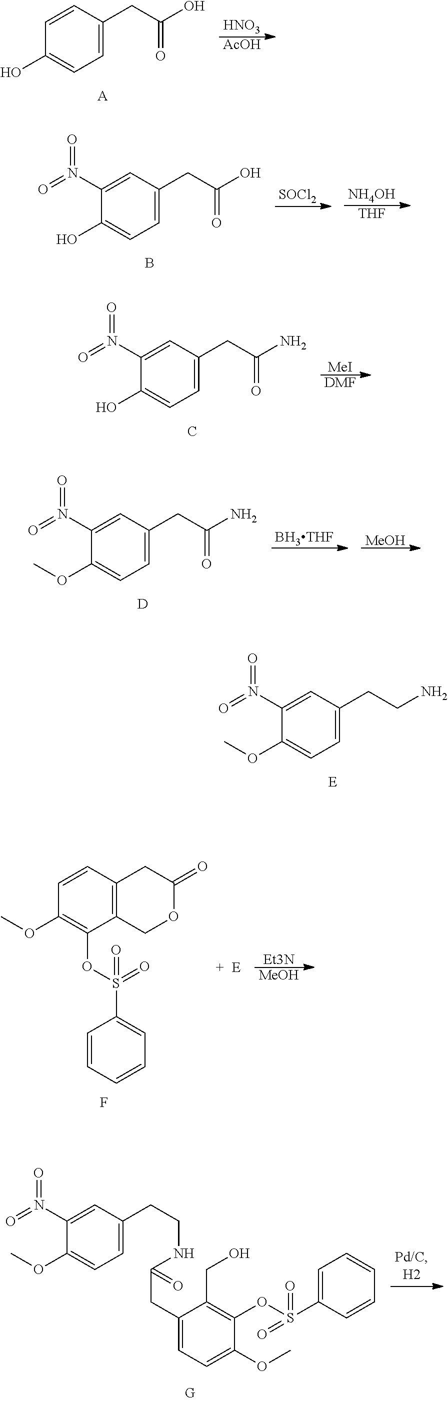

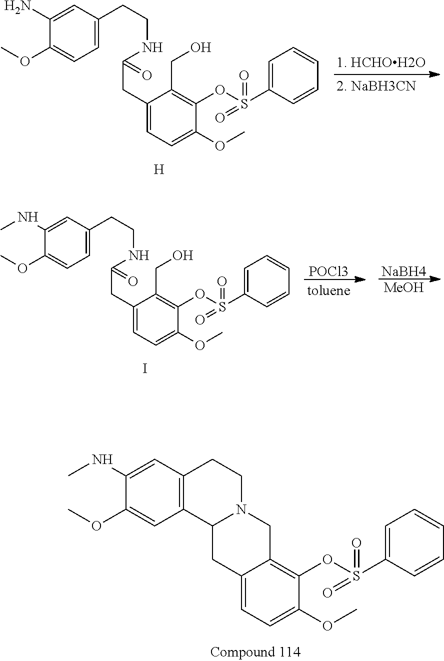

### A→B

A solution of nitric acid (2.5 mL, 40 mmol) in acetic acid (2 mL) was slowly added drop-wise to a solution of (4-hydroxyphenyl)acetic acid (5.0 g, 33 mmol) in acetic acid (23 mL) cooled in an ice-bath such that the temperature of the reaction solution did not exceed 20° C. After the reaction solution was stirred for 2 h, water (100 mL) was added drop-wise and the precipitated crystals were collected by filtration. The solid was re-crystallized from EA/PE to afford B (5.0 g).

### B→C

(4-Hydroxy-3-nitrophenyl)acetic acid (1.3 g, 6.6 mmol) was dissolved in SOCl₂ (15 mL) at room temperature and stirred for 10 min. Then the reaction solution was heated to reflux and stirred for 2 hours. The solvent was removed and the residue was dissolved in THF and again concentrated. Aqueous ammonia solution (10 mL of 25% solution) and THF (5 mL) was added to the residue and the mixture was stirred for 30 min at room temperature. The obtained suspension was diluted with water and THF was evaporated under reduced pressure. The precipitate was collected by filtration, washed with water and dried under reduced pressure To give 1.1 g of C.

### C→D

C (1.1 g, 5.1 mmol) and potassium carbonate (1.1 g, 8.0 mmol) were suspended in DMF (20 mL) and methyl iodide (0.44 mL, 7.1 mL) was added at room temperature. The mixture was stirred for 3 h. The reaction mixture was diluted with EtOAc and washed with water. The organic layer was concentrated to give 1.0 g of D.

### D→E

To a solution of D (1.0 g, 4.8 mmol) in THF (5 mL) was slowly added 1.0 M borane-THF solution (15 mL) at room temperature, and the mixture was refluxed for 5 h. The reaction mixture was concentrated, the residue cooled to room temperature and methanol (20 mL) was added. The mixture was further refluxed overnight. The reaction mixture was extracted with EtOAc and water, the organic layer was collected and concentrated to give 700 mg of E.

### E+F→G

To the solution of E (1.6 g, 8.2 mmol), F (3.0 g, 9.0 mmol) in MeOH (40 mL) was added Et₃N (1.4 mL, 9.8 mmol) at 20° C. and the solution was stirred overnight. When TLC analysis indicated the completion of the reaction, the reaction mixture was diluted with EtOAc, and washed with water. The organic layer was collected, dried, concentrated and purified by chromatography to give 3.0 g of G.

### G→H

The reaction mixture of G (200 mg, 0.4 mmol) and Pd/C (50 mg) in MeOH (4 mL) was stirred at room temperature under H₂ for 8 h. Then the solid was filtered off, and the filtrate was concentrated to give crude product. The residue was further purified by preparative TLC to give 150 mg of H.

### H→I

To a stirred solution of H (150 mg, 0.3 mmol) in MeOH was added HCHO.H₂O (96 mg, 1.2 mmol) at room temperature for 1 h. The reaction solution was cooled to 0° C., NaBH₃CN (96 mg, 1.5 mmol) was added and stirred for 30 min at room temperature. When TLC analysis indicated the completion of the reaction, water and EtOAc were added, and the organic layer was collected, dried, concentrated and purified by chromatography to give 50 mg of I.

### I→Compound 114

To a stirred solution of I (50 mg, 0.1 mmol) in toluene (3 mL) was added POCl₃ (20 μL) at ambient temperature. The reaction mixture was refluxed for 2 h with stirring. When the reaction was completed, the solvent and excess POCl₃ were evaporated off. The residue was poured into ice-water and adjusted pH >7 with Na₂CO₃. The solution was extracted with EtOAc, and the organic layer was dried, and concentrated. The resulting residue was dissolved in MeOH (3 mL) and NaBH₄ (7.6 mg, 0.2 mmol) was added at 0° C. The reaction mixture was stirred for 30 min at this temperature. TLC analysis indicated the completion of the reaction. The solvent was removed and the residue was extracted with EtOAc and water. The organic layer was dried, concentrated and purified by chromatography to give 15 mg of compound 114.

Compound 114: ¹H NMR (CDCl₃, 300 MHz) δ 8.02-7.98 (m, 2H), 7.72-7.65 (m, 1H), 7.59-7.54 (m, 2H), 7.04 (d, J=8.4 Hz, 1H), 7.03 (d, J=8.4 Hz, 1H), 6.57 (s, 1H), 6.32 (s, 1H), 4.28 (d, J=18.3 Hz, 1H), 3.84 (s, 3H), 3.73 (s, 1H), 3.46 (s, 3H), 3.33-3.26 (m, 1H), 3.19-3.10 (m, 2H), 2.85 (m, 3H), 2.71-2.66 (m, 2H), 2.24-2.17 (m, 1H). MS: m/z=481.1 (M⁺+1).

### 33. Preparation of Compound 115

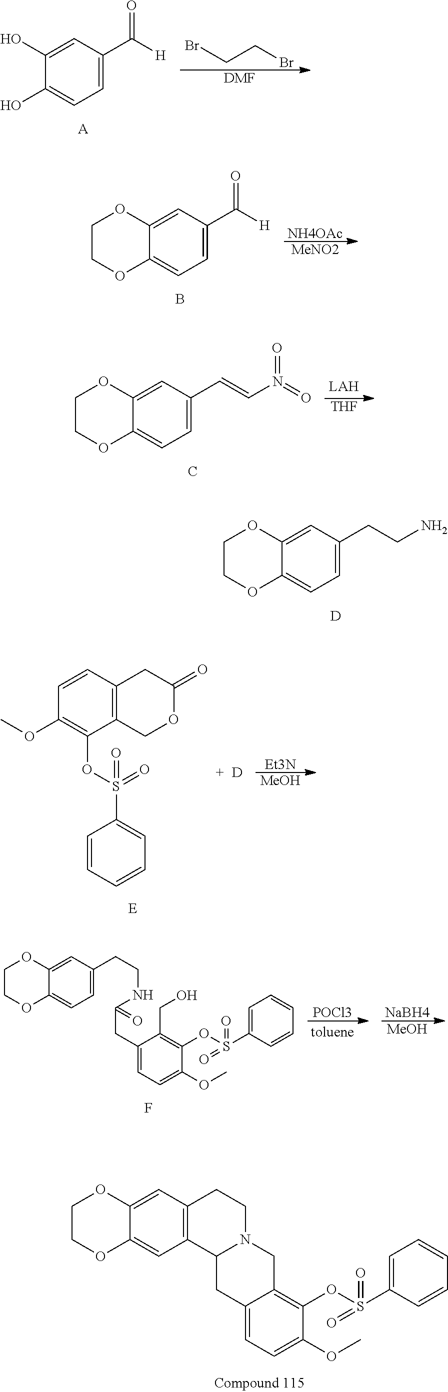

### A→B

A (5.0 g, 36.2 mmol), cesium carbonate (26 g, 72.4 mmol), 1,2-dibromoethane (7.0 mL, 72.4 mmol) and anhydrous DMF (50 mL) were stirred at 70° C. for 16 h. After cooling, the solvent was evaporated under reduced pressure and the residue was submitted to flash chromatography (SiO₂ column eluted with CH₂Cl₂), resulting in the isolation of 5.8 g of B.

### B→C

B (500 mg, 3.0 mmol) and AcONH₄ (23 mg, 0.3 mmol) was dissolved in CH₃NO₂ (5 mL). The mixture was stirred at reflux temperature for 2 h. Upon completion, the reaction mixture was cooled to rt. The solvent was removed under vacuum, and the residue was diluted with DCM, washed with water, dried over anhydrous Na₂SO₄. Solvent was removed to afford 600 mg of C.

### C→D

C (600 mg, 2.9 mmol) in THF (5 mL) was added to a solution of LiAlH₄ (550 mg, 14.5 mmol) in THF (5 mL). The mixture was stirred at reflux temperature for 8 h. Upon completion, the reaction mixture was cooled to rt. 0.5 mL of water, 0.5 mL of 25% NaOH (aq) and 1.5 mL of water was added. The solid was filtered. Solvent was removed and the residue purified on silica chromatography with eluent (MeOH: DCM=1:12) to afford 500 mg of D.

### D+E→F

To the solution of D (250 mg, 1.4 mmol), E (520 mg, 1.5 mmol) in MeOH (15 mL) was added Et₃N (250 μL, 1.7 mmol) at 20° C. and the solution was stirred overnight. When TLC analysis indicated the completion of the reaction, water was added, and the organic layer was collected, dried, concentrated and purified by chromatography to give 600 mg of F.

### F→Compound 115

To a stirred solution of F (100 mg, 0.2 mmol) in toluene (5 mL) was added POCl₃ (30 μL) at ambient temperature. The reaction mixture was refluxed for 2 h with stirring. When the reaction was completed, the solvent and excess POCl₃ were evaporated off. The residue was poured into ice-water and the pH adjusted to >7 with Na₂CO₃. The solution was extracted with EtOAc, and the organic layer was dried, concentrated and dissolved in MeOH (5 mL), NaBH₄ (15 mg, 0.4 mmol) was added at 0° C. The reaction mixture was stirred for 30 min at this temperature. TLC analysis indicated the completion of the reaction. The solvent was removed and the residue was extracted with EtOAc and water. The organic layer was dried, concentrated and purified by chromatography to give 30 mg of compound 115.

Compound 115: ¹H NMR (CDCl₃, 300 MHz) δ 8.01-7.98 (m, 2H), 7.70-7.65 (m, 1H), 7.59-7.54 (m, 2H), 7.02 (d, J=8.4 Hz, 1H), 6.73-6.70 (m, 2H), 6.63 (s, 1H), 4.28-4.24 (m, 5H), 3.72-3.62 (m, 2H), 3.44 (s, 3H), 3.27 (dd, J=3.9, 16.2 Hz 1H), 3.20-3.07 (m, 2H), 2.95-2.82 (m, 1H), 2.70-2.65 (m, 2H). MS: m/z=480.0 (M⁺+1).

### 34. Preparation of Compound 116

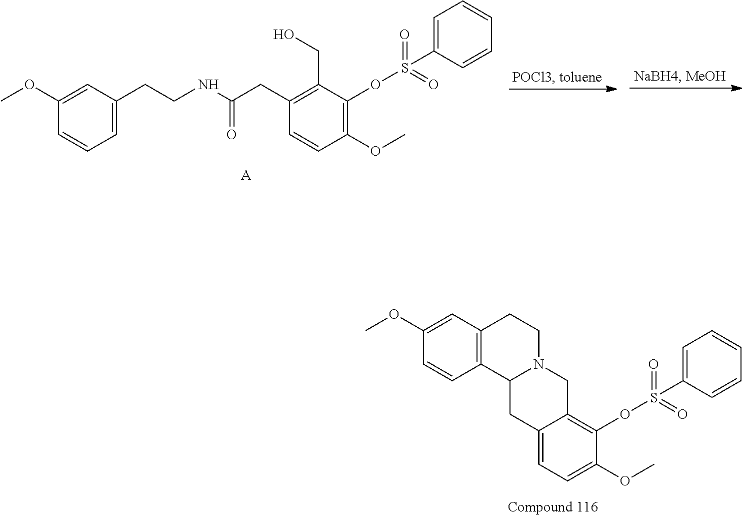

Compound A (200 mg, 0.41 mmol) was suspended in 5 mL of toluene. The mixture was stirred under nitrogen. Then 0.5 mL of phosphoryl chloride was added in one portion, and the mixture was heated under reflux for 2 h. The reaction mixture was cooled under nitrogen. Excess phosphoryl chloride and toluene was evaporated under vacuum. The residue was dissolved in methanol, and 100 mg of NaBH₄ was added in portions. The mixture was stirred at room temperature for 30 min. The solvent was removed, and the residue was diluted with EtOAc, washed with H₂O and brine, and dried over anhydrous Na₂SO₄. The solvent was removed under vacuum. The residue was purified with flash chromatography to give 120 mg of compound 116.

Compound 116: ¹H NMR (CDCl₃, 300 MHz), δ=8.02-7.99 (m, 1H), 7.70-7-65 (m, 1H), 7.59-7.54 (m, 2H), 7.16 (d, J=8.7 Hz), 7.02 (d, J=8.4 Hz), 6.80-6.65 (m, 3H), 4.22 (d, J=15.9 Hz, 1H), 3.80 (s, 3H), 3.63-3.57 (m, 2H), 3.44 (s, 3H), 3.34-3.12 (m, 3H), 2.87-2.58 (m, 3H); MS: m/z=452.1 (M⁺+1).

### 35. Preparation of Compound 117

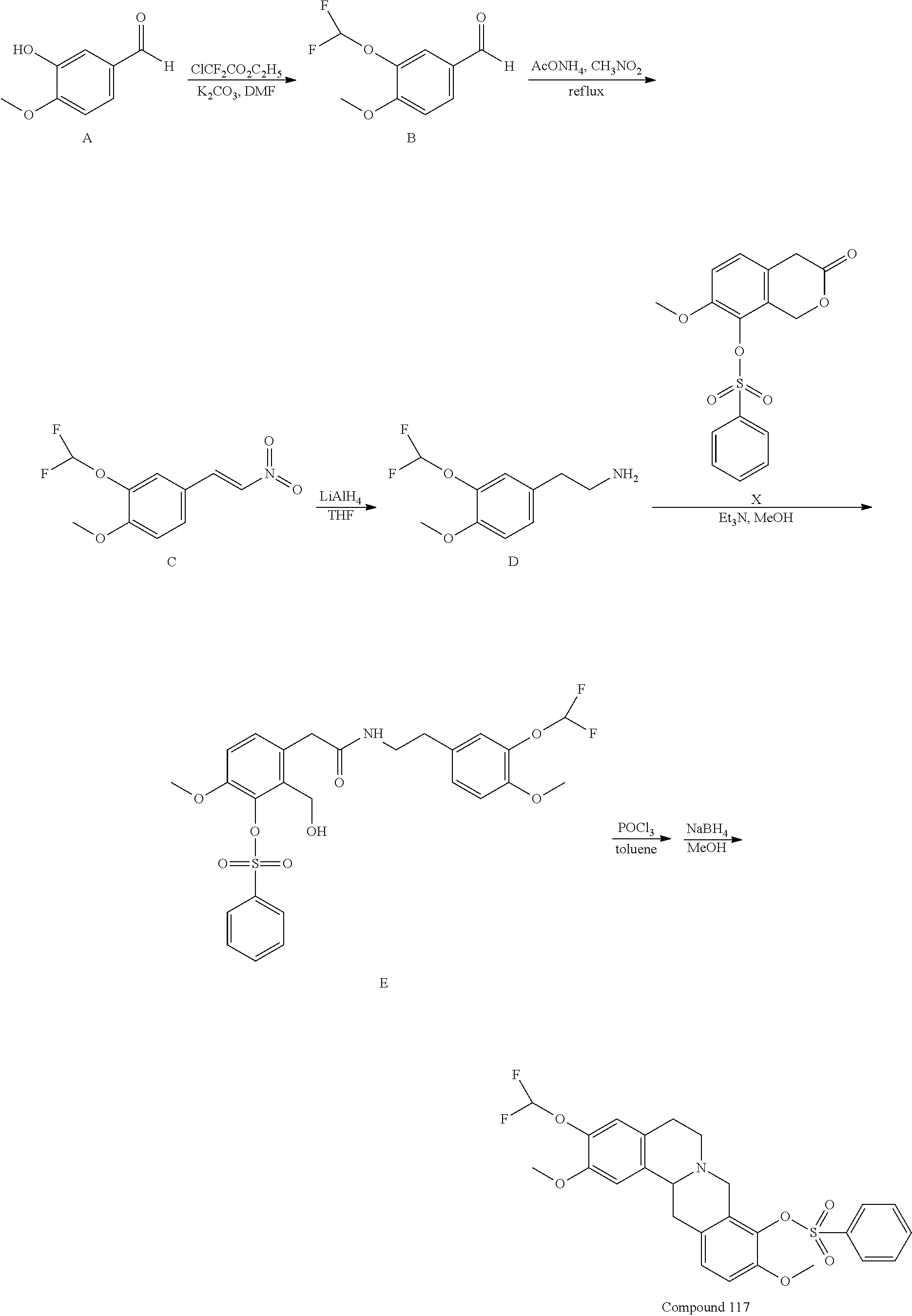

### A to B

A mixture of A (5.0 g, 32.9 mmol) and ethyl 2-chloro-2,2-difluoroacetate (5.7 g, 36.0 mmol), potassium carbonate (4.5 g, 32.9 mmol) in dry DMF (90 mL) were stirred at 60° C. for 6 hours under an N₂ atmosphere. Then the reaction was stirred at rt. for another 60 hours. Ether (200 mL) was added to the mixture and the layers separated. The organic phase was washed with water (100 mL*3), dried over Na₂SO₄, evaporated and purified on silica chromatography with eluent (EtOAc: PE=1:12) to afford B 2.3 g.

### B to C

B (500 mg, 2.48 mmol) and AcONH₄ (19 mg, 0.25 mmol) was dissolved in 15 mL CH₃NO₂. The mixture was stirred at reflux temperature for 2 h. Upon completion, the reaction mixture was cooled to rt. The solvent was removed under vacuum, and the residue was diluted with DCM, washed with water, dried over anhydrous Na₂SO₄. Solvent was removed to afford 590 mg of C.

### C to D

C (300 mg, 1.2 mmol) in 5 mL THF was added to a solution of LiAlH₄ (232 mg, 6.1 mmol) in 10 mL THF. The mixture was stirred at reflux temperature for 8 h. Upon completion, the reaction mixture was cooled to rt. 1.4 mL water, 1.4 mL 15% NaOH (aq) and 4.0 mL water was added. The solid was filtered. Solvent was removed and purified on silica chromatography with eluent (MeOH: DCM=1:10) to afford 85 mg of D.

### D to E

MeOH 10 mL was added to a mixture of X (147 mg, 0.4 mmol), D (80 mg, 0.4 mmol) and 1 mL Et₃N. The resulting solution was stirred at reflux temperature for 8 h. Then the reaction mixture was diluted with DCM, washed with water, aq. NaCl, dried over anhydrous Na₂SO₄. The solvent was removed under vacuum; the residue was purified by silica gel chromatography to afford 132 mg of E.

### E to Compound 117

A mixture of E (130 mg, 0.2 mmol) and POCl₃ (1 mL) in toluene (10 mL) was stirred at 120° C. for 2 h. The reaction was monitored by TLC (DCM: MeOH=10:1, v/v). Upon completion, the reaction mixture was cooled to rt. The solvent was removed under vacuum. 46 mg of NaBH₄ was added in portions under 5° C. The reaction mixture was stirred at room temperature for another 30 minutes. The solvent was removed, and the residue was diluted with DCM. The solution was washed with aq. NaCl, water, dried over anhydrous Na₂SO₄. The solvent was removed under vacuum; the residue was purified by silica gel chromatography to afford 20 mg of compound 117.

Compound 117: ¹H NMR (CDCl₃, 300 MHz) δ 7.97-8.02 (m, 2H), 7.54-7.71 (m, 3H), 6.71-7.05 (m, 4H), 6.54 (t, J=75.3, 1H), 4.22 (d, J=15.9 Hz, 1H), 3.88 (s, 3H), 3.60-3.66 (m, 2H), 3.45 (s, 3H), 3.24-3.31 (m, 1H), 3.04-3.16 (m, 2H), 2.81-2.90 (m, 1H), 2.57-2.72 (m, 2H). MS: m/z=518.1 (M⁺+1).

### 36. Preparation of Compound 118

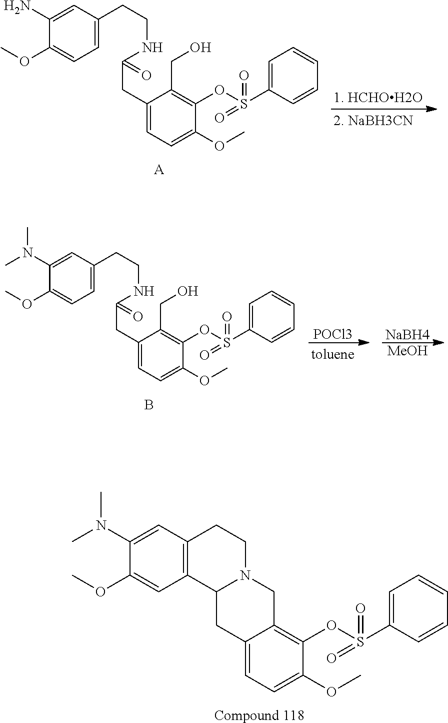

### A→B

To a stirred solution of A (150 mg, 0.3 mmol) in MeOH was added HCHO.H₂O (96 mg, 1.2 mmol) at room temperature for 3 h. The reaction solution was cooled to 0° C., NaBH₃CN (96 mg, 1.5 mmol) was added and stirred for 1 h at room temperature. When TLC analysis indicated the completion of the reaction, water and EtOAc were added, and the organic layer was collected, dried, concentrated and purified by chromatography to give 100 mg of B.

### B→Compound 118

To a stirred solution of I (100 mg, 0.19 mmol) in toluene (3 mL) was added POCl₃ (50 μL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (3 mL), NaBH₄ (22.8 mg, 0.6 mmol) was added at 0° C. The reaction mixture was stirred for 30 min at this temperature. TLC analysis indicated the completion of the reaction. The solvent was removed and the residue was extracted with EtOAc and water. The organic layer was dried, concentrated and purified by chromatography to give 10 mg of compound 118.

Compound 118: ¹H NMR (CDCl₃, 300 MHz) δ 8.02-7.99 (m, 2H), 7.68-7.66 (m, 1H), 7.58-7.54 (m, 2H), 7.04 (d, J=8.4 Hz, 1H), 6.73-6.67 (s, 3H), 4.21 (d, J=15.9 Hz, 1H), 3.89 (s, 3H), 3.61-3.56 (m, 2H), 3.45 (s, 3H), 3.32-3.27 (m, 1H), 3.13-3.07 (m, 2H), 2.90-2.83 (m, 1H), 2.78 (s, 6H), 2.67-2.57 (m, 2H). MS: m/z=495.1 (M⁺+1).

### 37. Preparation of Compound 119

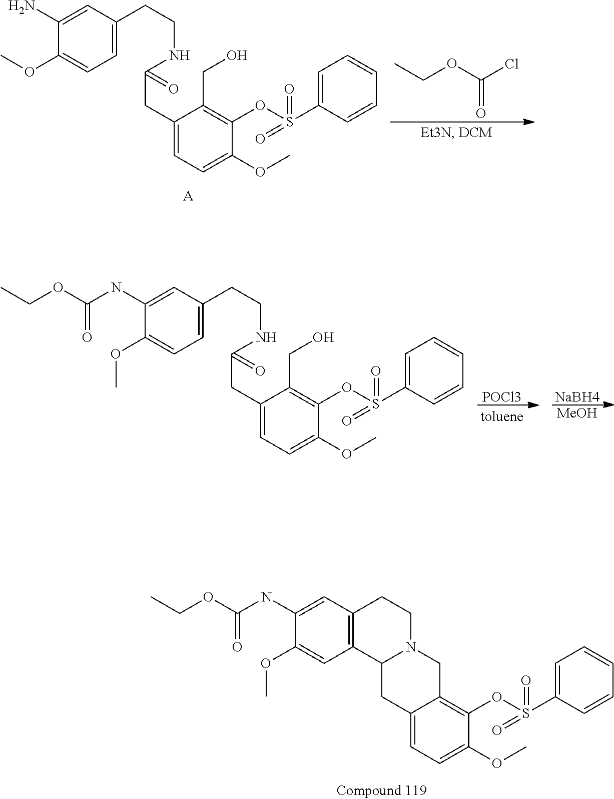

### A→B

To a stirred solution of A (150 mg, 0.3 mmol) in DCM (3 mL) was added Et₃N (100 μL), ethyl chloroformate (100 L) at room temperature for 3 h. When TLC analysis indicated the completion of the reaction, water was added, and the organic layer was collected, dried, concentrated and the resulting residue purified by chromatography to give 100 mg of B.

### B→Compound 119

To a stirred solution of I (100 mg, 0.17 mmol) in toluene (3 mL) was added POCl₃ (50 μL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (3 mL), NaBH₄ (22.8 mg, 0.6 mmol) was added at 0° C. The reaction mixture was stirred for 30 min at this temperature. TLC analysis indicated the completion of the reaction. The solvent was removed and the residue was extracted with EtOAc and water. The organic layer was dried, concentrated and purified by chromatography to give 70 mg of compound 119.

Compound 119: ¹H NMR (CDCl₃, 300 MHz) δ 7.99-7.96 (m, 2H), 7.86 (s, 1H), 7.68-7.66 (m, 1H), 7.59-7.54 (m, 2H), 7.17 (s, 1H), 7.05 (d, J=8.7 Hz, 1H), 6.76 (d, J=8.7 Hz, 1H), 6.68 (s, 1H), 4.40 (d, J=15.3 Hz, 1H), 4.22 (q, J=7.2, 14.1 Hz, 2H), 4.11-4.04 (m, 1H), 3.97-3.91 (m, 1H), 3.85 (s, 3H), 3.47 (s, 3H), 3.43-3.32 (m, 2H), 3.22-3.13 (m, 1H), 3.05-2.84 (m, 3H). MS: m/z=539.1 (M⁺+1).

### 38. Preparation of Compound 121

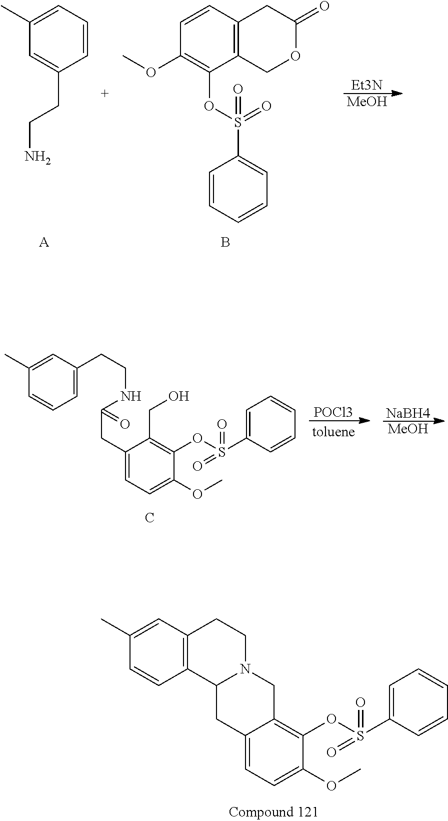

### A+B→C

To the solution of A (67.5 mg, 0.5 mmol), B (167 mg, 0.5 mmol) in MeOH (5 mL) was added Et₃N (83 μL, 0.6 mmol) at 20° C. and the solution was heated to reflux overnight. When TLC analysis indicated the completion of the reaction, water and EtOAc were added, and the organic layer was collected, dried, concentrated and the residue purified by chromatography to give 190 mg of C.

### C→Compound 121

To a stirred solution of C (94 mg, 0.2 mmol) in toluene (5 mL) was added POCl₃ (30 μL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (5 mL), NaBH₄ (15 mg, 0.4 mmol) was added at 0° C. The reaction mixture was stirred for 10 min at this temperature. TLC analysis indicated the completion of the reaction. The solvent was removed and the residue was extracted with EtOAc and water. The organic layer was dried, concentrated and purified by chromatography to give 30 mg of compound 121.

Compound 121: ¹H NMR (CDCl₃, 300 MHz) δ 8.02-7.99 (m, 2H), 7.68-7.65 (m, 1H), 7.59-7.54 (m, 2H), 7.14 (d, J=8.1 Hz, 1H), 7.04-6.96 (m, 3H), 6.72 (d, J=8.4 Hz, 1H), 4.22 (d, J=15.9 Hz, 1H), 3.65-3.58 (m, 2H), 3.44 (s, 3H), 3.32 (dd, J=4.2, 16.2 Hz 1H), 3.16-3.11 (m, 2H), 2.88-2.79 (m, 1H), 2.75-2.57 (m, 2H), 2.32 (s, 3H). MS: m/z=436.1 (M⁺+1).

### 39. Preparation of Compound 122

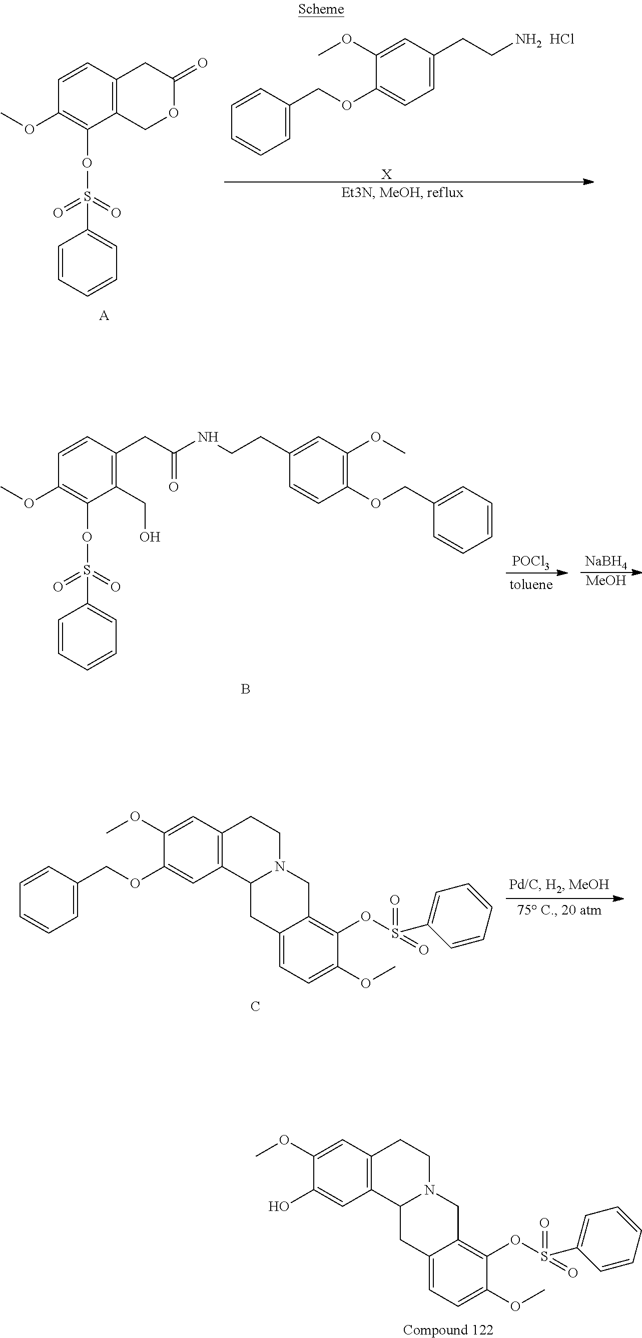

### Procedure

### A to B

MeOH 100 mL was added to a mixture of A (2.0 g, 6.0 mmol), X (2.1 g, 7.2 mmol) and 3 mL Et₃N. The resulting solution was stirred at reflux temperature for 5 h. Then the reaction mixture was diluted with DCM, washed with water, aq. NaCl, and dried over anhydrous Na₂SO₄. The solvent was removed under vacuum; the residue was purified by silica gel chromatography to afford 3.08 g of B.

### B to C

A mixture of B (2.7 g, 4.6 mmol) and POCl₃ (4 mL) in toluene (50 mL) was stirred at 120° C. for 2 h. The reaction was monitored by TLC (DCM: MeOH=10:1, v/v). Upon completion, the reaction mixture was cooled to rt. The solvent was removed under vacuum. 874 mg of NaBH₄ was added in portions under 5° C. The reaction mixture was stirred at room temperature for another 30 minutes. The solvent was removed, and the residue was diluted with DCM. The solution was washed with aq. NaCl, water, and dried over anhydrous Na₂SO₄. The solvent was removed under vacuum; the residue was purified by silica gel chromatography to afford 1 g of C.

### C to Compound 122

To a stirred solution of C (600 mg, 1.1 mmol) in MeOH (50 mL) was added Pd/C (120 mg) under 20 atm H₂. The reaction mixture was stirred at 75° C. overnight. Then the solid was filtered off. The solvent was removed under vacuum; the residue was purified by silica gel chromatography to afford 200 mg of compound 122.

Compound 122: ¹H NMR (CDCl₃, 300 MHz) δ 7.98-8.02 (m, 2H), 7.54-7.71 (m, 3H), 7.02 (d, J=8.4 Hz, 1H), 6.80 (s, 1H), 6.71 (d, J=8.4 Hz, 1H), 6.60 (s, 1H), 5.50 (s, 1H), 4.21 (d, J=15.9 Hz, 1H), 3.88 (s, 3H), 3.54-3.62 (m, 2H), 3.42 (s, 3H), 3.24-3.30 (m, 1H), 3.10-3.15 (m, 2H), 2.80-2.85 (m, 1H), 2.60-2.64 (m, 2H). MS: m/z=468.1 (M⁺+1).

### 40. Preparation of Compound 123 and Compound 124

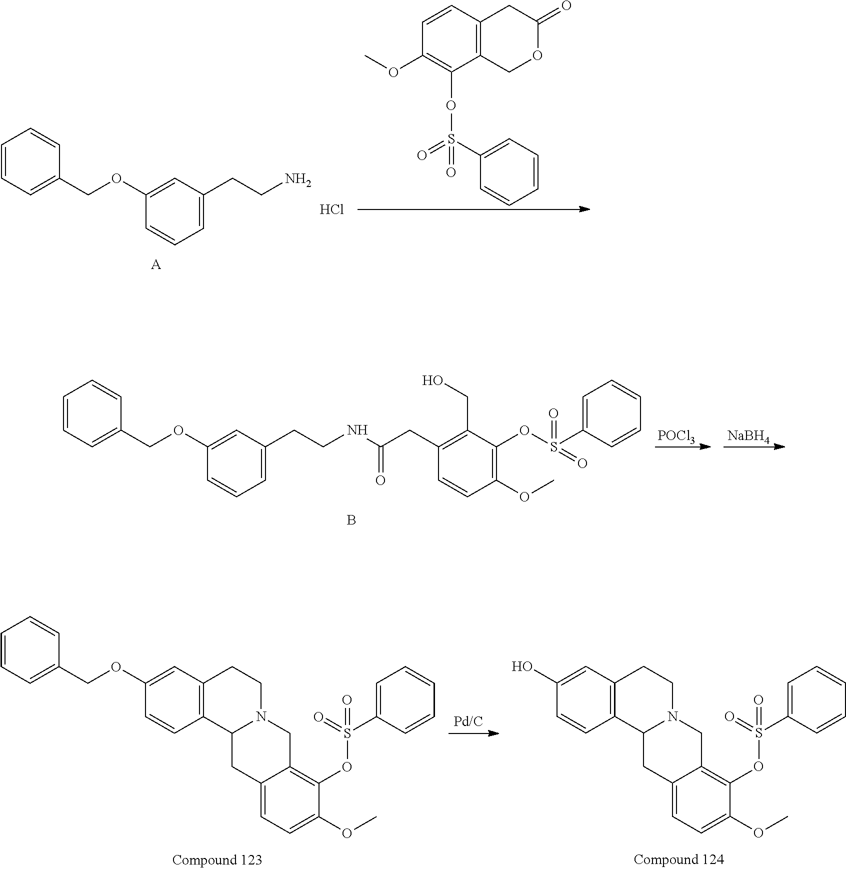

### A to B

To the solution of A (2.4 g, 9.0 mmol) and benzenesulfonate C (2.7 g, 8.1 mmol) in MeOH (20 mL) was added Et₃N (3 mL) at 20° C. and the solution was heated to reflux overnight. When TLC analysis indicated the completion of the reaction, water and EtOAc were added, and the organic layer was collected, dried, concentrated and purified by chromatography to give 3 g of B.

### B to Compound 123

To a stirred solution of B (1 g, 1.8 mmol) in toluene (60 mL) was added POCl₃ (3 mL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried and concentrated. The resulting residue was dissolved in MeOH (20 mL), and NaBH₄ (400 mg) was added at 0° C. The reaction mixture was stirred for 10 min at this temperature. TLC analysis indicated the completion of the reaction. The solvent was removed and the residue was extracted with EtOAc and water. The organic layer was dried, concentrated and the residue purified by chromatography to give 200 mg of compound 123.

### Compound 123 to Compound 124

To a solution of compound 123 (160 mg) in MeOH (50 mL) was added 10% of Pd/C (20 mg). The inner pressure was 2 MPa and the reaction stirred at 70° C. overnight. After stopping the reaction, filter and remove the solvent. Purify by preparative TLC to afford compound 124 about 30 mg.

Compound 123: ¹H NMR (CDCl₃, 300 MHz) δ 7.99-8.01 (d, J=9 Hz, 1H), 7.68-7.65 (m, 1H), 7.59-7.54 (m, 2H), 7.46-7.32 (m, 5H), 7.18-7.15 (d, J=9 Hz, 1H), 6.01-6.04 (d, J=9 Hz, 1H), 6.87-6.84 (m, 1H), 6.70-6.75 (m, 1H), 5.05 (s, 2H), 4.20-4.26 (d, J=9 Hz, 1H), 3.59-3.64 (m, 2H), 3.44 (s, 3H), 3.32 (dd, J=4.2, 16.2 Hz 1H), 3.16-3.11 (m, 2H), 2.88-2.58 (m, 3H). MS: m/z=528.1 (M⁺+1).

Compound 124: ¹H NMR (CDCl₃, 300 MHz) δ 7.99-8.01 (d, J=9 Hz, 1H), 7.68-7.65 (m, 1H), 7.59-7.54 (m, 2H), 7.18-7.15 (d, J=9 Hz, 1H), 6.01-6.04 (d, J=9 Hz, 1H), 6.87-6.84 (m, 1H), 6.70-6.75 (m, 1H), 4.20-4.26 (d, J=9 Hz, 1H), 3.59-3.64 (m, 2H), 3.44 (s, 3H), 3.32 (dd, J=4.2, 16.2 Hz 1H), 3.16-3.11 (m, 2H), 2.88-2.58 (m, 3H). MS: m/z=438.1 (M⁺+1).

### 41. Preparation of Compound 125

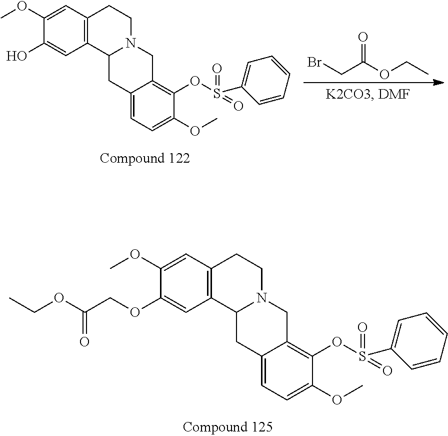

### Compound 122 to Compound 125:

To a stirred mixture of compound 122 (30 mg, 0.06 mmol) and K₂CO₃ (8 mg, 0.06 mmol) in DMF (5 mL) was added ethyl 2-bromoacetate (9 μL, 0.08 mmol). The reaction mixture was stirred at rt overnight. The solvent was removed, and the residue was diluted with DCM. The solution was washed with water, aq. NaCl, dried over anhydrous Na₂SO₄. The solvent was removed under vacuum; the residue was purified by silica gel chromatography to afford 10 mg of compound 125.

Compound 125: ¹H NMR (CDCl₃, 300 MHz) δ 7.98-8.02 (m, 2H), 7.54-7.71 (m, 3H), 7.01 (d, J=8.4 Hz, 1H), 6.64-6.75 (m, 3H), 4.68 (s, 2H), 4.26 (q, J=7.1 Hz, 2H), 4.17 (d, J=15.1 Hz, 1H), 3.57 (s, 3H), 3.54-3.64 (m, 2H), 3.43 (s, 3H), 3.07-3.27 (m, 3H), 2.57-2.85 (m, 3H), 1.31 (t, J=7.2 Hz, 3H). MS: m/z=554.1 (M⁺+1).

### 42. Preparation of Compound 126

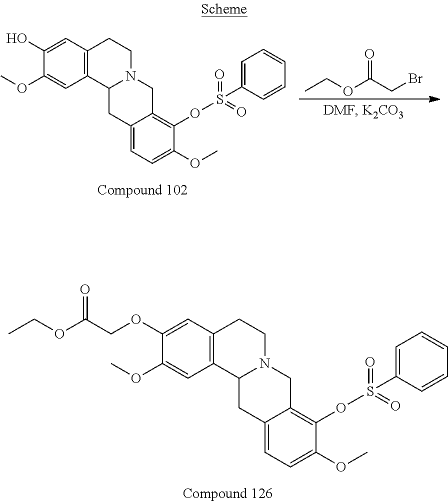

To a stirred mixture of compound 102 (200 mg, 0.43 mmol) and K₂CO₃ (70 mg, 0.50 mmol) in DMF (2 mL) was added ethyl 2-bromoacetate (50 μL, 0.45 mmol). The reaction mixture was stirred at rt. overnight. The solvent was removed under vacuum, and the residue was diluted with DCM. The solution was washed with water, brine, and dried over anhydrous Na₂SO₄. The solvent was removed under vacuum; the residue was purified by silica gel chromatography to afford 160 mg of compound 125.

Compound 125: ¹H NMR (CDCl₃, 300 MHz) δ 8.01-7.98 (m, 2H), 7.67-7.53 (m, 3H), 7.02 (d, J=8.3 Hz, 1H), 6.73-6.70 (m, 2H), 6.56 (s, 1H), 4.66 (s, 2H), 4.26 (q, J=7.1 Hz 2H), 4.17 (d, J=15.1 Hz, 1H), 3.88 (s, 3H), 3.64-3.53 (m, 2H), 3.43 (s, 3H), 3.28-3.01 (m, 3H), 2.85-2.60 (m, 3H), 1.28 (t, J=7.2 Hz, 3H). MS: m/z=554.1 (M⁺+1).

### 43. Preparation of Compound 127

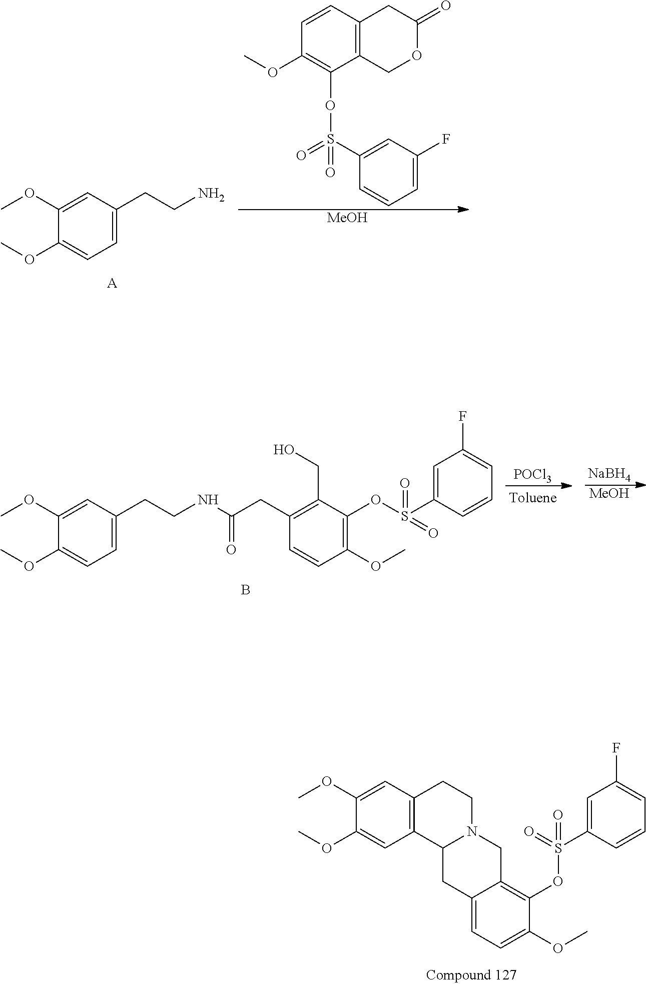

### A to B

To the solution of A (240 mg, 1.4 mmol) and the above benzenesulfonate (400 mg, 1.1 mmol) in MeOH (20 mL) was added Et₃N (0.5 mL) at 20° C. and then the solution was heated to reflux overnight. When TLC analysis indicated the completion of the reaction, water and EtOAc were added, and the organic layer was collected, dried, concentrated and purified by chromatography to give 500 mg of B.

### B to Compound 127

To a stirred solution of B (400 mg, 0.75 mmol) in toluene (60 mL) was added POCl₃ (2 mL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (20 mL), NaBH₄ (200 mg) was added at 0° C. The reaction mixture was stirred for 10 min at this temperature. TLC analysis indicated the completion of the reaction. The solvent was removed and the residue was extracted with EtOAc and water. The organic layer was dried, concentrated and purified by chromatography to give 130 mg of compound 127.

Compound 127: ¹H NMR (CDCl₃, 300 MHz) δ 7.82-7.79 (m, 2H), 7.75-7.71 (m, 1H), 7.60-7.52 (m, 1H), 7.42-7.36 (m, 1H), 7.07-7.04 (dd, J=9 Hz, 1H), 6.71-6.75 (m, 2H), 6.62 (s, 1H), 4.26-4.22 (d, J=12 Hz, 1H), 3.89 (s, 3H), 3.87 (s, 3H), 3.67-3.59 (m, 2H), 3.49 (s, 3H), 3.32-3.26 (dd, J=12 Hz, 1H), 3.19-3.08 (m, 2H), 2.88-2.79 (m, 1H), 2.70-2.63 (m, 2H). MS: m/z=500.1 (M⁺+1).

### 44. Preparation of Compound 128

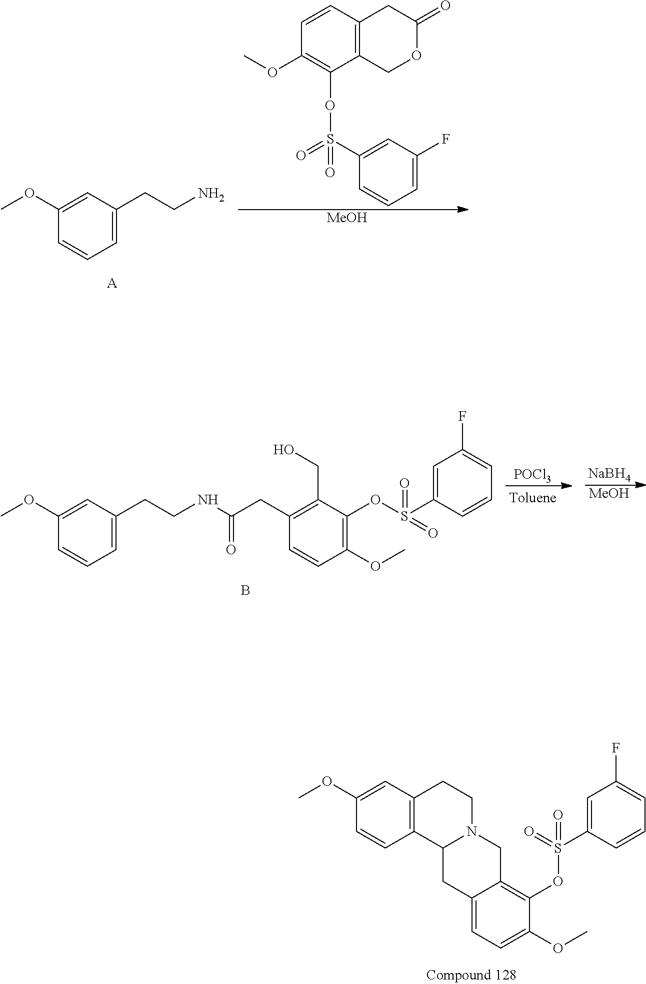

### A to B

To the solution of A (96.5 mg, 0.64 mmol) and the above benzenesulfonate (150 mg, 0.43 mmol) in MeOH (10 mL) was added Et₃N (0.5 mL) at 20° C. and then the solution was heated to reflux overnight. When TLC analysis indicated the completion of the reaction, water and EtOAc were added, and the organic layer was collected, dried, concentrated and purified by chromatography to give 230 mg of B.

### B to Compound 128

To a stirred solution of B (230 mg, 0.46 mmol) in toluene (30 mL) was added POCl₃ (105 mL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (20 mL), NaBH₄ (200 mg) was added at 0° C. The reaction mixture was stirred for 10 min at this temperature. TLC analysis indicated the completion of the reaction. The solvent was removed and the residue was extracted with EtOAc and water. The organic layer was dried, concentrated and purified by chromatography to give 32 mg of compound 128.

Compound 128: ¹H NMR (CDCl₃, 300 MHz) δ 7.82-7.80 (d, J=6 Hz, 1H), 7.75-7.71 (m, 1H), 7.60-7.52 (m, 1H), 7.42-7.36 (m, 1H), 7.17 (dd, J=9 Hz, 1H), 7.05-7.03 (d, J=9 Hz, 1H), 6.80-6.66 (m, 3H), 4.26-4.22 (d, J=12 Hz, 1H), 3.80 (s, 3H), 3.68-3.63 (m, 2H), 3.48 (s, 3H), 3.35-3.29 (dd, J=12 Hz, 1H), 3.19-3.14 (m, 2H), 2.87-2.77 (m, 3H). MS: m/z=470.1 (M⁺+1).

### 45. Preparation of Compound 129

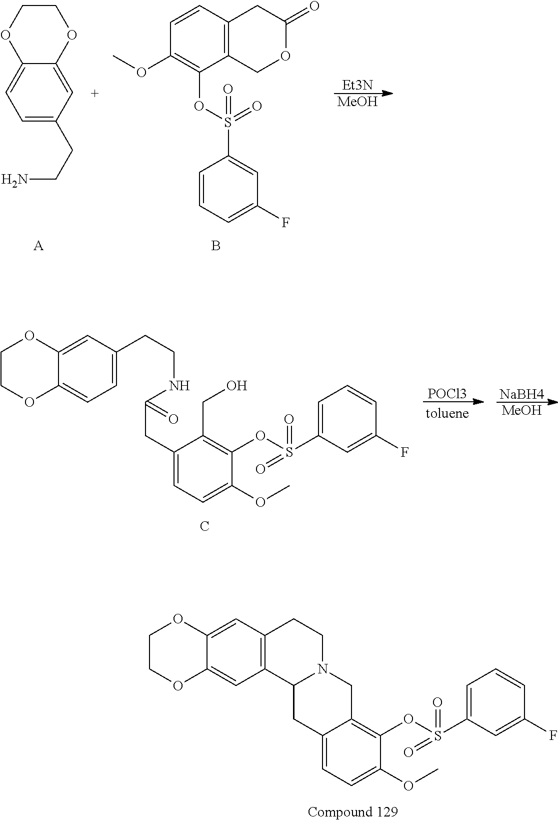

### A+B→C

To the solution of A (152 mg, 0.85 mmol), B (300 mg, 0.85 mmol) in MeOH (5 mL) was added Et₃N (130 μL, 0.94 mmol) at 20° C., and the solution was heated to reflux overnight. When TLC analysis indicated the completion of the reaction, water was added, and the organic layer was collected, dried, concentrated and purified by chromatography to give 300 mg of C.

### C→Compound 129

To a stirred solution of C (300 mg, 0.56 mmol) in toluene (5 mL) was added POCl₃ (300 μL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried and concentrated. The resulting residue was dissolved in MeOH (5 mL), and NaBH₄ (86 mg, 2.26 mmol) was added at 0° C. The reaction mixture was stirred for 30 min at this temperature. TLC analysis indicated the completion of the reaction. The solvent was removed and the residue was extracted with EtOAc and water. The organic layer was dried, concentrated and purified by chromatography to give 70 mg of compound 129.

Compound 129: ¹H NMR (CDCl₃, 300 MHz) δ 7.80-7.77 (m, 1H), 7.72-7.69 (m, 1H), 7.59-7.55 (m, 1H), 7.41-7.35 (m, 1H), 7.03 (d, J=8.4 Hz, 1H), 6.76-6.72 (m, 2H), 6.63 (s, 1H), 4.34 (d, J=15.6 Hz, 1H), 4.24-4.20 (m, 4H), 3.78-3.70 (m, 2H), 3.48 (s, 3H), 3.33-3.26 (m, 2H), 3.14-3.08 (m, 1H), 2.96-2.87 (m, 1H), 2.74-2.69 (s, 2H). MS: m/z=498.1 (M⁺+1).

### 46. Preparation of Compound 130

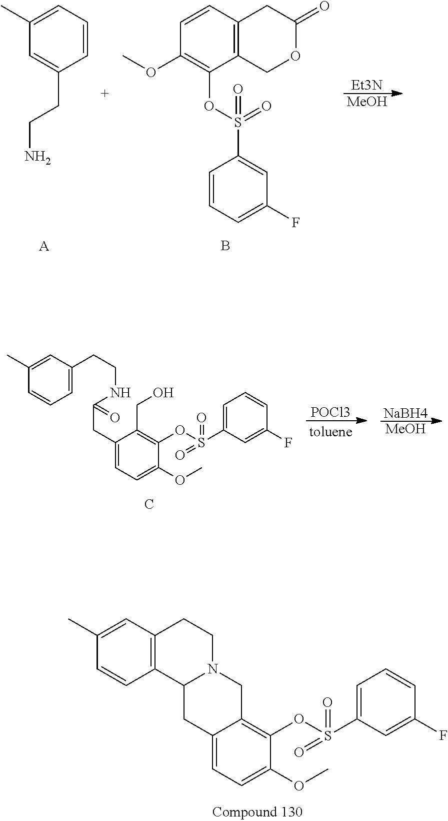

### A+B→C

To the solution of A (115 mg, 0.85 mmol), B (300 mg, 0.85 mmol) in MeOH (5 mL) was added Et₃N (130 μL, 0.94 mmol) at 20° C. and the solution was heated to reflux overnight. When TLC analysis indicated the completion of the reaction, water and EtOAc were added, and the organic layer was collected, dried, concentrated and purified by chromatography to give 300 mg of C.

### C→Compound 130

To a stirred solution of C (300 mg, 0.62 mmol) in toluene (5 mL) was added POCl₃ (300 μL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried and concentrated. The resulting residue was dissolved in MeOH (5 mL), and NaBH₄ (94 mg, 2.48 mmol) was added at 0° C. The reaction mixture was stirred for 10 min at this temperature. TLC analysis indicated the completion of the reaction. The solvent was removed and the residue was extracted with EtOAc and water. The organic layer was dried, concentrated and purified by chromatography to give 180 mg of compound 130.

Compound 130: ¹H NMR (CDCl₃, 300 MHz) δ 7.82-7.84 (m, 1H), 7.73-7.69 (m, 1H), 7.60-7.53 (m, 1H), 7.41-7.35 (m, 1H), 7.26-6.97 (m, 4H), 6.76 (d, J=8.7 Hz, 1H), 4.40 (d, J=15.9 Hz, 1H), 3.94-3.81 (m, 2H), 3.49 (s, 3H), 3.43-3.36 (m, 2H), 3.25-3.21 (m, 1H), 3.01-2.80 (m, 3H), 2.31 (s, 3H). MS: m/z=454.1 (M⁺+1).

### 47. Preparation of Compound 131 and Compound 132

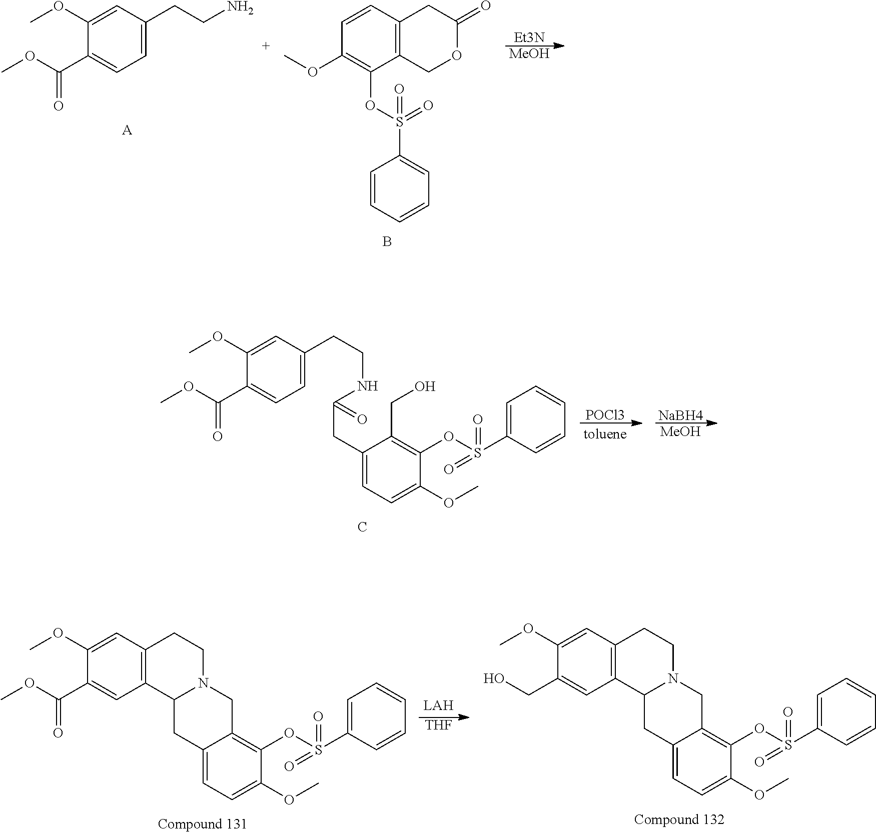

### A+B→C

To a solution of A (3.1 g, 15 mmol) and B (5.0 g, 15 mmol) in MeOH (80 mL) was added Et₃N (2.5 mL, 18 mmol) at 20° C. and the solution was heated to reflux overnight. When TLC analysis indicated the completion of the reaction, water and EtOAc were added, and the organic layer was collected, dried, concentrated and purified by chromatography to give 6.5 g of C.

### C→Compound 131

To a stirred solution of C (5.4 g, 10 mmol) in toluene (50 mL) was added POCl₃ (2 mL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (50 mL), NaBH₄ (1.6 g, 40 mmol) was added at 0° C. The reaction mixture was stirred for 10 min at this temperature. TLC analysis indicated the completion of the reaction. The solvent was removed and the residue was extracted with EtOAc and water. The organic layer was dried, concentrated and purified by chromatography to give 4.0 g of compound 131.

### Compound 131→Compound 132

To a stirred solution of 131 (509 mg, 1 mmol) in THF (5 mL) was slowly added LAH (114 mg, 3 mmol) at room temperature for 2 h. TLC analysis indicated the completion of the reaction. Water (0.1 mL), NaOH (25% aq, 0.1 mL) and water (0.3 mL) was added one by one, the mixture was filtered, and the solution was concentrated, extracted with EtOAc and water. The organic layer was dried, concentrated and purified by chromatography to give 430 mg of compound 132.

Compound 131: ¹H NMR (CDCl₃, 300 MHz) δ 8.01-7.98 (m, 2H), 7.72-7.65 (m, 2H), 7.59-7.53 (m, 2H), 7.03 (d, J=8.4 Hz, 1H), 6.72-6.70 (m, 2H), 4.24 (d, J=15.9 Hz, 1H), 3.90 (s, 3H), 3.89 (s, 3H), 3.65-3.57 (m, 2H), 3.41 (s, 3H), 3.39-3.33 (m, 1H), 3.23-3.13 (m, 2H), 2.85-2.75 (m, 2H), 2.67-2.58 (m, 1H). MS: m/z=510.1 (M⁺+1).

Compound 132: ¹H NMR (CDCl₃, 300 MHz) δ 8.01-7.98 (m, 2H), 7.70-7.65 (m, 1H), 7.59-7.54 (m, 2H), 7.14 (s, 1H), 7.02 (d, J=8.7 Hz, 1H), 6.71 (d, J=8.7 Hz, 1H), 6.63 (s, 1H), 4.68-4.66 (m, 2H), 4.22 (d, J=15.9 Hz, 1H), 3.85 (s, 3H), 3.64-3.57 (m, 2H), 3.41 (s, 3H), 3.36-3.30 (m, 1H), 3.17-3.12 (m, 2H), 2.85-2.57 (m, 3H), 2.35-2.29 (m, 1H). MS: m/z=482.1 (M⁺+1).

### 48. Preparation of Compound 133

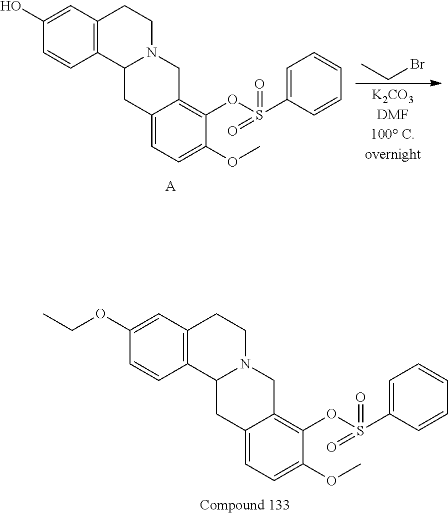

### A→133

To a solution of A (88 mg, 0.2 mmol) in 5 mL of dry DMF was added 0.1 mL of bromoethane, and K₂CO₃ (100 mg) at ambient temperature. The reaction mixture was heated at 100° C. overnight with stirring. When the reaction was completed, reaction mixture was cooled and solvent was removed by vacuum. The residue was extracted by DCM, and purify by preparative TLC to give about 30 mg of compound 133 (PE:EA=3:1).

Compound 133: ¹H NMR (CDCl₃, 300 MHz) δ 8.01-7.98 (d, J=9 Hz, 2H), 7.70-7.65 (m, 1H), 7.59-7.54 (m, 2H), 7.14 (d, J=8.4 Hz, 1H), 7.02 (d, J=8.4 Hz, 1H), 6.79-6.65 (m, 3H), 4.27-4.21 (d, J=18 Hz, 1H), 4.06-3.99 (m, 2H), 3.65-3.60 (m, 2H), 3.44 (s, 3H), 3.29-3.28 (m, 1H), 3.17-3.14 (m, 2H), 2.75-2.63 (m, 3H), 1.43-1.38 (t, 3H). MS: m/z=466.1 (M⁺+1).

### 49. Preparation of Compound 134

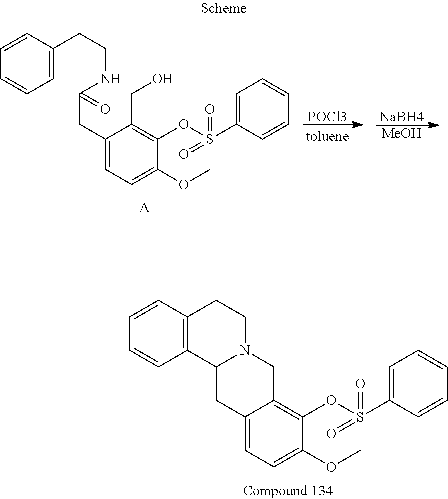

### A→Compound 134

To a white solid A (130 mg, 0.28 mmol) was added POCl₃ (300 μL) at ambient temperature. The reaction mixture was refluxed for 2 h with stirring. When the reaction was complete, excess POCl₃ were evaporated off. The residue was poured into ice-water and adjusted to pH >7 with Na₂CO₃. The solution was extracted with EtOAc, and the organic layer was dried, concentrated and dissolved in MeOH (5 mL), NaBH₄ (86 mg, 2.26 mmol) was then added at 0° C. The reaction mixture was stirred for 30 min at this temperature. TLC analysis indicated the completion of the reaction. The solvent was removed and the residue was extracted with EtOAc and water. The organic layer was dried, concentrated and purified by chromatography to give 10 mg of compound 134.

Compound 134: ¹H NMR (CDCl₃, 300 MHz) δ 8.02-7.99 (d, J=9 Hz, 2H), 7.68-7.66 (m, 1H), 7.59-7.57 (m, 2H), 7.26-7.13 (m, 4H), 7.03 (d, J=8.4 Hz, 1H), 6.74 (m, J=8.4 Hz, 1H), 4.30-4021 (d, J=15.6 Hz, 1H), 3.72-3.65 (m, 2H), 3.44 (s, 3H), 3.37-3.33 (m, 1H), 3.20-3.16 (m, 2H), 2.88-2.74 (m, 3H). MS: m/z=422.1 (M⁺+1).

### 50. Preparation of Compound 135 and Compound 136

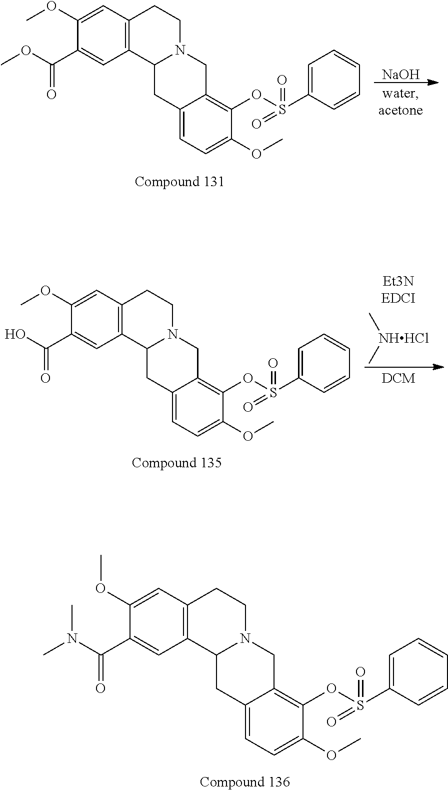

### Compound 131→Compound 135

To a stirred solution of NaOH (400 mg, 10 mmol) in water (4 mL) and acetone (2 mL) was added compound 131 (400 mg, 0.78 mmol) at room temperature, then heated to reflux for 2 h. When TLC analysis indicated completion of reaction, the reaction solution was adjusted to pH=3 with conc. HCl at room temperature. The mixture was extracted with EtOAc, dried, concentrated, and purified by silica gel to give 230 mg of compound 135.

### Compound 135→Compound 136

A mixture of compound 135 (49.5 mg, 0.1 mmol), dimethylamine hydrochloride (16.3 mg, 0.2 mmol), EDCI (38.4 mg, 0.2 mmol) and triethylamine (55 μL, 0.4 mmol) were added to DCM (5 mL) at room temperature and stirred overnight. When TLC analysis indicated the completion, the mixture was concentrated, extracted with EtOAc, dried, concentrated, and purified by silica gel to give 30 mg of compound 136

Compound 135: ¹H NMR (DMSO-d₆, 300 MHz) δ 11.74 (s, 1H), 7.96 (d, J=7.2 Hz, 2H), 7.89-7.84 (m, 1H), 7.75-7.70 (m, 3H), 7.31 (d, J=8.4 Hz, 1H), 7.12-7.04 (m, 2H), 4.86-4.83 (m, 1H), 4.72 (d, J=15.6 Hz, 1H), 4.54-4.51 (m, 1H), 3.95-3.81 (m, 5H), 3.59-3.43 (m, 2H), 3.38 (s, 3H), 3.16-3.03 (m, 2H). MS: m/z=494.0 (M⁺−1).

Compound 136: ¹H NMR (CDCl₃, 300 MHz) δ 8.00-7.97 (m, 2H), 7.69-7.64 (m, 1H), 7.58-7.53 (m, 2H), 7.13 (s, 1H), 6.99 (d, J=8.7 Hz, 1H), 6.70 (d, J=8.7 Hz, 1H), 6.63 (s, 1H), 4.24 (d, J=15.9 Hz, 1H), 3.81 (s, 3H), 3.64-3.61 (m, 2H), 3.41 (s, 3H), 3.30 (dd, J=3.9, 16.2 Hz, 1H), 3.19-3.14 (m, 2H), 3.11 (s, 3H), 2.87 (s, 3H), 2.84-2.60 (m, 3H). MS: m/z=523.1 (M⁺+1).

### 51. Preparation of Compound 137

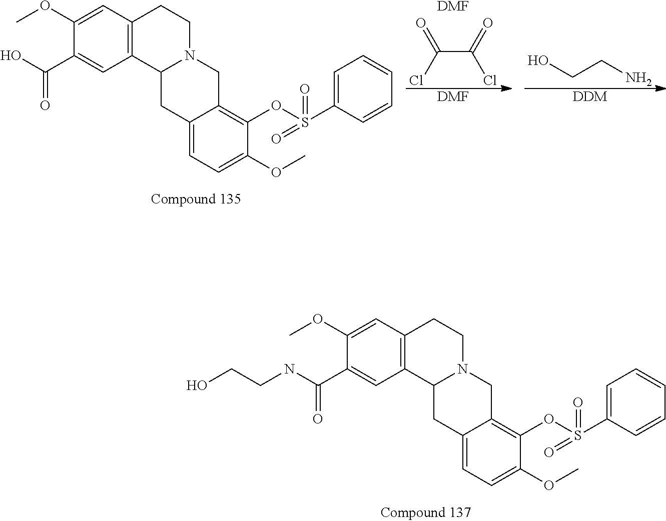

### Compound 135→Compound 137

To a stirred solution of 135 (49.5 mg, 0.1 mmol) and DMF (2 drops) in DCM (2 mL) was added drop-wise oxalyl chloride (1.0 mL) at ambient temperature, then heated to reflux for 1 h. The reaction solution was concentrated and re-dissolved in DCM (1.5 mL) and added to 2-aminoethan-1-ol (12 μL, 0.2 mmol) in DCM (1.5 mL) and maintained at rt for 2 h. When TLC analysis indicated the completion of reaction, the mixture was extracted with DCM, washed with water, dried, concentrated, and purified by silica gel to give 30 mg of compound 137.

Compound 137: ¹H NMR (CDCl₃, 300 MHz) δ 8.32 (t, J=5.7 Hz, 1H), 8.11 (s, 1H), 7.98 (d, J=7.8 Hz, 2H), 7.67 (t, J=7.5 Hz, 1H), 7.55 (t, J=8.1 Hz, 2H), 7.01 (d, J=8.1 Hz, 1H), 6.71-6.68 (m, 2H), 4.25 (d, J=15.6 Hz, 1H), 3.98 (s, 3H), 3.84-3.80 (m, 2H), 3.65-3.60 (m, 4H), 3.50-3.42 (m, 1H), 3.38 (s, 3H), 3.25-3.14 (m, 2H), 2.84-2.75 (m, 2H), 2.67-2.59 (m, 1H). MS: m/z=539.1 (M⁺+1).

### 52. Preparation of Compound 138

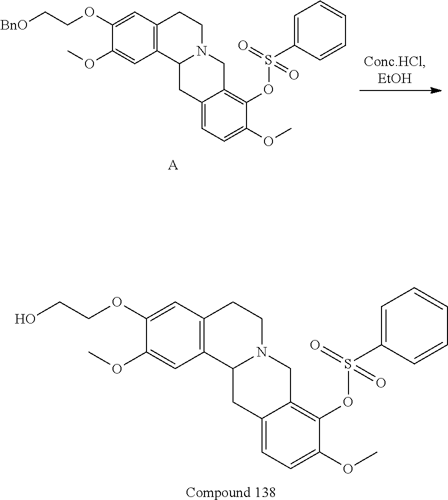

Compound A (0.35 mmol) was dissolved in 5 mL of EtOH and 5 mL of conc. HCl was added to it dropwise. The reaction mixture was refluxed for 2 h. Then it was neutralized with saturated aq. NaHCO₃ and extracted with EtOAc. The organic extract was dried with anhydrous Na₂SO₄ and evaporated to give an oily residue, which was purified with silica gel to furnish compound 138.

Compound 138: ¹H NMR (CDCl₃, 300 MHz) δ=8.02-7.99 (m, 2H), 7.71-7.66 (m, 2H), 7.60-7.54 (m, 2H), 7.03 (d, J=8.4 Hz, 1H), 6.73-6.68 (m, 3H), 4.22 (d, J=16.0 Hz, 1H), 4.13-4.10 (m, 2H), 3.92 (br, 2H), 3.87 (s, 3H), 3.64-3.59 (m, 2H), 3.43 (s, 3H), 3.31-3.05 (m, 3H), 2.88-2.57 (m, 3H); MS: m/z=512.1 (M⁺+1).

### 53. Preparation of Compound 141

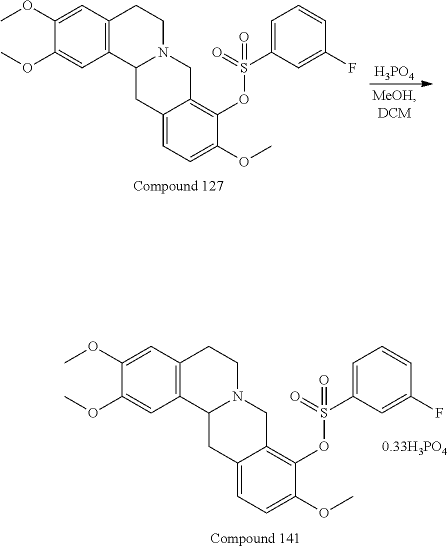

To a stirred solution of compound 127 (1.497 g, 3.0 mmol) in MeOH (10 mL) and DCM (20 mL) was added 85% of H₃PO₄ (115 mg, 1.0 mmol) at room temperature and stirred for 2 h. The solvent was removed to give compound 141.

Compound 141: ¹H NMR (DMSO-d₆, 300 MHz) δ 7.82-7.70 (m, 4H), 7.13 (d, J=8.7 Hz, 1H), 6.96 (d, J=8.7 Hz, 1H), 6.88 (s, 1H), 6.69 (s, 1H), 4.01 (d, J=15.9 Hz, 1H), 3.75 (s, 3H), 3.73 (s, 3H), 3.48-3.38 (m, 6H), 3.04-2.88 (m, 2H), 2.64-2.58 (m, 2H), 2.53-2.42 (m, 1H). MS: m/z=500.1 (M⁺+1).

### 54. Preparation of Compound 142

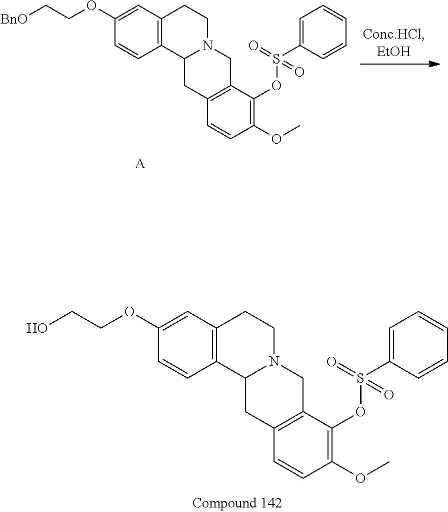

Compound A (200 mg, 0.35 mmol) was dissolved in 5 mL of EtOH, and 5 mL of conc. HCl was added dropwise. The reaction mixture was refluxed for 2 h. Then it was neutralized with saturated aq. NaHCO₃ and extracted with EtOAc. The organic layer was dried with anhydrous Na₂SO₄ and evaporated to give an oily residue, which was purified with silica gel to afford compound 142.

Compound 142: ¹H NMR (CDCl₃, 300 MHz) δ=8.02-7.99 (m, 2H), 7.68-7.66 (m, 1H), 7.59-7.54 (m, 2H), 7.18-7.15 (d, J=8.4 Hz, 1H), 7.04-7.01 (m, 1H), 6.82-6.68 (m, 3H), 4.27 (d, J=16.0 Hz, 1H), 4.10-4.07 (m, 2H), 3.97-3.95 (m, 2H), 3.80-3.66 (m, 2H), 3.44 (s, 3H), 3.34-3.30 (m, 1H), 3.18-3.13 (m, 2H), 2.84-2.64 (m, 3H); MS: m/z=482.1 (M⁺+1).

### 55. Preparation of Compound 143

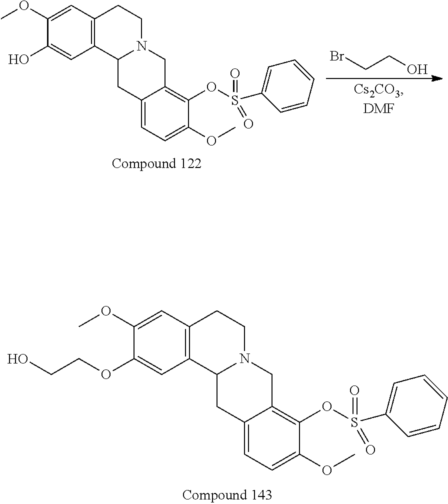

### Compound 122 to Compound 143:

To a stirred mixture of compound 122 (100 mg, 0.21 mmol) and Cs₂CO₃ (68 mg, 0.21 mmol) in DMF (10 mL) was added 2-bromoethan-1-ol (31 mg, 0.25 mmol). The reaction mixture was stirred at 80° C. overnight. The solvent was removed, and the residue was diluted with DCM. The solution was washed with water and dried over anhydrous Na₂SO₄. The solvent was removed under vacuum; the residue was purified by silica gel chromatography to afford 10 mg of compound 143.

Compound 143: ¹H NMR (CDCl₃, 300 MHz) δ7.99-8.02 (m, 2H), 7.66-7.71 (m, 1H), 7.55-7.60 (m, 2H), 7.03 (d, J=8.4 Hz, 1H), 6.82 (s, 1H), 6.71 (s, 1H), 6.64 (s, 1H), 4.24 (d, J=15.9 Hz, 1H), 4.11-4.20 (m, 2H), 3.92 (t, J=4.2 Hz, 2H), 3.86 (s, 3H), 3.58-3.66 (m, 2H), 3.44 (s, 3H), 3.27 (dd, J=15.9 Hz, 1H), 3.12-3.17 (m, 2H), 2.62-2.89 (m, 4H). MS: m/z=512.1 (M⁺+1).

### 56. Preparation of Compound 144

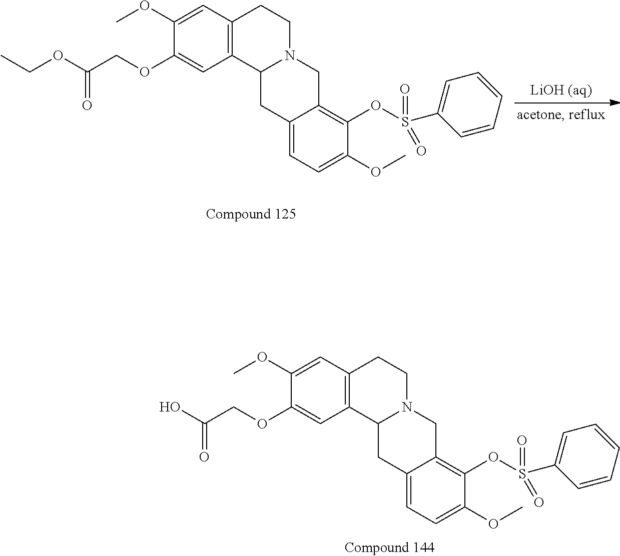

### Compound 125 to Compound 144:

To a stirred solution of compound 125 (100 mg, 0.18 mmol) in acetone (5 mL) was added 2N LiOH (0.1 mL, 0.18 mmol) at room temperature, then heated to reflux for 2 h. When TLC analysis indicated completion of reaction, the reaction solution was adjusted pH=3 with conc. HCl at room temperature. The mixture was extracted with DCM, dried, concentrated, and purified by silica gel to give 90 mg of compound 144.

Compound 144: ¹H NMR (MeOH-d₄, 300 MHz) δ7.92-7.95 (m, 2H), 7.74-7.80 (m, 1H), 7.62-7.67 (m, 2H), 7.14 (d, J=8.4 Hz, 1H), 6.90 (s, 1H), 6.87 (s, 1H), 6.79 (s, 1H), 4.39 (s, 2H), 4.26 (d, J=15.9 Hz, 1H), 3.87 (s, 3H), 3.63-3.71 (m, 2H), 3.47-3.53 (m, 2H), 3.40 (s, 3H), 3.10-3.23 (m, 2H), 2.68-2.82 (m, 2H). MS: m/z=524.1 (M⁻−1).

### 57. Preparation of Compound 145

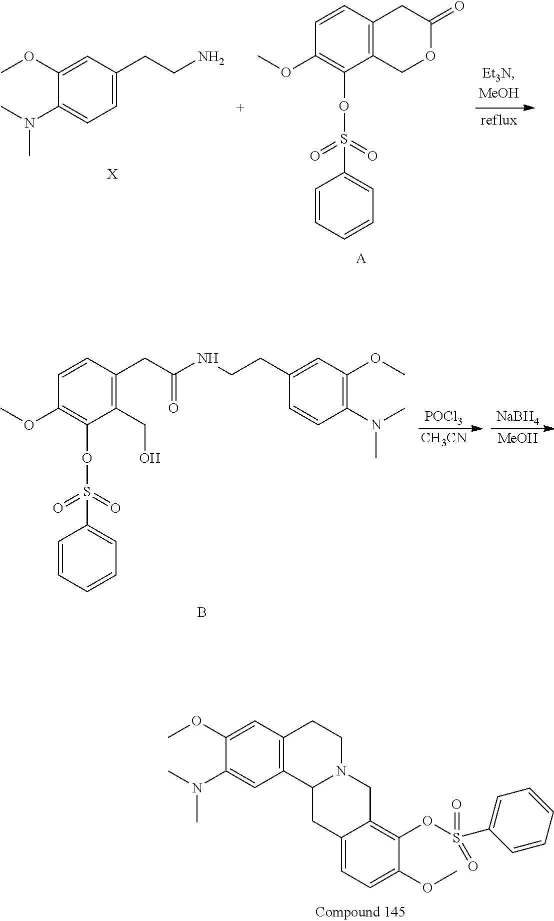

### A to B

MeOH 20 mL was added to a mixture of A (500 mg, 1.5 mmol), X (349 mg, 1.8 mmol) and 1 mL Et₃N. The resulting solution was stirred at reflux temperature for 2 h. Then the reaction mixture was diluted with DCM, washed with water, dried over anhydrous Na₂SO₄. The solvent was removed under vacuum and the residue was purified by silica gel chromatography to afford 700 mg of B.

### B to Compound 145

To a stirred solution of B (200 mg, 0.4 mmol) in CH₃CN (15 mL) was added POCl₃ (1 mL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with DCM, and the organic layer was dried, concentrated and dissolved in MeOH (20 mL) to which NaBH₄ (72 mg, 1.9 mmol) was added at 0° C. The reaction mixture was stirred for 30 min at this temperature. TLC analysis indicated the completion of the reaction. The solvent was removed and the residue was extracted with DCM and water. The organic layer was dried, concentrated and purified by chromatography to give 50 mg of compound 145.

Compound 145: ¹H NMR (CDCl₃, 300 MHz) δ 7.93 (d, J=7.5 Hz, 2H), 7.60 (t, J=7.5 Hz, 1H), 7.50 (t, J=7.5 Hz, 2H), 6.97 (d, J=8.7 Hz, 1H), 6.71 (s, 1H), 6.65 (d, J=8.7 Hz, 1H), 6.52 (s, 1H), 4.15 (d, J=16.2 Hz, 1H), 3.80 (s, 3H), 3.50-3.55 (m, 2H), 3.37 (s, 3H), 3.19-3.25 (m, 1H), 3.01-3.09 (m, 2H), 2.72 (s, 6H), 2.50-2.62 (m, 3H). MS: m/z=495.2 (M⁺+1).

### 58. Preparation of Compound 146 and Compound 147

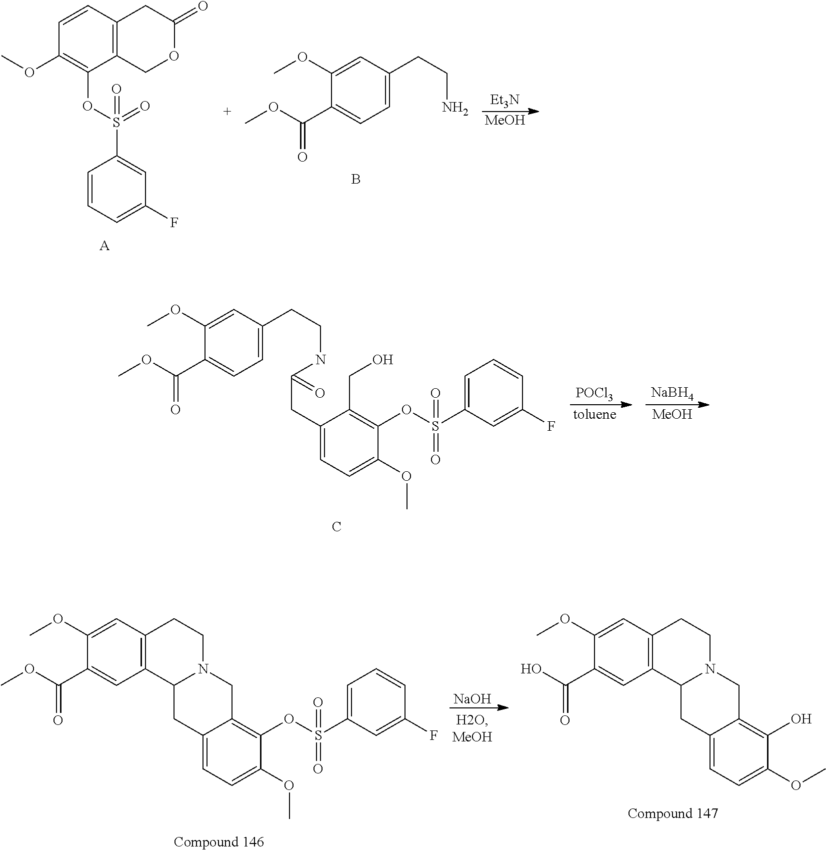

### A+B→C

To the solution of A (1.27 g, 3.6 mmol) and B (627 mg, 3.0 mmol) in MeOH (30 mL) was added Et₃N (600 μL, 4.5 mmol) at 20° C. and the solution was heated to reflux overnight. When TLC analysis indicated completion of the reaction, water and EtOAc were added, and the organic layer was collected, dried, concentrated and purified by chromatography to give 1.2 g of C.

### C→Compound 146

To a stirred solution of C (1.2 g, 2.14 mmol) in toluene (50 mL) was added POCl₃ (0.6 mL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (50 mL) to which NaBH₄ (244 mg, 6.42 mmol) was added at 0° C. The reaction mixture was stirred for 10 min at this temperature. TLC analysis indicated completion of the reaction. The solvent was removed and the residue was extracted with EtOAc and water. The organic layer was dried, concentrated and purified by preparative TLC to give 800 mg of compound 146.

### Compound 146→Compound 147

To a stirred solution of NaOH (600 mg, 15 mmol) in water (4 mL) and MeOH (2 mL) was added 146 (400 mg, 0.76 mmol) at room temperature, then heated to reflux for 4 h. When TLC analysis indicated completion of the reaction, the reaction solution was adjusted pH=3 with conc. HCl at room temperature. The mixture was extracted with EtOAc, dried, concentrated, and purified by silica gel to give 200 mg of compound 147.

Compound 146: ¹H NMR (CDCl₃, 300 MHz) δ 7.82-7.79 (m, 1H), 7.74-7.71 (m, 2H), 7.60-7.53 (m, 1H), 7.43-7.36 (m, 1H), 7.06 (d, J=8.7 Hz, 1H), 6.75-6.72 (m, 2H), 4.26 (d, J=15.9 Hz, 1H), 3.91 (s, 3H), 3.90 (s, 3H), 3.68-3.59 (m, 2H), 3.47 (s, 3H), 3.37 (dd, J=3.9, 15.9 Hz, 1H), 3.23-3.14 (m, 2H), 2.87-2.76 (m, 2H), 2.70-2.61 (m, 1H). MS: m/z=528.2 (M⁺+1).

Compound 147: ¹H NMR (DMSO-d₆, 300 MHz) δ 12.74 (br, 1H), 12.16-12.14 (m, 1H), 9.22 (s, 1H), 7.72 (s, 1H), 7.03 (s, 1H), 6.95 (d, J=8.7 Hz, 1H), 6.76 (d, J=8.7 Hz, 1H), 4.71 (t, J=9.0 Hz, 1H), 4.50 (d, J=15.0 Hz, 1H), 4.26-4.18 (m, 1H), 3.83 (s, 3H), 3.80 (s, 3H), 3.72-3.42 (m, 4H), 3.18-2.98 (m, 2H). MS: m/z=354.1 (M⁺−1).

### 59. Modification on R4

To make R4 deletion, the following synthetic route was developed as shown in the Scheme.

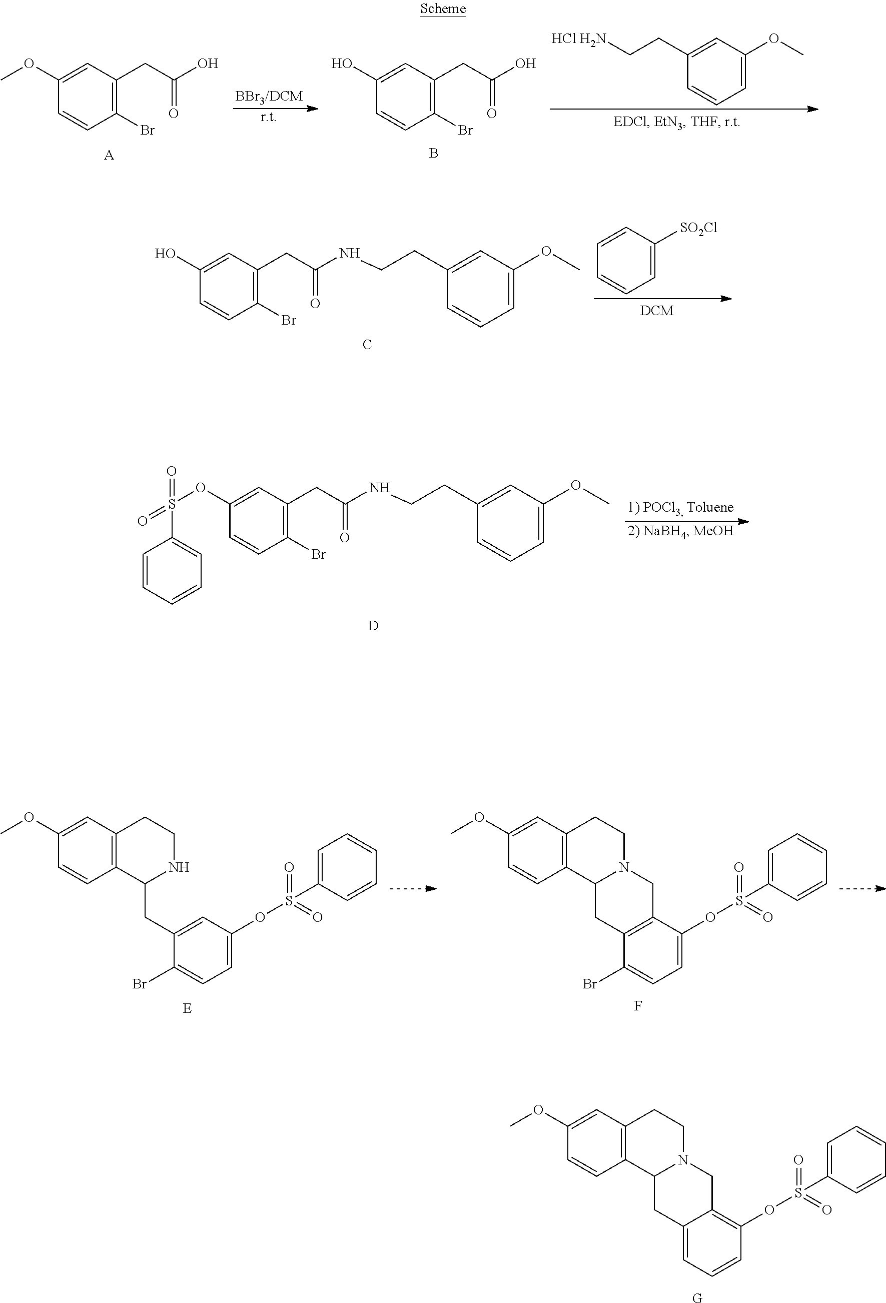

(1) Preparation of Compound 149, Compound 150 and Compound 151

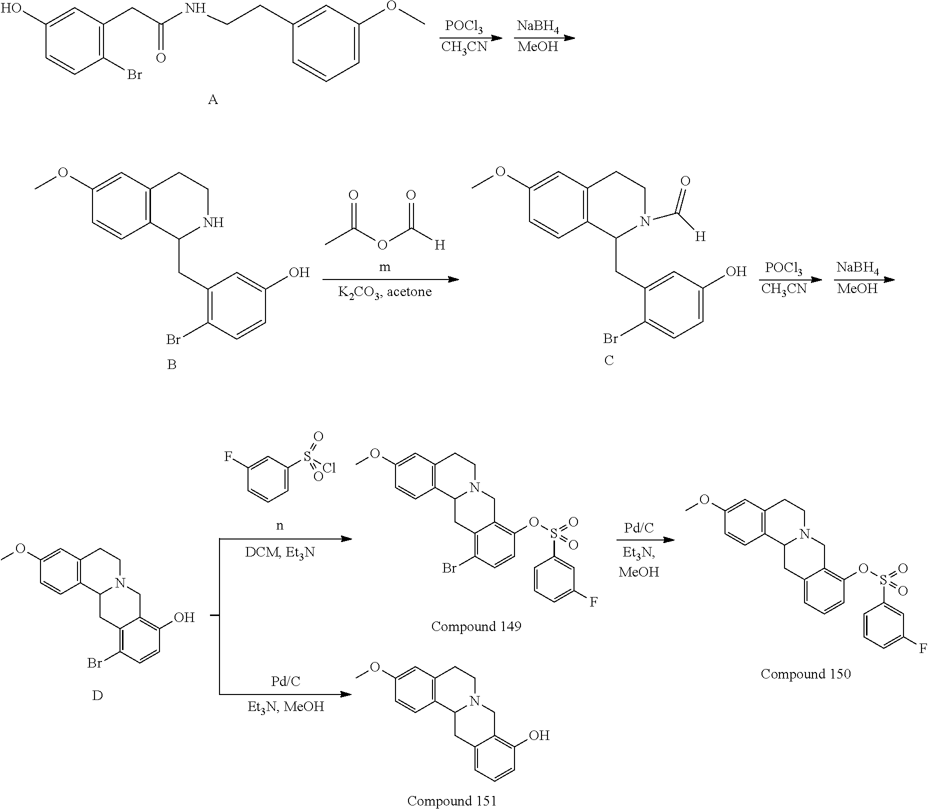

### A to B

To a stirred solution of A (5.0 g, 13.7 mmol) in CH₃CN (350 mL) was added POCl₃ (5 mL) at ambient temperature. The reaction mixture was refluxed for 2 h with stirring. When the reaction was complete, the solvent and excess POCl₃ were evaporated off. The solution was extracted with DCM and water, and the organic layer was dried, concentrated and dissolved in MeOH (300 mL) to which NaBH₄ (2.6 g, 68.5 mmol) was added at 0° C. The reaction mixture was stirred for 30 min at this temperature. TLC analysis indicated completion of the reaction. The solvent was removed and the residue was extracted with DCM and water. The organic layer was dried, concentrated and purified by chromatography to g 4.3 g of B.

### B to C

To a stirred mixture of B (2.0 g, 5.8 mmol) and K₂CO₃ (2.4 g, 17.3 mmol) in acetone (100 mL) was added m (0.5 g, 5.8 mmol) at rt. The mixture was stirred for 3 h. The solvent was removed and the residue was extracted with DCM and water. The organic layer was dried, concentrated and purified to give 1.3 g of C.

### C to D

To a stirred solution of C (1.3 g, 3.5 mmol) in CH₃CN (150 mL) was added POCl₃ (1.5 mL) at ambient temperature. The reaction mixture was refluxed for 2 h with stirring. When the reaction was complete, the solvent and excess POCl₃ were evaporated off. The solution was extracted with DCM and water, and the organic layer was dried, concentrated and dissolved in MeOH (120 mL) to which NaBH₄ (0.67 g, 17.7 mmol) was added at 0° C. The reaction mixture was stirred for 30 min at this temperature. TLC analysis indicated completion of the reaction. The solvent was removed and the residue was extracted with DCM and water. The organic layer was dried, concentrated and purified by chromatography to give 0.78 g of D.

### D to Compound 149

To a stirred solution of D (100 mg, 0.3 mmol) in DCM (10 mL) was added n (60 mg, 0.3 mmol) and Eₜ₃N (0.5 mL) at rt. The mixture was stirred for 2 h. The mixture was washed with water. The organic layer was dried, concentrated and purified to give 120 mg of compound 149.

Compound 149: ¹H NMR (CDCl₃, 300 MHz) δ 7.70-7.74 (m, 1H), 7.62-7.66 (m, 1H), 7.55-7.61 (m, 1H), 7.40-7.46 (m, 1H), 7.38 (d, J=8.7 Hz, 1H), 7.21 (d, J=8.7 Hz, 1H), 6.81 (dd, J=8.7 Hz, 1H), 6.66-6.70 (m, 2H), 4.07 (d, J=15.6 Hz, 1H), 3.81 (s, 3H), 3.51-3.56 (m, 1H), 3.29-3.41 (m, 2H), 3.10-3.13 (m, 2H), 2.56-2.77 (m, 3H). MS: m/z=518.0 (M⁺+1).

### Compound 149 to Compound 150

To a stirred mixture of 149 (50 mg, 0.1 mmol) and Pd/C (20 mg) in MeOH (10 mL) was added Eₜ₃N (0.5 mL) at rt. The mixture was stirred overnight. Then the solid was filtered off. The solvent was removed under vacuum and the residue was extracted with DCM and water. The organic layer was dried, concentrated and purified to give 30 mg of compound 150.

Compound 150: ¹H NMR (CDCl₃, 300 MHz) δ 7.72 (d, J=7.8 Hz, 1H), 7.52-7.65 (m, 2H), 7.37-7.44 (m, 1H), 7.09-7.16 (m, 3H), 6.77-6.80 (m, 2H), 7.66 (s, 1H), 4.08 (d, J=15.9 Hz, 1H), 3.80 (s, 3H), 3.51-3.56 (m, 1H), 3.30-3.35 (m, 2H), 3.07-3.20 (m, 2H), 2.53-2.90 (m, 3H). MS: m/z=440.1 (M⁺+1).

### D to Compound 151

To a stirred mixture of D (100 mg, 0.3 mmol) and Pd/C (40 mg) in MeOH (15 mL) was added Eₜ₃N (1 mL) at rt. The mixture was stirred overnight. Then the solid was filtered off. The solvent was removed under vacuum and the residue was extracted with DCM and water. The organic layer was dried, concentrated and purified to give 80 mg of compound 151.

Compound 151: ¹H NMR (CDCl₃, 300 MHz) δ 7.19 (d, J=8.7 Hz, 1H), 6.99 (t, J=7.8 Hz, 1H), 6.72-6.81 (m, 2H), 6.67 (s, 1H), 6.51 (d, J=7.8 Hz, 1H), 4.19 (d, J=15.6 Hz, 1H), 3.81 (s, 3H), 3.46-3.66 (m, 2H), 3.18-3.36 (m, 3H), 2.64-2.93 (m, 3H). MS: m/z=282.1 (M⁺+1).

### 60. Approach to 13-Substituted Compound

To synthesis 13-substituted compounds, the following route was developed shown in the Scheme below.

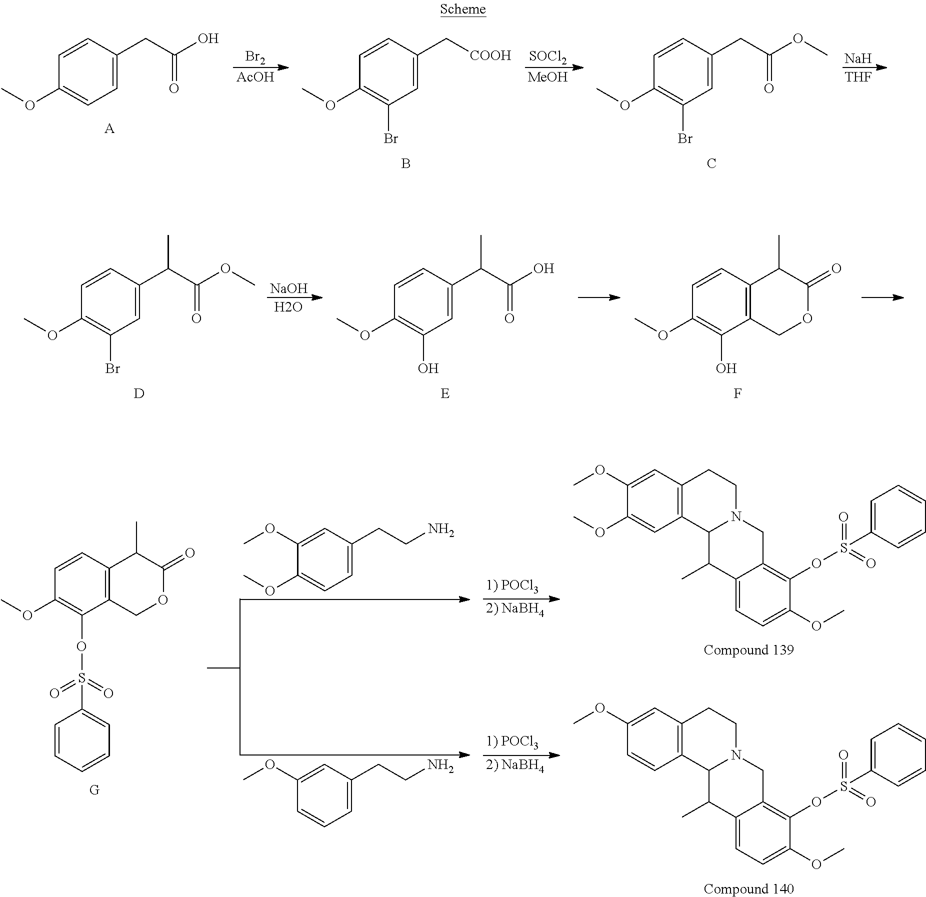

(1) Preparation of Compound 139

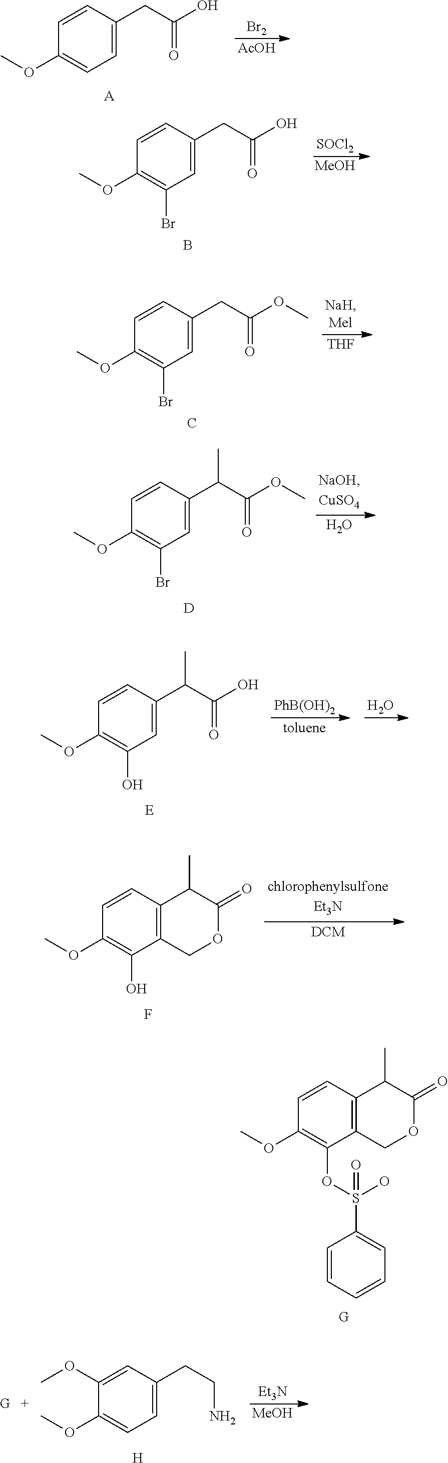

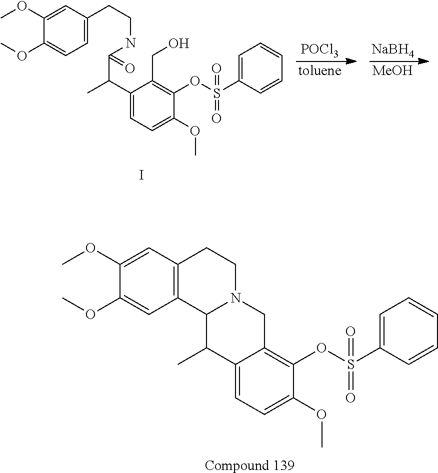

### A→B

To a stirred solution of A (166 g, 1.0 mol) in AcOH (2.0 L) was added bromine (56 mL, 1.1 mol) drop-wise at room temperature. The solution was stirred overnight. When the reaction was completed, the solvent was evaporated off. The residue was extracted with EtOAc, washed with water and concentrated to give 225 g of B.

### B→C

To a stirred solution of B (150 g, 0.6 mol) in MeOH (1.0 L) was added SOCl₂ (87 mL, 1.2 mol) slowly at rt and heated to reflux for 2 h. The solvent and excess SOCl₂ were evaporated off to give 150 g of C.

### C→D

To a stirred solution of C (51.8 g, 0.2 mol) in THF (700 mL) was added 60% of NaH (8.8 g, 0.22 mol) at room temperature and stirred for 30 min. MeI (13 mL, 0.21 mol) was then added and stirred for 1 h. When TLC analysis indicated completion of the reaction, water was added carefully. The mixture was concentrated, the residue was extracted with water and EtOAc and the organic layer was collected, dried, concentrated and purified to give 46 g of D.

### D→E

To a stirred solution of CuSO₄ (2.7 g, 17 mmol), NaOH (100 g, 2.5 mol) in water (1.0 L) was added D (46 g, 168 mol) under N₂ in a steel bomb at room temperature. Then the solution was heated to 150° C. overnight. When the reaction was complete, the reaction solution was adjusted to pH=3 with conc. HCl at room temperature. The solution was extracted with EtOAc, dried, concentrated, and purified to give 30 g E.

### E→F

To 30 g of E (153 mmol) was added phenylboric acid (56 g, 306 mmol) and toluene (1.0 L). The mixture was heated at reflux for 1 h, and water was collected in a dean-stark trap. The hot solution was poured over molecular sieves (10 g) in a stainless steel bomb. Paraformaldehyde (10 g, 306 mmol) was added. The bomb was sealed and heated on an oil-bath at 110° C. for 48 h. The bomb was opened and the hot solution was filtered. Toluene was evaporated and water (500 mL) was added to the residue. After heating at reflux for 2 h, the mixture was cooled to room temperature and extracted with DCM. The solution was dried and the solvent was removed. The residue was washed with ether to obtain 13 g of F.

### F→G

To a stirred solution of F (13 g, 62.5 mmol) in DCM (200 mL) was added chlorophenylsulfone (16.2 mL, 125 mmol) at rt: Et₃N (25 mL) was then added and stirred for 4 h. When TLC analysis indicated the completion of the reaction, the solvent was removed under reduced pressure. The residue was further purified to give 5 g of G.

### G+H→I

To the solution of G (69.6 mg, 0.2 mmol), H (54.3 mg, 0.3 mmol) in MeOH (5 mL) was added Et₃N (41.5 μA, 0.3 mmol) at 20° C. and the solution was heated to reflux overnight. When TLC analysis indicated completion of the reaction, water and EtOAc were added, and the organic layer was collected, dried, concentrated and purified by chromatography to give 86 mg of I.

### I→Compound 139

To a stirred solution of I (86 mg, 0.16 mmol) in toluene (5 mL) was added POCl₃ (0.1 mL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (5 mL) to which NaBH₄ (15.2 mg, 0.4 mmol) was added at 0° C. The reaction mixture was stirred for 10 min at this temperature. TLC analysis indicated completion of the reaction. The solvent was removed and the residue was extracted with EtOAc and water. The organic layer was dried, concentrated and purified by preparative TLC to give 50 mg of compound 139.

Compound 139: ¹H NMR (CDCl₃, 300 MHz) δ 7.94 (d, J=7.5 Hz, 2H), 7.69-7.64 (m, 1H), 7.57-7.52 (m, 2H), 7.10 (d, J=8.7 Hz, 1H), 6.78 (m, J=8.7 Hz, 1H), 6.70 (s, 1H), 6.61 (s, 1H), 4.22 (dd, J=16.5, 28.5 Hz, 2H), 3.94 (d, J=7.8 Hz, 1H), 3.84 (s, 3H), 3.82 (s, 3H), 3.47 (s, 3H), 3.15-3.03 (m, 4H), 2.90-2.84 (m, 1H), 1.50 (d, J=6.9 Hz, 3H). MS: m/z=496.1 (M⁺+1).

(2) Preparation of Compound 140

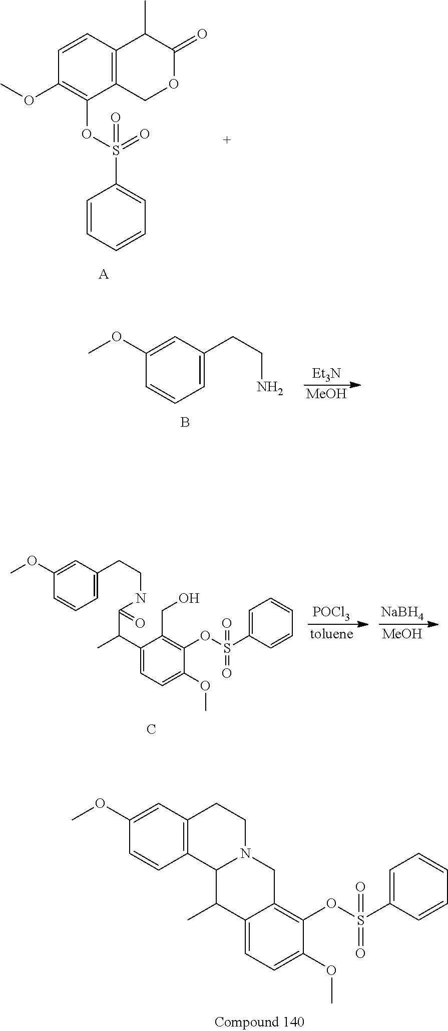

### A+B→C

To the solution of A (69.6 mg, 0.2 mmol) and B (45.3 mg, 0.3 mmol) in MeOH (5 mL) was added Et₃N (41.5 μA, 0.3 mmol) at 20° C. and the solution was heated to reflux overnight. When TLC analysis indicated completion of the reaction, water and EtOAc were added, and the organic layer was collected, dried, concentrated and purified by chromatography to give 50 mg of C.

### C→Compound 140

To a stirred solution of C (50 mg, 0.1 mmol) in toluene (5 mL) was added POCl₃ (0.1 mL) at ambient temperature. The reaction mixture was refluxed for 2 h with stirring. When the reaction was completed, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (5 mL) to which NaBH₄ (11.4 mg, 0.3 mmol) was added at 0° C. The reaction mixture was stirred for 10 min at this temperature. TLC analysis indicated completion of the reaction. The solvent was removed and the residue was extracted with EtOAc and water. The organic layer was dried, concentrated and purified by preparative TLC to give 7.3 mg of compound 140.

Compound 140: ¹H NMR (CDCl₃, 300 MHz) δ 7.99-7.96 (m, 2H), 7.70-7.64 (m, 1H), 7.58-7.53 (m, 2H), 7.15 (d, J=5.6 Hz, 1H), 7.08 (m, J=5.6 Hz, 1H), 6.74-6.65 (m, 3H), 4.08-3.86 (m, 2H), 3.78 (s, 3H), 3.61 (d, J=8.4 Hz, 1H), 3.47 (s, 3H), 3.03-2.89 (m, 4H), 2.76-2.70 (m, 1H), 1.45 (d, J=6.9 Hz, 3H). MS: m/z=466.1 (M⁺+1).

(3) Preparation of Compound 148

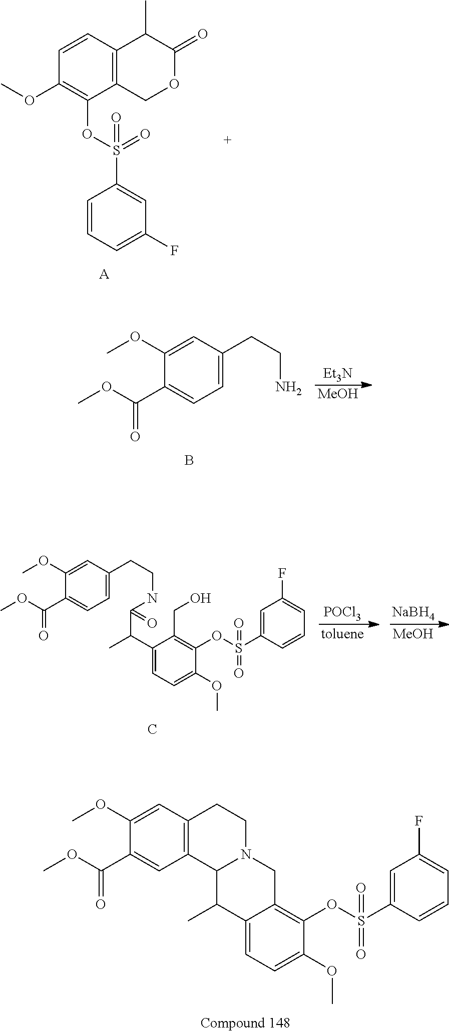

### A+B→C

To a solution of A (3.66 g, 10 mmol) and B (2.09 g, 10 mmol) in MeOH (50 mL) was added Et₃N (2.1 mL, 15 mmol) at 20° C. and the solution was heated to reflux overnight. When TLC analysis indicated completion of the reaction, water and EtOAc were added, and the organic layer was collected, dried, concentrated and purified by chromatography to give 4.0 g of C.

### C→Compound 148

To a stirred solution of C (4.0 g, 6.96 mmol) in toluene (100 mL) was added POCl₃ (2 mL) at ambient temperature. The reaction mixture was refluxed for 2 h with stirring. When the reaction was complete, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (100 mL) to which NaBH₄ (793 mg, 20.87 mmol) was added at 0° C. The reaction mixture was stirred for 10 min at this temperature. TLC analysis indicated completion of the reaction. The solvent was removed and the residue was extracted with EtOAc and water. The organic layer was dried, concentrated and purified by preparative TLC to give 2.0 g of compound 148.

Compound 148: ¹H NMR (CDCl₃, 300 MHz) δ 7.78-7.68 (m, 3H), 7.58-7.51 (m, 1H), 7.40-7.26 (m, 1H), 7.11 (d, J=11.7 Hz, 1H), 4.14-3.94 (m, 2H), 3.88 (s, 3H), 3.87 (s, 3H), 3.62 (d, J=8.4 Hz, 1H), 3.48 (s, 3H), 3.04-2.92 (m, 4H), 2.80-2.74 (m, 1H), 1.47 (d, J=3.9 Hz, 3H). MS: m/z=542.1 (M⁺+1).

(4) Preparation of Compound 152 and Compound 153

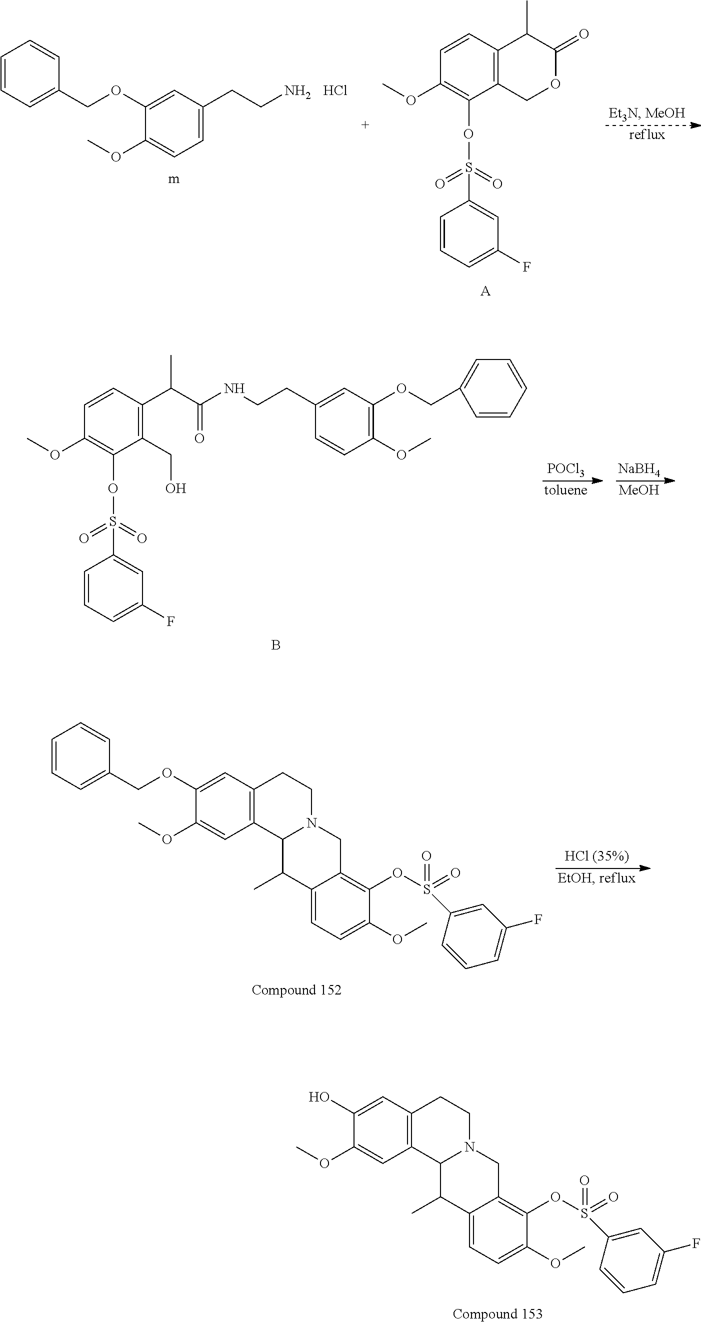

### A to B

MeOH 30 mL was added to a mixture of m (0.88 g, 3.0 mmol), A (1.0 g, 2.8 mmol) and 1 mL Et₃N. The resulting solution was stirred at reflux temperature for 2 h. Then the solvent was removed, and the residue was diluted with DCM, washed with water and dried over anhydrous Na₂SO₄. The solvent was removed under vacuum and the residue was purified by silica gel chromatography to afford 930 mg of B.

### B to Compound 152

To a stirred solution of B (930 mg, 1.5 mmol) in toluene (60 mL) was added POCl₃ (1 mL) at ambient temperature. The reaction mixture was refluxed for 2 h with stirring. When the reaction was completed, the solvent and excess POCl₃ were evaporated off. The solution was extracted with DCM and water, and the organic layer was dried, concentrated and dissolved in MeOH (50 mL) to which NaBH₄ (283 mg, 7.5 mmol) was added at 0° C. The reaction mixture was stirred for 30 min at this temperature. TLC analysis indicated completion of the reaction. The solvent was removed and the residue was extracted with DCM and water. The organic layer was dried, concentrated and purified by chromatography to give 330 mg of compound 152.

Compound 152: ¹H NMR (CDCl₃, 300 MHz) δ 7.77-7.80 (m, 1H), 7.68-7.73 (m, 1H), 7.51-7.58 (m, 1H), 7.29-7.45 (m, 6H), 7.11 (d, J=9.0 Hz, 1H), 6.74-6.77 (m, 2H), 6.65 (s, 1H), 5.12 (s, 2H), 3.95-4.13 (m, 2H), 3.88 (s, 3H), 3.62 (d, J=8.7 Hz, 1H), 3.51 (s, 3H), 2.73-3.04 (m, 5H), 1.49 (d, J=6.9 Hz, 3H). MS: m/z=590.2 (M⁺+1).

### Compound 152 to Compound 153

10 mL conc. HCl was added dropwise to a solution of 152 (330 mg, 0.6 mmol) in 10 mL EtOH. The reaction mixture was refluxed for 2 h. Then it was neutralized with saturated aq. NaHCO₃ and extracted with DCM. The organic layer was dried, concentrated and purified by chromatography to give 130 mg of compound 153.

Compound 153: ¹H NMR (CDCl₃, 300 MHz) δ 7.78-7.81 (m, 1H), 7.67-7.73 (m, 1H), 7.53-7.59 (m, 1H), 7.35-7.41 (m, 1H), 7.11 (d, J=8.7 Hz, 1H), 6.76 (d, J=8.7 Hz, 1H), 6.69 (d, J=7.5 Hz, 2H), 5.56 (s, 1H), 3.96-4.14 (m, 2H), 3.88 (s, 3H), 3.61 (d, J=8.4 Hz, 1H), 3.52 (s, 3H), 2.72-3.03 (m, 5H), 1.48 (d, J=6.9 Hz, 3H). MS: m/z=500.1 (M⁺+1).

(5) Preparation of Compound 154 and Compound 155

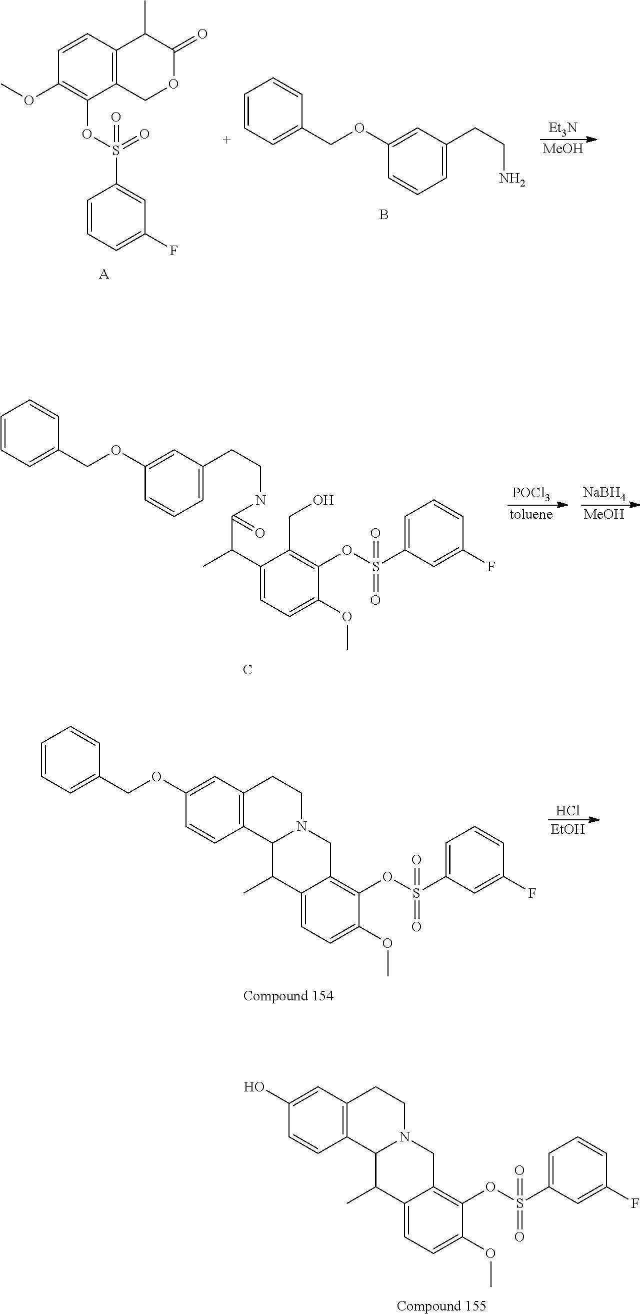

### A+B→C

To the solution of A (1.27 g, 3.6 mmol) and B (627 mg, 3.0 mmol) in MeOH (30 mL) was added Et₃N (600 μL, 4.5 mmol) at 20° C. and the solution was heated to reflux overnight. When TLC analysis indicated completion of the reaction, water and EtOAc were added, and the organic layer was collected, dried, concentrated and purified by chromatography to give 1.8 g of C.

### C→Compound 154

To a stirred solution of C (1.8 g, 30.4 mmol) in toluene (50 mL) was added POCl₃ (2 mL) at ambient temperature. The reaction mixture was refluxed for 2 h with stirring. When the reaction was complete, the solvent and excess POCl₃ were evaporated off. The residue was poured into ice-water and adjusted to pH >7 with Na₂CO₃. The solution was extracted with EtOAc, and the organic layer was dried, concentrated and dissolved in MeOH (50 mL) to which NaBH₄ (244 mg, 6.42 mmol) was added at 0° C. The reaction mixture was stirred for 10 min at this temperature. TLC analysis indicated completion of the reaction. The solvent was removed and the residue was extracted with EtOAc and water. The organic layer was dried, concentrated and purified by preparative TLC to give 500 mg of compound 154.

### Compound 154→Compound 155

Compound 154 (400 mg, 0.71 mmol) was dissolved in 5 mL of EtOH, and 5 mL of conc. HCl was added to it dropwise. The reaction mixture was refluxed for 2 h. Then it was neutralized with saturated aq. NaHCO₃ and extracted with EtOAc. The organic layer was dried with anhydrous Na₂SO₄ and evaporated to give a white residue which was purified with silica gel to afford compound 155.

Compound 154: ¹H NMR (CDCl₃, 300 MHz) δ 7.84-7.80 (m, 2H), 7.76-7.73 (m, 1H), 7.47-7.33 (m, 6H), 7.12-7.05 (m, 2H), 6.88-6.86 (d, J=8.7 Hz, 1H), 6.75-6.72 (m, 2H), 5.06 (s, 2H), 4.23-4.17 (d, J=15.9 Hz, 1H), 3.76-3.75 (m, 1H), 3.62-3.56 (d, J=18 Hz, 1H), 3.49 (s, 3H), 3.29-3.26 (m, 1H), 3.16-3.05 (m, 2H), 2.64-2.56 (m, 2H), 0.94-0.92 (d, J=6.0 Hz, 1H). MS: m/z=560.1 (M⁺+1).

Compound 155: ¹H NMR (DMSO-d₆, 300 MHz) δ 7.84-7.72 (m, 2H), 7.60-7.52 (m, 1H), 7.38-7.26 (m, 1H), 7.08-7.04 (m, 2H), 6.78-6.69 (m, 2H), 6.58-6.57 (m, 1H), 4.22-4.17 (d, J=15.9 Hz, 1H), 3.74 (m, 1H), 3.61-3.56 (d, J=15 Hz, 1H), 3.50 (s, 3H), 3.12-3.09 (m, 1H), 3.16-3.05 (m, 2H), 2.62-2.56 (m, 2H), 0.93-0.91 (d, J=6.0 Hz, 1H). MS: m/z=470.1 (M⁺+1).

### 61. Preparation of Compounds 91, 127, 162 and 163 (Alternate Method)

### Berberine→Compound AA

To a solution of berberine (250 g, 0.67 mol) in dry CHCl₃ (2 L) was added BBr₃ (200 mL, 2 mol) dropwise at 0° C. After stirring overnight, 500 mL of MeOH was added to quench the reaction. The reaction mixture was stirred for 30 min and then evaporated to dryness under reduced pressure. The solid residue was re-crystallized from ethanol to obtain Compound AA (200 g), (80% yield).

Compound AA: ¹H NMR (MD₃OD, 300 MHz) δ 9.65 (s, 1H), 8.43 (s, 1H), 7.73-7.70 (d, J=9 Hz, 1H), 7.61 (dd, J=9 Hz, 1H), 7.46 (s, 1H), 6.80 (s, 1H), 4.83-4.81 (m, 2H), 3.31 (s, 4H), 3.16-3.14 (m, 2H).

### Compound AA→Compound BB

To a solution of Compound AA (200 g, 0.53 mol) in 2 L of anhydrous acetone was added 300 g of K₂CO₃ with stirring at room temperature for about 20 min. 154 mL of (CH₃)₂SO₄ was then added into the mixture. The mixture was refluxed overnight. After the reaction was complete, 500 mL of 15% NaOH solution was added to quench the reaction and the crude product was purified by silica column to give 190 g of Compound BB (82% yield).

Compound BB: ¹H NMR (CDCl₃, 300 MHz) δ 9.77 (s, 1H), 8.51 (s, 1H), 8.00-7.97 (d, J=9 Hz, 1H), 7.65-7.62 (dd, J=9 Hz, 1H), 7.40 (s, 1H), 6.66 (s, 1H), 4.98-4.96 (m, 2H), 4.22 (s, 3H), 4.08 (s, 3H), 3.99 (s, 3H), 3.95 (s, 3H), 3.78 (s, 3H), 3.23-3.20 (m, 2H).

### Compound BB→Compound DD

500 g of Urea was heated to dissolve at 150° C. with stirring, and 180 g of Compound BB was added by portions. The reaction mixture was then heated to 205° C. in 10 min, and kept at this temperature for about 35 min. The mixture turned red and melted. It was cooled under N₂ to ambient temperature. MeOH (2 L) was added and heated to reflux until all solid was dissolved. The mixture was cooled under ice water and undissolved solid was filtered. The filtrate was washed with DCM (500 mL×2) and the organic layer was evaporated. The residue was dissolved in 2 L of DCM and washed with H₂O twice. It was then dried with anhydrous Na₂SO₄ and the solvent was evaporated to give a red oil. 1 L of MeOH was added to dissolve the oil and NaBH₄ was added by portions to reduce the product at 0° C. until the solution was colorless. MeOH was removed and the residue was extracted with DCM. The organic layer was dried and concentrated and the crude product was purified to afford 27 g of Compound DD (20% yield for 2 steps).

Compound DD: ¹H NMR (CDCl₃, 300 MHz) δ 6.75-6.62 (m, 4H), 5.29 (s, 1H), 4.29-4.24 (m, 1H), 3.87 (s, 9H), 3.63-3.47 (m, 2H), 3.30-3.21 (m, 3H), 2.80-2.78 (m, 1H), 2.72-2.66 (m, 2H).

### Compound DD→Compound 91

To a solution of Compound DD (6.8 g, 0.02 mol) in 60 mL of DCM was added benzenesulfonyl chloride (4.3 g, 0.024 mol) and Et₃N (3.5 mL), and the mixture was stirred at ambient temperature overnight. When the reaction was complete as indicated by TLC, the reaction mixture was diluted with 50 mL DCM and washed with H₂O twice. The organic layer was dried with anhydrous Na₂SO₄ and evaporated to provide the crude product. The crude product was purified by chromatography to afford Compound 91 (80% yield).

Compound 91: ¹H NMR (CDCl₃, 300 MHz), δ 8.02-8.00 (m, 2H), 7.68-7.66 (s, 1H), 7.60-7.54 (m, 2H), 7.05-7.02 (m, 1H), 6.73-6.71 (m, 2H), 6.62 (s, 1H), 4.22-4.20 (m, 1H), 3.88 (s, 6H), 3.63-3.59 (m, 2H), 3.44 (s, 3H), 3.33-3.26 (m, 1H), 3.16-3.10 (m, 2H), 2.86-2.80 (m, 3H). MS (M⁺+1)=482.0. HPLC retention time=3.474 min (HPLC-MS was performed with an Agilent 1200 LCMSD instrument using a 4.6×50 mm, XB-C18 column with UV detection at 214 nm. Analysis was accomplished using a gradient of 40-95% acetonitrile in water over 6 min.)

### Compound DD→Compound 127

To a solution of Compound DD (20 g, 0.06 mol) in 100 mL of DCM, was added 3-fluorobenzenesulfonyl chloride (10 mL) and Et₃N (10 mL), and stirred at ambient temperature overnight. When the reaction was complete as indicated by TLC, the reaction mixture was diluted with 100 mL of DCM and washed with H₂O twice. The organic layer was dried with anhydrous Na₂SO₄ and evaporated to give the crude product. The crude product was purified by chromatography to produce Compound 127 (75% yield).

Compound 127: ¹H NMR (CDCl₃, 300 MHz), δ 7.84-7.71 (m, 2H), 7.60-7.52 (m, 1H), 7.42-7.35 (m, 1H), 7.07-7.04 (d, J=9 Hz, 1H), 6.75-6.71 (m, 2H), 6.62 (s, 1H), 4.27-4.20 (m, 1H), 3.88 (s, 6H), 3.63-3.59 (m, 2H), 3.44 (s, 3H), 3.33-3.26 (m, 1H), 3.16-3.06 (m, 2H), 2.89-2.80 (m, 1H), 2.72-2.61 (m, 2H). MS (M⁺+1)=500.0. HPLC retention time=3.655 min. (HPLC-MS conditions were as for compound 91.)

Resolution of Compound 127 into Compounds 162 and 163

Compound 127 was resolved into its enantiomers via chiral HPLC under the following conditions (see Table 2).

TABLE 2

| Column | CHIRALPAK IC |
|---|---|
| Column size | 0.46 cm I.D. × 15 cm L |
| Injection | 5 μL |
| Mobile phase | MeOH/DCM/DEA (90/10/0.1) |
| Flow rate | 0.5 ml/min |
| Wave length | UV 220 nm |
| Temperature | 35° C. |
| Sample solution | x mg/mL in MP |
| HPLC equipment | Shimadzu LC 20 with UV detector SPD-20A |

The enantiomers, Compounds 162 and 163 were characterized by optical rotation and LC/MS as described in the Table 3 below, as well as by ¹H NMR.

TABLE 3

| | MW | MW | Optical | | | |
|---|---|---|---|---|---|---|
| Name | free base | salt form | rotation | Purity | Molecular Formula | LC/MS - m/e |
| Compound 162 | 499.55 | 597.55 | +182.1° | 100% | C₂₆H₂₆FNO₆S•H₃O₄P | 500.0 (M + 1) |
| Compound 163 | 499.55 | 597.55 | −180.7° | 99.10% | C₂₆H₂₆FNO₆S•H₃O₄P | 500.0 (M + 1) |

### Example 3

The following compounds of Table 4 were synthesized according the above procedures or slight modifications thereof and were characterized by mass spectroscopy. Each compound gave the expected MH peak in the mass spectrum.

TABLE 4

| Cmpd No. | Structure | Chemical Name | Molecular Formula | Molecular Weight | MS: m/z [M⁺ + 1]* |
|---|---|---|---|---|---|
| 1 | 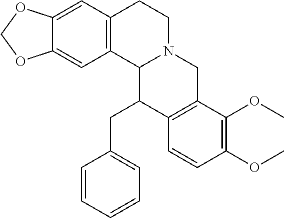 | 9,10-dimethoxy-13-benzyl- 5,6,7,8,13,13a-hexahydro-2H- 1,3-dioxolano[4,5- g]isoquinolino[3,2- a]isoquinoline | C27H27NO4 | 429.51 | 430.2 |
| 2 | 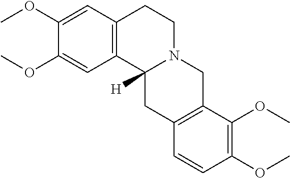 | (13aR)-2,3,9,10-tetramethoxy- 5,6,7,8,13,13a- hexahydroisoquinolino[3,2- a]isoquinoline hydrochloride | C21H25NO4•HCl | 355.43** | 356.2 |
| 3 | 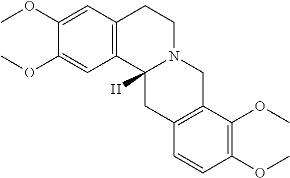 | (13aR)-2,3,9,10-tetramethoxy- 5,6,7,8,13,13a- hexahydroisoquinolino[3,2- a]isoquinoline sulfate | C21H25NO4•H2SO4 | 355.43** | 356.2 |
| 4 | 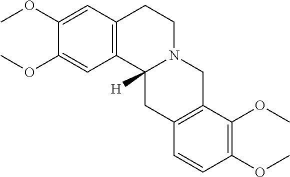 | (13aR)-2,3,9,10-tetramethoxy- 5,6,7,8,13,13a- hexahydroisoquinolino[3,2- a]isoquinoline citrate | C21H25NO4•citrate | 355.43** | 356.2 |
| 5 | 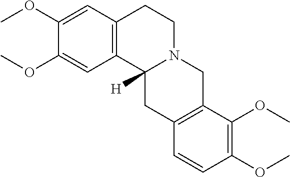 | (13aR)-2,3,9,10-tetramethoxy- 5,6,7,8,13,13a- hexahydroisoquinolino[3,2- a]isoquinoline maleate | C21H25NO4•maleate | 355.43** | 356.2 |
| 6 | 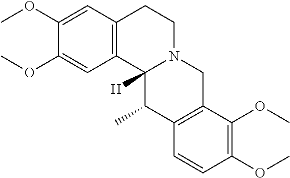 | (13S,13aR)-2,3,9,10- tetramethoxy-13-methyl- 5,6,7,8,13,13a- hexahydroisoquinolino[2,1- b]isoquinoline hydrochloride | C22H27NO4•HCl | 369.45** | 370.2 |
| 7 | 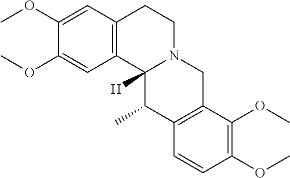 | (13S,13aR)-2,3,9,10- tetramethoxy-13-methyl- 5,6,7,8,13,13a- hexahydroisoquinolino[2,1- b]isoquinoline sulfate | C22H27NO4•H2SO4 | 369.45** | 370.2 |
| 8 | 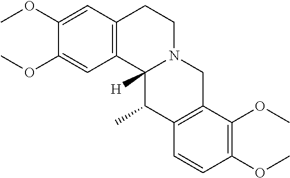 | (13S,13aR)-2,3,9,10- tetramethoxy-13-methyl- 5,6,7,8,13,13a- hexahydroisoquinolino[2,1- b]isoquinoline citrate | C22H27NO4•citrate | 369.45** | 370.2 |
| 9 | 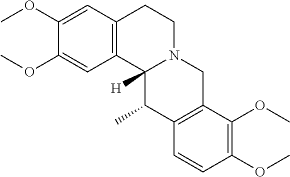 | (13S,13aR)-2,3,9,10- tetramethoxy-13-methyl- 5,6,7,8,13,13a- hexahydroisoquinolino[2,1- b]isoquinoline maleate | C22H27NO4•maleate | 369.45** | 370.2 |
| 10 | 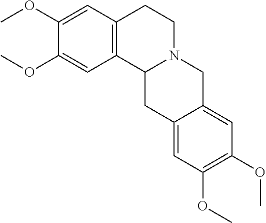 | 2,3,10,11-tetramethoxy- 5,6,7,8,13,13a- hexahydroisoquinolino[3,2- a]isoquinoline | C21H25NO4 | 355.43 | 356.1 |
| 11 | 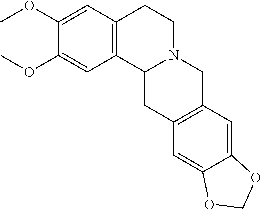 | 2,3-dimethoxy-5,6,7,8,14,14a- hexahydro-11H-1,3- dioxoleno[4,5- g]isoquinolino[2,1- b]isoquinoline | C20H21NO4 | 339.39 | 340.1 |
| 12 | 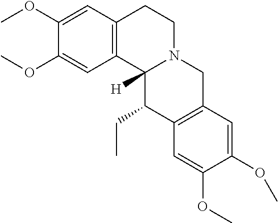 | (13S,13aR)-13-ethyl-2,3,10,11- tetramethoxy-5,6,7,8,13,13a- hexahydroisoquinolino[2,1- b]isoquinoline | C23H29NO4 | 383.48 | 384.2 |
| 13 | 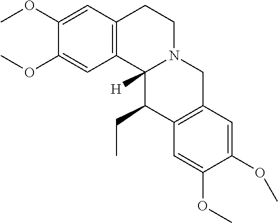 | (13R,13aR)-13-ethyl- 2,3,10,11-tetramethoxy- 5,6,7,8,13,13a- hexahydroisoquinolino[2,1- b]isoquinoline | C23H29NO4 | 383.48 | 384.2 |
| 14 | 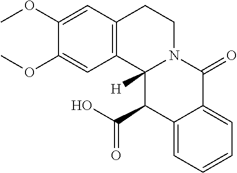 | (13R,13aR)-2,3-dimethoxy-8- oxo-5,6,7,13,13a- pentahydroisoquinolino[2,1- b]isoquinoline-13-carboxylic acid | C20H19NO5 | 353.37 | 354.1 |
| 15 | 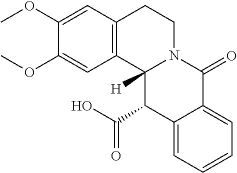 | (13S,13aR)-2,3-dimethoxy-8- oxo-5,6,7,13,13a- pentahydroisoquinolino[2,1- b]isoquinoline-13-carboxylic acid | C20H19NO5 | 353.37 | 354.1 |
| 16 | 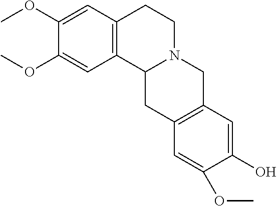 | 2,3,11-trimethoxy- 5,6,7,8,13,13a- hexahydroisoquinolino[2,1- b]isoquinolin-10-ol | C20H23NO4 | 341.40 | 342.1 |
| 17 | 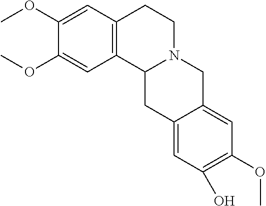 | 2,3,10-trimethoxy- 5,6,7,8,13,13a- hexahydroisoquinolino[2,1- b]isoquinolin-11-ol | C20H23NO4 | 341.40 | 342.1 |
| 18 | 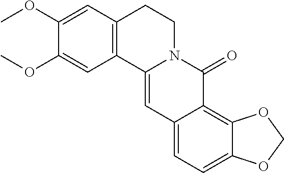 | 8,9-dimethoxy-6,11,12,13,6a- pentahydro-2H-1,3- dioxoleno[4,5- h]isoquinolino[2,1- b]isoquinolin-14-one | C20H17NO5 | 351.10 | 352.0 |
| 19 | 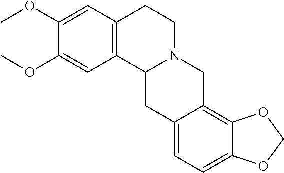 | 8,9-dimethoxy- 6,11,12,13,14,6a-hexahydro- 2H-1,3-dioxoleno[4,5- h]isoquinolino[2,1- b]isoquinoline | C20H21NO4 | 339.14 | 340.1 |
| 20 | 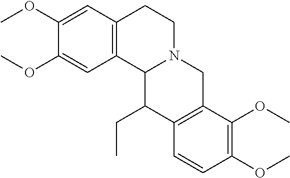 | 13-ethyl-2,3,9,10- tetramethoxy-5,6,7,8,13,13a- hexahydroisoquinolino[2,1- b]isoquinoline | C23H29NO4 | 383.49 | 402.1 [M⁺ + 1 + H₂O] |
| 21 | 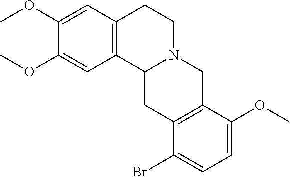 | 12-bromo-2,3,9-trimethoxy- 5,6,7,8,13,13a- hexahydroisoquinolino[2,1- b]isoquinoline | C20H22BrNO3 | 404.31 | 403.9, 405.9 |
| 22 | 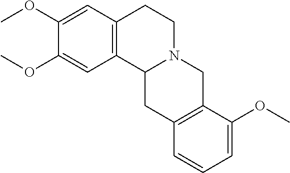 | 2,3,9-trimethoxy- 5,6,7,8,13,13a- hexahydroisoquinolino[3,2- a]isoquinoline | C20H23NO3 | 325.41 | 326.1 |
| 23 | 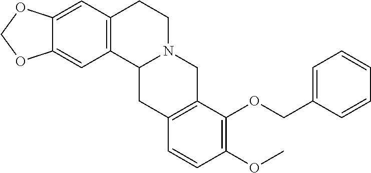 | 10-methoxy-9- (phenylmethoxy)- 5,6,7,8,13,13a-hexahydro-2H- 1,3-dioxolano[4,5- g]isoquinolino[3,2- a]isoquinoline | C26H25NO4 | 415.48 | 416.0 |
| 24 | 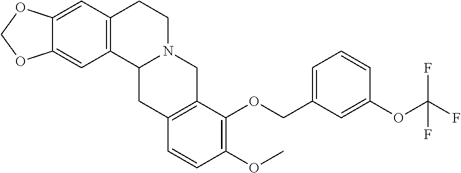 | 10-methoxy-9-{[3- (trifluoromethoxy)phenyl] methoxy}-5,6,7,8,13,13a- hexahydro-2H-1,3- dioxolano[4,5- g]isoquinolino[3,2- a]isoquinoline | C27H24F3NO5 | 499.48 | 500.0 |
| 25 | 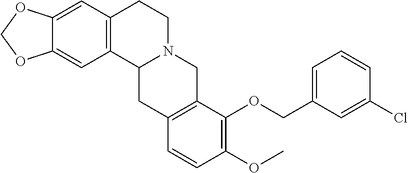 | 9-[(3-chlorophenyl)methoxy]- 10-methoxy-5,6,7,8,13,13a- hexahydro-2H-1,3- dioxolano[4,5- g]isoquinolino[3,2- a]isoquinoline | C26H24ClNO4 | 449.93 | 450.0, 452.0 |
| 26 | 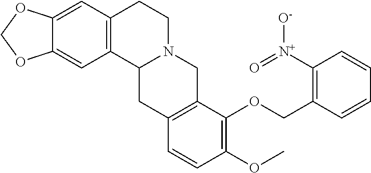 | 10-methoxy-9-[(2- nitrophenyl)methoxy]- 5,6,7,8,13,13a-hexahydro-2H- 1,3-dioxolano[4,5- g]isoquinolino[3,2- a]isoquinoline | C26H24N2O6 | 460.48 | 461.0 |
| 27 | 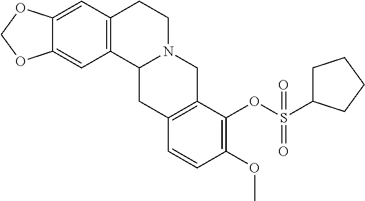 | 10-methoxy-5,6,7,8,13,13a- hexahydro-2H-1,3- dioxolano[4,5- g]isoquinolino[3,2- a]isoquinolin-9-yl cyclopentanesulfonate | C24H27NO6S | 457.54 | 458.1 |
| 28 | 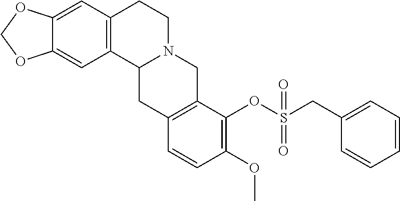 | 10-methoxy-5,6,7,8,13,13a- hexahydro-2H-1,3- dioxolano[4,5- g]isoquinolino[3,2- a]isoquinolin-9-yl benzylsulfonate | C26H25NO6S | 479.54 | 480.0 |
| 29 | 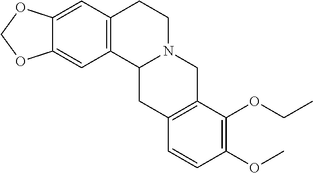 | 9-ethoxy-10-methoxy- 5,6,7,8,13,13a-hexahydro-2H- 1,3-dioxolano[4,5- g]isoquinolino[3,2- a]isoquinoline | C21H23NO4 | 353.41 | 354.1 |
| 30 | 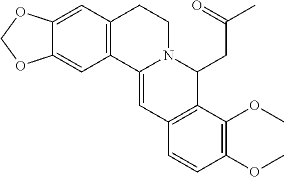 | 1-(9,10-dimethoxy-5,6,7,8- tetrahydro-2H-1,3- dioxolano[4,5- g]isoquinolino[3,2- a]isoquinolin-8-yl)acetone | C23H23NO5 | 393.43 | 336.0 [M⁺ + 1-acetone] |
| 31 | 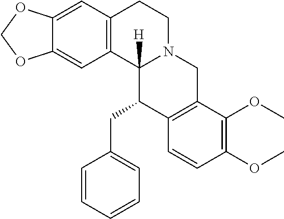 | (13S,13aR)-9,10-dimethoxy- 13-benzyl-5,6,7,8,13,13a- hexahydro-2H-1,3- dioxolano[4,5- g]isoquinolino[3,2- a]isoquinoline | C27H27NO4 | 429.51 | 430.1 |
| 32 | 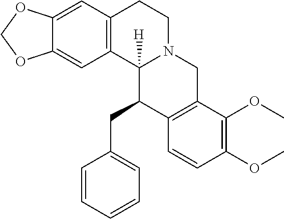 | (13aS,13R)-9,10-dimethoxy- 13-benzyl-5,6,7,8,13,13a- hexahydro-2H-1,3- dioxolano[4,5- g]isoquinolino[3,2- a]isoquinoline | C27H27NO4 | 429.51 | 430.1 |
| 33 | 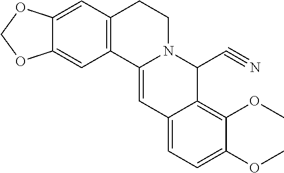 | 9,10-dimethoxy-5,6,7,8- tetrahydro-2H-1,3- dioxolano[4,5- g]isoquinolino[3,2- a]isoquinoline-8-carbonitrile | C21H18N2O4 | 362.38 | 336.0 [M⁺ − CN] |
| 34 | 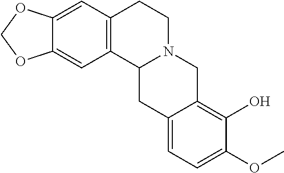 | 10-methoxy-5,6,7,8,13,13a- hexahydro-2H-1,3- dioxolano[4,5- g]isoquinolino[3,2- a]isoquinoline-9-ol | C19H19NO4 | 325.13 | 326.0 |
| 35 | 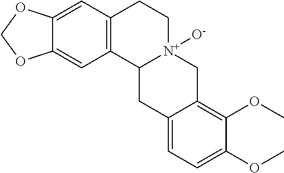 | 9,10-dimethoxy- 5,6,7,8,13,13a-hexahydro-2H- 1,3-dioxolano[4,5- g]isoquinolino[3,2- a]isoquinoline-N-oxide | C20H21NO5 | 355.38 | 356.0 |
| 36 | 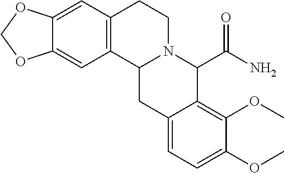 | 9,10-dimethoxy- 5,6,7,8,13,13a-hexahydro-2H- 1,3-dioxolano[4,5- g]isoquinolino[3,2- a]isoquinoline-8-carboxamide | C21H22N2O5 | 382.41 | 383.0 |
| 37 | 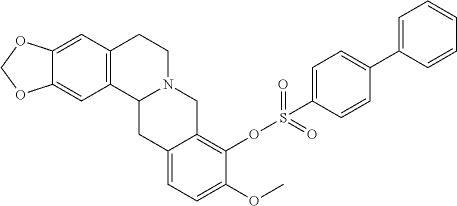 | 10-methoxy-5,6,7,8,13,13a- hexahydro-2H-1,3- dioxolano[4,5- g]isoquinolino[3,2- a]isoquinolin-9-yl 4- phenylbenzenesulfonate | C31H27NO6S | 541.61 | 542.0 |
| 38 | 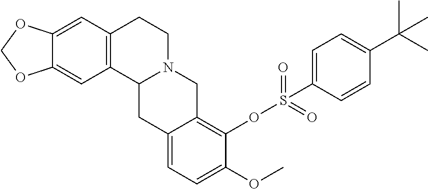 | 10-methoxy-5,6,7,8,13,13a- hexahydro-2H-1,3- dioxolano[4,5- g]isoquinolino[3,2- a]isoquinolin-9-yl 4-(tert- butyl)benzenesulfonate | C29H31NO6S | 521.62 | 522.1 |
| 39 | 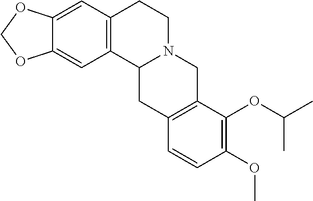 | 10-methoxy-9-(methylethoxy)- 5,6,7,8,13,13a-hexahydro-2H- 1,3-dioxolano[4,5- g]isoquinolino[3,2- a]isoquinoline | C22H25NO4 | 367.44 | 368.1 |
| 41 | 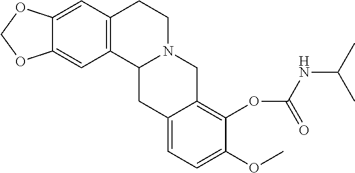 | (10-methoxy(5,6,7,8,13,13a- hexahydro-2H-1,3- dioxolano[4,5- g]isoquinolino[3,2- a]isoquinolin-9-yloxy))-N- (methylethyl)carboxamide | C23H26N2O5 | 410.46 | 411.1 |
| 42 | 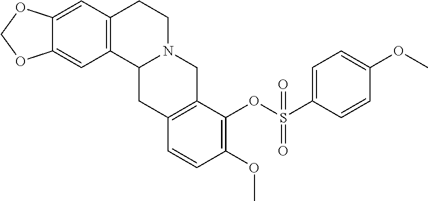 | 10-methoxy-5,6,7,8,13,13a- hexahydro-2H-1,3- dioxolano[4,5- g]isoquinolino[3,2- a]isoquinolin-9-yl 4- methoxybenzenesulfonate | C26H25NO7S | 495.54 | 496.0 |
| 43 | 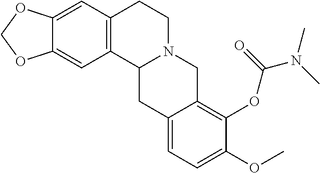 | (10-methoxy(5,6,7,8,13,13a- hexahydro-2H-1,3- dioxolano[4,5- g]isoquinolino[3,2- a]isoquinolin-9-yloxy))-N,N- dimethylcarboxamide | C22H24N2O5 | 396.44 | 397.1 |
| 44 | 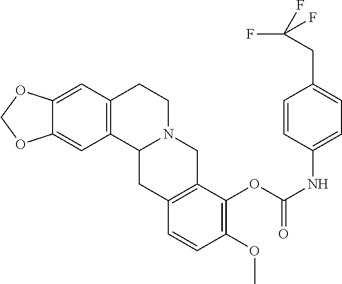 | (10-methoxy(5,6,7,8,13,13a- hexahydro-2H-1,3- dioxolano[4,5- g]isoquinolino[3,2- a]isoquinolin-9-yloxy))-N-[4- (trifluoromethoxy)phenyl] carboxamide | C27H23F3N2O6 | 528.41 | 529.1 |
| 45 | 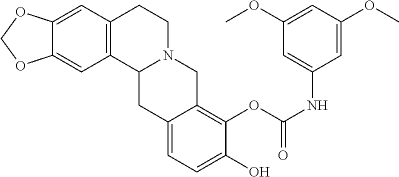 | N-(3,5-dimethoxyphenyl)(10- methoxy(5,6,7,8,13,13a- hexahydro-2H-1,3- dioxolano[4,5- g]isoquinolino[3,2- a]isoquinolin-9- yloxy))carboxamide | C28H28N2O7 | 504.53 | 505.1 |
| 46 | 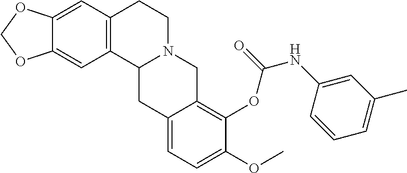 | (10-methoxy(5,6,7,8,13,13a- hexahydro-2H-1,3- dioxolano[4,5- g]isoquinolino[3,2- a]isoquinolin-9-yloxy))-N-(3- methylphenyl)carboxamide | C27H26N2O5 | 458.51 | 459.1 |
| 47 | 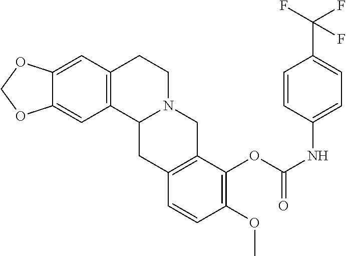 | (10-methoxy(5,6,7,8,13,13a- hexahydro-2H-1,3- dioxolano[4,5- g]isoquinolino[3,2- a]isoquinolin-9-yloxy))-N-[3- (trifluoromethyl)phenyl] carboxamide | C27H23F3N2O5 | 512.48 | 513.0 |
| 48 | 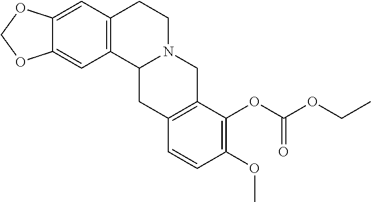 | 10-methoxy-5,6,7,8,13,13a- hexahydro-2H-1,3- dioxolano[4,5- g]isoquinolino[3,2- a]isoquinolin-9-yl ethoxyformate | C22H23NO6 | 397.42 | 398.1 |
| 49 | 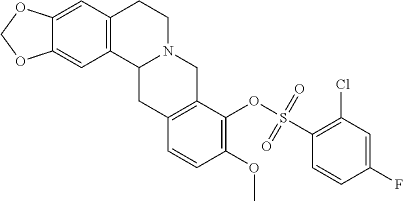 | 10-methoxy-5,6,7,8,13,13a- hexahydro-2H-1,3- dioxolano[4,5- g]isoquinolino[3,2- a]isoquinolin-9-yl 2-chloro-4- fluorobenzenesulfonate | C25H21ClFNO6S | 517.95 | 517.9, 519.9 |
| 50 | 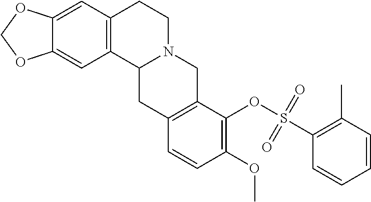 | 10-methoxy-5,6,7,8,13,13a- hexahydro-2H-1,3- dioxolano[4,5- g]isoquinolino[3,2- a]isoquinolin-9-yl 2- methylbenzenesulfonate | C26H25NO6S | 479.54 | 480.0 |
| 51 | 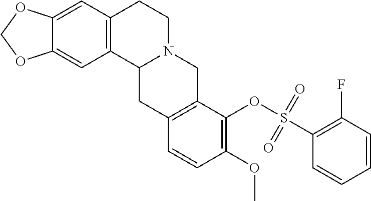 | 10-methoxy-5,6,7,8,13,13a- hexahydro-2H-1,3- dioxolano[4,5- g]isoquinolino[3,2- a]isoquinolin-9-yl 2- fluorobenzenesulfonate | C25H22FNO6S | 483.51 | 484.0 |
| 52 | 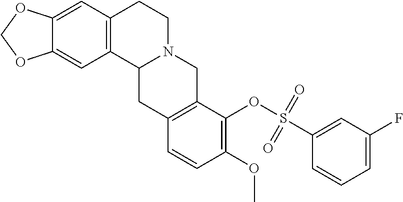 | 10-methoxy-5,6,7,8,13,13a- hexahydro-2H-1,3- dioxolano[4,5- g]isoquinolino[3,2- a]isoquinolin-9-yl 3- fluorobenzenesulfonate | C25H22FNO6S | 483.51 | 484.0 |
| 53 | 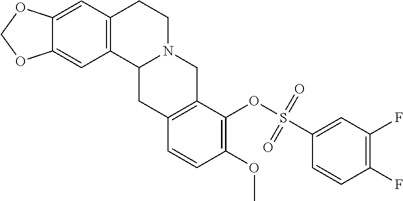 | 10-methoxy-5,6,7,8,13,13a- hexahydro-2H-1,3- dioxolano[4,5- g]isoquinolino[3,2- a]isoquinolin-9-yl 3,4- difluorobenzenesulfonate | C25H21F2NO6S | 501.50 | 502.0 |
| 54 | 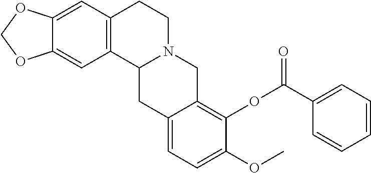 | 10-methoxy-5,6,7,8,13,13a- hexahydro-2H-1,3- dioxolano[4,5- g]isoquinolino[3,2- a]isoquinolin-9-yl benzoate | C26H23NO5 | 429.46 | 430.0 |
| 55 | 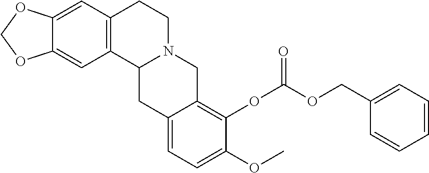 | 10-methoxy-5,6,7,8,13,13a- hexahydro-2H-1,3- dioxolano[4,5- g]isoquinolino[3,2- a]isoquinolin-9-yl (phenylmethoxy)formate | C27H25NO6 | 459.49 | 460.1 |
| 56 | 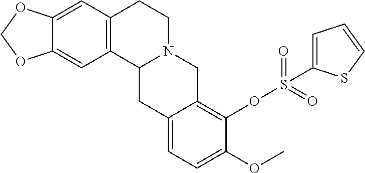 | 10-methoxy-5,6,7,8,13,13a- hexahydro-2H-1,3- dioxolano[4,5- g]isoquinolino[3,2- a]isoquinolin-9-yl thiophene-2- sulfonate | C23H21NO6S2 | 471.55 | 472.0 |
| 57 | 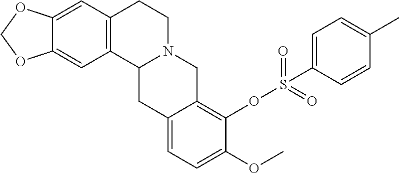 | 10-methoxy-5,6,7,8,13,13a- hexahydro-2H-1,3- dioxolano[4,5- g]isoquinolino[3,2- a]isoquinolin-9-yl 4- methylbenzenesulfonate | C26H25NO6S | 479.54 | 480.0 |
| 58 | 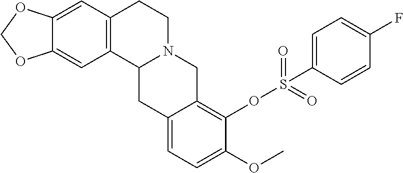 | 10-methoxy-5,6,7,8,13,13a- hexahydro-2H-1,3- dioxolano[4,5- g]isoquinolino[3,2- a]isoquinolin-9-yl 4- fluorobenzenesulfonate | C25H22FNO6S | 483.51 | 484.0 |
| 59 | 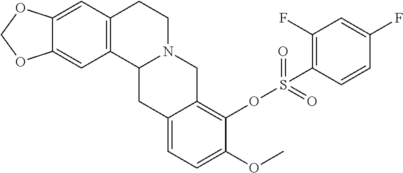 | 10-methoxy-5,6,7,8,13,13a- hexahydro-2H-1,3- dioxolano[4,5- g]isoquinolino[3,2- a]isoquinolin-9-yl 2,4- difluorobenzenesulfonate | C25H21F2NO6S | 501.50 | 502.0 |
| 60 | 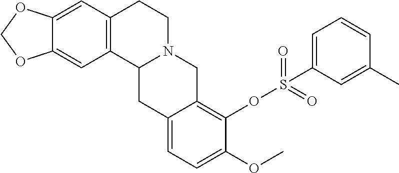 | 10-methoxy-5,6,7,8,13,13a- hexahydro-2H-1,3- dioxolano[4,5- g]isoquinolino[3,2- a]isoquinolin-9-yl 3- methylbenzenesulfonate | C26H25NO6S | 479.54 | 480.0 |
| 61 | 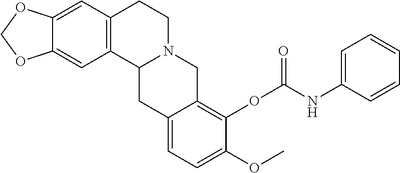 | (10-methoxy(5,6,7,8,13,13a- hexahydro-2H-1,3- dioxolano[4,5- g]isoquinolino[3,2- a]isoquinolin-9-yloxy))-N- benzamide | C26H24N2O5 | 444.48 | 445.0 |
| 62 | 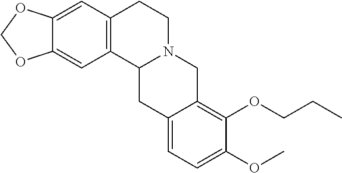 | 10-methoxy-9-propoxy- 5,6,7,8,13,13a-hexahydro-2H- 1,3-dioxolano[4,5- g]isoquinolino[3,2- a]isoquinoline | C22H25NO4 | 367.44 | 368.1 |
| 63 | 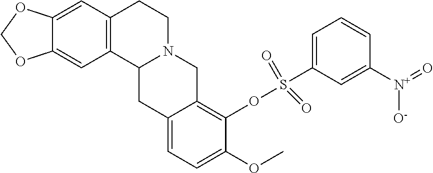 | 10-methoxy-5,6,7,8,13,13a- hexahydro-2H-1,3- dioxolano[4,5- g]isoquinolino[3,2- a]isoquinolin-9-yl 3- nitrobenzenesulfonate | C25H22N2O8S | 510.52 | 511.0 |
| 64 | 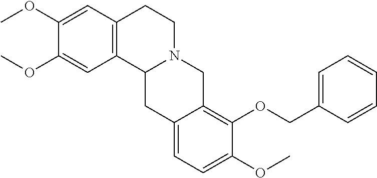 | 2,3,10-trimethoxy-9- (phenylmethoxy)- 5,6,7,8,13,13a- hexahydroisoquinolino[3,2- a]isoquinoline | C27H29NO4 | 431.52 | 432.1 |
| 65 | 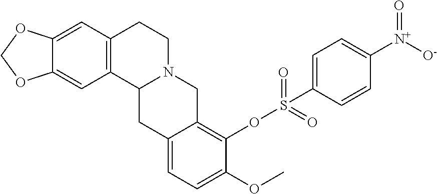 | 10-methoxy-5,6,7,8,13,13a- hexahydro-2H-1,3- dioxolano[4,5- g]isoquinolino[3,2- a]isoquinolin-9-yl 4- nitrobenzenesulfonate | C25H22N2O8S | 510.52 | 511.0 |
| 66 | 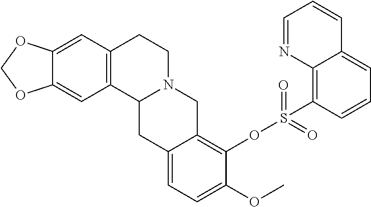 | 10-methoxy-5,6,7,8,13,13a- hexahydro-2H-1,3- dioxolano[4,5- g]isoquinolino[3,2- a]isoquinolin-9-yl quinoline- 8-sulfonate | C28H24N2O6S | 516.56 | 517.0 |
| 67 | 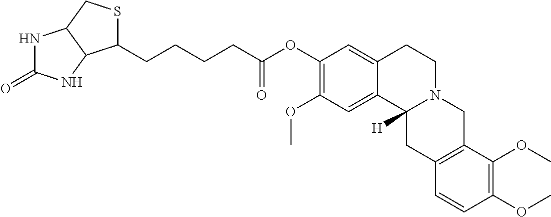 | [3,9,10-trimethoxy-5,8,13,13a- tetrahydro-6H-isoquino[3,2- a]isoquinolin-3-yl]-5-(2-oxo- hexahydro-1H-thieno[3,4- d]imidazol-4-yl)pentanoic ester | C30H37N3O6S | 567.24 | 568.3 |
| 68 | 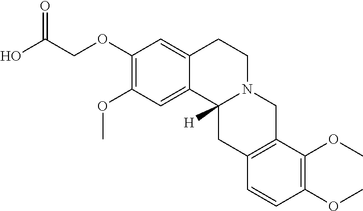 | 2-[3,9,10-trimethoxy- 5,8,13,13a-tetrahydro-6H- isoquino[3,2-a]isoquinolin-3- yl]-acetic acid | C22H25NO6 | 399.17 | 400.2 |
| 69 | 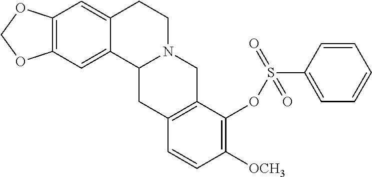 | 9-benzenesulfonyloxy-10- methoxy-5,8,13,13a- tetrahydro-6H- [1,3]dioxolo[4,5- g]isoquino[3,2-a]isoquinoline | C25H23NO6S | 465.12 | 466.2 |
| 70 | 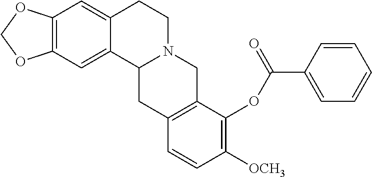 | 9-benzoxy-10-methoxy- 5,8,13,13a-tetrahydro-6H- [1,3]dioxolo[4,5- g]isoquino[3,2-a]isoquinoline | C26H23NO5 | 429.16 | 430.1 |
| 71 | 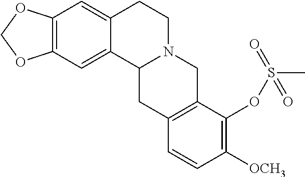 | 9-methanesulfonyloxy-10- methoxy-5,8,13,13a- tetrahydro-6H- [1,3]dioxolo[4,5- g]isoquino[3,2-a]isoquinoline | C20H21NO6S | 403.11 | 404.1 |
| 72 | 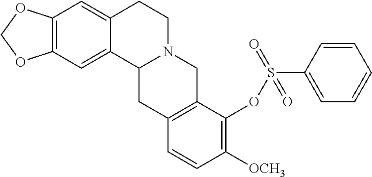 | 9-benzenesulfonyloxy-10- methoxy-5,8,13,13a- tetrahydro-6H- [1,3]dioxolo[4,5- g]isoquino[3,2-a]isoquinoline hydrochloride | C25H24ClNO6S | 466.13 | 466.2 |
| 73 | 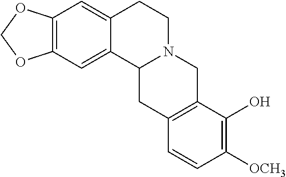 | 10-methoxy-5,8,13,13a- tetrahydro-6H- [1,3]dioxolo[4,5- g]isoquino[3,2-a]isoquinoline- 9-ol | C19H19NO4 | 325.13 | 326.2 |
| 74 | 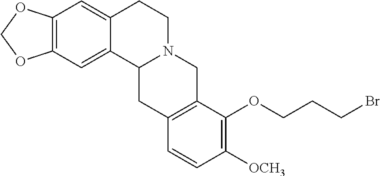 | 9-O-3-(1′-bromo-propyl)-10- methoxy-5,8,13,13a- tetrahydro-6H- [1,3]dioxolo[4,5- g]isoquino[3,2-a]isoquinoline | C22H24BrNO4 | 445.09 | 446.2 |
| 75 | 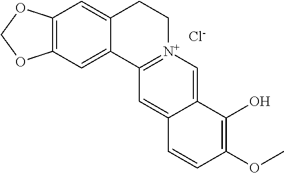 | 9-hydroxy-10-methoxy-5,6- dihydro-[1,3]dioxolo[4,5- g]isoquino[3,2- a]isoquinolinylium | C19H16ClNO4 | 357.08 | 323.1 |
| 76 | 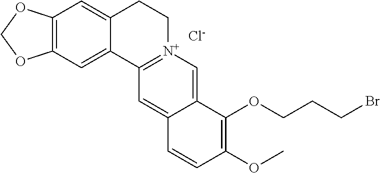 | 9-O-3-(1′-bromo-propyl)-10- methoxy-5,6-dihydro- [1,3]dioxolo[4,5- g]isoquino[3,2- a]isoquinolinylium | C22H21BrClNO4 | 479.03 | 443.9 |
| 77 | 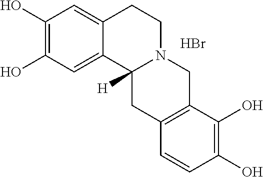 | 5,8,13,13a-Tetrahydro-6H- isoquino[3,2-a]isoquinoline- 2,3,9,10-tetraol; hydrobromide | C17H18BrNO4 | 379.04 | 298.3 |
| 78 | 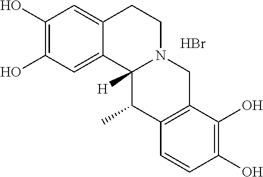 | 13-methyl-5,8,13,13a- Tetrahydro-6H-isoquino[3,2- a]isoquinoline-2,3,9,10-tetraol; hydrobromide | C18H20BrNO4 | 393.06 | 313.8 |
| 79 | 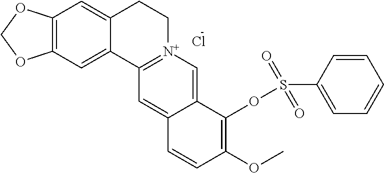 | 9-benzenesulfonyloxy-10- methoxy-5,6-dihydro- [1,3]dioxolo[4,5- g]isoquino[3,2- a]isoquinolinylium | C25H20ClNO6S | 497.07 | 462.2 |
| 80 | 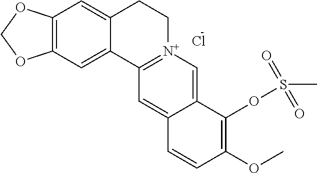 | 9-methanesulfonyloxy-10- methoxy-5,6-dihydro- [1,3]dioxolo[4,5- g]isoquino[3,2- a]isoquinolinylium | C20H18ClNO6S | 435.05 | 399.5 |
| 81 | 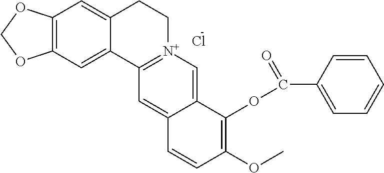 | 9-benzoxy-10-methoxy-5,6- dihydro-[1,3]dioxolo[4,5- g]isoquino[3,2- a]isoquinolinylium | C26H20ClNO5 | 461.1 | 426.1 |
| 82 | 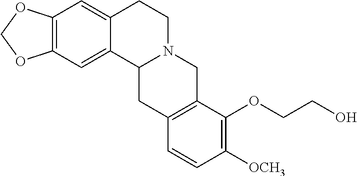 | 9-O-2-(1′-hydroxy-ethyl)-10- methoxy-5,8,13,13a- tetrahydro-6H- [1,3]dioxolo[4,5- g]isoquino[3,2-a]isoquinoline | C21H23NO5 | 369.16 | 370.1 |
| 83 | 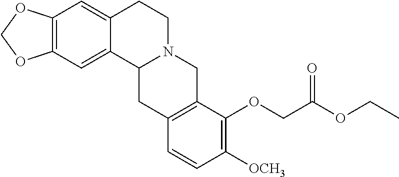 | 2-[10-methoxy-5,8,13,13a- tetrahydro-6H- [1,3]dioxolo[4,5- g]isoquino[3,2-a]isoquinoline- 9-yl]-acetic esate | C23H25NO6 | 411.17 | 412.2 |
| 84 | 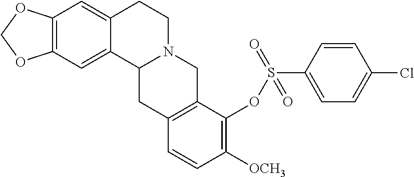 | 9-(4-chloro- benzenesulfonyloxy)-10- methoxy-5,8,13,13a- tetrahydro-6H- [1,3]dioxolo[4,5- g]isoquino[3,2-a]isoquinoline | C25H22ClNO6S | 499.09 | 500.3 |
| 85 | 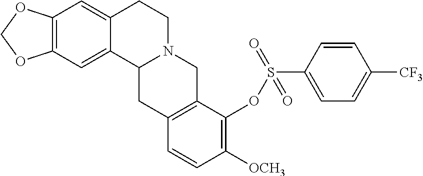 | 9-(4-trifluoromethyl- benzenesulfonyloxy)-10- methoxy-5,8,13,13a- tetrahydro-6H- [1,3]dioxolo[4,5- g]isoquino[3,2-a]isoquinoline | C26H22F3NO6S | 533.52 | 534.2 |
| 86 | 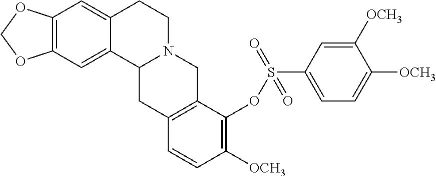 | 9-(3,4-dimethoxy- benzenesulfonyloxy)-10- methoxy-5,8,13,13a- tetrahydro-6H- [1,3]dioxolo[4,5- g]isoquino[3,2-a]isoquinoline | C27H27NO8S | 525.57 | 526.3 |
| 87 | 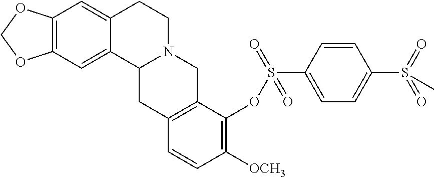 | 9-(4-methylsulfonyl- benzenesulfonyloxy)-10- methoxy-5,8,13,13a- tetrahydro-6H- [1,3]dioxolo[4,5- g]isoquino[3,2-a]isoquinoline | C26H25NO8S2 | 543.61 | 544.3 |
| 88 | 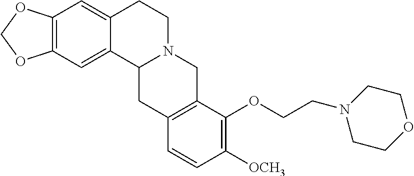 | 9-O-2-(1′-morpholine-ethyl)- 10-methoxy-5,8,13,13a- tetrahydro-6H- [1,3]dioxolo[4,5- g]isoquino[3,2-a]isoquinoline | C25H30N2O5 | 438.52 | 439.2 |
| 89 | 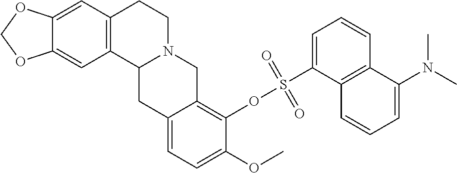 | 10-methoxy-5,6,7,8,13,13a- hexahydro-2H-1,3-dioxolano [4,5-g]isoquinolino[3,2-a] isoquinolin-9-yl 5- (dimethylamino)naphthalene- sulfonate | C31H30N2O6S | 558.64 | 559.1 |
| 90 | 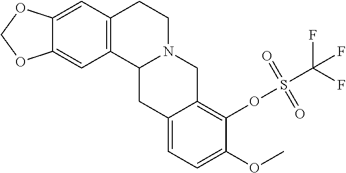 | 10-methoxy-5,6,7,8,13,13a- hexahydro-2H-1,3- dioxolano[4,5-g] isoquinolino[3,2-a] isoquinolin-9-yl (trifluoromethyl)sulfonate | C20H18F3NO6S | 457.42 | 458.0 |
| 91 | 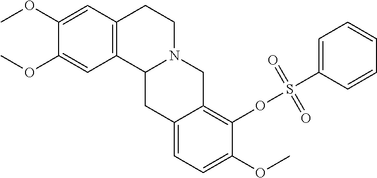 | 2,3,10-trimethoxy- 5,6,7,8,13,13a- hexahydroisoquinolino[2,1- b]isoquinolin-9-yl benzenesulfonate | C26H27NO6S | 481.56 | 482.0 |
| 92 | 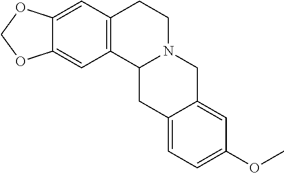 | 10-methoxy-5,6,7,8,13,13a- hexahydro-2H-1,3- dioxolano[4,5- g]isoquinolino[3,2- a]isoquinoline | C19H19NO3 | 309.36 | 310.0 |
| 93 | 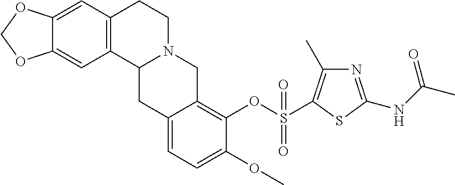 | 10-methoxy-5,6,7,8,13,13a- hexahydro-2H-1,3-dioxolano [4,5-g]isoquinolino[3,2- a]isoquinolin-9-yl 2- (acetylamino)-4-methyl-1,3- thiazole-5-sulfonate | C25H25N3O7S2 | 543.61 | 543.9 |
| 94 | 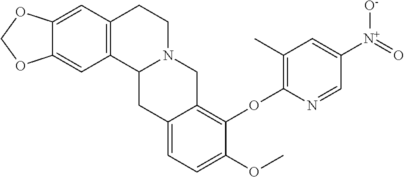 | 10-methoxy-9-(3-methyl-5- nitro(2-pyridyl)oxy)- 5,6,7,8,13,13a-hexahydro-2H- 1,3-dioxolano[4,5- g]isoquinolino[3,2- a]isoquinoline | C25H23N3O6 | 461.47 | 462.0 |
| 95 | 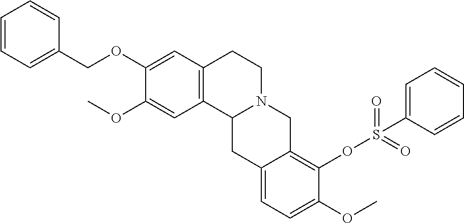 | 2,10-dimethoxy-3- (phenylmethoxy)- 5,6,7,8,13,13a- hexahydroisoquinolino[2,1- b]isoquinolin-9-yl benzenesulfonate | C32H31NO6S | 557.66 | 558.0 |
| 96 | 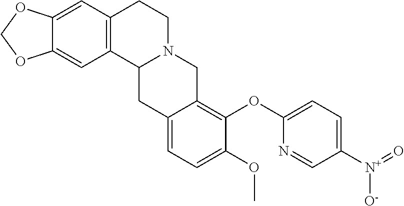 | 10-methoxy-9-(5-nitro(2- pyridyl)oxy)-5,6,7,8,13,13a- hexahydro-2H-1,3-dioxolano [4,5-g]isoquinolino[3,2- a]isoquinoline | C24H21N3O6 | 447.40 | 448.0 |
| 97 | 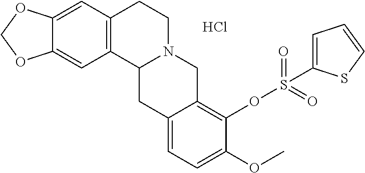 | 10-methoxy-5,6,7,8,13,13a- hexahydro-2H-1,3-dioxolano [4,5-g]isoquinolino[3,2- a]isoquinolin-9-yl thiophene-2- sulfonate hydrochloride | C23H22ClNO6S2 | 508.00 | 471.9 |
| 98 | 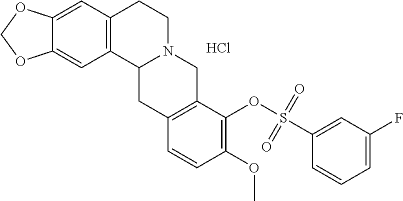 | 10-methoxy-5,6,7,8,13,13a- hexahydro-2H-1,3-dioxolano [4,5-g]isoquinolino[3,2-a] isoquinolin-9-yl 3- fluorobenzenesulfonate hydrochloride | C25H23ClFNO6S | 519.96 | 484.0 |
| 99 | 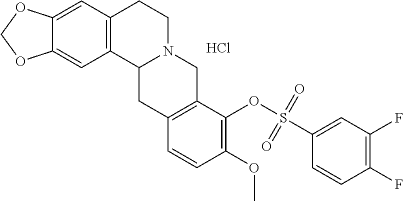 | 10-methoxy-5,6,7,8,13,13a- hexahydro-2H-1,3-dioxolano [4,5-g]isoquinolino[3,2-a] isoquinolin-9-yl 3,4- difluorobenzenesulfonate hydrochloride | C25H22ClF2NO6S | 537.95 | 502.0 |
| 100 | 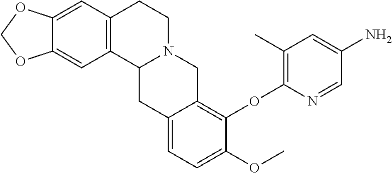 | 6-(10-methoxy(5,6,7,8,13,13a- hexahydro-2H-1,3- dioxolano[4,5- g]isoquinolino[3,2- a]isoquinolin-9-yloxy))-5- methyl-3-pyridylamine | C25H25N3O4 | 431.48 | 432.1 |
| 101 | 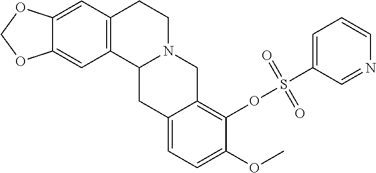 | 10-methoxy-5,6,7,8,13,13a- hexahydro-2H-1,3-dioxolano [4,5-g]isoquinolino[3,2- a]isoquinolin-9-yl pyridine-3- sulfonate | C24H22N2O6S | 466.51 | 467.0 |
| 102 | 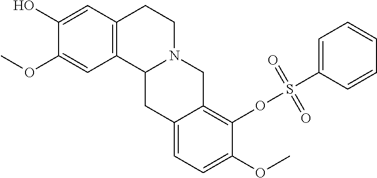 | 3-Hydroxy-2,10-dimethoxy- 5,6,7,8,13,13a- hexahydroisoquinolino[2,1- b]isoquinolin-9-yl benzenesulfonate | C25H25NO6S | 467.53 | 468.0 |
| 103 | 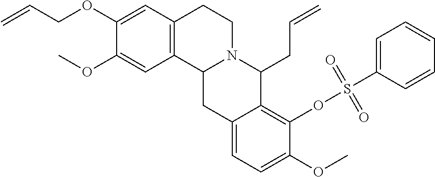 | 2,10-Dimethoxy-8-prop-2- enyl-3-prop-2-enyloxy- 5,6,7,8,13,13a- hexahydroisoquinolino[2,1- b]isoquinolin-9-yl benzenesulfonate | C31H33NO6S | 547.66 | 548.1 |
| 104 | 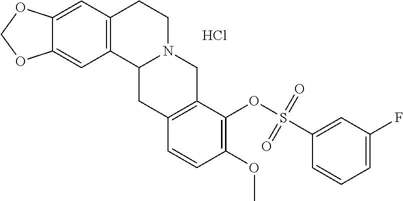 | 10-methoxy-5,6,7,8,13,13a- hexahydro-2H-1,3-dioxolano [4,5-g]isoquinolino[3,2-a] isoquinolin-9-yl 3- fluorobenzenesulfonate hydrochloride | C25H23ClFNO6S | 519.97 | 512.0 |
| 105 | 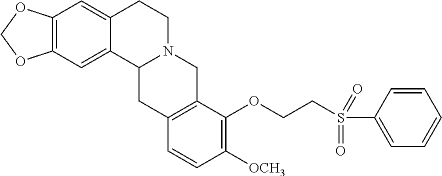 | 9-O-2-(1′-phenylsulfonyl- ethyl)-10-methoxy-5,8,13,13a- tetrahydro-6H- [1,3]dioxolo[4,5- g]isoquino[3,2-a]isoquinoline | C27H27NO6S | 493.57 | 494.3 |
| 112 | 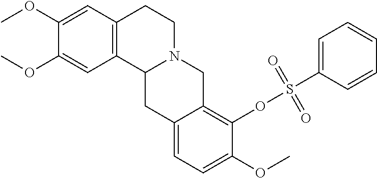 | 2,3,10-Trimethoxy- 5,6,7,8,13,13a- hexahydroisoquinolino[2,1- b]isoquinolin-9-yl benzenesulfonate tartrate | C26H27NO6S | 631.65 | 482.1 |
| 113 | 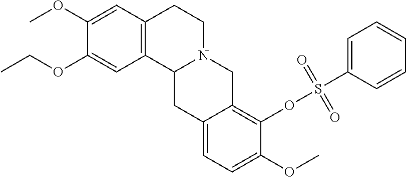 | 2-ethoxy-3,10-dimethoxy- 5,6,7,8,13,13a- hexahydroisoquinolino[2,1- b]isoquinolin-9-yl benzenesulfonate | C₂₇H₂₉NO₆S | 495.59 | 496.1 |
| 114 | 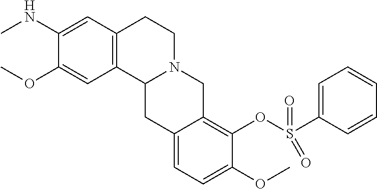 | 2,10-Dimethoxy-3- (methylamino)-5,6,7,8,13,13a- hexahydro isoquinolino[2,1- b]isoquinolin-9-yl benzenesulfonate | C26H28N2O5S | 480.58 | 481.1 |
| 115 | 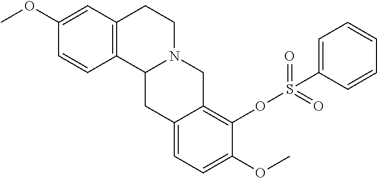 | 11-Methoxy-6,7,8,9,14,14a- hexahydro-2H,3H-1,4- dioxano[5,6- g]isoquinolino[3,2- a]isoquinolin-10-yl benzenesulfonate | C26H25NO6S | 479.54 | 480.0 |
| 116 | 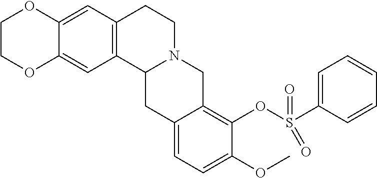 | 3,10-Dimethoxy- 5,6,7,8,13,13a- hexahydroisoquinolino[2,1- b]isoquinolin-9-yl benzenesulfonate | C25H25NO5S | 451.53 | 452.1 |
| 117 | 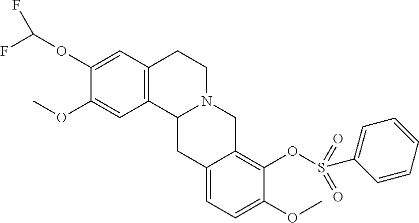 | 3-(Difluoromethoxy)-2,10- dimethoxy-5,6,7,8,13,13a- hexahydro isoquinolino[2,1- b]isoquinolin-9-yl benzenesulfonate | C₂₆H₂₅F₂NO₆S | 517.54 | 518.1 |
| 118 | 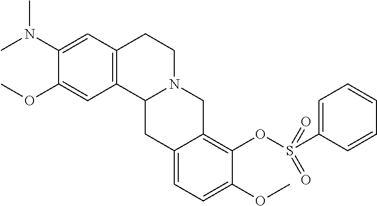 | 3-(Dimethylamino)-2,10- dimethoxy-5,6,7,8,13,13a- hexahydro isoquinolino[2,1- b]isoquinolin-9-yl benzenesulfonate | C₂₇H₃₀N₂O₅S | 494.6 | 495.1 |
| 119 | 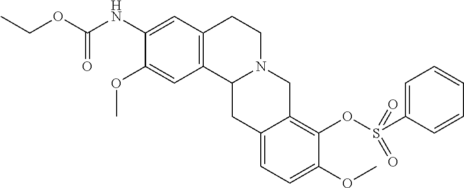 | 3-(Ethoxycarbonylamino)- 2,10-dimethoxy- 5,6,7,8,13,13a- hexahydroisoquinolino[2,1- b]isoquinolin-9-yl benzenesulfonate | C₂₈H₃₀N₂O₇S | 538.61 | 539.1 |
| 120 | 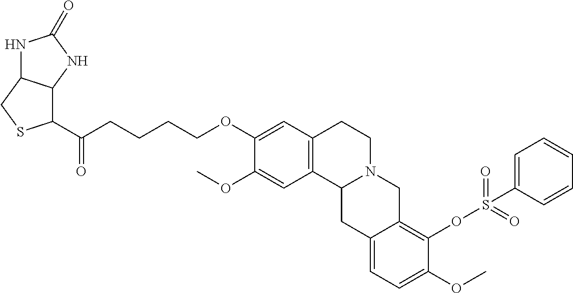 | 2,10-Dimethoxy-9- (phenylsulfonyloxy)- 5,6,7,8,13,13a- hexahydroisoquinolino[3,2- a]isoquinolin-3-yl 5- ((5S,1R,2R)-7-oxo-3-thia-6,8- diazabicyclo[3.3.0]oct-2- yl)pentanoate | C35H39N3O8S2 | 693.83 | 694.1 |
| 121 | 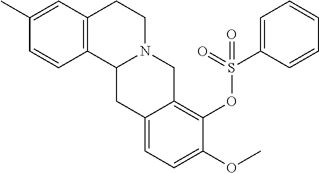 | 10-Methoxy-3-methyl- 5,6,7,8,13,13a- hexahydroisoquinolino[2,1- b]isoquinolin-9-yl benzenesulfonate | C₂₅H₂₅NO₄S | 435.54 | 436.1 |
| 122 | 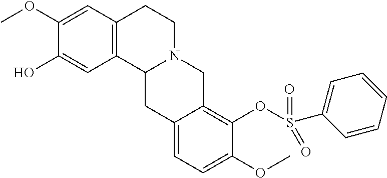 | 2-hydroxy-3,10-dimethoxy- 5,6,7,8,13,13a- hexahydroisoquinolino[2,1- b]isoquinolin-9-yl benzenesulfonate | C₂₅H₂₅NO₆S | 467.53 | 468.1 |
| 123 | 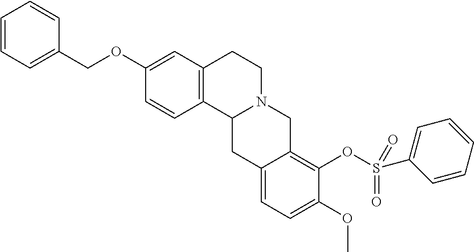 | 10-Methoxy-3- (phenylmethoxy)- 5,6,7,8,13,13a- hexahydroisoquinolino[2,1- b]isoquinolin-9-yl benzenesulfonate | C31H29NO5S | 527.63 | 528.1 |
| 124 | 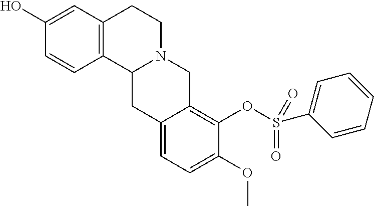 | 3-Hydroxy-10-methoxy- 5,6,7,8,13,13a- hexahydroisoquinolino[2,1- b]isoquinolin-9-yl benzenesulfonate | C24H23NO5S | 437.51 | 438.1 |
| 125 | 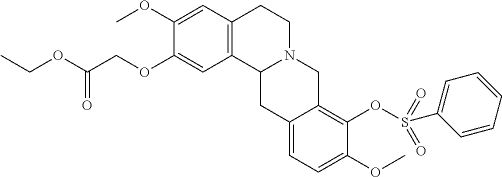 | Ethyl 2-[3,10-dimethoxy-9- (phenylsulfonyloxy)- 5,6,7,8,13,13a- hexahydroisoquinolino[3,2- a]isoquinolin-2-yloxy]acetate | C29H31NO8S | 553.62 | 554.1 |
| 126 | 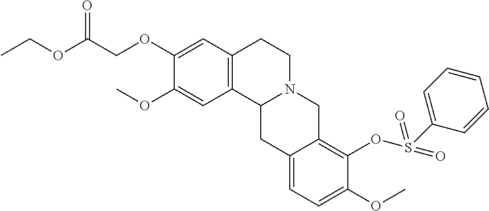 | Ethyl 2-[2,10-dimethoxy-9- (phenylsulfonyloxy)- 5,6,7,8,13,13a- hexahydroisoquinolino[3,2- a]isoquinolin-2-yloxy]acetate | C29H31NO8S | 553.62 | 554.1 |
| 127 | 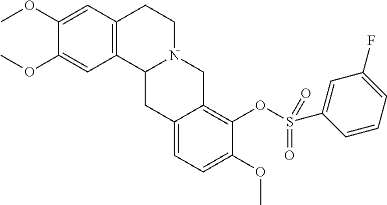 | 2,3,10-Trimethoxy- 5,6,7,8,13,13a- hexahydroisoquinolino[2,1- b]isoquinolin-9-yl 3- fluorobenzenesulfonate | C₂₆H₂₆FNO₆S | 499.55 | 500.1 |
| 128 | 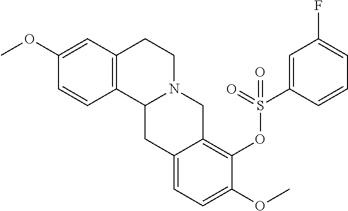 | 3,10-Dimethoxy- 5,6,7,8,13,13a- hexahydroisoquinolino[2,1- b]isoquinolin-9-yl 3- fluorobenzenesulfonate | C₂₅H₂₄FNO₅S | 469.53 | 470.1 |
| 129 | 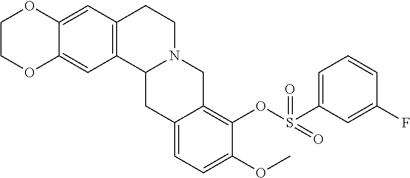 | 11-Methoxy-6,7,8,9,14,14a- hexahydro-2H,3H-1,4- dioxano[5,6- g]isoquinolino[3,2- a]isoquinolin-10-yl 3- fluorobenzenesulfonate | C₂₆H₂₄FNO₆S | 497.54 | 498.1 |
| 130 | 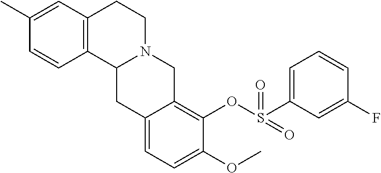 | 10-Methoxy-3-methyl- 5,6,7,8,13,13a- hexahydroisoquinolino[2,1- b]isoquinolin-9-yl 3- fluorobenzenesulfonate | C₂₅H₂₄FNO₄S | 453.53 | 454.1 |
| 131 | 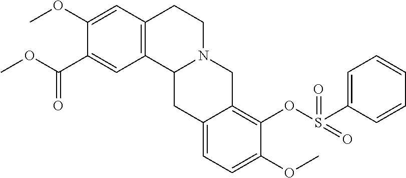 | Methyl 3,10-dimethoxy-9- (phenylsulfonyloxy)- 5,6,7,8,13,13a- hexahydroisoquinolino[3,2- a]isoquinoline-2-carboxylate | C27H27NO7S | 509.57 | 510.1 |
| 132 | 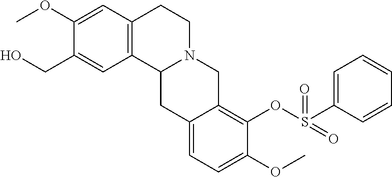 | 2-(Hydroxymethyl)-3,10- dimethoxy-5,6,7,8,13,13a- hexahydro isoquinolino[2,1- b]isoquinolin-9-yl benzenesulfonate | C26H27NO6S | 481.56 | 482.1 |
| 133 | 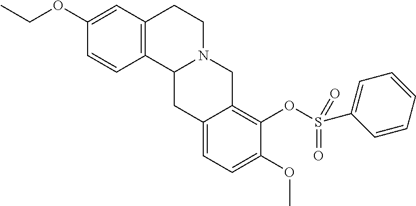 | 3-Ethoxy-10-methoxy- 5,6,7,8,13,13a- hexahydroisoquinolino[2,1- b]isoquinolin-9-yl benzenesulfonate | C26H27NO5S | 465.56 | 466.1 |
| 134 | 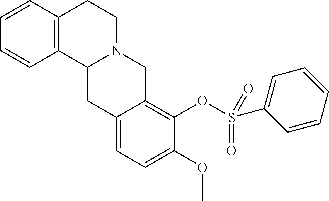 | 10-Methoxy- 5,6,7,8,13,13a- hexahydroisoquinolino[2,1- b]isoquinolin-9-yl benzenesulfonate | C25H23NO4S | 421.51 | 422.1 |
| 135 | 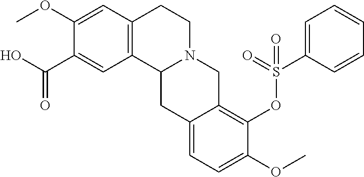 | 3,10-Dimethoxy-9- (phenylsulfonyloxy)- 5,6,7,8,13,13a-hexahydro isoquinolino[3,2- a]isoquinoline-2-carboxylic acid | C26H25NO7S | 495.54 | 494.0 (M⁻ − 1) |
| 136 | 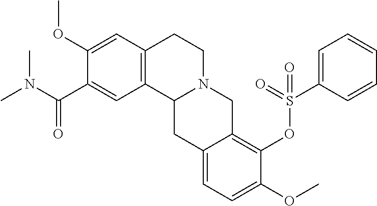 | 2-(N,N-dimethylcarbamoyl)- 3,10-dimethoxy- 5,6,7,8,13,13a- hexahydroisoquinolino[2,1- b]isoquinolin-9-yl benzenesulfonate | C28H30N2O6S | 522.61 | 523.1 |
| 137 | 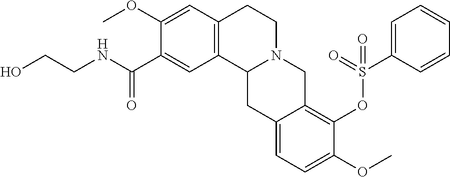 | 2-[N-(2- hydroxyethyl)carbamoyl]-3,10- dimethoxy-5,6,7,8,13,13a- hexahydroisoquinolino[2,1- b]isoquinolin-9-yl benzenesulfonate | C28H30N2O7S | 538.61 | 539.1 |
| 138 | 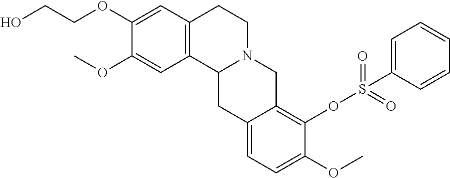 | 3-(2-Hydroxyethoxy)-2,10- dimethoxy-5,6,7,8,13,13a- hexahydroisoquinolino[2,1- b]isoquinolin-9-yl benzenesulfonate | C27H29NO7S | 511.59 | 512.1 |
| 139 | 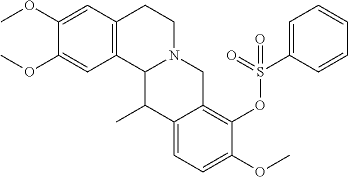 | 2,3,10-Trimethoxy-13-methyl- 5,6,7,8,13,13a-hexahydro isoquinolino[2,1-b]isoquinolin- 9-yl benzenesulfonate | C₂₇H₂₉NO₆S | 495.59 | 496.1 |
| 140 | 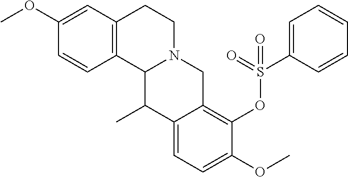 | 3,10-Dimethoxy-13-methyl- 5,6,7,8,13,13a-hexahydro isoquinolino[2,1-b]isoquinolin- 9-yl benzenesulfonate | C₂₆H₂₇NO₅S | 465.56 | 466.1 |
| 141 | 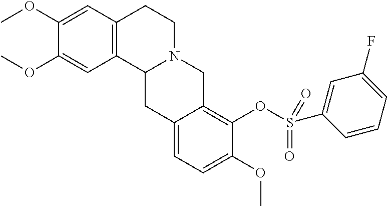 | 2,3,10-Trimethoxy- 5,6,7,8,13,13a- hexahydroisoquinolino[2,1- b]isoquinolin-9-yl 3- fluorobenzenesulfonate phosphate Salt | C₂₆H₂₆FNO₆S | 597.55 | 500.1 |
| 142 | 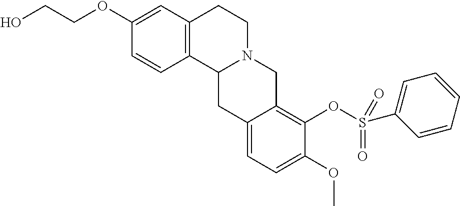 | 3-(2-Hydroxyethoxy)-10- methoxy-5,6,7,8,13,13a- hexahydroisoquinolino[2,1- b]isoquinolin-9-yl benzenesulfonate | C₂₆H₂₇NO₆S | 481.56 | 482.1 |
| 143 | 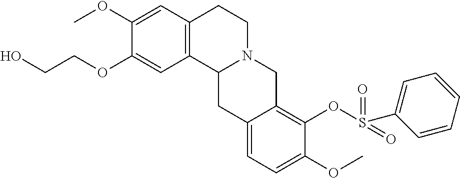 | 2-(2-hydroxyethoxy)-3,10- dimethoxy-5,6,7,8,13,13a- hexahydroisoquinolino[2,1- b]isoquinolin-9-yl benzenesulfonate | C₂₇H₂₉NO₇S | 511.59 | 512.1 |
| 144 | 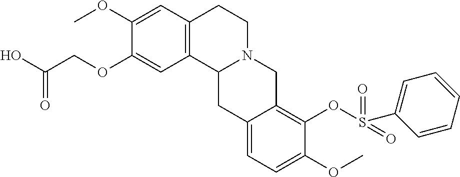 | 2-[3,10-dimethoxy-9- (phenylsulfonyloxy)- 5,6,7,8,13,13a- hexahydroisoquinolino[3,2- a]isoquinolin-2-yloxy]acetic acid | C₂₇H₂₇NO₈S | 525.57 | 524.1 (M − 1) |
| 145 | 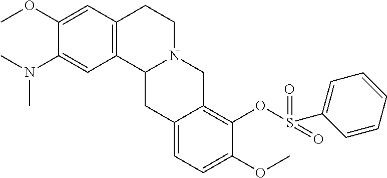 | 2-(dimethylamino)-3,10- dimethoxy-5,6,7,8,13,13a- hexahydroisoquinolino[2,1- b]isoquinolin-9-yl benzenesulfonate | C27H30N2O5S | 494.6 | 495.2 |
| 146 | 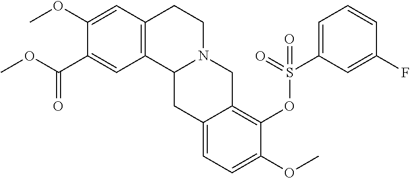 | Methyl 9-[(3- fluorophenyl)sulfonyloxy]- 3,10-dimethoxy- 5,6,7,8,13,13a- hexahydroisoquinolino[3,2- a]isoquinoline-2-carboxylate | C27H26FNO7S | 527.56 | 528.2 |
| 147 | 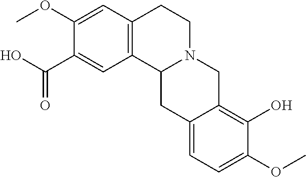 | 9-Hydroxy-3,10-dimethoxy- 5,6,7,8,13,13a-hexahydro isoquinolino[3,2- a]isoquinoline-2-carboxylic acid | C₂₀H₂₁NO₅ | 355.38 | 354.1 |
| 148 | 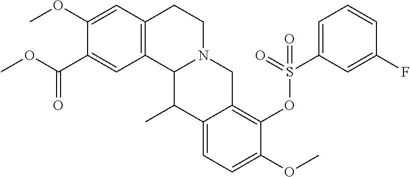 | Methyl 9-[(3- fluorophenyl)sulfonyloxy]- 3,10-dimethoxy-13-methyl- 5,6,7,8,13,13a- hexahydroisoquinolino[3,2- a]isoquinoline-2-carboxylate | C28H28FNO7S | 541.59 | 542.1 |
| 149 | 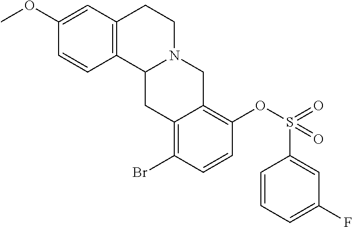 | 12-bromo-3-methoxy- 5,6,7,8,13,13a- hexahydroisoquinolino[2,1- b]isoquinolin-9-yl 3- fluorobenzenesulfonate | C24H21BrFNO4S | 518.39 | 518.0 |
| 150 | 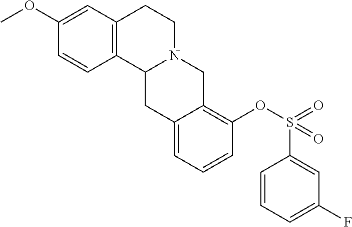 | 3-methoxy-5,6,7,8,13,13a- hexahydroisoquinolino[2,1- b]isoquinolin-9-yl 3- fluorobenzenesulfonate | C24H22FNO4S | 439.5 | 440.1 |
| 151 | 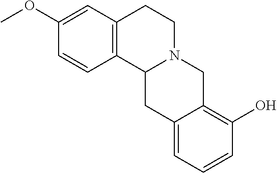 | 3-methoxy-5,6,7,8,13,13a- hexahydroisoquinolino[2,1- b]isoquinolin-9-ol | C18H19NO2 | 281.35 | 282.1 |
| 152 | 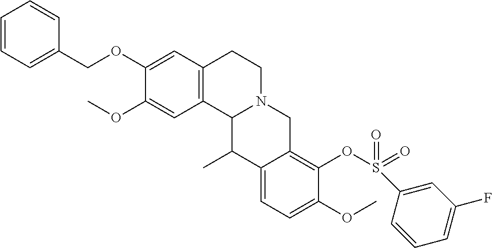 | 2,10-dimethoxy-13-methyl-3- (phenylmethoxy)- 5,6,7,8,13,13a- hexahydroisoquinolino[2,1- b]isoquinolin-9-yl 3- fluorobenzenesulfonate | C33H32FNO6S | 589.67 | 590.2 |
| 153 | 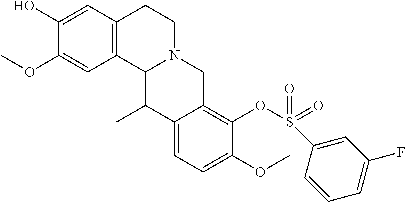 | 3-hydroxy-2,10-dimethoxy-13- methyl-5,6,7,8,13,13a- hexahydroisoquinolino[2,1- b]isoquinolin-9-yl 3- fluorobenzenesulfonate | C26H26FNO6S | 499.55 | 500.1 |
| 154 | 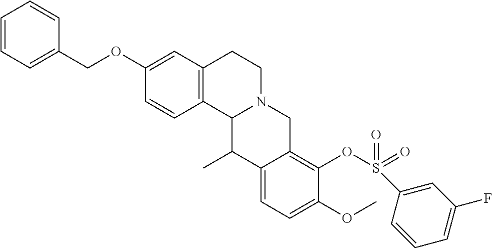 | 10-Methoxy-13-methyl-3- (phenylmethoxy)- 5,6,7,8,13,13a- hexahydroisoquinolino[2,1- b]isoquinolin-9-yl 3- fluorobenzenesulfonate | C32H30FNO5S | 559.64 | 560.1 |
| 155 | 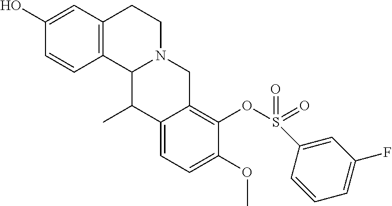 | 3-Hydroxy-10-methoxy-13- methyl-5,6,7,8,13,13a- hexahydroisoquinolino[2,1- b]isoquinolin-9-yl 3- fluorobenzenesulfonate | C25H24FNO5S | 469.53 | 470.1 |
| 156 | 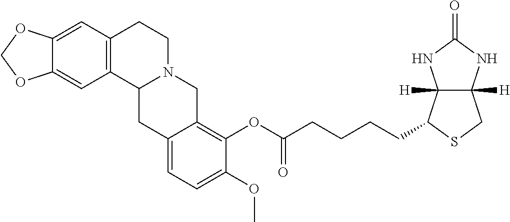 | 10-Methoxy-5,6,7,8,13,13a- hexahydro-2H-1,3- dioxolano[4,5- g]isoquinolino[3,2- a]isoquinolin-9-yl 5- ((5S,1R,2R)-7-oxo-3-thia-6,8- diazabicyclo[3.3.0]oct-2- yl)pentanoate | C29H33N3O6S | 551.65 | 552.1 |
| 157 | 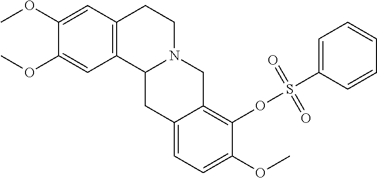 | 2,3,10-Trimethoxy- 5,6,7,8,13,13a- hexahydroisoquinolino[2,1- b]isoquinolin-9-yl benzenesulfonate hydrochloride | C₂₆H₂₇NO₆S | 518.01 | 482.1 |
| 158 | 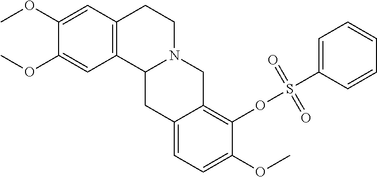 | 2,3,10-Trimethoxy- 5,6,7,8,13,13a- hexahydroisoquinolino[2,1- b]isoquinolin-9-yl benzenesulfonate sulfate | C₂₆H₂₇NO₆S | 579.64 | 482.1 |
| 159 | 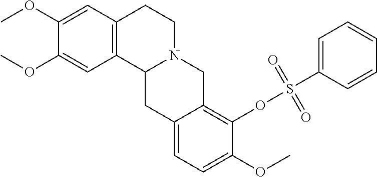 | 2,3,10-Trimethoxy- 5,6,7,8,13,13a- hexahydroisoquinolino[2,1- b]isoquinolin-9-yl benzenesulfonate phosphate | C26H27NO6S | 579.56 | 482.1 |
| 160 | 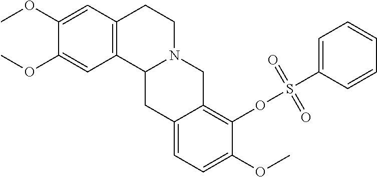 | 2,3,10-Trimethoxy- 5,6,7,8,13,13a- hexahydroisoquinolino[2,1- b]isoquinolin-9-yl benzenesulfonate lactate | C26H27NO6S | 571.64 | 482.1 |
| 161 | 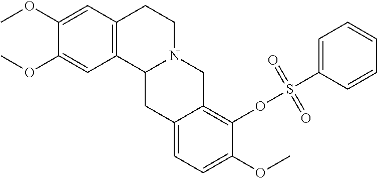 | 2,3,10-Trimethoxy- 5,6,7,8,13,13a- hexahydroisoquinolino[2,1- b]isoquinolin-9-yl benzenesulfonate citrate | C26H27NO6S | 673.68 | 482.1 |
| 162 | 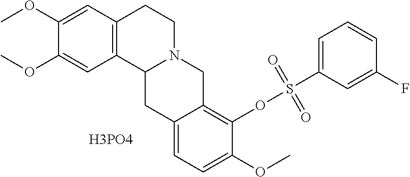 | 2,3,10-Trimethoxy- 5,6,7,8,13,13a- hexahydroisoquinolino[2,1- b]isoquinolin-9-yl 3- fluorobenzenesulfonate phosphate Salt (Enantiomer having positive optical rotation) | C26H26FNO6S•H3PO4 | 597.55 | 500.0 |
| 163 | 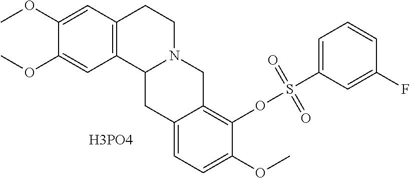 | 2,3,10-Trimethoxy- 5,6,7,8,13,13a- hexahydroisoquinolin[2,1- b]isoquinolin-9-yl 3- fluorobenzenesulfonate phosphate Salt (Enantiomer having negative optical rotation) | C26H26FNO6S•H3PO4 | 597.55 | 500.0 |

*unless a different ion is indicated
**molecular weight of free base

### Example 4

### Evaluation of Pharmacokinetics of New Compound 72 Enantiomers in Male Wister Rats Following Intravenous and Oral Administration

The objective of this study was to collect plasma samples from male Wister rats at various time points following intravenous and oral administration of test compounds (72(+) and 72(−)). These samples were used later for the determination of plasma compound levels by LC/MS/MS for estimating pharmacokinetic parameters.

Male Wister rats (body weight: 100 to 200 g) were used in this study. Before the pharmacokinetic studies, animals were randomly assigned to 4 groups (3 animals per time point). The treatment condition is shown in Table 5.

For intravenous administration, blood samples were collected via retro-orbital puncture into heparinized tubes at pre-dose and 0.083, 0.25, 0.5, 1, 2, 6, 8, and 24 hours (hr) post-dose.

For oral administration, blood samples were collected at following time points: 0, 0.167, 0.417, 0.75, 1, 2, 4, 6, 8, and 24 hr. After sample collection, plasma samples were stored at −20° C. until bioanalysis. Concentrations of 72(+) and 72(−) in plasma samples were determined using a high performance liquid chromatography/mass spectrometry (HPLC/MS/MS) method.

Collectively, the results indicate that both enantiomers of new compound 72 are relatively stable with half-lives of systemic clearance >2.5 hours and bioavailability higher than 45%. The bioavailability (F*) was calculated by applying the following formula:

*F(%)=(Doseᵢᵥ×AUCₒᵣₐₗ₍₀₋ₜ₎)/(Doseₒᵣₐₗ×AUCᵢᵥ₍₀₋ₜ₎)×100%.

TABLE 5
Experimental Design

| | | | | | Treatment | | |
|---|---|---|---|---|---|---|---|
| | Number of | | Dose | Dose | Dose | | |
| | Animals | | Level | Conc | Volume | | Dosing |
| Group | Male | Test Article | (mg/kg) | (mg/ml) | (mL/kg) | Vehicle | Route |
| 1 | 3 | 72(+) | 1 | 0.5 | 2 | 1% DMSO in 0.1 M | IV |
| | | | | | | phosphate buffer, pH 3.0 | |
| 2 | 3 | 72(+) | 5 | 0.5 | 10 | 1% DMSO in 0.1 M | PO |
| | | | | | | phosphate buffer, pH 3.0 | |
| 3 | 3 | 72(−) | 1 | 0.5 | 2 | 1% DMSO in 0.1 M | IV |
| | | | | | | phosphate buffer, pH 3.0 | |
| 4 | 3 | 72(−) | 5 | 0.5 | 10 | 1% DMSO in 0.1 M | PO |
| | | | | | | phosphate buffer, pH 3.0 | |
| | | | | | | buffer, pH 3.0) | |

### In-Vitro Study Results

### Example 5

### Upregulation of LDLR mRNA Expression in Human Hepatoma Derived Cell Line HepG2 by 6 Active Compounds Derived from Corydalis Genus that are all d-(+) Enantiomers

### A: Compound Structures

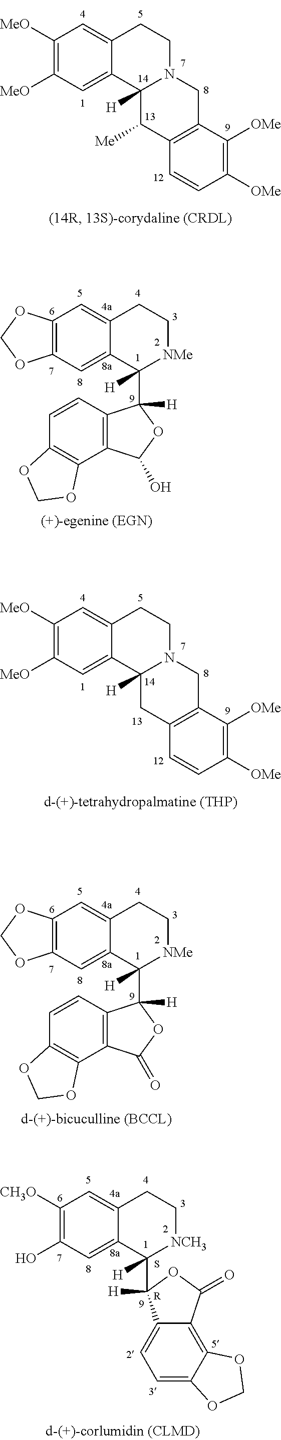

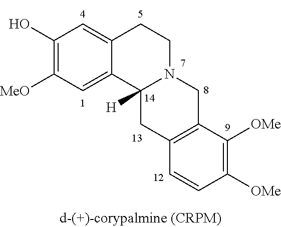

### B: Biological Activity

HepG2 cells obtained from American Tissue Culture Collection (Manassas, Va., USA) were seeded in 6-well culture plates at a density of 0.8×10⁶ cells/well cultured in EMEM containing 0.5% FBS and were treated with each purified compound at indicated doses for 8 hours. Total RNA was isolated, and 2 μg per sample was reverse transcribed with random primers using M-MLV (Promega) at 37° C. for 1 hour. PCR was carried out at 94° C. for 30 sec, 60° C. for 30 sec, and 72° C. for 30 sec with initial activation of the enzyme at 94° C. for 1 minute. Thirty cycles were performed for LDLR and GAPDH. PCR was performed using primers HLDLR-up and HLDLR-lo for LDLR and primers HGAPDH-up and HGAPDH-lo for GAPDH. The PCR products were separated on a 1% agarose gel and the band intensity was quantitated. LDLR mRNA levels were corrected by measuring GAPDH mRNA levels.

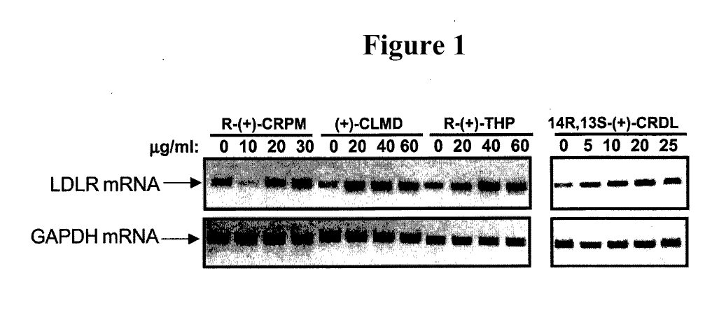

The potent and dose-dependent effects of (+)-CLMD, 14R-(+)-CRPM, 14R,13S-(+)-CRDL, and 14R-(+)-THP on LDLR mRNA expression in HepG2 cells by a semi-quantitative RT-PCR analysis are shown in FIG. 1.

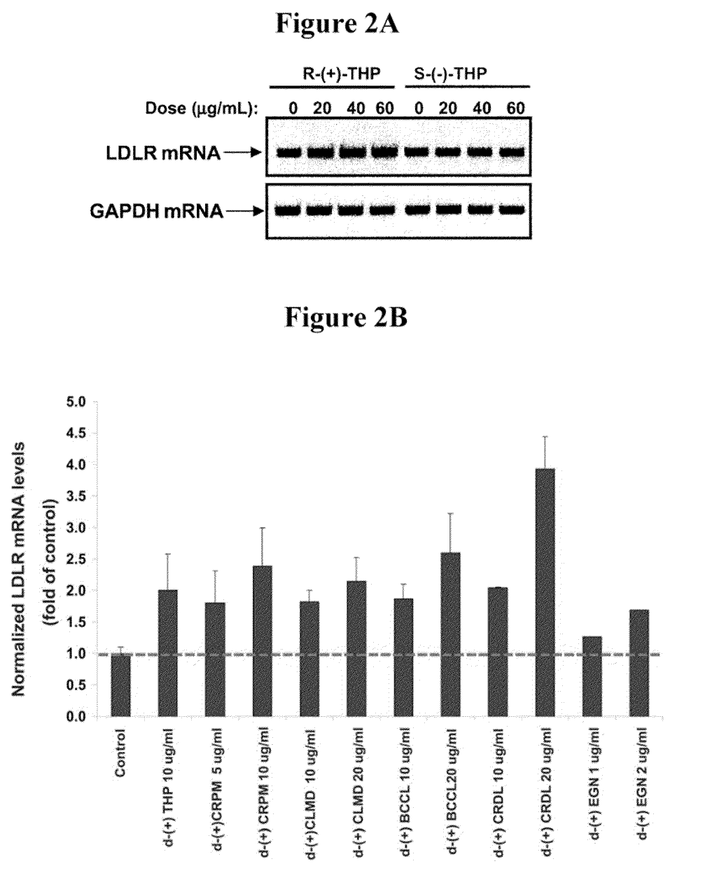

Specific stereochemical requirements of +1-THP in the upregulation of LDLR mRNA expression were determined by a similar experiment. HepG2 cells were treated with the pure 14R-(+)-THP or the pure 145-(−)-THP at the indicated concentrations for 24 hours. The levels of LDLR mRNA and GAPDH mRNA in untreated and the compound-treated HepG2 cells were assessed by semi-quantitative RT-PCR. The results are as shown in FIG. 2.

In yet another experiment, HepG2 cells were treated with various compounds for 24 hours at the indicated doses. Total RNA was isolated and 2 μg was used to generate cDNA in a reaction containing random primers and M-MLV at 37° C. for 1 hour in a volume of 25 μL. Real-time PCR was performed on the cDNA using MCEP REALPLEX 2 SYSTEM (Eppendorf) and Universal MasterMix (Applied Biosystems). Human LDLR and GAPDH Pre-Developed TaqMan Assay Reagents (Applied Biosystems) were used to assess the levels of mRNA expressions in HepG2. The levels of LDLR mRNA were normalized to that of GAPDH. Each RNA samples was assayed in triplicate. The abundance of LDLR mRNA in untreated cells was defined as 1, and the amounts of LDLR mRNA from compound-treated cells were plotted relative to that value in FIG. 2B. The data shown are mean±s.d. The results showed that all 6 compounds with the specific d-(+) enantiomeric configurations elevated LDLR mRNA levels in a dose-dependent manner.

### Example 6

### Stimulation of LDLR Ligand Uptake Activity in HepG2 Cells by corydalis-Derived Compounds and by Some New Compounds of Formula I, II, III, and IV

HepG2 cells (2×10⁵ cells/well) in 24-well culture plates were treated with various compounds at indicated concentrations for 20 hours. The fluorescent 1.1′-dioctadecyl-3,3,3′,3′-tetramethylindocarbocyanin perchlorate (DiI-LDL) (Biomedical Technologies, Stoughton, Mass.) at a concentration of 2 μg/mL was added to cells at the end of treatment. After 4 hours, the medium was removed, cells were washed with cold PBS, and were examined immediately under a fluorescent microscope (Nikon) at 200× amplification. The fluorescent intensity in compound-treated cells was compared to that in untreated control cells. The compound activity was graded as follows: +, slightly increased fluorescent intensity over control; ++ modestly increased fluorescent intensity over control; +++, strongly increased fluorescent intensity over control. The results are summarized below in Table 6. In these experiments berberine chloride was used for comparison.

TABLE 6

| | Activity on DiI-LDL | A, compound dose ≦10 μM |
|---|---|---|
| Compound No. | uptake | B, compound dose ≦40 μM |
| Berberine | + | B |
| d-(+)-THP | ++ | B |
| d-(+)-CRPM | ++ | A |
| d-(+)-CRDL | ++ | B |
| d-(+)-EGN | + | A |
| d-(+)-CLMD | + | B |
| 1 | + | A |
| 26 | ++ | A |
| 30 | + | A |
| 34 | ++ | A |
| 38 | ++ | A |
| 41 | ++ | A |
| 43 | ++ | A |
| 44 | ++ | A |
| 45 | ++ | A |
| 46 | ++ | A |
| 50 | +++ | A |
| 56 | +++ | A |
| 69 | +++ | A |

### Example 7

Activities of some compounds of Formulas I, II and EE on LDLR mRNA expression were determined. HepG2 cells were treated with new compounds individually at a dose less than or equal to 10 μM for 24 hours. Total RNA was harvested for quantitative real-time RT-PCR analysis using the method described in Example 5B. The fold activity was derived by dividing the amount of normalized LDLR mRNA in compound-treated cells over the amount of LDLR mRNA in untreated control cells. In these experiments, berberine chloride was included for comparison. Results are shown in Table 7.

TABLE 7

| | Activity on LDLR mRNA |
|---|---|
| | expression |
| Compound No. | (Fold of control) |
| BBR | 1.30 ± 0.11 |
| 1 | 1.56 ± 0.86 |
| 26 | 1.29 ± 0.10 |
| 30 | 1.47 ± 0.13 |
| 34 | 1.76 ± 0.17 |
| 37 | 2.47 ± 0.07 |
| 38 | 1.82 ± 0.10 |
| 41 | 1.60 ± 0.03 |
| 43 | 1.95 ± 0.18 |
| 44 | 2.20 ± 0.06 |
| 45 | 2.09 ± 0.11 |
| 46 | 1.80 ± 0.16 |
| 47 | 1.63 ± 0.09 |
| 48 | 1.45 ± 0.15 |
| 50 | 4.74 ± 0.96 |
| 51 | 3.79 ± 0.33 |
| 52 | 3.63 ± 0.17 |
| 53 | 3.96 ± 0.40 |
| 56 | 3.18 ± 0.63 |
| 57 | 2.62 ± 0.17 |
| 58 | 3.90 ± 0.01 |
| 59 | 3.73 ± 0.10 |
| 60 | 3.46 ± 0.75 |
| 61 | 1.33 ± 0.53 |
| 62 | 0.69 ± 0.02 |
| 63 | 2.94 ± 0.13 |
| 64 | 2.82 ± 0.45 |
| 65 | 2.58 ± 0.46 |
| 66 | 2.54 ± 0.06 |
| 69 | 2.21 ± 0.09 |
| 85 | 1.42 ± 0.30 |
| 86 | 2.48 ± 0.62 |
| 88 | 1.93 ± 0.24 |
| 89 | 1.92 ± 0.40 |
| 90 | 1.21 ± 0.07 |
| 91 | 3.77 ± 0.20 |
| 92 | 1.52 ± 0.07 |
| 93 | 1.42 ± 0.16 |
| 94 | 1.22 ± 0.18 |
| 95 | 1.43 ± 0.04 |
| 96 | 1.33 ± 0.05 |
| 100 | 1.44 ± 0.59 |
| 101 | 1.97 ± 0.00 |

### Example 8

### Demonstration of Reduction of Intracellular Triglyceride in HepG2 Cells Treated with Some of the NCEs

Berberine chloride was used for comparison. The amount of TG in untreated control cells was defined as 100% and the amounts of TG in compound-treated cells were divided to that value. Results are shown in Table 8.

TABLE 8

| | | A, compound |
|---|---|---|
| | Activity on reduction of | dose ≦10 μM |
| Compound | cellular TG accumulation | B, compound |
| No. | (% of control) | dose ≦40 μM |
| Control | 100 | |
| Berberine | 70.9 ± 5.4 | B |
| 69 | 60.4 ± 7.34 | A |
| 82 | 53.2 ± 21.3 | B |
| 83 | 63.0 ± 0.90 | B |
| 22 | 53.35 ± 12.68 | B |
| 91 | 71.17 ± 2.23 | B |

### Example 9

### Effect of Compounds of Present Technology on PCSK9 mRNA Expression

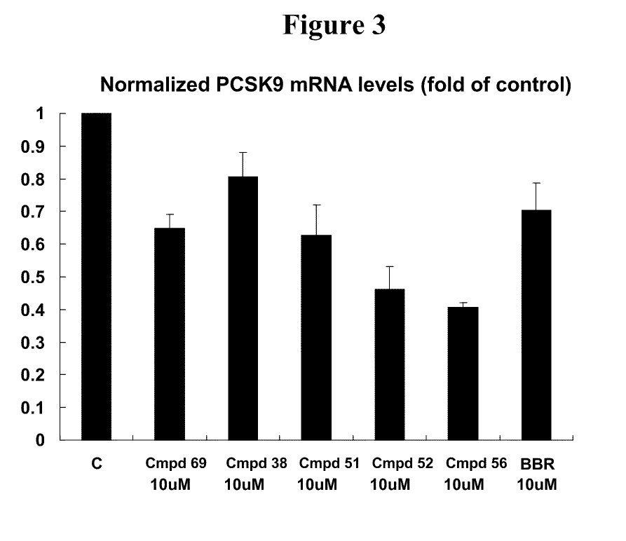

The effects of compounds of Formula I, II and EE on the inhibition of PCSK9 mRNA expression was examined (Horton J D, Cohen J C, Hobbs H H. “Molecular biology of PCSK9: its role in LDL metabolism” TRENDS in Biochemical Sciences 2007; 32:71-77; Cameron J, Ranheim T, Kulseth M A, Leren T P, Berge K E. “Berberine decreases PCSK9 expression in HepG2 cells” Atherosclerosis, 2008 online publication). HepG2 cells were treated with new compounds individually at 10 uM dose for 24 hours. Total RNA was harvested for quantitative real-time RT-PCR analysis using method described in Example 5B. The fold activity was derived by dividing the amount of normalized PCSK9 mRNA in compound-treated cells over the amount of PCSK9 mRNA in untreated control cells. In these experiments, berberine chloride (10 uM) was included for comparison (see FIG. 3). These results showed that these new compounds strongly inhibit the mRNA expression of PCSK9, thereby providing another means to increase LDLR expression by reducing PCSK9-mediated degradation of LDLR protein.

### Example 10

### Comparisons of Simvastatin and New Compound 91 on LDLR and PCSK9 mRNA Expressions

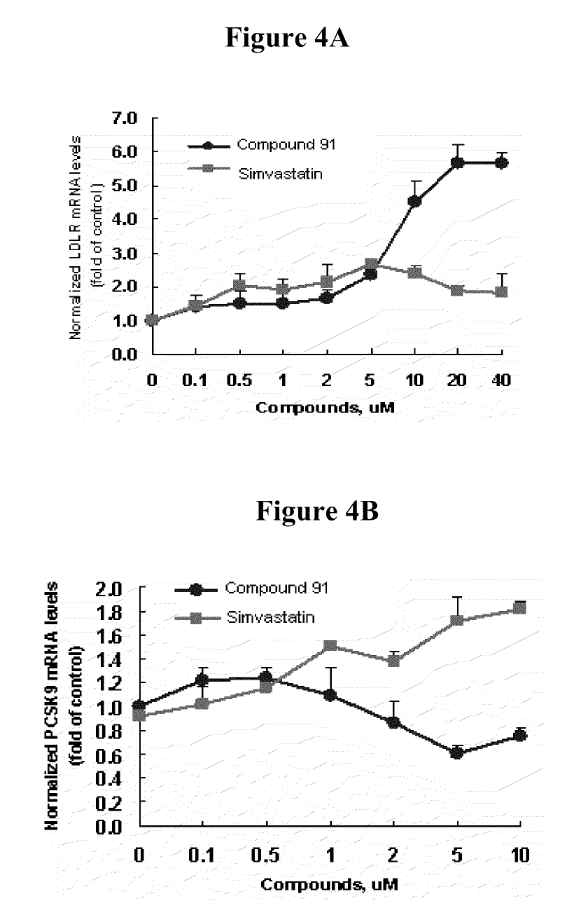

HepG2 cells were treated with simvastatin or compound 91 at the indicated concentrations for 24 hours. Total RNA was harvested for quantitative real-time RT-PCR analysis using method described in Examples 5B. The fold activity was derived by dividing the amount of normalized LDLR or PCSK9 mRNA in compound-treated cells over the amount of LDLR or PCSK9 mRNA in untreated control cells. These results as shown in FIG. 4A and FIG. 4B demonstrated that the new compound 91 dose-dependently increases LDLR mRNA expression but inhibits the PCSK9 mRNA expression, whereas simvastatin increases both LDLR and PCSK9 mRNA expression.

### Example 11

### Assessment of Stability of New Compound 69 Under Cell Culture Conditions

HepG2 cells were cultured in 6-well cell culture plate in EMEM containing 0.5% FBS overnight. Compound 69+ was added to the culture medium to a final concentration of 20 μM. Medium was collected at 0 (control), 4, 8, or 24 hours after the compound addition.

The collected media were analyzed by LC-MS on a Thermo Fisher Scientific Surveyor HPLC system and LCQ ion trap MS with electrospray ionization source. A 50×2. 1 mm Hypersil Gold C18 3 um column was used with a flow rate of 200 uL/min. The method conditions were 98% A (0.1% formic acid in water)/2% B (0.1% formic acid in acetonitrile) then a gradient to 95% B in 10 minutes. The injection volume was 10 uL.

LC-MS shows that a single peak with elution time of 7 of HPLC and a strong signal at 466.1 (M⁺+H) of MS were detected in all medium samples. Table 9 shows the peak area of 69 in medium collected at different time point.

All together, these results suggest the new compound 69 is fairly stable under cell culture conditions and that the stability of the compound is a contributing factor to its strong effect on the upregulation of LDLR expression in HepG2 cells.

TABLE 9
Peak Area m/z 466.1

| Sample ID | Peak Area m/z 466.1 |
|---|---|
| 052008-Ctrl | 2.38e8 |
| 052008-4 h | 9.67e7 |
| 052008-8 h | 9.22e7 |
| 052008-24 h | 7.03e7 |

### Example 12

The effects of compounds on the mRNA expressions of LDLR and PCSK9 were examined using quantitative real-time RT-PCR assays. HepG2 cells were treated with various compounds at 10 and 40 uM concentrations for 24 hours. Total RNA was isolated and 2 ng was used to generate cDNA in a reaction containing random primers and M-MLV at 37° C. for 1 h in a volume of 25 μL. Real-time PCR was performed on the cDNA using MCEP REALPLEX 2 SYSTEM (Eppendorf) and Universal MasterMix (Applied Biosystems). Human LDLR, PCSK9, and GAPDH Pre-Developed TaqMan Assay Reagents (Applied Biosystems) were used to assess the levels of mRNA expressions in HepG2. The levels of LDLR and PCSK9 mRNA were normalized to that of GAPDH. Each RNA samples was assayed in duplicate. The fold activity was calculated by dividing mRNA abundance in compound treated cells with that of control. The data (mean±s. d.) are summarized in Table 10 columns 2-5. These results showed that these synthetic compounds strongly increase LDLR mRNA expression and also exhibit inhibitory activity on PCSK9 mRNA expression in dose-dependent manners.

The combined beneficial effects of these novel compounds on elevation of LDLR mRNA levels likely through the mechanism of mRNA stabilization and their inhibitory activity towards PCSK9, the secreted protease that degrades LDLR protein, result in strong increases in LDL ligand uptake activity through increased receptor surface abundance. The ligand uptake activity was demonstrated measuring the intracellular accumulation of fluorescent labeled LDL. Briefly, HepG2 cells (2×10⁵ cells/well) in 24-well culture plates were treated with various compounds at 10 and 40 uM concentrations for 20 hours. The fluorescent 1.1′-dioctadecyl-3,3,3′,3′-tetramethylindocarbocyanin perchlorate (DiI-LDL) (Biomedical Technologies, Stoughton, Mass.) at a concentration of 2 μg/ml was added to cells at the end of treatment. After 4 hours, the medium was removed; cells were washed with cold PBS, and were examined immediately under a fluorescent microscope (Nikon) at 200× amplification. The fluorescent intensity in compound-treated cells was compared to that in untreated control cells. The compound activity was graded as follows: +, slightly increased fluorescent intensity over control; ++ modestly increased fluorescent intensity over control; +++, strongly increased fluorescent intensity over control. The results are summarized below in Table 10.

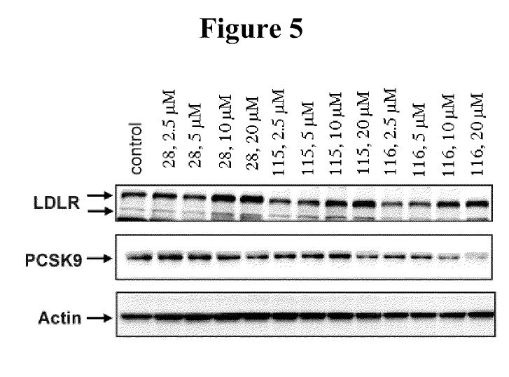

To further demonstrate the stimulating effects of compounds on LDLR protein expression and their inhibitory activities on PCSK9 expression western blot analyses were performed using antibodies directed to LDLR and PCSK9. Chicken anti-LDLR was purchased from Abcam and the mouse anti-PCSK9 was purchased from Cayman Chemicals. HepG2 cells cultured in 60-mm dishes were incubated overnight in medium containing 0.5% fetal bovine serum prior to the addition of compounds at various concentration. After 24 hours, total cell lysate was isolated and 50 μg of protein was separated on SDS-PAGE and transferred to nitrocellulose membrane for immunoblotting with anti-LDLR. The medium of control and treated cells were collected and 20 μl per sample was loaded on SDS-PAGE for western blotting with anti-PCSK9. Western blot was performed using anti-phosphorylated ERK and anti-phosphorylated AMPK antibodies to detect the activation of ERK and AMPK. After LDLR detection, the membranes were probed again with anti-actin antibody to demonstrate equal loadings of cellular proteins. Examples of western blotting are shown in FIG. 5. These results corroborate the data obtained from mRNA analysis and ligand uptake assays, and clearly demonstrate the bioactivities of this series of compounds in enhancing LDLR protein expression and reducing PCSK9 protease levels.

TABLE 10

| | | | | Activity on | |
|---|---|---|---|---|---|
| | | | Activity on | PCSK9 | |
| | Activity on | Activity on | PCSK9 mRNA | mRNA | Activity in LDL |
| | LDLR mRNA | LDLR mRNA | reduction at 10 | reduction at | uptake at 10 and |
| Compound | at 10 uM (fold | at 40 uM (fold | uM (fold of | 40 uM (fold | 40 uM (relative to |
| No. | of control) | of control) | control) | of control) | control) |
| 115 | 0.51 ± 0.11 | 2.65 ± 0.06 | n.d. | n.d. | −/+++ |
| 116 | 1.41 ± 0.04 | 7.19 ± 1.29 | n.d. | n.d. | +++/+++ |
| 121 | 3.56 ± 0.03 | 3.43 ± 0.81 | n.d. | n.d. | +++/+ |
| 127 | 1.80 ± 0.13 | 2.38 ± 0.38 | 0.197 ± 0.014 | 0.192 ± 0.016 | +++/+++ |
| 128 | 7.52 ± 0.26 | 13.36 ± 0.46 | 0.96 ± 0.00 | 0.86 ± 0.029 | +++/+++ |
| 129 | 2.49 ± 0.18 | 1.99 ± 0.13 | 1.57 ± 0.07 | 1.68 ± 0.41 | +/++ |
| 130 | 4.29 ± 0.65 | 9.00 ± 1.49 | 2.11 ± 0.11 | 0.84 ± 0.12 | ++/+ |
| 131 | 0.67 ± 0.05 | 2.25 ± 0.13 | 0.87 ± 0.04 | 0.72 ± 0.10 | +/+ |
| 132 | 2.33 ± 0.04 | 6.62 ± 0.90 | 1.85 ± 0.12 | 0.73 ± 0.15 | ++/+ |
| 133 | 4.92 ± 0.00 | 6.61 ± 0.16 | 1.04 ± 0.08 | 0.49 ± 0.01 | ++/+ |
| 134 | 1.41 ± 0.07 | 6.23 ± 0.64 | 1.25 ± 0.03 | 0.86 ± 0.02 | ±/+ |
| 135 | 1.72 ± 0.13 | 1.86 ± 0.00 | 0.97 ± 0.04 | 1.10 ± 0.14 | ±/+ |
| 136 | 0.91 ± 0.05 | 1.92 ± 0.32 | 0.48 ± 0.03 | 0.62 ± 0.02 | +/++ |
| 137 | 1.61 ± 0.19 | 2.66 ± 0.04 | 0.45 ± 0.06 | 0.34 ± 0.00 | +/+++ |
| 138 | 1.60 ± 0.29 | 2.45 ± 0.20 | 0.63 ± 0.14 | 0.35 ± 0.02 | +/+++ |
| 139 | 11.5 ± 1.29 | 3.02 ± 0.34 | 0.46 ± 0.04 | 0.27 ± 0.02 | ++/++ |
| 140 | 6.65 ± 0.68 | 8.60 ± 2.00 | 0.43 ± 0.08 | 0.51 ± 0.08 | ++/++ |
| 142 | 2.64 ± 0.01 | 12.98 ± 1.96 | 0.62 ± 0.07 | 0.52 ± 0.02 | +/++ |
| 143 | 1.87 ± 0.03 | 14.97 ± 1.68 | 1.09 ± 0.09 | 0.48 ± 0.06 | −/++ |
| 144 | 1.02 ± 0.09 | 2.69 ± 0.31 | 0.99 ± 0.09 | 1.84 ± 0.15 | −/− |
| 149 | n.d. | n.d. | n.d. | n.d. | +/+ |
| 150 | n.d. | n.d. | n.d. | n.d. | +/++ |
| 151 | n.d. | n.d. | n.d. | n.d. | −/+ |

### Example 13

### Biological Activities of Compound 162 and Compound 163, the Enantiomers of Compound 127

1. Increased LDLR mRNA Expression by Compounds 162 and 163

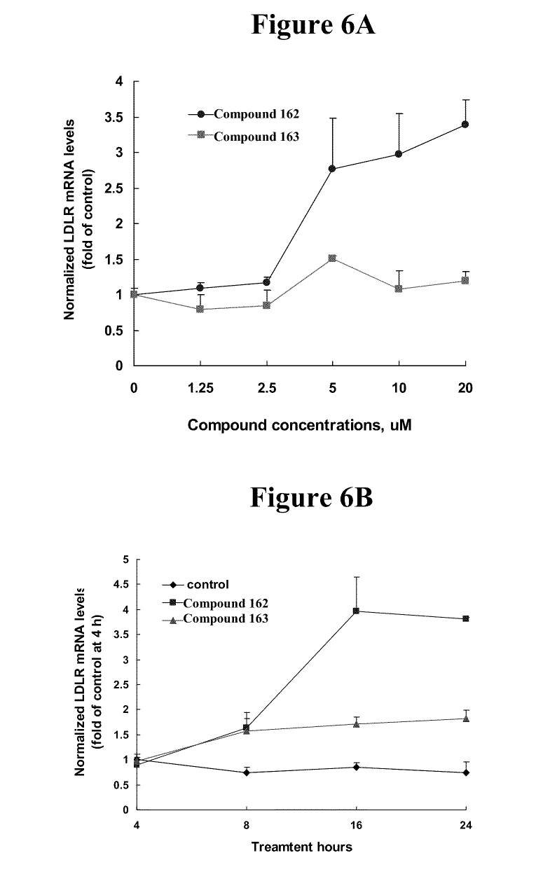

The time- and dose-dependent effects of the enantiomers of Compound 127 (Compounds 162 and 163) on LDLR mRNA expression were examined using quantitative real-time RT-PCR assays (FIGS. 6A and 6B). HepG2 cells were treated with Compound 162 and Compound 163 at indicated concentrations for various times. Total RNA was isolated and 2 was used to generate cDNA in a reaction containing random primers and M-MLV at 37° C. for 1 h in a volume of 25 μL. Real-time PCR was performed on the cDNA using MCEP REALPLEX 2 SYSTEM (Eppendorf) and Universal MasterMix (Applied Biosystems). Human LDLR and GAPDH Pre-Developed TaqMan Assay Reagents (Applied Biosystems) were used to assess the levels of mRNA expressions in HepG2. The levels of LDLR mRNA were normalized to that of GAPDH. Each RNA samples was assayed in triplicate. The fold activity was calculated by dividing mRNA abundance in compound treated cells with that of control. These results showed that while both enantiomers of Compound 127 upregulate LDLR mRNA expression, Compound 162, the enantiomer showing a positive optical rotation is more active than Compound 163, the compound with a negative optical rotation.

2. Western Blots of Proteins after Incubation of Cells with Compounds 162 and 163

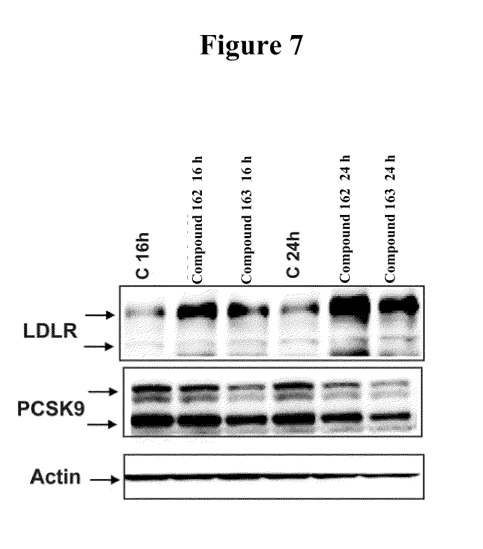

To further demonstrate the stimulating effects of these single enantiomers on LDLR protein expression, western blot analyses were performed using an antibody directed to LDLR. Chicken anti-LDLR was purchased from Abcam. HepG2 cells cultured in 60-mm dishes were incubated overnight in medium containing 0.5% fetal bovine serum prior to the addition of compounds at 20 μM concentration. After 16 or 24 hours of treatment, total cell lysate was isolated and 50 μg of protein was separated on SDS-PAGE and transferred to nitrocellulose membrane for immunoblotting with anti-LDLR. Examples of western blotting are shown in FIG. 7. These results corroborate the data obtained from mRNA analysis and clearly demonstrate the bioactivities of these compounds in enhancing LDLR protein expression.

### 3. Inhibition of PCSK9 Protein Expression in the Presence of Compounds 162 and 163

Recent studies have identified the liver secreted serine protease PCSK9 (proprotein convertase subtilisin type 9) as a natural inhibitor of LDLR protein expression that degrades hepatic LDLR protein, thus increasing circulating LDL-C levels. Example 12 above shows that Compound 127 strongly inhibits PCSK9 expression, thereby preventing LDLR protein degradation mediated by this protease. In this example, the effects of single enantiomers on PCSK9 protein expression is demonstrated using anti-PCSK9 antibody purchased from Cayman Chemicals. As shown in FIG. 7, PCSK9 cellular levels were greatly decreased in Compound 162/Compound 163 treated cells in a time-dependent manner. After LDLR and PCSK9 detection, the membranes were probed again with anti-actin antibody to demonstrate equal loadings of cellular proteins. Taken together, these results demonstrate that Compounds 162 and 163 significantly enhance LDLR mRNA and protein expression in human liver derived HepG2 cells through mechanisms involving mRNA stabilization and PCSK9 inhibition.

### 4. Formulation of Compounds 141, 162, and 163

For testing the efficacy of Compound 141 (phosphate salt of Compound 127) or its enantiomers (Compound 162/Compound 163) the following suspension formulation procedure was developed to make 100 ml of Compound 141 in suspension at 10 mg/ml concentration in 0.1 M tartaric buffer pH. 3 containing 0.5% hydroxypropylcellulose H-type (HPC-H):

Step 1: 1.50 g L-(+)-tartaric acid was dissolved in 100 mL water to prepare a tartaric acid solution. The solution was adjusted to pH 3 with 1.2 mL 5N NaOH solution to provide the tartaric acid buffer (100 mM).
Step 2: 0.5 g HPC-H powder was added to 10 mL tartaric acid buffer (100 mM) at 50° C. The slurry was stirred for 30 minutes, and then diluted with 90 mL tartaric acid buffer (100 mM) at room temperature to provide 0.5% HPC-H. Agitation was continued until all particles were dissolved and the solution was completely free of gels.
Step 3: 1.20 g Compound 141 (or Compound 162 or Compound 163) and 1.20 g mannitol were mixed and ground through a 120 mesh sieve, then the mixture was ground slightly again. The mixture was dispersed in 100 mL 0.5% HPC-H-L-(+)-Tartaric acid buffer (pH=3), then stirred until the drug dispersed evenly. The final pH was 2.6. All the above procedures were completed at room temperature (˜25° C.). The final concentration of Compound 141 was 10 mg/ml after correction of the salt weight.

### 5. Pharmacokinetics Profiles of Compound 141 (Racemic) and Compounds 162 and 163 (Single Enantiomers) in Rodents

The pharmacokinetic profile and bioavailability of Compound 141 (phosphate salt of Compound 127) in male Wistar rats following single intravenous (iv) and oral administration (p.o.) were measured according to the procedures of Example 4 and are summarized in Table 11. The pharmacokinetic profile of Compound 162 in male Golden Syrian masters following oral administration (pox) is summarized in Table 12. These results showed that the racemic Compound 141 and one of its enantiomers, Compound 163, have excellent pharmacokinetic properties and good bioavailability.

TABLE 11
Selected Pharmacokinetics Parameters of Compound 141 in Male Rats Following
Intravenous and Oral Administration

| | | | | | | | | | |
|---|---|---|---|---|---|---|---|---|---|
| AUC₍₀₋ₜ₎ | AUC_{(0-∞)} | MRT₍₀₋ₜ₎ | MRT_{(0-∞)} | t_{1/2z} | Tₘₐₓ | Vz | CLz | Cₘₐₓ | F* |
| μg/L * hr | μg/L * hr | hr | hr | hr | hr | L/kg | L/hr/kg | μg/L | % |

IV (2 mg/kg)

| | | | | | | | | | |
|---|---|---|---|---|---|---|---|---|---|
| Mean | 1070.39 | 1080.07 | 1.46 | 1.62 | 3.16 | 0.083 | 8.31 | 1.87 | 1548.91 |
| SD | 122.62 | 120.45 | 0.25 | 0.29 | 1.58 | 0 | 3.47 | 0.22 | 245.08 |

PO.a (10 mg/kg)

| | | | | | | | | | | |
|---|---|---|---|---|---|---|---|---|---|---|
| Mean | 3358.41 | 3384.31 | 4.25 | 4.44 | 3.62 | 0.33 | NA | NA | 826.35 | 62.75 |
| SD | 702.36 | 715.70 | 0.82 | 0.85 | 0.39 | 0.14 | NA | NA | 78.30 | 13.12 |

TABLE 12
Selected Pharmacokinetics Parameters of Compound 162 in Male Hamsters
following Oral Administration

| | | | | | | | | |
|---|---|---|---|---|---|---|---|---|
| AUC₍₀₋ₜ₎ | AUC_{(0-∞)} | MRT₍₀₋ₜ₎ | MRT_{(0-∞)} | t_{1/2z} | Tₘₐₓ | Vz | CLz | Cₘₐₓ |
| μg/L * hr | μg/L * hr | hr | hr | hr | hr | L/kg | L/hr/kg | μg/L |

PO (15 mg/kg)

| | | | | | | | | | |
|---|---|---|---|---|---|---|---|---|---|
| Mean | 888.66 | 914.23 | 6.06 | 6.95 | 5.21 | 0.5 | NA | NA | 195.33 |
| SD | 453.85 | 451.12 | 1.64 | 0.98 | 2.59 | 0 | NA | NA | 81.36 |

PO (30 mg/kg)

| | | | | | | | | | |
|---|---|---|---|---|---|---|---|---|---|
| Mean | 2021.53 | 2113.36 | 3.81 | 5.17 | 6.85 | 0.5 | NA | NA | 847.88 |
| SD | 264.05 | 305.57 | 0.32 | 0.78 | 3.34 | 0 | NA | NA | 364.03 |

### 6. Administration of Compound 162 to Hyperlipidemic Hamsters Improved Lipid Profiles by Reducing TC, TG, LDL-C, and Increasing HDL-C.

Male Golden Syrian Hamsters were used as an animal model to examine the in vivo effects of Compound 162 in plasma lipid levels. In one experiment, 40 male hamsters under a high fat and high cholesterol diet were divided into 4 groups of 10 hamsters per group. Group 1 was given vehicle only and groups 2-4 were given Compound 162 at 30, 60, and 100 mg/kg/day by an orogastric tube for 2 weeks. At the end of treatment, hamsters were sacrificed and blood and sera were collected to measure lipid levels and several critical parameters of liver function and kidney function.

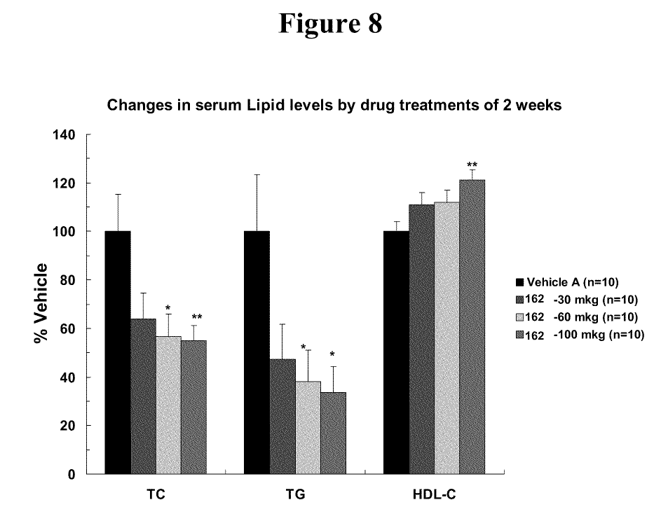

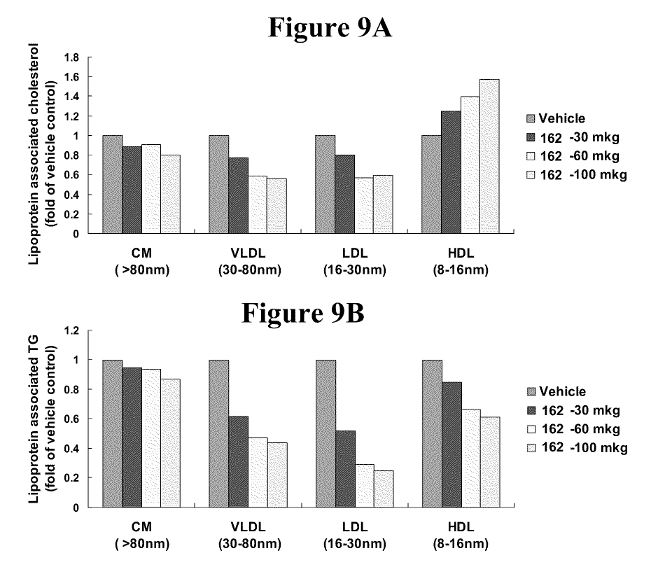

FIG. 8 shows the results of measuring individual serum samples of TC, TG, and HDL-C. Compound 162 treatment resulted in a strong dose-dependent reductions of TC and TG while increasing HDL-C. At 100 mg/kg, Compound 162 reduced serum TC and TG by 46% and 63.7% respectively and increased HDL-C by 21%. FIGS. 9A and 9B show the results of analysis (FIG. 9A) and quantification (FIG. 9B) of lipoprotein cholesterol and triglyceride in pooled serum samples from vehicle and Compound 162 treated hamsters fed the HFHC diet. It clearly demonstrated that Compound 162 dose-dependently lowered VLDL-C, LDL-C, and increased HDL-C.

Table 13 lists the results of biochemical analysis of serum samples from different treatment groups. It shows that in additional to improve lipid parameters, the serum levels of AST and ALT were statistically lower in Compound 162-treated group than control group, indicating that liver function was improved with statistical significance. Blood glucose level was not changed by the treatment while kidney function was also improved.

To directly correlate the LDL-C and VLDL-C lowering effects of Compound 162 with its ability to upregulate hepatic LDLR expression and down-regulation of PCSK9, at the end of treatment, animals from vehicle and Compound 162 100 mg/kg treated groups were euthanized and levels of liver LDLR and PCSK9 mRNA were individually assessed by quantitative real-time RT-PCR using hamster specific probes. The results represent mean±SEM of 10 animals per group. The liver expression of LDLR mRNA was increased 1.7-fold over control by Compound 162 treatment (p<0.001). Conversely, the mRNA levels of PCSK9 were reduced by Compound 162 treatment to 57% of vehicle control. Thus, these in vivo data confirmed the in vitro effects of Compound 162 in upregulating LDLR mRNA while inhibiting PCSK9 mRNA expressions.

TABLE 13
Biochemical analysis of sera after 2 weeks of Compound 162 treatment under HFHC feeding

| | TC | TG | HDL-C | ALT | AST | Glucose | Bil-T | Creatinine | BUN |
|---|---|---|---|---|---|---|---|---|---|
| | mmol/L | mmol/L | mmol/L | IU/L | IU/L | mmol/L | umol/L | umol/L | mmol/L |
| Vehicle | 29.8 ± 14.2 | 20.0 ± 14.8 | 3.5 ± 0.5 | 724 ± 612 | 235.0 ± 141 | 4.8 ± 1.8 | 1.3 ± 1.2 | 24.8 ± 8.2 | 5.3 ± 0.9 |
| (n = 10) | | | | | | | | | |
| Cmpd 162, | 19.0 ± 10.1 | 9.4 ± 9.2 | 3.9 ± 0.5 | 441 ± 401 | 202.0 ± 135 | 3.8 ± 0.8 | 0.6 ± 1.0 | 17.8 ± 6.1* | 4.5 ± 0.9 |
| 30 mkg | | | | | | | | | |
| (n = 10) | | | | | | | | | |
| Cmpd 162, | 16.9 ± 8.7* | 7.6 ± 8.1* | 3.9 ± 0.5 | 353 ± 250 | 151.5 ± 61 | 5.0 ± 1.0 | 0.3 ± 0.3* | 15.7 ± 4.2** | 5.0 ± 0.7 |
| 60 mkg | | | | | | | | | |
| (n = 10) | | | | | | | | | |
| Cmpd 162, | 16.4 ± 5.8*** | 6.7 ± 5.2* | 4.2 ± 0.5** | 376 ± 262 | 150.4 ± 42 | 5.0 ± 1.3 | 0.2 ± 0.2* | 15.3 ± 3.2** | 4.8 ± 0.7 |
| 100 mkg | | | | | | | | | |
| (n = 10) | | | | | | | | | |

*p < 0.05,
**p < 0.01,
***p < 0.001 compared to respective vehicle

The conversion of cholesterol to bile acids by the liver and their subsequent excretion in the feces represent a major route for the elimination of sterol from the body. The cholesterol 7α-hydroxylase is the first and rate-limiting enzyme in the bile acid biosynthetic pathway. The measurement of the mRNA expression level of cholesterol 7α-hydroxylase (CYP7A) is widely used as a means to monitor bile acid synthesis in liver. To assess the effect of Compound 162 on liver bile acid synthesis, the mRNA levels of CYP7A were measured in vehicle and liver samples from hamsters treated with 100 mg/kg Compound 162 by real-time PCR as described above. Int was found that CPY7A mRNA expression was 2.7-fold higher (p<0.01) in Compound 162 treated liver samples than vehicle control samples. These results suggest that Compound 162 treatment may increase the conversion of hepatic cholesterol to bile acid followed by increased excretion of bile acids into feces, which might partially explain the hypolipidemic effects of this compound in plasma and in liver.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure. Also, as used herein and in the appended claims, singular articles such as “a”, “an” and “one” are intended to refer to singular or plural. While the present technology has been described herein in conjunction with a preferred aspect, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect described above can also have included or incorporated therewith such variations or aspects as disclosed in regard to any or all of the other aspects. The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

What is claimed is:

1. A method comprising exposing a compound of structure DD, 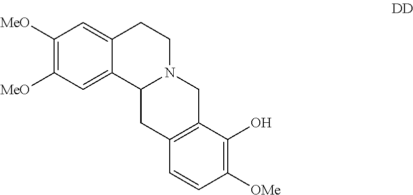
to R″—SO₂X under sulfonylation conditions to give a compound of structure EE, 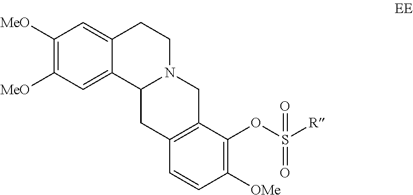
wherein
R″ is a substituted or unsubstituted alkyl, cycloalkyl, cycloalkylalkyl, alkenyl, aryl, aralkyl, heterocyclyl, heterocyclylalkyl, heteroaryl, or heteroarylalkyl group; and
X is a leaving group.

2. The method of claim 1 wherein the sulfonylation conditions comprise a suitable base.

3. The method of claim 2 wherein the suitable base is a tertiary organoamine, a pyridine, a carbonate or a bicarbonate.

4. The method of claim 1 wherein X is a halide.

5. The method of claim 4 wherein the halide is chloride.

6. The method of claim 1 wherein R″ is a substituted or unsubstituted alkyl, cycloalkyl, aryl or aralkyl group.

7. The method of claim 6 wherein R″ is phenyl or 3-fluorophenyl.

8. The method of claim 1 further comprising exposing a compound of structure CC, 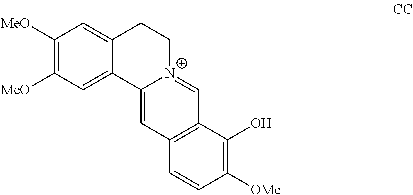
to reducing conditions to give the compound of structure DD.

9. The method of claim 8 wherein the reducing conditions comprise a borohydride in an alcohol or a transition metal and hydrogen gas.

10. The method of claim 9 wherein the borohydride is sodium borohydride and the alcohol is methanol.

11. The method of claim 9 wherein the transition metal is palladium, palladium hydroxide, platinum or platinum oxide.

12. The method of claim 8 further comprising heating a compound of structure BB 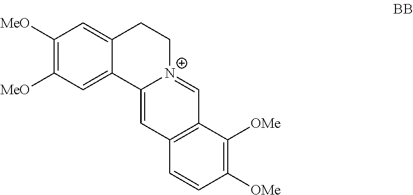
to give a compound of structure CC.

13. The method of claim 12 wherein the compound of structure BB is heated in the presence of N,N-dimethylformamide, urea or in vacuo.

14. The method of claim 12 further comprising exposing a compound of structure AA 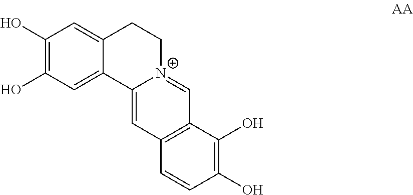
to a methylating agent to give a compound of structure BB.

15. The method of claim 14 wherein the methylating agent is dimethylsulfate or methyl iodide.

16. The method of claim 14 further comprising exposing beberine to an acid of sufficient strength to provide a compound of structure AA.

17. The method of claim 16 wherein the acid is HBr, BBr₃, or AlCl₃.
